(19) United States
(12) Patent Application Publication
(10) Pub. No.: US 2023/0167511 A1
(43) Pub. Date: Jun. 1, 2023

(54) AN ULTRASENSITIVE RAPID AND PORTABLE CASE13D-BASED DIAGNOSTIC ASSAY

(71) Applicant: The Regents of the University of California, Oakland, CA (US)

(72) Inventor: Omar Akbari, La Jolla, CA (US)

(21) Appl. No.: 17/922,860

(22) PCT Filed: May 5, 2021
(86) PCT No.: PCT/US2021/030953

§ 371 Date: Nov. 2, 2022

Related U.S. Application Data
(60) Provisional application No. 63/021,052, filed on May 6, 2020.
(60) Provisional application No. 63/091,209, filed on Oct. 13, 2020.

(51) Int. Cl. C12Q 1/70; C12Q 1/6865; C12N 9/22; C12Q 1/682; C12Q 1/6876; B01L 3/00

(52) U.S. Cl. CPC C12Q 1/701; C12Q 1/6865; C12N 9/22; C12Q 1/682; C12Q 1/6876; B01L 3/5023; C12N 2310/20; B01L 2300/0825

(57) ABSTRACT

Provided herein is a viral RNA detection system, utilizing the RNA-targeting properties of the optimized Cas13d enzyme, CasRx, to detect SARS-CoV-2 RNA, e.g., synthetic SARS-CoV-2 RNA. The system detects novel target sequences conserved within the actively evolving genome, to provide a panel of diagnostic target sites least likely to result in false negatives due to genomic variation. Successful detection of viral RNA through both a fluorescence-based readout assay as well as a rapid paper dipstick lateral flow assay requiring no specialized laboratory equipment was shown. Low viral titers can be detected within minutes following only minutes of sample processing.

### CROSS-REFERENCE TO RELATED APPLICATIONS

This application claims priority under 35 U.S.C. § 119(e) to U.S. Provisional Application Nos. 63/021,052, filed May 6, 2020 and 63/091,209, filed Oct. 13, 2020, the content of each of which is hereby incorporated by reference in its entirety.

### STATEMENT OF GOVERNMENT SUPPORT

This invention was made with government support under Grant No. HR0011-17-2-0047 awarded by the Defense Advanced Research Projects Agency (DARPA). The government has certain rights in the invention.

### BACKGROUND OF THE DISCLOSURE

Following emergence from a market in Wuhan, China in December 2019 (Zhu et al. N. Engl. J. Med. 382, 727-733, 2020; Mallapaty et al. Nature (2020) doi:10.1038/d41586-020-01449-8; Andersen et al. Nat. Med. 26, 450-452 (2020); and Q. Li et al. The New England Journal of Medicine, January. doi.org/10.1056/NEJMoa2001316, 2020), severe acute respiratory syndrome coronavirus 2 (SARS-CoV-2) (Wang et al. The Lancet vol. 395 470-473, 2020; and Zhu et al. N. Engl. J. Med. 382, 727-733, 2020) has spread to 204 countries and territories with over 1 million confirmed cases despite unprecedented control efforts (WHO. Coronavirus disease 2019 (COVID-19) Situation Report-93. www.who.int/docs/default-source/coronaviruse/situation-reports/20200422-sitrep-93-covid-19.pdf?sfvrsn=35cf80d7_4 (2020)). Compared to H1N1, Ebola, MERS and SARS-CoV-1 outbreaks of recent decades, this novel coronavirus represents the first characterized by wide-spread global transmission coupled with significant mortality. Successful identification and isolation of infected individuals can drastically curtail virus spread and limit outbreaks.

However, during the early stages of global transmission, point-of-care diagnostics were largely unavailable and continue to remain difficult to procure, greatly inhibiting public health efforts to mitigate spread. Furthermore, the most prevalent testing kits rely on reagent- and time-intensive protocols to detect viral RNA, preventing rapid and cost-effective diagnosis. Pre-symptomatic and asymptomatic carriers have been identified and as major contributors to the rapid spread of SARS-CoV-2 (Bai et al. JAMA (2020) doi:10.1001/jama.2020.2565; Wang et al. The Lancet vol. 395 470-473, 2020; and Lai et al. J. Microbiol. Immunol. Infect. (2020) doi:10.1016/j.jmii.2020.02.012.). However in many areas, these patients go largely unidentified and unisolated, thereby unknowingly exacerbating the spread of disease.

Thus, robust identification and isolation of all infected individuals is essential for controlling disease spread and necessitates development of novel testing protocols. The pandemic of SARS-CoV-2 presents an unparalleled global public health emergency, requiring urgent development of novel molecular diagnostics and therapeutics for timely patient identification, isolation and treatment. The economic, health, and societal damage wrought by SARS-CoV-2, highlights the importance of expanding and improving on current diagnostic technologies to identify and prevent future pandemics. Therefore the development of an extensive toolkit for point-of-care diagnostics that is expeditiously adaptable to new emerging pathogens is of critical public health importance.

While the majority of early-phase tests detect SARS-CoV-2 infection through amplification of viral RNA (vRNA) by real-time reverse transcription polymerase chain reaction (RT-PCR) (Corman et al., Euro Surveill. 25, (2020); Office of the Commissioner, Coronavirus (COVID-19) Update: FDA Authorizes First Test for Patient At-Home Sample Collection, accessible at www.fda.gov/news-events/press-announcements/coronavirus-covid-19-update-fda-authorizes-first-test-patient-home-sample-collection (2020); and Shen et al., J Pharm Anal 10, 97-101 (2020)), this test is time consuming (CDC 2020, Coronavirus Disease 2019 (COVID-19), Centers for Disease Control and Prevention, accessible at www.cdc.gov/coronavirus/2019-ncov/lab/index.html?CDC_AA_refVal=https%3A%2F%2Fwww.cdc.gov%2Fcoronavirus%2 F2019-ncov%2Flab%2Frt-pcr-detection-instructions.html (2020)), limited by reagents (Alice Kan, The Science Advisory Board contributing writer 2020, Shortage of RNA extraction kits threatens coronavirus testing. Scienceboard.net accessible at www.scienceboard.net/index.aspx?sec=sup&sub=can&pag=dis&ItemID=564 (2020)), required advanced equipment, and yielded significant false-negatives (Li et al., Korean J. Radiol. 21, 505-508 (2020); Kucirka et al., medRxiv 2020.04.07.20051474 (2020); Yang et al., MedRxiv (2020) doi:10.1101/2020.02.11.20021493; and Whitman et al., Test performance evaluation of SARS-CoV-2 serological assays, 2020) possibly exacerbated by genetic variation within the targeted viral genomic sequences, and high temporal and tissue specific variation in intra- and interpatient viral load (Pan et al. 2020, The Lancet Infectious Diseases, February. doi.org/10.1016/S1473-3099(20)30113-4; Zou et al. 2020, The New England Journal of Medicine 382(12):1177-79). Next generation sequencing-based diagnostics reduced false-negative rates, but still require specialized equipment and are slow (˜12 hours) (Office of the Commissioner, Coronavirus (COVID-19) Update: FDA Authorizes First Next Generation Sequence Test for Diagnosing COVID-19, accessible at www.fda.gov/news-events/press-announcements/coronavirus-covid-19-update-fda-authorizes-first-next-generation-sequence-test-diagnosing-covid-19 (2020)). Diagnostics which reduce the probability of false-negatives are therefore a critically necessary tool. Furthermore, with a typical turnaround time of >24 hours, some patients have died waiting for results. Even next generation RT-PCR based strategies still require specialized laboratory equipment and significant time (>12 hours) (Octant SwabSeq Testing), demanding development of alternate, non-PCR-based, point-of-care diagnostics.

Accordingly, what remains needed is developing alternative technologies with the potential to yield cost- and time-effective point-of-care diagnostics for infection SARS-CoV-2 or other pathogens.

### SUMMARY OF THE DISCLOSURE

Disclosed herein is a novel test for coronavirus disease 2019 (Covid-19), based on Cas13d isothermal detection assay that leads to faster and more reliable testing and can be adapted to both fluorescence readout as well as a rapid paper dipstick lateral flow. Thus, the system and methods do not necessitate use of specialized laboratory equipment. The test is optimized to detect the E Covid-19 gene, S gene, and N gene and it utilizes optimized gRNAs. This approach is superior to known CRISPR-Cas based tests as it does not require protospacer adjacent motif (PAM) or protospacer flanking sequence (PFS) or both sequences so it is inherently more flexible. It can easily be tuned to detect other virus strains and it can be multiplexed.

A CRISPR system is provided that comprises, or consists essentially of, or yet further consists of a severe acute respiratory syndrome coronavirus 2 (SARS-CoV-2) gene guide RNA (i.e., gRNA, such as an envelope (E) gene gRNA, a nucleocapsid (N) gRNA, a spike (S) gRNA, or any combination thereof) and CRISPR reagents necessary to detect the SARS-CoV-2 gene, such as S gene, E gene or N gene in a sample. In one aspect, the system also comprises a promoter sequence permitting in vitro transcription of the gene, such as S, E or N gene, an example of which is a T7 promoter. In a further aspect, the CRISPR system comprises an E gene gRNA and an N gene gRNA. In yet further aspect, the CRISPR system further comprise an S gene gRNA. Non-limiting examples of such gRNAs are disclosed herein.

In one aspect, provided is a clustered regularly interspaced short palindromic repeats (CRISPR) system. In some embodiments, the system comprises, or consists essentially of, or yet further consists of: a gRNA targeting a target sequence and CRISPR reagents necessary to detect the SARS-CoV-2 sequence in a sample. In one embodiment, the target sequence is an RNA. In a further embodiment, the target sequence is a genomic RNA sequence (for example a gene). In another embodiment, the target sequence is a DNA. In yet another embodiment, the target sequence is a hybrid of DNA and RNA. In some embodiments, the target sequence is a pathogen sequence (DNA or RNA or a hybrid thereof), for example, a sequence of bunyaviruses, zoonotic viruses such as Ebola, hanta, and Lassa, arboviruses such as dengue, chikungunya, and Zika; coronaviruses such as MERS, SARS-CoV-1, SARS-CoV-2; or other pathogen as disclosed herein. In some embodiments, the target sequence is a severe acute respiratory syndrome coronavirus 2 (SARS-CoV-2) sequence. In further embodiments, the target sequence is selected from one or more of an envelope (E) gene, a nucleocapsid (N) gene, an Orf1ab gene, a Spike (S) gene, an Orf3a gene, an M matrix protein gene, an Orf6 gene, an Orf7a gene, an Orf7b gene, an Orf8 gene, any ORF gene listed herein, such as in Table 2, or a fragment of each thereof. In some embodiments, the gene is in a RNA viral genome, thus is an RNA sequence.

In some embodiments, the target sequence is about 25 nt long to about 35 nt long. In one embodiment, the target sequence is about 30 nt long. In some embodiments, the target sequence is not adjacent to a PAM or PFS in the genome or the pathogen to be detected or a RNA (genomic or messenger RNA) of the pathogen.

In some embodiments, the gRNA comprises, or consists essentially of, or yet further consists of a direct repeat and a polynucleotide (such as RNA, DNA or a hybrid thereof) sequence complimentary to the target sequence optionally having 0, 1, 2 or 3 mismatches. In some embodiments, the direct repeat is a 5′ direct repeat. In a further embodiment, the direct repeat is as disclosed herein, such as in Table 5 or in FIG. 10.

In some embodiments, the system and/or the CRISPR reagents comprise, or consist essentially of, or yet further consist of a Cas13 enzyme. In further embodiments, the Cas13 enzyme is a Cas13d enzyme. In some embodiment, the Cas13d is Ruminococcus flavefaciens Cas13d (CasRx). In some embodiments, the system and/or the CRISPR reagents comprise, or consist essentially of, or yet further consist of a fusion protein comprising, or alternatively consisting essentially of, or yet further consisting of the Cas13d enzyme, an optional protein cleavage site (such as a TEV protease cleavage sequence), a purification marker or tag (such as a 6×His tag), and an optional Maltose-binding protein or a fragment thereof. In yet further embodiment, the system and/or the CRISPR reagents further comprise an accessory protein comprising, or alternatively consisting essentially of, or yet further consisting of a WYL1-domain.

In some embodiments, the method further comprises a reporting reagent. In some embodiments, the reporting reagent comprises, or consists essentially of, or yet further consists of a probe. In further embodiments, the reporting reagent comprises, or consists essentially of, or yet further consists of a probe conjugated with one or more purification or detectable markers (such as radioisotopes, fluorochromes, chemiluminescent compounds, dyes, and proteins, including enzymes). In some embodiments, the reporting reagent comprises, or consists essentially of, or yet further consists of a fluorophore and a quencher. In further embodiments, the fluorophore can be placed in close proximity to the quencher. In yet further embodiments, the system permits release of the fluorophore from the close proximity to the quencher upon detection of the target sequence. In some embodiments, the probe is a collateral cleavage probe, for example, the probe can be cleaved due to the collateral cleavage activity of the Cas13 enzyme as disclosed herein. In some embodiment, such cleavage allowing releasing of the purification or detectable markers, or releasing of the fluorophore from the close proximity to the quencher. In further embodiments, the probe comprises, or consists essentially of, or yet further consists of a poly U sequence, such as having about 4 to about 20 U residues. In one embodiment, the probe comprises, or consists essentially of, or yet further consists of a 6-nt poly-U. In some embodiments, the reporting reagent comprises, or consists essentially of, or yet further consists of a probe (optionally a poly U as disclosed herein) conjugated to a fluorescence marker (such as a 5′ fluorescent marker and/or a 6-FAM) and a quencher (such as a 3′ quencher and/or optionally an IABlkFQ). In some embodiments, the reporting reagent comprises, or consists essentially of, or yet further consists of a probe (optionally a poly U as disclosed herein) conjugated to a biotin and/or a fluorescent marker).

In some embodiments, the CasRx or Cas13d facilitates fluorescence-based readouts of RNase activity. In some embodiments, the system further comprises a means for visual indication of activity, such as to be read out visually under UV, or quantitatively by a fluorometer. In some embodiments, the CasRx enzyme is modified to detect SARS-Cov-2 genetic material by lateral flow assay.

In another aspect, the system further comprises CasRx or Cas13. In a yet further aspect, the CasRx or Cas13 facilitates fluorescence-based readouts of RNase activity. In another aspect, the CasRx enzyme is modified to detect SARS-Cov-2 genetic material by lateral flow assay.

In another embodiment, the system further comprising a fluorophore and a quencher, wherein optionally the fluorophore can be placed in close proximity to the quencher.

In another aspect, the system further comprises a means for visual indication of activity, optionally to be read out visually under UV, or quantitatively by a fluorometer.

In another aspect, the system comprises one or more of gRNA as disclosed herein, such as gRNA-S1, gRNA-S2, gRNA-S3, gRNA-N1, gRNA-N2, gRNA-N3, gRNA-T, gRNA-R, gRNA-V, gRNA-Z, gRNA-AA, gRNA-AC, or any other gRNA as disclosed herein. See, for example, Table 5.

The system is useful in a method to detect SARS-CoV-2 in a sample, by contacting the sample with the system as described herein. Non-limiting examples are disclosed herein and include samples isolated from one or more of the lungs, oral cavity or nasal cavity of a subject. In one embodiment, the subject is a mammal that is susceptible to infection by SARS-CoV-2, e.g., a bat, a simian, a human, a feline, or a canine. The method also comprises detecting the presence of SARS-CoV-2, in the sample by detecting the presence of a SARS-CoV-2 gene, such as the E gene, the S gene, or the N gene or alternatively the presence of the E gene and the N gene or the S gene and the N gene.

In one aspect, provided is a method to detect SARS-CoV-2 in a sample. In some embodiments, the method comprises, or consists essentially of, or yet further consists of contacting the sample with the system as disclosed herein. In some embodiments, the sample is isolated from one or more of the lungs, oral cavity or nasal cavity of a subject. In some embodiments, the subject is a mammal that is susceptible to infection by SARS-CoV-2. In some embodiments, the mammal is a bat, a simian, a human, a feline, or a canine, a murine, a rat, a rabbit, a bovine, an ovine, a porcine, an equine, and a primate. In some embodiments, the method further comprises detecting the presence of the pathogen, such as SARS-CoV-2, in the sample by detecting the presence of the target sequence, such as the S gene, the E gene, the N gene, or any combination thereof. In some embodiments, the method further comprises detecting the presence of SARS-CoV-2, in the sample by detecting the presence of the E gene and the N gene. In some embodiments, the limit of detection (LOD) of the method about 10 to about 1000 copies (optionally 100 copies) per RT-RPA reaction or per microliter, for example of the reaction system. In some embodiments, the specificity and/or the concordance of the method is at least about 50%, or at least about 60%, or at least about 70%, or at least about 80%, or at least about 90%, or at least about 91%, or at least about 92%, or at least about 93%, or at least about 94%, or at least about 95%, or at least about 96%, or at least about 97%, or at least about 98%, or at least about 99%, or about 100%.

Further provided herein is a kit comprising, or consisting essentially of, or yet further consisting of the system as disclosed herein and instructions for use. In one aspect, the instructions are to perform the methods as disclosed herein. In a further aspect, the kit further comprises an anti-SARS-CoV-2 therapeutic (for example, remdesivir (Gilead Sciences, Inc.)) or vaccine composition or therapeutic to treat symptoms of SARS-CoV-2 infection (e.g., an anti-inflammatory). In some embodiments, the kit further comprises one or more of: a negative control, a positive control, an off-target gRNA and an anti-SARS-CoV-2 therapeutic or vaccine composition.

CRISPR-Cas systems are easily programmable and can be adapted to detect various nucleic acid sequences, making these systems prime technological candidates for the detection of viral genetic material, while also overcoming the technological hurdles of RT-PCR-based screening. Recently, several CRISPR based viral detection systems relying on either DNA-targeting (Mukama et al. 2020, Biosensors and Bioelectronics. doi.org/10.1016/j.bios.2020.112143; Ding et al. 2020, doi.org/10.1101/2020.03.19.998724; Lucia, et al. 2020. doi.org/10.1101/2020.02.29.971127; Broughton et al. 2020, Nat. Biotechnol. (2020) doi:10.1038/s41587-020-0513-4; and Broughton et al. 2020, Infectious Diseases (except HIV/AIDS). medRxiv. doi.org/10.1101/2020.03.06.20032334.) or RNA-targeting (Metsky et al. 2020, bioRxiv 2020.02.26.967026 (2020) doi:10.1101/2020.02.26.967026; Zhang, Abudayyeh, and Jonathan 2020, A protocol for detection of COVID-19 using CRISPR diagnostics. (2020)) have been developed using either LbCas12a or LwaCas13a, respectively, while exploiting their collateral cleavage activity to report detection of sequences specific to SARS-CoV-2. Herein, Applicant reports on a viral RNA detection system, utilizing the RNA-targeting properties of the optimized Cas13d enzyme, CasRx (Konermann et al. 2018, Cell 173 (3): 665-76.e14), to detect SARS-CoV-2 RNA, e.g., synthetic SARS-CoV-2 RNA. Applicant demonstrated cleavage of viral genomic targets previously validated by others (Broughton et al. 2020, Nat. Biotechnol. (2020) doi:10.1038/s41587-020-0513-4), in addition to bioinformatically identifying novel target sequences conserved within the actively evolving genome by analyzing the first 433 publicly available novel SARS-CoV-2 genomic sequences (GenBank), yielding a panel of diagnostic target sites least likely to result in false negatives due to genomic variation. Applicant demonstrated successful detection of viral RNA through both a fluorescence-based readout assay as well as a rapid paper dipstick lateral flow assay requiring no specialized laboratory equipment. Applicant demonstrated that low viral titers can be detected within minutes following only minutes of sample processing.

Due to the inherent flexibility of CRISPR-based detection systems, this tool can easily be adapted to detect not only SARS-CoV-2, but also other global emerging viral threats around the world. The CasRx is considered even more inherently flexible than other Cas proteins given it does not require a PAM nor PFS, making almost every sequence potentially targetable. Developing a wide-ranging and well-characterized suite of CRISPR diagnostic tools today is critical to enable faster and more robust testing of viral threats likely to emerge in the future. Having these tools within reach may give scientists and governments the upper hand to thwart future viral pathogens with speed and resound, avoiding repeating the global fate beset by the current pandemic.

### BRIEF DESCRIPTION OF THE DRAWINGS

FIGS. 1A-1B provide gel results of samples obtained after 1 hour run on a SHERLOCK fluorescence assay for fluorescence detection of collateral cleavage by Cas13d. CasRx and gRNA-P were mixed in two different ratios 3:1 (FIG. 1A) and 1:2 (FIG. 1B). Different amounts of a synthetic 269 bp SARS-CoV RNA produced by in vitro transcription were used as templates. The RNAse alert probe was included in all reactions as indicator of collateral cleavage by fluorescence reads but it was not detected on the gel (see negative controls). There was no significant fluorescence detection but the gel showed cleavage of a 700 bp non-target template used as additional control (rectangle). In addition, cleavage occurred in different amount of templates including 1000 ng but it was obvious at lower amounts. Samples were run on TBE 2% agarose gel stained with ethidium bromide.

FIG. 2 provides gel results of samples obtained after 1 hour run on a SHERLOCK mastermix assay for detection on gel of collateral cleavage by Cas13d. CasRx and gRNA-P were mixed in two different ratios 3:1 (SHERLOCK) and 1:2. Different amounts of a synthetic 269 bp SARS-CoV RNA produced by in vitro transcription were used as templates. The RNAse alert probe WAS NOT included in all reactions because the assays was intended for detection on a gel. Instead detected was cleavage of a 700 bp non-target template used as additional control (rectangle). Cleavage occurred in different amount of templates including 1000 ng but it was obvious at lower amounts. The ratio of 1:2 CasRx:gRNA (1 pmol: 2 pmol) seems to cleave more efficiently than the recommended 3:1 (110 pmole: 0.3 pmole).

FIG. 3 shows in vitro cleavage of RNA template using CasRx and several gRNAs. All gRNAs were tested with CasRx batch 2. The last three lanes show the negative controls without gRNA for CasRx Batch 2 and 1 and for GFP-CasRx. 2 μL of CasRx 032820 (0.55 ng/μL) were mixed with 1 μL gRNA (32 ng/μL) incubated at 37° C. for 10 minutes, then 9 μL of a mastermix containing 75 ng template and 1× buffer NEB 2.1.

FIG. 4 provides schematics of CasRx protein purification.

FIG. 5 shows cleavage activity of CasRx on six different target sites (gRNA-K to gRNA-P) of a 269 nt synthetic SARS-CoV-2 RNA template diluted in protein storage buffer.

FIG. 6 shows collateral cleavage activity of CasRx on two ˜1500 nt (NTT-1 and NTT-2) and one ˜100 nt FAM-labeled (probe) bystander RNAs after cleaving a 269 nt synthetic SARS-CoV-2 RNA template diluted in protein storage buffer.

FIG. 7 provides a summary of current CRISPR-based anti-COVID technologies organized by Cas enzyme used and by role as diagnostic or detection tool, or as a putative prophylactic. Those technologies shown in black font have been demonstrated to have explicit activity against SARS-CoV-2, while those technologies shown in grey font have been publicly discussed or proposed to be technological candidates for SARS-CoV-2 detection or prophylaxis, however have not yet been fully demonstrated/optimized for said purpose. Some technologies have not yet been given a formal name by their authors, and are therefore denoted as ‘unnamed’ followed by the acronym for the primary institutional affiliation behind the work. To better identify and distinguish these technologies, the DOI has been provided. Diagnostics/detection systems/Mass testing: For all technologies viral RNA is extracted, reverse transcribed into cDNA, followed by template amplification by either PCR, RPA or Loop-Mediated Isothermal Amplification (LAMP), then input into subsequent reactions with the exception of CRISPR-Chip which does not require an amplification step. Cas12-based enzymes, as well as many other Cas proteins (including Cas9) recognize DNA species, while Cas13-based enzymes recognize ssRNA, and all can be used to detect evidence of specific sequences by fluorescence or lateral flow. The detection method for each technology is noted with the presence of an icon adjacent. The majority of the technologies summarized here use fluorescence or lateral flow or both. The glow icon indicates readout by fluorescence, while the grey bar indicates read-out by lateral flow. Some of the other technologies can be read out by different detection methods. Notably because FELUDA relies on direct sequence cleavage, and not the collateral cleavage property shared amongst the Cas12- and Cas13-based technologies, it can also be analyzed by gel electrophoresis as evidence of distinct band cleavage (gel icon). Also, CRISPR-Chip has been discussed as a SARS-CoV-2 mass detection candidate, though these findings have not yet been made publicly available. Read-out by this technology is achieved via a graphene-based transistor (icon with graphene structure adjacent). Other different technologies include CREST, which achieves detection via fluorescence using a distinctly do-it-yourself (DIY) BioHacking approach, by utilizing equipment easily procured and operated by novice scientists, as well as CARMEN-Cas13 which detects evidence of SARS-CoV-2 sequences by microarray. The technology outlined here is an addition to current SHERLOCK detection utilizing Cas13d (CasRx). The technical details of most technologies is summarized in Table 1.

FIGS. 8A-8D show CasRx protein purification workflow and quality control. As shown in FIG. 8A, CasRx protein was produced and purified essentially as described in (Konermann et al. 2018, Cell 173 (3): 665-76.e14.). A CasRx protein-expression plasmid was generated with CasRx downstream of a Maltose-binding protein (MBP) domain with an N-terminal 6×His tag, connected by a TEV protease cleavage sequence. Origin of replication and KanR cassette are as labeled. Transformation of the plasmid into ROSETTA2™ (DE3) Competent E. coli was followed by large scale culture growth. Cell lysate supernatant containing 6×His-MBP-CasRx soluble hybrid protein was run on a Ni-NTA column for purification by affinity chromatography. CasRx protein was then released from the 6×His-MBP moiety by TEV protease cleavage during O/N dialysis, and was further purified by cation exchange and size exclusion using Fast Protein Liquid Chromatography (FPLC). FIG. 8B provides the cation exchange elution profile of CasRx with the concentration of NaCl (mM). Peak containing CasRx recombinant protein (boxed) elutes at ˜700 mM NaCl. FIG. 8C provides the SEC elution profile of CasRx recombinant protein following Size Exclusion Chromatography (SEC). Peak containing CasRx recombinant protein highlighted boxed. As shown in FIG. 8D, a 10% SDS-PAGE gel showing protein species present at different stages of the purification protocol. Lane 1 is SEEBLUE™ Plus2 Pre-stained Protein Standard with the predicted final CasRx protein size marked at right at approximately 112 kDa. Lane 2 is a resuspended cell pellet. Lane 3 is cell lysate supernatant. Lane 4 is cell lysate pellet. Lane 5 is Ni-NTA flow through. Lane 6 is Ni-NTA Elution. Lane 7 is the sample post O/N Dialysis. Lane 8 is the sample after SEC, and Lane 9 is the concentrated Final Sample post-SEC.

FIGS. 9A-9H show harnessing SENSR to detect evidence of SARS-CoV-2 viral transcripts. FIG. 9A provides an overview of assay workflow. Following extraction of viral RNA, the detection protocol requires three distinct steps, the last of which differs based on desired output detection method. In the first reaction, specific target sequences within the viral RNA are reverse transcribed (RT) into cDNA and amplified by RPA at 42° C. for 45 min, while also adding T7 promoter sequences to the 5′ terminus (T7). In the next reaction, in vitro transcription occurs simultaneously with CasRx collateral cleavage activation by recognition and cleavage of the target RNA sequence through the sequence-specific targeting activity of the gRNA (the hairpin-shaped marks). Addition of a probe conjugated to fluorescein and a quencher can facilitate readout by fluorescence following probe cleavage (top right). Addition of a probe conjugated to fluorescein and biotin facilitates readout by lateral flow assay (bottom right). FIG. 9B provides schematic of the SARS-CoV-2 genome, RPA reagents, gRNA target sites, and synthetic amplicon position. The S (Spike), E (envelope) and N (nucleocapsid) genes are enlarged to depict the design schematic in more detail. The schematic depicts the relative position of the RT-RPA primers and gRNAs used in the amplification step. The gRNA target map outlines all gRNAs tested, with their relative positions. The E-gene encompasses 227 total nt, the S-gene encompasses 3,822 total nt, while the N-gene encompasses 1,260 nt, and gRNA lengths and positions are shown to scale. Synthetic target amplicon denotes the length of the synthetic viral genome fragment used to test assay cleavage and its relative position within the gene coding sequence. FIG. 9C provides characterization of CasRx fluorescence detection by S-targeting gRNAs (gRNA-S1, -S2, -S3) with the addition of 300 fM dsDNA template (S-gene template) to an IVT-coupled cleavage reaction. Shown as Background Subtracted Units, A.U. over time (4 technical replicates each, Mean and SEM). FIG. 9D provides characterization of CasRx fluorescence detection by N-targeting gRNAs (gRNA-N1, -N2, -N3) with the addition of 300 fM dsDNA template (N-gene template) to an IVT-coupled cleavage reaction. Plotted as Background Subtracted Units, A.U. over time (4 technical replicates each, Mean and SEM). FIG. 9E provides characterization of CasRx fluorescence detection by N-targeting gRNAs (gRNA-Z, -AA, -AC) with the addition of 300 fM dsDNA template (N-gene template, 524 bp) to an IVT-coupled cleavage reaction. Shown as Background Subtracted Units, A.U. over time (4 technical replicates each, Mean and SEM). gRNA-Z was selected for further downstream analysis. FIG. 9F provides characterization of CasRx fluorescence detection by E-targeting gRNAs (gRNA-R, -T, -V) with the addition of 300 fM dsDNA template (E-gene template, 253 bp) to an IVT-coupled cleavage reaction. Plotted as Background Subtracted Units, A.U. over time (4 technical replicates each, Mean and SEM). gRNA-T was selected for further downstream analysis. FIG. 9G shows the in vitro cleavage properties of the E-targeting gRNA-T. In Lane 1 the absence of gRNA-T results in no cleavage of the E-gene fragment (228 nt). In Lane 2 the addition of gRNA-T results in cleavage of the E-gene template resulting in loss of the distinct 228 nt band and appearance of lower molecular weight cleavage species. In Lane 3, the off-target N-gene template (500 nt) remains uncleaved in the presence of CasRx and the E-targeting gRNA-T. In Lane 4, the absence of CasRx results in no cleavage of either E or N-gene templates. In Lane 5, the on-target cleavage of the E-gene template results in additional collateral cleavage of the N-gene template, resulting in the loss of both bands from the gel and the accumulation of lower molecular weight cleavage products. FIG. 9H shows the in vitro cleavage properties of the N-targeting gRNA-Z. In Lane 1, the absence of gRNA-Z results in no cleavage of the N-gene fragment (500 nt). In Lane 2, the addition of gRNA-Z results in cleavage of the N-gene template resulting in loss of the distinct 500 nt band and appearance of lower molecular weight cleavage species. In Lane 3, the off-target E-gene template (228 nt) remains uncleaved in the presence of CasRx and the N-targeting gRNA-Z. In Lane 4, the absence of CasRx results in no cleavage of either E or N-gene templates. In Lane 5, on-target cleavage of the N-gene template results in additional collateral cleavage of the E-gene template, resulting in the loss of both bands from the gel and the accumulation of lower molecular weight cleavage products.

FIG. 10 provides molecular scale overview of CasRx detection protocol workflow. Bracketed into the two base component reactions, RT-RPA or IVT+cleavage. In step 1, viral ssRNA is extracted and reverse transcribed into cDNA. A fragment of the viral E-gene ssRNA, with the final CasRx target site highlighted. Reverse transcriptase actively reverse transcribes the viral template into cDNA. In step 2, in the simultaneous RPA reaction, recombinase binds to the primer with D-loops formed at binding sites. Recombinase helps anneal the primer to the target site, and single strand binding protein (SSB) begins annealing to ssDNA to stabilize the strand and reaction. In step 3, strand-displacing DNA polymerase amplifies the target DNA, with continued binding of SSB to ssDNA for stabilization. In step 4, DNA amplicon extension is completed, while polymerase simultaneously dislodges SSB, and the T7 promoter region is added to the amplicon via primer extension. In step 5, the final product of the RT-RPA reaction is a small amplified fragment of target DNA encompassing the CasRx target site, with a T7 promoter added for subsequent IVT. In step 6, T7 RNA polymerase-based in vitro transcription occurs, initiated from the T7 promoter in order to generate ssRNA required as the activation substrate of CasRx. Elongation of the ssRNA template takes place. In step 7, CasRx detection of on-target sequence, activation of collateral cleavage activity, and detection. The CasRx/gRNA complex recognize, bind to, and cleave (larger scissors) the ssRNA viral target template. This action activates the collateral cleavage property of CasRx (smaller scissors). Following activation, collateral cleavage of an included small ssRNA probe can be analyzed via fluorescence or lateral flow assay.

FIG. 11 provides depiction of unique gRNA target sequences across SAR-CoV-2 genome. Spread of unique and specific 30 nt putative gRNA target sequences (Tables 3-5) displayed across the SARS-CoV-2 genome, smoothened over a 301 nt window. Higher specificity score indicates higher density of unique and conserved targets. Representative genome map shown at bottom with ORFs and genes annotated. E-gene ORF marked as E with vertical bar highlights the zero specificity score of the gene.

FIGS. 12A-12E provide in vitro cleavage assays for guides tested. FIG. 12A provides a summary map of gRNA target location within the SARS-CoV-2 genome. gRNA-R, -T, -V target the Envelope (E) gene, and gRNA-Z, -AA, -AC target the Nucleocapsid (N) gene. FIG. 12B shows in vitro on-target cleavage assays for the guides developed in the first cohort (N-targeting gRNA-Z, -AA, -AC). Equimolar amounts of N and E synthetic RNA fragments (500 nt and 228 nt respectively) were incubated at room temperature (RT) in the presence or absence of their on-target gRNA, named above. The on-target gene is denoted with (E) or (N) or (S) for each gRNA, and is shown mapped in FIG. 12A and FIG. 9B. The presence of the gRNA initiates cleavage of both the on-target template in addition to collateral cleavage of the off-target template. FIG. 12C shows on-target in vitro cleavage assays for the conserved and specific E-targeting gRNA-R, -T, -V. In each reaction the 228 nt E-gene synthetic RNA fragment is provided, and the presence or absence of CasRx initiates on-target cleavage of the template into smaller fragments. FIG. 12D shows on-target in vitro cleavage assays for the S-targeting gRNA-S1, -S2, -S3. In each reaction the S-gene synthetic RNA fragment is provided, and the presence or absence of CasRx initiates on-target cleavage of the template into smaller fragments. FIG. 12E shows on-target in vitro cleavage assays for the N-targeting gRNA-N1, -N2, -N3. In each reaction the N-gene synthetic RNA fragment is provided, and the presence or absence of CasRx initiates on-target cleavage of the template into smaller fragments.

FIGS. 13A-13E show optimizing SENSR fluorescence detection assay. FIG. 13A provides a schematic representation of experiment determining the collateral cleavage, di-nucleotide preference for CasRx. FIG. 13B provides a heat map layout of background subtracted fluorescence from CasRx collateral cleavage for all di-nucleotide probe combinations. The more saturated the signal the larger the increase in fluorescence. FIG. 13C provides a fluorescence-over-time plot demonstrating CasRx preferentially cleaves collateral targets containing a poly-U nucleotide stretch. No significant increase in fluorescence was observed for any other probes tested compared to the poly-U probe (One-way ANOVA, Dunnett's test: p<0.0001). FIG. 13D shows determining the optimal input volume of RT-RPA sample into SENSR reaction. In all cases the trial sample is equivalent to 100 copies/μl (labeled as “Sample”), the positive control is established with 10,000/μl (labeled as “Pos Ctrl”), and the negative control is no-template control (labeled as Neg Ctrl). FIG. 13E shows determining optimal sequence for collateral cleavage probe. Collateral cleavage of Poly-A probe is tested in the first group, Poly-U is tested in the second, and RNase Alert in the third group. Within each group, gRNA-T and gRNA-Z are incubated in a reaction saturated with 10,000 (10k) target copies, or 0 (Neg Ctrl) as a negative control. Performing a two-tailed t-test of unequal variance comparing each 10k group to the respective Neg Ctrl groups a significant increase in fluorescence was only found for the Poly-U probe 10k groups (gRNA-T 10k: p<0.0001, gRNA-Z 10k: p<0.0001). No significant increase in fluorescence was observed for the 10k Poly-A or 10k RNase Alert groups compared to the respective Neg Ctrl groups for gRNA-T (Poly-A: p=0.5953, RNase Alert: p=0.4294) or gRNA-Z (Poly-A: p=0.7935, RNase Alert: p=0.1510). Performing a one-way ANOVA followed by a Dunnett's test comparing the 10k groups of the Poly-A and RNase Alert groups to the 10k Poly-U groups a significant difference was found for both gRNA-T (Poly-A: p<0.0001, RNase Alert: p<0.0001) and gRNA-Z (Poly-A: p<0.0001, RNase Alert: p<0.0001). Bars indicate mean SD of background subtracted fluorescence from three technical replicates. In the left most grouping the RT-RPA template sample input into the SENSR reaction comprises 10% of the preamplification reaction, in the middle grouping it composes 20% of the preamplification reaction, and in the final grouping it comprises 50% of the preamplification reaction. Performing a one-way ANOVA followed by a Dunnett's test within each % input group significance was only found for both the sample (p<0.0001) and positive control (p<0.0001) in the 50% group (10%: Sample—p=0.8925, Pos Ctrl—p<0.0001; 20%: Sample—p=0.8160, Pos Ctrl—p<0.0001). Performing a second one-way ANOVA followed by a Dunnett's test comparing the 50% sample group to the 10% and 20% sample groups, a significant increase in fluorescence was observed (10%: p<0.0001, 20%: p<0.0001). Bars indicate mean±SD of background subtracted fluorescence from three technical replicates.

FIGS. 14A-14F show SARS-CoV-2 detection by SENSR via fluorescence and lateral flow assay. FIG. 14A shows cleavage properties of N-targeting gRNA-Z in fluorescence context. Fluorescence detection of N-gene in an IVT-coupled coupled context. gRNA-T incubated in three different contexts: with E-gene template, with N-gene template, or with both E- and N-gene templates. Accumulation of fluorescence occurs when gRNA-Z is incubated with the N-gene target template leading to cleavage of the ssRNA probe. Collateral cleavage is not observed when gRNA-Z is incubated with the non-target N-gene template. FIG. 14B shows cleavage properties of E-targeting gRNA-T in fluorescence context. Fluorescence detection of E-gene in an IVT-coupled coupled context. gRNA-T incubated in three different contexts: with N-gene template, with E-gene template, or with both N- and E-gene template. Accumulation of fluorescence occurs when gRNA-T is incubated with the E-gene target template leading to cleavage of the ssRNA probe. Collateral cleavage is not observed when gRNA-T is incubated with the non-target E-gene template. FIG. 14C shows CasRx nucleic acid limit of detection (LOD) for gRNA-Z detection via fluorescence following cleavage of N-gene target. Total copy number of 10000, 1000, 100, 10, or 0 of viral RNA template input into initial RT-RPA reaction, followed by CasRx detection by fluorescence. Performing a one-way ANOVA followed by a Dunnett's test call copy numbers to the NTC significance was found for 10000, 1000, and 100 copies (p<0.0001) and no significance was found for 10 copies (p=0.9999). Results shown as background-subtracted fluorescence (A.U.) following 90 minute fluorescent readout (4 technical replicates each, Mean and SD). FIG. 14D shows CasRx nucleic acid LOD for gRNA-T detection via fluorescence following cleavage of E-gene target. Total copy number of 10000, 1000, 100, 10, or 0 of viral RNA template input into initial RT-RPA reaction, followed by CasRx detection by fluorescence. Performing a one-way ANOVA followed by a Dunnett's test call copy numbers to the NTC significance was found for 10000, 1000, and 100 copies (p<0.0001) and no significance was found for 10 copies (p>0.9999). Results shown as background-subtracted fluorescence (A.U.) following 90 minute fluorescent readout (4 technical replicates each, Mean and SD). FIG. 14E shows LOD of the gRNA-Z recognition of N-gene target by lateral flow assay. CasRx detection reaction incubated for 1 h prior to lateral flow assay. FIG. 14F shows LOD of the gRNA-T recognition of E-gene target by lateral flow assay. CasRx detection reaction incubated for 1 h prior to lateral flow assay.

FIGS. 15A-15D show assessment of gRNA specificity within the SENSR system. Schematic representation of gRNA mismatches for the four most closely related sequences (off-targets, OT1-OT4) identified via BLAST for gRNA-T (FIG. 15A) and gRNA-Z (FIG. 15B). FIG. 15C shows gRNA-T demonstrated no off-target activity on the 4 closely related sequences. Bars indicate mean±SD of background subtracted fluorescence from three technical replicates. Performing a one-way ANOVA followed by a Dunnett's test comparing to the NTC fluorescence signal was found to be significant for the E-gene compared (p<0.0001), however no significant fluorescence signal increase was found for the four off-targets (OT1: p=0.9998, OT2: p=0.5242, OT3: p=0.6633, OT4: p=0.8475). Performing a one-way ANOVA followed by a Dunnett's test comparing to the E-gene positive control significant fluorescence signal increase was found when targeting the E-gene compared to the four off-targets (OT1, OT2, OT3, OT4: p<0.0001). FIG. 15D shows collateral cleavage was observed for two of the closely related off-targets for gRNA-Z. Bars indicate mean±SD of background subtracted fluorescence from three technical replicates. Performing a one-way ANOVA followed by a Dunnett's test comparing to the NTC fluorescence signal increase was found to be significant for the N-gene positive control, OT1, and OT2 (N, OT1, OT2: p<0.0001), however no significant fluorescence signal increase was found for OT3 and OT4 (OT3: p=0.8877, OT4: p>0.9999). Performing a one-way ANOVA followed by a Dunnett's test comparing to the N-gene positive control, significant fluorescence signal increase was found when targeting the N-gene compared to OT3 and OT4 (OT3, OT4: p<0.0001), but no significant difference was found targeting the N-gene compared to OT1 and OT2 (OT1: p=0.0786, OT2: p=0.0833).

FIGS. 16A-16C show SENSR detection of positive SARS-CoV-2 validated patient samples. FIG. 16A shows SENSR fluorescence analysis of RT-qPCR validated patient samples using gRNA-Z for detection. Samples 1-36 are patient samples positive for SARS-CoV-2 listed in order of ascending RT-qPCR Cₜ values for the N-gene where low Cₜ value is equal to a high viral load. The black and gray rings represent the Cₜ values for N, S, and Orf1ab genes, where black represents the N-gene Cₜ values and gray represents the S and Orf1ab Cₜ values. Samples N1-N5 are the five samples negative for SARS-CoV-2 with the highest recorded signal-to-noise ratios (S/N) of all negative samples analyzed with the highest S/N=2.4. The dashed line represents the S/N=2 threshold used to determine a positive detection of SARS-CoV-2. The asterisks indicate a positive detection of SARS-CoV-2 in the patient sample. FIG. 16B shows lateral flow based detection of the 36 samples from FIG. 16A that resulted in a positive detection of SARS-CoV-2 (S/N>2). The top band represents the test band and the bottom the control band. An increase in saturation of the top band indicates a positive detection of SARS-CoV-2 in the sample. A positive result was also determined by comparing to the NTC, which served as a baseline for a negative result. FIG. 16C provides a summary of fluorescence and lateral flow detection results for the RT-qPCR validated positive and negative patient samples.

FIG. 17 show that in addition to RNA-based templates (top row), SENSR technology could also be used for DNA-based templates, including, but not limited to, bacterial samples (shown) as well as DNA viruses such as herpes (shown, bottom). DNA-based sample templates can be input directly into the RPA amplification reaction, negating the need for a simultaneous reverse transcription (RT) reaction as is required for RNA-based templates. Like SENSR on RNA-based templates, the reaction occurs in two steps. The first amplification step, RT-RPA or RPA, is followed by the SENSR reaction which is identical regardless of initial input molecular species. This reaction is composed of simultaneous in vitro transcription (IVT) as well as CasRx cleavage and activation of collateral cleavage activity. The assay can be read out by fluorescence or lateral flow in the same fashion regardless of DNA or RNA sample input.

### BRIEF DESCRIPTION OF THE TABLES

Table 1 is a summary of CRISPR-based anti-COVID technologies.

Table 2 provides identifies 30 nt gRNA target sites conserved across, and specific to the SARS-CoV-2 genome.

Table 3 provides predicted unique and conserved 30 nt CasRx gRNA target sequences to SARS-CoV-2.

Table 4 provides analysis of inter-SARS-CoV-2 conservation (433 genomes) and Pan-coronavirus specificity (3164 genomes) on the three E-targeting gRNAs (R,T,V).

Table 5 provides a list and sequences of reagents generated and used, such as primers for cloning, gRNA prep, and RT-RPA, as well as gRNA sequences, viral gene templates, plasmid sequences and probes.

Table 6 provides top four naturally-occurring off-target sequences for gRNA T and gRNA Z.

Table 7 illustrates data from RT-qPCR and SENSR fluorescence analysis of patient samples for detection of SARS-CoV-2.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0, 0.7, 0.5, 0.3, 0.1, or 0.01, as appropriate, or alternatively by a variation of +/−15%, or alternatively 10%, or alternatively 5%, or alternatively 2%. It is to be understood, although not always explicitly stated, that all numerical designations are preceded by the term “about” and the appropriate range is included within the use of the term. The term “about,” as used herein when referring to a measurable value such as an amount or concentration and the like, is meant to encompass variations of 20%, 15%, 10%, 7%, 5%, 3%, 1%, 0.5%, 0.1% or even 0.01% of the specified amount. It also is to be understood, although not always explicitly stated, that the reagents described herein are merely exemplary and that equivalents of such are known in the art.

### Definitions

As it would be understood, the section or subsection headings as used herein is for organizational purposes only and are not to be construed as limiting and/or separating the subject matter described.

As used in the description of the invention and the appended claims, the singular forms “a,” “an” and “the” are intended to include the plural forms as well, unless the context clearly indicates otherwise.

“Optional” or “optionally” means that the subsequently described circumstance may or may not occur, so that the description includes instances where the circumstance occurs and instances where it does not.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. In some embodiments, Clustal Omega (accessible at www.ebi.ac.uk/Tools/msa/clustalo/) is used to generate the alignment and identity percentage. In further embodiments, default setting is applied.

The terms or “acceptable,” “effective,” or “sufficient” when used to describe the selection of any components, ranges, dose forms, etc. disclosed herein intend that said component, range, dose form, etc. is suitable for the disclosed purpose.

As will be understood by one skilled in the art, for any and all purposes, all ranges disclosed herein also encompass any and all possible subranges and combinations of subranges thereof. Furthermore, as will be understood by one skilled in the art, a range includes each individual member.

Also as used herein, “and/or” refers to and encompasses any and all possible combinations of one or more of the associated listed items, as well as the lack of combinations when interpreted in the alternative (“or”).

The term “cell” as used herein may refer to either a prokaryotic or eukaryotic cell, optionally obtained from a subject or a commercially available source.

The term “cell” or “host cell” as used herein may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a gene editing system or technique relying on CRISPR-based, sequence-specific genetic or epigenetic manipulation. Epigenetic manipulation includes modifications to nucleotides or higher order chromatin structure that can alter expression patterns of genes in the absence of changes to the underlying DNA sequence. Epigenetic modifications can occur on multiple levels, such as 5-methyl-cytosine (5-meC) DNA methylation, post-translational modifications of histones bound by protein domains that serve as epigenetic writers, readers and erasers, and noncoding RNAs that assist in the recruitment of chromatin modifying proteins to DNA. For example, a CRISPR-based gene editing system can be utilized in a sequence-specific manner to reduce levels of DNA methylation near the regulatory elements of a gene of interest to promote expression of the gene of interest. A CRISPR-based gene editing system can also be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359; Int'l. Publ. Nos. WO 2017/091630 A1, WO 2017/180915 A2, WO 2018/035503 A1, and WO 2018/170015 A1; Hsu et al. (2014) Cell 156(6): 1262-78; and Urbano et al. (2019) Cancers 11(10):E1515.

In some embodiments, the term “CRISPR” refers to a technique of sequence specific genetic manipulation relying on the clustered regularly interspaced short palindromic repeats pathway, which unlike RNA interference regulates gene expression at a transcriptional level. The term “guide” as used herein refers to the guide polynucleotide sequences used to target specific genes employing the CRISPR technique. In some embodiments, the guide is a guide RNA (gRNA). Techniques of designing gRNAs and donor therapeutic polynucleotides for target specificity are well known in the art. See, e.g., Doench et al. (2014) Nature Biotechnol. 32(12):1262-7 and Graham et al. (2015) Genome Biol. 16: 260, incorporated by reference herein.

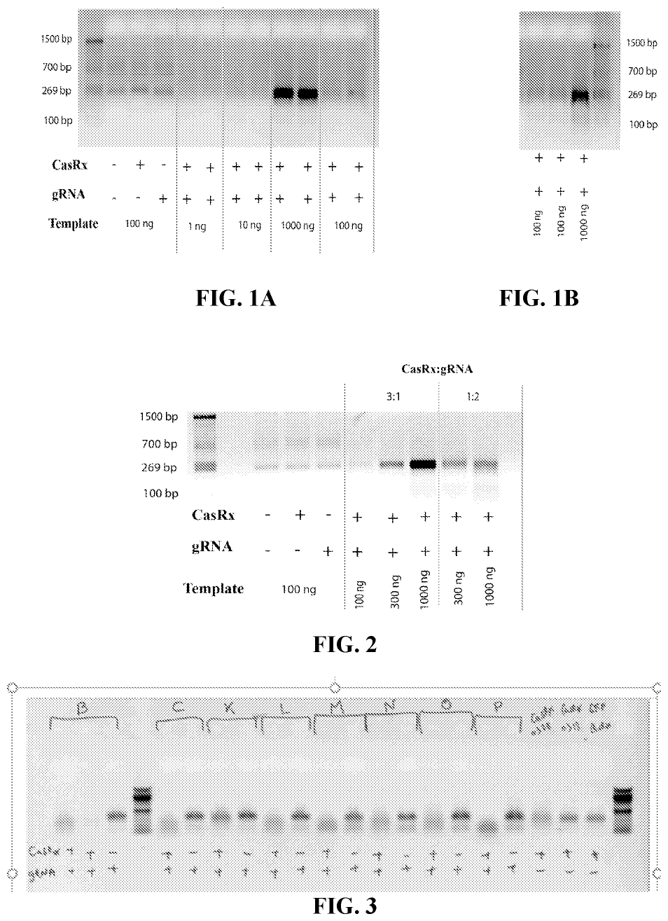

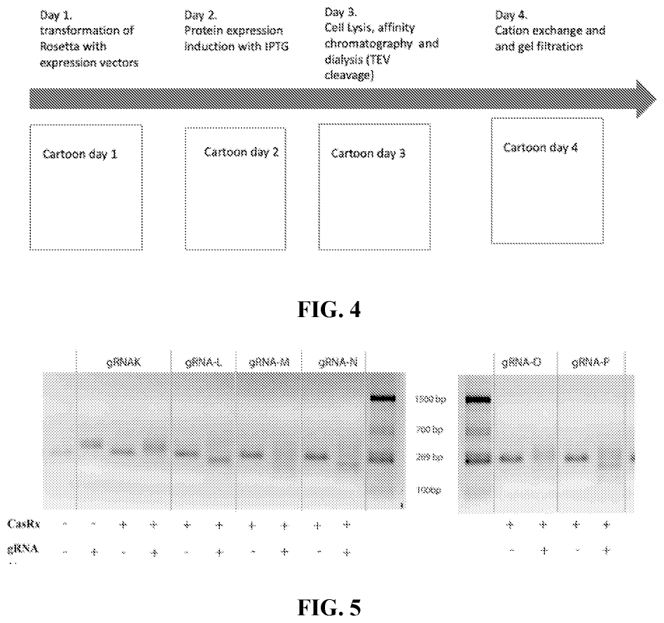

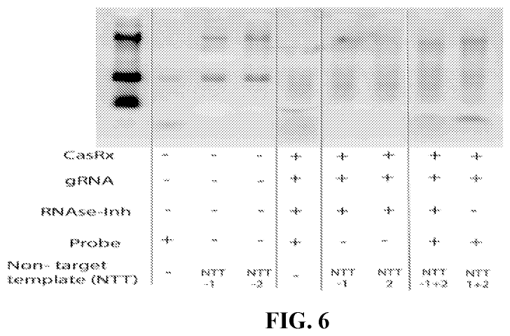

Recently, a number of novel CRISPR-based diagnostics have been developed to detect COVID-19. CRISPR-Cas nucleases can be easily programmed to target nucleic acids in a sequence-specific manner (Jinek et al., Science 337, 816-821 (2012); Abudayyeh et al., Science 353, aaf5573 (2016); and Zetsche et al., Cell 163, 759-771 (2015)), making them prime candidates for the detection and diagnosis of viral genetic material, and forming the CRISPR-based diagnostics (CRISPRDx) pipeline (Gootenberg et al., Science vol. 356 438-442 (2017); Gootenberg et al., Science 360, 439-444 (2018); Chen et al., Science 360, 436-439 (2018); and Li et al., Cell Discov 4, 20 (2018)). These systems rely on Type II Cas enzymes to physically bind target sequences (Azhar et al. bioRxiv 2020.04.07.028167 (2020) doi:10.1101/2020.04.07.028167), or collateral cleavage by Type V or Type VI enzymes to detect DNA (Chen et al., 2018; Li et al., 2018; and Harrington et al., 2018, Science 362, 839-842) or RNA species, respectively (Gootenberg et al., 2017; Gootenberg et al., 2018; and Freije et al., 2019, Mol. Cell 76, 826-837.el 1). Since pandemic onset, an array of innovative diagnostics and prophylactics relying on these technologies have been adapted to detect or target SARS-CoV-2 with unprecedented speed (Azhar et al., 2020; Mukama et al., Biosensors and Bioelectronics 112143 (2020) doi:10.1016/j.bios.2020.112143; Hajian et al. Nat Biomed Eng 3, 427-437 (2019); Patchsung et al., 2020; Lucia et al., 2020; Joung et al., 2020; Ding et al., 2020; Broughton et al., 2020; Rauch et al. bioRxiv 2020.04.20.052159 (2020) doi:10.1101/2020.04.20.052159; Ackerman et al. Nature (2020) doi:10.1038/s41586-020-2279-8; Zhang et al., 2020; Metsky et al., 2020; and Abbott et al., 2020), most notably represented by the DETECTR (DNA Endonuclease Targeted CRISPR Trans Reporter) (Chen et al., 2018; and Li et al., 2018) and SHERLOCK (Specific High-Sensitivity Enzymatic Reporter unLOCKing) (Gootenberg et al., 2017; and Gootenberg et al., 2018) systems (Summarized in FIG. 1, and Table 1).

The SHERLOCK system combines isothermal amplification of target sequences, followed by target recognition via Leptotrichia wadei Cas13a (LwaCas13a) and collateral cleavage of a bystander ssRNA probe to report the presence of a target (Gootenberg et al., 2017). This system has undergone significant optimization since its first development in 2017. This includes improvement of i) sensitivity, by the inclusion of an accessory protein to amplify signal or substitution of RPA with LAMP (Gootenberg et al., 2018; Howson et al., 2017; Hinton, D. M. Sherlock CRISPR SARS-CoV-2 Kit. (2020)), ii) specificity, by primer and guide optimization (Gootenberg et al., 2017; and Gootenberg et al., 2018), iii) throughput, by multiplexing detection using additional enzymes (including a cocktail of LwaCas13a, PsmCas13b (Prevotella sp. MA2016), CcaCas13b (Capnocytophaga canimorsus Cc5), and AsCas12a (Acidaminococcus sp. BV3L6)) (Gootenberg et al., 2018), and iv) validation as a point-of-care diagnostic by using lateral flow and ultrafast RNA extraction methods (Gootenberg et al., 2018; Patchsung et al., 2020; Joung et al., 2020; and Myhrvold et al., 2018, Science 360, 444-448). Ideally, to maximize all the capabilities of SHERLOCK and expand the CRISPRDx toolkit, it is important to evaluate alternative Cas enzymes that can complement or supplement the system.

Similar to Cas ribonucleases used in other CRISPRDx systems, Cas13d enzymes such as RfxCas13d (CasRx), exclusively target RNA species that trigger subsequent collateral cleavage of bystander RNA (Konermann et al., 2018; Buchman et al., 2020; and Yan et al, 2018). Collateral cleavage is initiated, following on-target ssRNA cleavage, by the HEPN domain-based endoRNase heterodimer, which activates trans-cleavage of nonspecific bystander RNAs (Abudayyeh et al., 2016; Konermann et al., 2018; Yan et al., 2018; and Zhang et al. 2018, Cell 175, 212-223.e17). Furthermore, Cas13d enzymes are approximately 20% smaller than Cas13a-Cas13c effectors, and do not require a Protospacer Flanking Sequence (PFS) (Abudayyeh et al., 2016; Konermann et al., 2018; Yan et al., 2018; and Kellner et al., 2019), presenting an advantage for protein production and flexible targeting. While the genetic modulatory effects of CasRx have been thoroughly characterized in Drosophila, zebrafish, and human cells (Konermann et al., 2018; Buchman et al., 2020; and Kushawah, et al. CRISPR-Cas13d induces efficient mRNA knock-down in animal embryos. bioRxiv (2020)), and its putative prophylactic properties against SARS-CoV-2 have been demonstrated (Abbott et al., 2020), its potential as a diagnostic system has not yet been explored.

TABLE 1
Summary Table of current CRISPR-based anti-Covid technologies.

| | | | | | | | | |
|---|---|---|---|---|---|---|---|---|
| | What does | | | | | | | |
| | acronym | Diagnostic or | | | RNA or DNA | SARS-CoV-2 | Mode of | Mode of |
| Tool name | stand for? | Treatment? | Time | Enzyme | Target? | Target Gene(s) | Detection | Amplification |

Cas12-based

| | | | | | | | | |
|---|---|---|---|---|---|---|---|---|
| DETECTR | DNA | Diagnostic | ~40 | Cas12a | DNA | E and N | Collateral | Yes. |
| | Endonuclease- | | m | | | | cleavage | RT-RPA |
| | Targeted | | | | | | | |
| | CRISPR | | | | | | | |
| | Trans Reporter | | | | | | | |
| AIOD- | All-In-One | Diagnostic | ~90 | Cas12a | DNA | N | Collateral | Yes, |
| CRISPR | Dual | | m | | | | cleavage | RPA |
| | CRISPR- | | | | | | | |
| | Cas12a | | | | | | | |
| CASdetec | CRISPR- | Diagnostic | ~40- | Cas12b | DNA | Rd Rp | | Yes, |
| | assisted | | 60 m | | | | | RT-RAA |
| | detection | | | | | | | |
| STOPCovid | SHERLOCK | Diagnostic | ~15- | Cas12b | DNA | N | | Yes, |
| | Testing in | | 45 m | | | | | RT-LAMP |
| | One Pot | | | | | | | |

Cas13-based

| | | | | | | | | |
|---|---|---|---|---|---|---|---|---|
| SHERLOCK | Sensitive | Diagnostic | ~35- | Cas13a, | RNA | N, S, and | Collateral | Yes, |
| | Enzymatic | | 70 m | Cas13b | | Orf1ab | Cleavage | RT-RPA |
| | Nucleic acid | | | | | | | |
| | Sequence | | | | | | | |
| | Reporter | | | | | | | |
| SherlockTM | See above | Diagnostic | ~1 h | LwaCas13a | RNA | N and Orf1ab | Collateral | Yes, |
| CRISPR | | | | | | | Cleavage | RT-LAMP |
| SARS-CoV-2 | | | | | | | | |
| kit | | | | | | | | |
| SENSR | Sensitive | Diagnostic | ~105 | RfxCas13d | RNA | E, S and N | Collateral | Yes, |
| | Enzymatic | | m | | | | Cleavage | RT-RPA |
| | Nucleic acid | | | | | | | |
| | Sequence | | | | | | | |
| | Reporter | | | | | | | |
| CREST | Cas13-based, | Diagnostic | ~1- | Cas13a | RNA | N | Collateral | Yes, |
| | Rugged, | | 2 h | | | | cleavage | RPA or PCR |
| | Equitable, | | | | | | | |
| | Sealable | | | | | | | |
| | Testing | | | | | | | |
| CARMEN- | Combinatorial | Diagnostic | N/A | Cas13a | RNA | N/A | Collateral | Yes, |
| Cas13 | Arrayed | | | | | | cleavage | PCR or RPA |
| | Reactions for | | | | | | | |
| | Multiplexed | | | | | | | |
| | Evaluation of | | | | | | | |
| | Nucleic acids | | | | | | | |
| PAC-MAN | Prophylactic | Prophylactic | N/A | Cas13d | RNA | Rd Rp and N | N/A | No |
| | Antiviral | | | | | | | |
| | CRISPR in | | | | | | | |
| | huMAN cells | | | | | | | |
| [no name] | N/A | Prophylactc | N/A | Cas13a | RNA | N and Orf1ab | N/A | |
| Georgia | | | | | | | | |
| Institute | | | | | | | | |
| of Tech, | | | | | | | | |
| Blanchard | | | | | | | | |
| et al | | | | | | | | |

Other Cas's

| | | | | | | | | |
|---|---|---|---|---|---|---|---|---|
| FELUDA | FnCas9 Edit | Dagnostic | ~1- | FnCas9 | DNA | N | Binding or | Yes, |
| | or Linked | | 2 h | (dCas9) | | | Cleavage | PCR or RPA |
| | Uniform | | | | | | | |
| | Detection | | | | | | | |
| | Assay | | | | | | | |

| | Total | | Reference: | FDA | Reference: |
|---|---|---|---|---|---|
| Tool name | Reactions | Read out | Covid Application | Approved? | Original Tech |
| Cas12-based | | | | | |
| DETECTR | 2 | Fluorescence | www.nature.com/ | Yes | science.sciencemag.org/ |
| | | & Lateral flow | articles/s41587- | | content/early/2018/02/14/ |
| | | | 020-0513-4 | | science.aar6245?versioned=true |
| | | | | | www.nature.com/articles/ |
| | | | | | s41421-018-0028-z |
| AIOD- | 1 | Fluorescence | www.nature.com/ | No | |
| CRISPR | | | articles/s41467- | | |
| | | | 020-18575-6 | | |
| CASdetec | 1 | Fluorescence | www.nature.com/ | No | |
| | | | articles/s41421- | | |
| | | | 020-0174-y | | |
| STOPCovid | 1 | Lateral Flow | www.nejm.org/ | No | |
| | | | doi/10.1056/ | | |
| | | | NEJMc2026172 | | |
| Cas13-based | | | | | |
| SHERLOCK | 2 | Fluorescence | www.nature.com/ | No | science.sciencemag.org/ |
| | | & Lateral flow | articles/s41551- | | content/356/6336/438 |
| | | | 020-00603-x | | science.sciencemag.org/ |
| | | | | | content/360/6387/439 |
| SherlockTM | 2 | Fluorescence | www.fda.gov/media/ | Yes | See above |
| CRISPR | | & Lateral flow | 137747/download; | | |
| SARS-CoV-2 | | | www.fda.gov/media/ | | |
| kit | | | 137746/download | | |
| SENSR | 2 | Fluorescence | This disclosure | No | This disclosure |
| | | & Lateral flow | | | |
| CREST | 3+ | Fluorescence | www.biorxiv.org/ | No | |
| | | & Lateral flow | content/10.1101/ | | |
| | | | 2020.04.20.052159v1 | | |
| CARMEN- | 2 | Fluorescence | www.nature.com/ | No | |
| Cas13 | | | articles/s41586- | | |
| | | | 020-2279-8 | | |
| PAC-MAN | N/A | N/A | www.cell.com/cell/pdf/ | No | |
| | | | S0092-8674(20)30483-9.pdf | | |
| [no name] | N/A | N/A | www.biorxiv.org/ | No | |
| Georgia | | | content/10.1101/ | | |
| Institute | | | 2020.04.24.060418v1 | | |
| of Tech, | | | | | |
| Blanchard | | | | | |
| et al | | | | | |
| Other Cas's | | | | | |
| FELUDA | 2 | Agarose | www.biorxiv.org/ | No | |
| | | Capillary | content/10.1101/ | | |
| | | electorphoresis | 2020.04.07.028167v2 | | |
| | | (cleavage) | | | |
| | | Lateral flow | | | |
| | | Fluorescence | | | |
| | | Read out | | | |

As used herein, the term “Cas”, which is an abbreviation for CRISPR Associated Protein, generally refers to an effector protein of the CRISPR/Cas system or complex, and can be without limitation a Cas9, or other enzymes such as Cpf1, C2c1, C2c2, C2c3, group 29, group 30 protein, Cas13a, Cas13b, Cas13c or Cas13. The term “Cas” may be used herein interchangeably with the terms “CRISPR” protein, “CRISPR/Cas protein”, “CRISPR effector”, “CRISPR/Cas effector”, “CRISPR enzyme”, “CRISPR/Cas enzyme” and the like, unless otherwise apparent, such as by specific and exclusive reference to Cas13d. It is to be understood that the term “CRISPR protein” may be used interchangeably with “CRISPR enzyme”, irrespective of whether the CRISPR protein has altered, such as increased or decreased (or no) enzymatic activity, compared to the wild type CRISPR protein. Likewise, as used herein, in certain embodiments, where appropriate and which will be apparent to the skilled person, the term “nuclease” may refer to a modified nuclease wherein catalytic activity has been altered, such as having increased or decreased nuclease activity, or no nuclease activity at all, as well as nickase activity, as well as otherwise modified nuclease as defined herein elsewhere, unless otherwise apparent, such as by specific and exclusive reference to unmodified nuclease. In some embodiments, the CRISPR effector protein is a RNA-targeting CRISPR effector protein. In some embodiments, the CRISPR effector protein is a Type-VI CRISPR effector protein such as Cas13a, Cas13b, Cas13c, or Cas13d.

The term “Cas13” refers to one of a family of novel type of RNA targeting enzymes. The diverse Cas13 family contains at least four known subtypes, including Cas13a (formerly C2c2), Cas13b, Cas13c, and Cas13d. Cas13's function similarly to Cas9, using a ˜64-nt guide RNA to encode target specificity. The Cas13 protein complexes with the guide RNA via recognition of a short hairpin in the crRNA, and target specificity is encoded by a 28-30-nt spacer that is complementary to the target region. In addition to programmable RNase activity, all Cas13s exhibit collateral activity after recognition and cleavage of a target transcript, leading to non-specific degradation of any nearby transcripts regardless of complementarity to the spacer. Wessels, H.-H. et al. Nature Biotechnol. doi.org/10.1038/s41587-020-0456-9 (Published Mar. 16, 2020). In one aspect, the term also includes optimized versions of Cas13d and Cas13d orthologs.

As used herein, Cas13d refers to type VI-D CRISPR-associated RNA-guided ribonuclease Cas13d. In contrast to other RNA-targeting systems, target RNA cleavage by CRISPR/Cas13d is PFS-independent (Konermann et al., 2018; Yan et al., 2018; and Zhang et al. Cell 175, 212.e7-223.e7.). In some embodiments, Cas13d refers to the Cas13d from Ruminococcus flavefaciens (CasRx). In some embodiments, the sequence of CasRx is as disclosed in Table 5 as well as NCBI Reference Sequences: WP_009985792.1 or WP_075424065.1. Other Cas13d orthologs may be used, such as Cas13d from Ruminococcus bicirculans (see, e.g., NCBI Reference Sequences WP_195551251.1, WP_195518215.1, WP 195388575.1, WP 195249857.1, WP 195247626.1, WP_195221164.1, WP_186490282.1, or WP_041337480.1), Eubacterium sp. An11 (see, e.g., NCBI Reference Sequences WP_191531982.1 or WP_162611874.1), Eubacterium sp. An3 (see, e.g., NCBI Reference Sequence WP_158097005.1), Ruminococcus sp. KGMB03662 (see, e.g., NCBI Reference Sequence WP_138338249.1), Ruminococcus sp. AM47-2BH (see, e.g., NCBI Reference Sequence WP_118164717.1 or WP_118164714.1), Ruminococcus sp. AM54-1NS (see, e.g., NCBI Reference Sequence WP_118160305.1); Ruminococcus sp. AM31-15AC (see, e.g., NCBI Reference Sequence WP_118158110.1), Ruminococcus sp. AM43-6 (see, e.g., NCBI Reference Sequence WP_118125476.1), unclassified Ruminococcus (see, e.g., NCBI Reference Sequence WP_118053168.1 or WP_117897534.1), Ruminococcus sp. AF18-29 (see, e.g., NCBI Reference Sequence WP_117939725.1), Ruminococcus sp. AF25-19 (see, e.g., NCBI Reference Sequence WP_117928365.1), Ruminococcus sp. AM28-13 (see, e.g., NCBI Reference Sequence WP_117925375.1), Ruminococcus sp. AF37-20 (see, e.g., NCBI Reference Sequence WP_117903863.1), Ruminococcus sp. AF19-15 (see, e.g., NCBI Reference Sequence WP_117893310.1) Ruminococcus sp. AF21-11 (see, e.g., NCBI Reference Sequence WP_117878260.1), Ruminococcus sp. AF16-50 (see, e.g., NCBI Reference Sequence WP_117864390.1), Ruminococcus sp. AF34-12 (see, e.g., NCBI Reference Sequence WP_117858671.1), or Ruminococcus albus (see, e.g., NCBI Reference Sequence WP_041337480.1). Each of the NCBI reference sequences is incorporated herein by reference in its entirety. In some embodiments, a Cas13d as disclosed herein also intents an equivalent thereof, for example, having about 99%, or about 98%, or about 97%, or about 96%, or about 95%, or about 94%, or about 93%, or about 92%, or about 91%, or about 90%, or about 89%, or about 88%, or about 87% or about 86%, or about 85%, or about 80% identity to the wildtype Cas13d and substantially retaining the function of the wildtype, for example, of complexing with a gRNA, locating to a target sequence, and cleaving the target sequence.

The term “CasRx” intends a Ruminococcus flavefaciens Cas13d that in one aspect is fused to a nuclear localization sequences. See, e.g., Larochelle, Nature Methods, 15:312 (2018) doi.org/10.1038/nmeth.4681.

As used herein, the term “gRNA” refers to a guide RNA sequence, known in the art to be used with the CRISPR-Cas system to facilitate targeting of the gene. gRNAs typically comprises a gRNA scaffold and a target specific sequence for example complementary to the target sequence). In some embodiments, a scaffold sequence refers to the sequence within the gRNA that is responsible for Cas enzyme binding, it does not include the 20 bp spacer/targeting sequence that is used to guide Cas enzyme to target polynucleotide. In further embodiments, a scaffold sequence comprises, or consists essentially of, or yet further consists of a direct repeat. More than one gRNA may be present in a construct, i.e., multiple spacers may be used to ensure gene targeting. Non-limiting exemplary scaffolds are disclosed herein. The target specific sequences may be experimentally determined or found on one of many publically available databases, such as Addgene (www.addgene.org).

As used herein, direct repeats (also referred to herein as DR) refer to a polynucleotide which is about 20 to about 60 nt (such as about 21 nt to about 47 nt) long with weak dyad symmetry. DR combined with its adjacent spacer encodes a guide. The DR regions comprise, or consist essentially of, or yet further consist of sequences required for processing into mature guide, or guide binding to a Cas enzyme, or both. In some embodiments, DR comprise, or consist essentially of, or further consist of gcaaguaaaccccuaccaacuggucgggguuugaaac (SEQ ID NO:). In some embodiments, DR comprise, or consist essentially of, or further consist of caaguaaaccccuaccaacuggucgggguuugaaac (SEQ ID NO:).

In some embodiments, the term “spacer” refers to a target specific sequence, i.e., a polynucleotide complementary to the target sequence, optionally with about 1, about 2, about 3, about 4, about 5, about 6, about 7, about 8, or about 9 mismatches. Accordingly, a guide as disclosed herein comprises, or consists essentially of, or yet further consists of direct repeats and a spacer.

As used herein, Protospacer Adjacent Motif or PAM refers to a sequence adjacent to the target sequence that is necessary for Cas enzymes to bind target polynucleotide.

As used herein, PFS stands for protospacer flanking site, which is adjacent to the 3′ end of the protospacer and affects the efficacy of CRISPR-C2c2 targeting. The CRISPR-C2c2 system prefers H (A, U, or C) for the PFS sequence of one single base length to mediate single-strand RNA cleavage.

As used herein, the term “target” or “target sequence” refers to the section of the polynucleotide recognized by a CRISPR-guide complex. Such target can be in a pathogen genome or a RNA transcribed therefrom.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” For example, the gene editing systems described herein may consist essentially of the recited materials and additional materials that do not affect the ability of the at least one gRNA to hybridize to a nucleotide sequence complementary to a target sequence or to associate with the E gene or N gene. “Consisting of” shall mean excluding more than trace elements of other ingredients and substantial method steps for administering the compositions disclosed herein. Aspects defined by each of these transition terms are within the scope of the present disclosure.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

A “gene” refers to a polynucleotide containing at least one open reading frame (ORF) that is capable of encoding a particular polypeptide or protein after being transcribed and translated.

The term “express” refers to the production of a gene product. As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The terms “equivalent” or “biological equivalent” are used interchangeably when referring to a particular molecule, biological, or cellular material and intend those having certain sequence identity (such as about 99%, or about 98%, or about 97%, or about 96%, or about 95%, or about 94%, or about 93%, or about 92%, or about 91%, or about 90%, or about 89%, or about 88%, or about 87% or about 86%, or about 85%, or about 80%, or about 75%, or about 70%, or about 60%, or about 50% identity) while still substantially maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

As used herein, the term “functional” may be used to modify any molecule, biological, or cellular material to intend that it accomplishes a particular, specified effect.

The term “isolated” as used herein refers to molecules or biologicals or cellular materials being substantially free from other materials or contaminations.

As used herein, the terms “nucleic acid sequence,” “nucleotide sequence,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful in the methods disclosed herein include any of the serotypes, e.g., AAV2 and AAV8.

As used herein, the term “organ” a structure which is a specific portion of an individual organism, where a certain function or functions of the individual organism is locally performed and which is morphologically separate. Non-limiting examples of organs include the skin, blood vessels, cornea, thymus, kidney, heart, liver, umbilical cord, intestine, nerve, lung, placenta, pancreas, thyroid and brain.

The term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CMV promoter, a T7 promoter, U6 promoter, and EF-1α promoter. Non-limiting exemplary promoter sequences are provided herein below:

CMV Promoter

ATACGCGTTGACATTGATTATTGACTAGTTATTAAT
AGTAATCAATTACGGGGTCATTAGTTCATAGCCCA
TATATGGAGTTCCGCGTTACATAACTTACGGTAAA
TGGCCCGCCTGGCTGACCGCCCAACGACCCCCGCC
CATTGACGTCAATAATGACGTATGTTCCCATAGTA
ACGCCAATAGGGACTTTCCATTGACGTCAATGGGT
GGAGTATTTACGGTAAACTGCCCACTTGGCAGTAC
ATCAAGTGTATCATATGCCAAGTACGCCCCCTATT
GACGTCAATGACGGTAAATGGCCCGCCTGGCATTA
TGCCCAGTACATGACCTTATGGGACTTTCCTACTT
GGCAGTACATCTACGTATTAGTCATCGCTATTACC
ATGGTGATGCGGTTTTGGCAGTACATCAATGGGCG
TGGATAGCGGTTTGACTCACGGGGATTTCCAAGTC
TCCACCCCATTGACGTCAATGGGAGTTTGTTTTGG
CACCAAAATCAACGGGACTTTCCAAAATGTCGTAA
CAACTCCGCCCCATTGACGCAAATGGGCGGTAGGC
GTGTACGGTGGGAGGTCTATATAAGCAGAGCTCGT
TTAGTGAACCGTCAGATCGCCTGGAGACGCCATCC
ACGCTGTTTTGACCTCCATAGAAGACACCGGGACC
GATCCAGCCTCCGGACTCTAGAGGATCGAACCCTT

or a biological equivalent thereof.

U6 Promoter

GAGGGCCTATTTCCCATGATTCCTTCATATTTGCATATACGATACAAGGC
TGTTAGAGAGATAATTAGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACC

or a biological equivalent thereof.

EF1α Promoter

CGTGAGGCTCCGGTGCCCGTCAGTGGGCAGAGCGCACATCGCCCACAGTC
CCCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCTT
GAATTACTTCCACGCCCCTGGCTGCAGTACGTGATTCTTGATCCCGAGCT
TCGGGTTGGAAGTGGGTGGGAGAGTTCGAGGCCTTGCGCTTAAGGAGCCC
CTTCGCCTCGTGCTTGAGTTGAGGCCTGGCCTGGGCGCTGGGGCCGCCGC
GTGCGAATCTGGTGGCACCTTCGCGCCTGTCTCGCTGCTTTCGATAAGTC
TCTAGCCATTTAAAATTTTTGATGACCTGCTGCGACGCTTTTTTTCTGGC
AAGATAGTCTTGTAAATGCGGGCCAAGATCTGCACACTGGTATTTCGGTT
TTTGGGGCCGCGGGCGGCGACGGGGCCCGTGCGTCCCAGCGCACATGTTC
GGCGAGGCGGGGCCTGCGAGCGCGGCCACCGAGAATCGGACGGGGGTAGT
CTCAAGCTGGCCGGCCTGCTCTGGTGCCTGGCCTCGCGCCGCCGTGTATC
GCCCCGCCCTGGGCGGCAAGGCTGGCCCGGTCGGCACCAGTTGCGTGAGC
GGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGG
GCCTTTCCGTCCTCAGCCGTCGCTTCATGTGACTCCACGGAGTACCGGGC
GCCGTCCAGGCACCTCGATTAGTTCTCGAGCTTTTGGAGTACGTCGTCTT
TAGGTTGGGGGGAGGGGTTTTATGCGATGGAGTTTCCCCACACTGAGTGG
GTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAG
TGGTTCAAAGTTTTTTTCTTCCATTTCAGGTGTCGTGAG

or a biological equivalent thereof.

In some embodiments, a T7 promoter comprises, or consists essentially of, or yet further consists of a sequence of DNA 18 base pairs long up to transcription start site at +1 (5′-TAATACGACTCACTATAG-3′) that is recognized by T7 RNA polymerase. The T7 promoter is commonly used to regulate gene expression of recombinant proteins, which can be subsequently used for a variety of downstream research applications. See, for example, Rong et al., (1998), Proc Natl Acad Sci USA 95, 515-519; and Komura et al., (2018), PLOS ONE 13, e0196905.

A number of effector elements can be used in these vectors; e.g., a tetracycline response element (e.g., tetO), a tet-regulatable activator, T2A, VP64, Rta, KRAB, and a miRNA sensor circuit. The nature and function of these effector elements are commonly understood in the art and a number of these effector elements are commercially available. In one aspect, the systems further comprise an effector element.

The terms “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “recombinant expression system” refers to a genetic construct for the expression of certain genetic material formed by recombination.

As used herein, the term “subject” is intended to mean any animal. In some embodiments, the subject may be a mammal; in further embodiments, the subject may be a bat, bovine, equine, feline, murine, porcine, canine, human, or rat. They may be adult, a juvenile or a fetal subject as appropriate. In some embodiments, they refer to and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate (e.g., apes, gibbons, chimpanzees, orangutans, monkeys, macaques, and the like), particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. In some embodiments, a subject has or is diagnosed of having or is suspected of having an infection by a pathogen, such as SARS-CoV-2. In some embodiments, the subject is pre-symptomatic, i.e., having being infected by the pathogen but not yet developed a symptom. In some embodiments, the subject is asymptomatic, i.e., having being infected by the pathogen but does not develop a symptom. The subject can be a male or a female, adult, an infant or a pediatric subject. In an additional aspect, the subject is an adult. In some instances, the adult is an adult human, e.g., an adult human greater than 18 years of age.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the route of administration, and the physical delivery system in which it is carried.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the nasal passages, the throat, lung tissue, skeletal tissue, and/or muscle tissue. Exemplary tissues include, but are not limited to those derived from nose, sinus, oral cavity, lungs, heart, liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention or prophylaxis.

In some embodiments, the term “disease” or “disorder” as used herein refers to a pathogen infection, a status of being diagnosed with such infection, a status of being suspect of having such infection, a status of having being exposed to a pathogen, or a status of at high risk of being exposed to a pathogen. In some embodiments, the pathogen is a virus (such as a DNA virus or a RNA virus), a bacterium, or a fungi that may cause a disease in a subject. In further embodiments, the pathogen is coronavirus. In one embodiment, the term “disease” or “disorder” as used herein refers to a coronavirus infection, a status of being diagnosed with such infection, a status of being suspect of having such infection, a status of having being exposed to a coronavirus, or a status of at high risk of being exposed to a coronavirus. In one embodiment, the coronavirus is a respiratory virus. In a further embodiment, the disease is Coronavirus disease 2019 (COVID-19) caused by SARS-CoV-2. In yet a further embodiment, the disease is Severe acute respiratory syndrome (SARS) caused by SARS-CoV-1.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

In some embodiments, the coronavirus as used herein refers to a severe acute respiratory syndrome (SARS) associated coronavirus (SARS-CoV). In some embodiments, the coronavirus is either or both of SARS-CoV-1 and SARS-CoV-2. In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus.

Severe acute respiratory syndrome coronavirus 2 (SARS-CoV-2) is the virus that causes COVID-19 (coronavirus disease 2019), the respiratory illness responsible for the COVID-19 pandemic. SARS-CoV-2 is a positive-sense single-stranded RNA virus (and hence Baltimore class IV) that is contagious in humans. In some embodiments, the viral genome of SARS-CoV-2 is NCBI Reference Sequence NC_045512.2. In further embodiments, the viral genome of SARS-CoV-2 comprises, or consists essentially of, or yet further consists of

(SEQ ID NO: 1)
ATTAAAGGTTTATACCTTCCCAGGTAACAAACCAACCAACTTTCGATCTCTTGTAGATCTGTTCTCTAAACGAACTTTAAAATC
TGTGTGGCTGTCACTCGGCTGCATGCTTAGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATCAGCACATCTAGGTTTCGTCCGGGTGTG
ACCGAAAGGTAAGATGGAGAGCCTTGTCCCTGGTTTCAACGAGAAAACACACGTCCAACTCAGTTTGCCTGTTTTACAGGTTCG
CGACGTGCTCGTACGTGGCTTTGGAGACTCCGTGGAGGAGGTCTTATCAGAGGCACGTCAACATCTTAAAGATGGCACTTGTGG
CTTAGTAGAAGTTGAAAAAGGCGTTTTGCCTCAACTTGAACAGCCCTATGTGTTCATCAAACGTTCGGATGCTCGAACTGCACC
TCATGGTCATGTTATGGTTGAGCTGGTAGCAGAACTCGAAGGCATTCAGTACGGTCGTAGTGGTGAGACACTTGGTGTCCTTGT
CCCTCATGTGGGCGAAATACCAGTGGCTTACCGCAAGGTTCTTCTTCGTAAGAACGGTAATAAAGGAGCTGGTGGCCATAGTTA
CGGCGCCGATCTAAAGTCATTTGACTTAGGCGACGAGCTTGGCACTGATCCTTATGAAGATTTTCAAGAAAACTGGAACACTAA
ACATAGCAGTGGTGTTACCCGTGAACTCATGCGTGAGCTTAACGGAGGGGCATACACTCGCTATGTCGATAACAACTTCTGTGG
CCCTGATGGCTACCCTCTTGAGTGCATTAAAGACCTTCTAGCACGTGCTGGTAAAGCTTCATGCACTTTGTCCGAACAACTGGA
CTTTATTGACACTAAGAGGGGTGTATACTGCTGCCGTGAACATGAGCATGAAATTGCTTGGTACACGGAACGTTCTGAAAAGAG
CTATGAATTGCAGACACCTTTTGAAATTAAATTGGCAAAGAAATTTGACACCTTCAATGGGGAATGTCCAAATTTTGTATTTCC
CTTAAATTCCATAATCAAGACTATTCAACCAAGGGTTGAAAAGAAAAAGCTTGATGGCTTTATGGGTAGAATTCGATCTGTCTA
TCCAGTTGCGTCACCAAATGAATGCAACCAAATGTGCCTTTCAACTCTCATGAAGTGTGATCATTGTGGTGAAACTTCATGGCA
GACGGGCGATTTTGTTAAAGCCACTTGCGAATTTTGTGGCACTGAGAATTTGACTAAAGAAGGTGCCACTACTTGTGGTTACTT
ACCCCAAAATGCTGTTGTTAAAATTTATTGTCCAGCATGTCACAATTCAGAAGTAGGACCTGAGCATAGTCTTGCCGAATACCA
TAATGAATCTGGCTTGAAAACCATTCTTCGTAAGGGTGGTCGCACTATTGCCTTTGGAGGCTGTGTGTTCTCTTATGTTGGTTG
CCATAACAAGTGTGCCTATTGGGTTCCACGTGCTAGCGCTAACATAGGTTGTAACCATACAGGTGTTGTTGGAGAAGGTTCCGA
AGGTCTTAATGACAACCTTCTTGAAATACTCCAAAAAGAGAAAGTCAACATCAATATTGTTGGTGACTTTAAACTTAATGAAGA
GATCGCCATTATTTTGGCATCTTTTTCTGCTTCCACAAGTGCTTTTGTGGAAACTGTGAAAGGTTTGGATTATAAAGCATTCAA
ACAAATTGTTGAATCCTGTGGTAATTTTAAAGTTACAAAAGGAAAAGCTAAAAAAGGTGCCTGGAATATTGGTGAACAGAAATC
AATACTGAGTCCTCTTTATGCATTTGCATCAGAGGCTGCTCGTGTTGTACGATCAATTTTCTCCCGCACTCTTGAAACTGCTCA
AAATTCTGTGCGTGTTTTACAGAAGGCCGCTATAACAATACTAGATGGAATTTCACAGTATTCACTGAGACTCATTGATGCTAT
GATGTTCACATCTGATTTGGCTACTAACAATCTAGTTGTAATGGCCTACATTACAGGTGGTGTTGTTCAGTTGACTTCGCAGTG
GCTAACTAACATCTTTGGCACTGTTTATGAAAAACTCAAACCCGTCCTTGATTGGCTTGAAGAGAAGTTTAAGGAAGGTGTAGA
GTTTCTTAGAGACGGTTGGGAAATTGTTAAATTTATCTCAACCTGTGCTTGTGAAATTGTCGGTGGACAAATTGTCACCTGTGC
AAAGGAAATTAAGGAGAGTGTTCAGACATTCTTTAAGCTTGTAAATAAATTTTTGGCTTTGTGTGCTGACTCTATCATTATTGG
TGGAGCTAAACTTAAAGCCTTGAATTTAGGTGAAACATTTGTCACGCACTCAAAGGGATTGTACAGAAAGTGTGTTAAATCCAG
AGAAGAAACTGGCCTACTCATGCCTCTAAAAGCCCCAAAAGAAATTATCTTCTTAGAGGGAGAAACACTTCCCACAGAAGTGTT
AACAGAGGAAGTTGTCTTGAAAACTGGTGATTTACAACCATTAGAACAACCTACTAGTGAAGCTGTTGAAGCTCCATTGGTTGG
TACACCAGTTTGTATTAACGGGCTTATGTTGCTCGAAATCAAAGACACAGAAAAGTACTGTGCCCTTGCACCTAATATGATGGT
AACAAACAATACCTTCACACTCAAAGGCGGTGCACCAACAAAGGTTACTTTTGGTGATGACACTGTGATAGAAGTGCAAGGTTA
CAAGAGTGTGAATATCACTTTTGAACTTGATGAAAGGATTGATAAAGTACTTAATGAGAAGTGCTCTGCCTATACAGTTGAACT
CGGTACAGAAGTAAATGAGTTCGCCTGTGTTGTGGCAGATGCTGTCATAAAAACTTTGCAACCAGTATCTGAATTACTTACACC
ACTGGGCATTGATTTAGATGAGTGGAGTATGGCTACATACTACTTATTTGATGAGTCTGGTGAGTTTAAATTGGCTTCACATAT
GTATTGTTCTTTCTACCCTCCAGATGAGGATGAAGAAGAAGGTGATTGTGAAGAAGAAGAGTTTGAGCCATCAACTCAATATGA
GTATGGTACTGAAGATGATTACCAAGGTAAACCTTTGGAATTTGGTGCCACTTCTGCTGCTCTTCAACCTGAAGAAGAGCAAGA
AGAAGATTGGTTAGATGATGATAGTCAACAAACTGTTGGTCAACAAGACGGCAGTGAGGACAATCAGACAACTACTATTCAAAC
AATTGTTGAGGTTCAACCTCAATTAGAGATGGAACTTACACCAGTTGTTCAGACTATTGAAGTGAATAGTTTTAGTGGTTATTT
AAAACTTACTGACAATGTATACATTAAAAATGCAGACATTGTGGAAGAAGCTAAAAAGGTAAAACCAACAGTGGTTGTTAATGC
AGCCAATGTTTACCTTAAACATGGAGGAGGTGTTGCAGGAGCCTTAAATAAGGCTACTAACAATGCCATGCAAGTTGAATCTGA
TGATTACATAGCTACTAATGGACCACTTAAAGTGGGTGGTAGTTGTGTTTTAAGCGGACACAATCTTGCTAAACACTGTCTTCA
TGTTGTCGGCCCAAATGTTAACAAAGGTGAAGACATTCAACTTCTTAAGAGTGCTTATGAAAATTTTAATCAGCACGAAGTTCT
ACTTGCACCATTATTATCAGCTGGTATTTTTGGTGCTGACCCTATACATTCTTTAAGAGTTTGTGTAGATACTGTTCGCACAAA
TGTCTACTTAGCTGTCTTTGATAAAAATCTCTATGACAAACTTGTTTCAAGCTTTTTGGAAATGAAGAGTGAAAAGCAAGTTGA
ACAAAAGATCGCTGAGATTCCTAAAGAGGAAGTTAAGCCATTTATAACTGAAAGTAAACCTTCAGTTGAACAGAGAAAACAAGA
TGATAAGAAAATCAAAGCTTGTGTTGAAGAAGTTACAACAACTCTGGAAGAAACTAAGTTCCTCACAGAAAACTTGTTACTTTA
TATTGACATTAATGGCAATCTTCATCCAGATTCTGCCACTCTTGTTAGTGACATTGACATCACTTTCTTAAAGAAAGATGCTCC
ATATATAGTGGGTGATGTTGTTCAAGAGGGTGTTTTAACTGCTGTGGTTATACCTACTAAAAAGGCTGGTGGCACTACTGAAAT
GCTAGCGAAAGCTTTGAGAAAAGTGCCAACAGACAATTATATAACCACTTACCCGGGTCAGGGTTTAAATGGTTACACTGTAGA
GGAGGCAAAGACAGTGCTTAAAAAGTGTAAAAGTGCCTTTTACATTCTACCATCTATTATCTCTAATGAGAAGCAAGAAATTCT
TGGAACTGTTTCTTGGAATTTGCGAGAAATGCTTGCACATGCAGAAGAAACACGCAAATTAATGCCTGTCTGTGTGGAAACTAA
AGCCATAGTTTCAACTATACAGCGTAAATATAAGGGTATTAAAATACAAGAGGGTGTGGTTGATTATGGTGCTAGATTTTACTT
TTACACCAGTAAAACAACTGTAGCGTCACTTATCAACACACTTAACGATCTAAATGAAACTCTTGTTACAATGCCACTTGGCTA
TGTAACACATGGCTTAAATTTGGAAGAAGCTGCTCGGTATATGAGATCTCTCAAAGTGCCAGCTACAGTTTCTGTTTCTTCACC
TGATGCTGTTACAGCGTATAATGGTTATCTTACTTCTTCTTCTAAAACACCTGAAGAACATTTTATTGAAACCATCTCACTTGC
TGGTTCCTATAAAGATTGGTCCTATTCTGGACAATCTACACAACTAGGTATAGAATTTCTTAAGAGAGGTGATAAAAGTGTATA
TTACACTAGTAATCCTACCACATTCCACCTAGATGGTGAAGTTATCACCTTTGACAATCTTAAGACACTTCTTTCTTTGAGAGA
AGTGAGGACTATTAAGGTGTTTACAACAGTAGACAACATTAACCTCCACACGCAAGTTGTGGACATGTCAATGACATATGGACA
ACAGTTTGGTCCAACTTATTTGGATGGAGCTGATGTTACTAAAATAAAACCTCATAATTCACATGAAGGTAAAACATTTTATGT
TTTACCTAATGATGACACTCTACGTGTTGAGGCTTTTGAGTACTACCACACAACTGATCCTAGTTTTCTGGGTAGGTACATGTC
AGCATTAAATCACACTAAAAAGTGGAAATACCCACAAGTTAATGGTTTAACTTCTATTAAATGGGCAGATAACAACTGTTATCT
TGCCACTGCATTGTTAACACTCCAACAAATAGAGTTGAAGTTTAATCCACCTGCTCTACAAGATGCTTATTACAGAGCAAGGGC
TGGTGAAGCTGCTAACTTTTGTGCACTTATCTTAGCCTACTGTAATAAGACAGTAGGTGAGTTAGGTGATGTTAGAGAAACAAT
GAGTTACTTGTTTCAACATGCCAATTTAGATTCTTGCAAAAGAGTCTTGAACGTGGTGTGTAAAACTTGTGGACAACAGCAGAC
AACCCTTAAGGGTGTAGAAGCTGTTATGTACATGGGCACACTTTCTTATGAACAATTTAAGAAAGGTGTTCAGATACCTTGTAC
GTGTGGTAAACAAGCTACAAAATATCTAGTACAACAGGAGTCACCTTTTGTTATGATGTCAGCACCACCTGCTCAGTATGAACT
TAAGCATGGTACATTTACTTGTGCTAGTGAGTACACTGGTAATTACCAGTGTGGTCACTATAAACATATAACTTCTAAAGAAAC
TTTGTATTGCATAGACGGTGCTTTACTTACAAAGTCCTCAGAATACAAAGGTCCTATTACGGATGTTTTCTACAAAGAAAACAG
TTACACAACAACCATAAAACCAGTTACTTATAAATTGGATGGTGTTGTTTGTACAGAAATTGACCCTAAGTTGGACAATTATTA
TAAGAAAGACAATTCTTATTTCACAGAGCAACCAATTGATCTTGTACCAAACCAACCATATCCAAACGCAAGCTTCGATAATTT
TAAGTTTGTATGTGATAATATCAAATTTGCTGATGATTTAAACCAGTTAACTGGTTATAAGAAACCTGCTTCAAGAGAGCTTAA
AGTTACATTTTTCCCTGACTTAAATGGTGATGTGGTGGCTATTGATTATAAACACTACACACCCTCTTTTAAGAAAGGAGCTAA
ATTGTTACATAAACCTATTGTTTGGCATGTTAACAATGCAACTAATAAAGCCACGTATAAACCAAATACCTGGTGTATACGTTG
TCTTTGGAGCACAAAACCAGTTGAAACATCAAATTCGTTTGATGTACTGAAGTCAGAGGACGCGCAGGGAATGGATAATCTTGC
CTGCGAAGATCTAAAACCAGTCTCTGAAGAAGTAGTGGAAAATCCTACCATACAGAAAGACGTTCTTGAGTGTAATGTGAAAAC
TACCGAAGTTGTAGGAGACATTATACTTAAACCAGCAAATAATAGTTTAAAAATTACAGAAGAGGTTGGCCACACAGATCTAAT
GGCTGCTTATGTAGACAATTCTAGTCTTACTATTAAGAAACCTAATGAATTATCTAGAGTATTAGGTTTGAAAACCCTTGCTAC
TCATGGTTTAGCTGCTGTTAATAGTGTCCCTTGGGATACTATAGCTAATTATGCTAAGCCTTTTCTTAACAAAGTTGTTAGTAC
AACTACTAACATAGTTACACGGTGTTTAAACCGTGTTTGTACTAATTATATGCCTTATTTCTTTACTTTATTGCTACAATTGTG
TACTTTTACTAGAAGTACAAATTCTAGAATTAAAGCATCTATGCCGACTACTATAGCAAAGAATACTGTTAAGAGTGTCGGTAA
ATTTTGTCTAGAGGCTTCATTTAATTATTTGAAGTCACCTAATTTTTCTAAACTGATAAATATTATAATTTGGTTTTTACTATT
AAGTGTTTGCCTAGGTTCTTTAATCTACTCAACCGCTGCTTTAGGTGTTTTAATGTCTAATTTAGGCATGCCTTCTTACTGTAC
TGGTTACAGAGAAGGCTATTTGAACTCTACTAATGTCACTATTGCAACCTACTGTACTGGTTCTATACCTTGTAGTGTTTGTCT
TAGTGGTTTAGATTCTTTAGACACCTATCCTTCTTTAGAAACTATACAAATTACCATTTCATCTTTTAAATGGGATTTAACTGC
TTTTGGCTTAGTTGCAGAGTGGTTTTTGGCATATATTCTTTTCACTAGGTTTTTCTATGTACTTGGATTGGCTGCAATCATGCA
ATTGTTTTTCAGCTATTTTGCAGTACATTTTATTAGTAATTCTTGGCTTATGTGGTTAATAATTAATCTTGTACAAATGGCCCC
GATTTCAGCTATGGTTAGAATGTACATCTTCTTTGCATCATTTTATTATGTATGGAAAAGTTATGTGCATGTTGTAGACGGTTG
TAATTCATCAACTTGTATGATGTGTTACAAACGTAATAGAGCAACAAGAGTCGAATGTACAACTATTGTTAATGGTGTTAGAAG
GTCCTTTTATGTCTATGCTAATGGAGGTAAAGGCTTTTGCAAACTACACAATTGGAATTGTGTTAATTGTGATACATTCTGTGC
TGGTAGTACATTTATTAGTGATGAAGTTGCGAGAGACTTGTCACTACAGTTTAAAAGACCAATAAATCCTACTGACCAGTCTTC
TTACATCGTTGATAGTGTTACAGTGAAGAATGGTTCCATCCATCTTTACTTTGATAAAGCTGGTCAAAAGACTTATGAAAGACA
TTCTCTCTCTCATTTTGTTAACTTAGACAACCTGAGAGCTAATAACACTAAAGGTTCATTGCCTATTAATGTTATAGTTTTTGA
TGGTAAATCAAAATGTGAAGAATCATCTGCAAAATCAGCGTCTGTTTACTACAGTCAGCTTATGTGTCAACCTATACTGTTACT
AGATCAGGCATTAGTGTCTGATGTTGGTGATAGTGCGGAAGTTGCAGTTAAAATGTTTGATGCTTACGTTAATACGTTTTCATC
AACTTTTAACGTACCAATGGAAAAACTCAAAACACTAGTTGCAACTGCAGAAGCTGAACTTGCAAAGAATGTGTCCTTAGACAA
TGTCTTATCTACTTTTATTTCAGCAGCTCGGCAAGGGTTTGTTGATTCAGATGTAGAAACTAAAGATGTTGTTGAATGTCTTAA
ATTGTCACATCAATCTGACATAGAAGTTACTGGCGATAGTTGTAATAACTATATGCTCACCTATAACAAAGTTGAAAACATGAC
ACCCCGTGACCTTGGTGCTTGTATTGACTGTAGTGCGCGTCATATTAATGCGCAGGTAGCAAAAAGTCACAACATTGCTTTGAT
ATGGAACGTTAAAGATTTCATGTCATTGTCTGAACAACTACGAAAACAAATACGTAGTGCTGCTAAAAAGAATAACTTACCTTT
TAAGTTGACATGTGCAACTACTAGACAAGTTGTTAATGTTGTAACAACAAAGATAGCACTTAAGGGTGGTAAAATTGTTAATAA
TTGGTTGAAGCAGTTAATTAAAGTTACACTTGTGTTCCTTTTTGTTGCTGCTATTTTCTATTTAATAACACCTGTTCATGTCAT
GTCTAAACATACTGACTTTTCAAGTGAAATCATAGGATACAAGGCTATTGATGGTGGTGTCACTCGTGACATAGCATCTACAGA
TACTTGTTTTGCTAACAAACATGCTGATTTTGACACATGGTTTAGCCAGCGTGGTGGTAGTTATACTAATGACAAAGCTTGCCC
ATTGATTGCTGCAGTCATAACAAGAGAAGTGGGTTTTGTCGTGCCTGGTTTGCCTGGCACGATATTACGCACAACTAATGGTGA
CTTTTTGCATTTCTTACCTAGAGTTTTTAGTGCAGTTGGTAACATCTGTTACACACCATCAAAACTTATAGAGTACACTGACTT
TGCAACATCAGCTTGTGTTTTGGCTGCTGAATGTACAATTTTTAAAGATGCTTCTGGTAAGCCAGTACCATATTGTTATGATAC
CAATGTACTAGAAGGTTCTGTTGCTTATGAAAGTTTACGCCCTGACACACGTTATGTGCTCATGGATGGCTCTATTATTCAATT
TCCTAACACCTACCTTGAAGGTTCTGTTAGAGTGGTAACAACTTTTGATTCTGAGTACTGTAGGCACGGCACTTGTGAAAGATC
AGAAGCTGGTGTTTGTGTATCTACTAGTGGTAGATGGGTACTTAACAATGATTATTACAGATCTTTACCAGGAGTTTTCTGTGG
TGTAGATGCTGTAAATTTACTTACTAATATGTTTACACCACTAATTCAACCTATTGGTGCTTTGGACATATCAGCATCTATAGT
AGCTGGTGGTATTGTAGCTATCGTAGTAACATGCCTTGCCTACTATTTTATGAGGTTTAGAAGAGCTTTTGGTGAATACAGTCA
TGTAGTTGCCTTTAATACTTTACTATTCCTTATGTCATTCACTGTACTCTGTTTAACACCAGTTTACTCATTCTTACCTGGTGT
TTATTCTGTTATTTACTTGTACTTGACATTTTATCTTACTAATGATGTTTCTTTTTTAGCACATATTCAGTGGATGGTTATGTT
CACACCTTTAGTACCTTTCTGGATAACAATTGCTTATATCATTTGTATTTCCACAAAGCATTTCTATTGGTTCTTTAGTAATTA
CCTAAAGAGACGTGTAGTCTTTAATGGTGTTTCCTTTAGTACTTTTGAAGAAGCTGCGCTGTGCACCTTTTTGTTAAATAAAGA
AATGTATCTAAAGTTGCGTAGTGATGTGCTATTACCTCTTACGCAATATAATAGATACTTAGCTCTTTATAATAAGTACAAGTA
TTTTAGTGGAGCAATGGATACAACTAGCTACAGAGAAGCTGCTTGTTGTCATCTCGCAAAGGCTCTCAATGACTTCAGTAACTC
AGGTTCTGATGTTCTTTACCAACCACCACAAACCTCTATCACCTCAGCTGTTTTGCAGAGTGGTTTTAGAAAAATGGCATTCCC
ATCTGGTAAAGTTGAGGGTTGTATGGTACAAGTAACTTGTGGTACAACTACACTTAACGGTCTTTGGCTTGATGACGTAGTTTA
CTGTCCAAGACATGTGATCTGCACCTCTGAAGACATGCTTAACCCTAATTATGAAGATTTACTCATTCGTAAGTCTAATCATAA
TTTCTTGGTACAGGCTGGTAATGTTCAACTCAGGGTTATTGGACATTCTATGCAAAATTGTGTACTTAAGCTTAAGGTTGATAC
AGCCAATCCTAAGACACCTAAGTATAAGTTTGTTCGCATTCAACCAGGACAGACTTTTTCAGTGTTAGCTTGTTACAATGGTTC
ACCATCTGGTGTTTACCAATGTGCTATGAGGCCCAATTTCACTATTAAGGGTTCATTCCTTAATGGTTCATGTGGTAGTGTTGG
TTTTAACATAGATTATGACTGTGTCTCTTTTTGTTACATGCACCATATGGAATTACCAACTGGAGTTCATGCTGGCACAGACTT
AGAAGGTAACTTTTATGGACCTTTTGTTGACAGGCAAACAGCACAAGCAGCTGGTACGGACACAACTATTACAGTTAATGTTTT
AGCTTGGTTGTACGCTGCTGTTATAAATGGAGACAGGTGGTTTCTCAATCGATTTACCACAACTCTTAATGACTTTAACCTTGT
GGCTATGAAGTACAATTATGAACCTCTAACACAAGACCATGTTGACATACTAGGACCTCTTTCTGCTCAAACTGGAATTGCCGT
TTTAGATATGTGTGCTTCATTAAAAGAATTACTGCAAAATGGTATGAATGGACGTACCATATTGGGTAGTGCTTTATTAGAAGA
TGAATTTACACCTTTTGATGTTGTTAGACAATGCTCAGGTGTTACTTTCCAAAGTGCAGTGAAAAGAACAATCAAGGGTACACA
CCACTGGTTGTTACTCACAATTTTGACTTCACTTTTAGTTTTAGTCCAGAGTACTCAATGGTCTTTGTTCTTTTTTTTGTATGA
AAATGCCTTTTTACCTTTTGCTATGGGTATTATTGCTATGTCTGCTTTTGCAATGATGTTTGTCAAACATAAGCATGCATTTCT
CTGTTTGTTTTTGTTACCTTCTCTTGCCACTGTAGCTTATTTTAATATGGTCTATATGCCTGCTAGTTGGGTGATGCGTATTAT
GACATGGTTGGATATGGTTGATACTAGTTTGTCTGGTTTTAAGCTAAAAGACTGTGTTATGTATGCATCAGCTGTAGTGTTACT
AATCCTTATGACAGCAAGAACTGTGTATGATGATGGTGCTAGGAGAGTGTGGACACTTATGAATGTCTTGACACTCGTTTATAA
AGTTTATTATGGTAATGCTTTAGATCAAGCCATTTCCATGTGGGCTCTTATAATCTCTGTTACTTCTAACTACTCAGGTGTAGT
TACAACTGTCATGTTTTTGGCCAGAGGTATTGTTTTTATGTGTGTTGAGTATTGCCCTATTTTCTTCATAACTGGTAATACACT
TCAGTGTATAATGCTAGTTTATTGTTTCTTAGGCTATTTTTGTACTTGTTACTTTGGCCTCTTTTGTTTACTCAACCGCTACTT
TAGACTGACTCTTGGTGTTTATGATTACTTAGTTTCTACACAGGAGTTTAGATATATGAATTCACAGGGACTACTCCCACCCAA
GAATAGCATAGATGCCTTCAAACTCAACATTAAATTGTTGGGTGTTGGTGGCAAACCTTGTATCAAAGTAGCCACTGTACAGTC
TAAAATGTCAGATGTAAAGTGCACATCAGTAGTCTTACTCTCAGTTTTGCAACAACTCAGAGTAGAATCATCATCTAAATTGTG
GGCTCAATGTGTCCAGTTACACAATGACATTCTCTTAGCTAAAGATACTACTGAAGCCTTTGAAAAAATGGTTTCACTACTTTC
TGTTTTGCTTTCCATGCAGGGTGCTGTAGACATAAACAAGCTTTGTGAAGAAATGCTGGACAACAGGGCAACCTTACAAGCTAT
AGCCTCAGAGTTTAGTTCCCTTCCATCATATGCAGCTTTTGCTACTGCTCAAGAAGCTTATGAGCAGGCTGTTGCTAATGGTGA
TTCTGAAGTTGTTCTTAAAAAGTTGAAGAAGTCTTTGAATGTGGCTAAATCTGAATTTGACCGTGATGCAGCCATGCAACGTAA
GTTGGAAAAGATGGCTGATCAAGCTATGACCCAAATGTATAAACAGGCTAGATCTGAGGACAAGAGGGCAAAAGTTACTAGTGC
TATGCAGACAATGCTTTTCACTATGCTTAGAAAGTTGGATAATGATGCACTCAACAACATTATCAACAATGCAAGAGATGGTTG
TGTTCCCTTGAACATAATACCTCTTACAACAGCAGCCAAACTAATGGTTGTCATACCAGACTATAACACATATAAAAATACGTG
TGATGGTACAACATTTACTTATGCATCAGCATTGTGGGAAATCCAACAGGTTGTAGATGCAGATAGTAAAATTGTTCAACTTAG
TGAAATTAGTATGGACAATTCACCTAATTTAGCATGGCCTCTTATTGTAACAGCTTTAAGGGCCAATTCTGCTGTCAAATTACA
GAATAATGAGCTTAGTCCTGTTGCACTACGACAGATGTCTTGTGCTGCCGGTACTACACAAACTGCTTGCACTGATGACAATGC
GTTAGCTTACTACAACACAACAAAGGGAGGTAGGTTTGTACTTGCACTGTTATCCGATTTACAGGATTTGAAATGGGCTAGATT
CCCTAAGAGTGATGGAACTGGTACTATCTATACAGAACTGGAACCACCTTGTAGGTTTGTTACAGACACACCTAAAGGTCCTAA
AGTGAAGTATTTATACTTTATTAAAGGATTAAACAACCTAAATAGAGGTATGGTACTTGGTAGTTTAGCTGCCACAGTACGTCT
ACAAGCTGGTAATGCAACAGAAGTGCCTGCCAATTCAACTGTATTATCTTTCTGTGCTTTTGCTGTAGATGCTGCTAAAGCTTA
CAAAGATTATCTAGCTAGTGGGGGACAACCAATCACTAATTGTGTTAAGATGTTGTGTACACACACTGGTACTGGTCAGGCAAT
AACAGTTACACCGGAAGCCAATATGGATCAAGAATCCTTTGGTGGTGCATCGTGTTGTCTGTACTGCCGTTGCCACATAGATCA
TCCAAATCCTAAAGGATTTTGTGACTTAAAAGGTAAGTATGTACAAATACCTACAACTTGTGCTAATGACCCTGTGGGTTTTAC
ACTTAAAAACACAGTCTGTACCGTCTGCGGTATGTGGAAAGGTTATGGCTGTAGTTGTGATCAACTCCGCGAACCCATGCTTCA
GTCAGCTGATGCACAATCGTTTTTAAACGGGTTTGCGGTGTAAGTGCAGCCCGTCTTACACCGTGCGGCACAGGCACTAGTACT
GATGTCGTATACAGGGCTTTTGACATCTACAATGATAAAGTAGCTGGTTTTGCTAAATTCCTAAAAACTAATTGTTGTCGCTTC
CAAGAAAAGGACGAAGATGACAATTTAATTGATTCTTACTTTGTAGTTAAGAGACACACTTTCTCTAACTACCAACATGAAGAA
ACAATTTATAATTTACTTAAGGATTGTCCAGCTGTTGCTAAACATGACTTCTTTAAGTTTAGAATAGACGGTGACATGGTACCA
CATATATCACGTCAACGTCTTACTAAATACACAATGGCAGACCTCGTCTATGCTTTAAGGCATTTTGATGAAGGTAATTGTGAC
ACATTAAAAGAAATACTTGTCACATACAATTGTTGTGATGATGATTATTTCAATAAAAAGGACTGGTATGATTTTGTAGAAAAC
CCAGATATATTACGCGTATACGCCAACTTAGGTGAACGTGTACGCCAAGCTTTGTTAAAAACAGTACAATTCTGTGATGCCATG
CGAAATGCTGGTATTGTTGGTGTACTGACATTAGATAATCAAGATCTCAATGGTAACTGGTATGATTTCGGTGATTTCATACAA
ACCACGCCAGGTAGTGGAGTTCCTGTTGTAGATTCTTATTATTCATTGTTAATGCCTATATTAACCTTGACCAGGGCTTTAACT
GCAGAGTCACATGTTGACACTGACTTAACAAAGCCTTACATTAAGTGGGATTTGTTAAAATATGACTTCACGGAAGAGAGGTTA
AAACTCTTTGACCGTTATTTTAAATATTGGGATCAGACATACCACCCAAATTGTGTTAACTGTTTGGATGACAGATGCATTCTG
CATTGTGCAAACTTTAATGTTTTATTCTCTACAGTGTTCCCACCTACAAGTTTTGGACCACTAGTGAGAAAAATATTTGTTGAT
GGTGTTCCATTTGTAGTTTCAACTGGATACCACTTCAGAGAGCTAGGTGTTGTACATAATCAGGATGTAAACTTACATAGCTCT
AGACTTAGTTTTAAGGAATTACTTGTGTATGCTGCTGACCCTGCTATGCACGCTGCTTCTGGTAATCTATTACTAGATAAACGC
ACTACGTGCTTTTCAGTAGCTGCACTTACTAACAATGTTGCTTTTCAAACTGTCAAACCCGGTAATTTTAACAAAGACTTCTAT
GACTTTGCTGTGTCTAAGGGTTTCTTTAAGGAAGGAAGTTCTGTTGAATTAAAACACTTCTTCTTTGCTCAGGATGGTAATGCT
GCTATCAGCGATTATGACTACTATCGTTATAATCTACCAACAATGTGTGATATCAGACAACTACTATTTGTAGTTGAAGTTGTT
GATAAGTACTTTGATTGTTACGATGGTGGCTGTATTAATGCTAACCAAGTCATCGTCAACAACCTAGACAAATCAGCTGGTTTT
CCATTTAATAAATGGGGTAAGGCTAGACTTTATTATGATTCAATGAGTTATGAGGATCAAGATGCACTTTTCGCATATACAAAA
CGTAATGTCATCCCTACTATAACTCAAATGAATCTTAAGTATGCCATTAGTGCAAAGAATAGAGCTCGCACCGTAGCTGGTGTC
TCTATCTGTAGTACTATGACCAATAGACAGTTTCATCAAAAATTATTGAAATCAATAGCCGCCACTAGAGGAGCTACTGTAGTA
ATTGGAACAAGCAAATTCTATGGTGGTTGGCACAACATGTTAAAAACTGTTTATAGTGATGTAGAAAACCCTCACCTTATGGGT
TGGGATTATCCTAAATGTGATAGAGCCATGCCTAACATGCTTAGAATTATGGCCTCACTTGTTCTTGCTCGCAAACATACAACG
TGTTGTAGCTTGTCACACCGTTTCTATAGATTAGCTAATGAGTGTGCTCAAGTATTGAGTGAAATGGTCATGTGTGGCGGTTCA
CTATATGTTAAACCAGGTGGAACCTCATCAGGAGATGCCACAACTGCTTATGCTAATAGTGTTTTTAACATTTGTCAAGCTGTC
ACGGCCAATGTTAATGCACTTTTATCTACTGATGGTAACAAAATTGCCGATAAGTATGTCCGCAATTTACAACACAGACTTTAT
GAGTGTCTCTATAGAAATAGAGATGTTGACACAGACTTTGTGAATGAGTTTTACGCATATTTGCGTAAACATTTCTCAATGATG
ATACTCTCTGACGATGCTGTTGTGTGTTTCAATAGCACTTATGCATCTCAAGGTCTAGTGGCTAGCATAAAGAACTTTAAGTCA
GTTCTTTATTATCAAAACAATGTTTTTATGTCTGAAGCAAAATGTTGGACTGAGACTGACCTTACTAAAGGACCTCATGAATTT
TGCTCTCAACATACAATGCTAGTTAAACAGGGTGATGATTATGTGTACCTTCCTTACCCAGATCCATCAAGAATCCTAGGGGCC
GGCTGTTTTGTAGATGATATCGTAAAAACAGATGGTACACTTATGATTGAACGGTTCGTGTCTTTAGCTATAGATGCTTACCCA
CTTACTAAACATCCTAATCAGGAGTATGCTGATGTCTTTCATTTGTACTTACAATACATAAGAAAGCTACATGATGAGTTAACA
GGACACATGTTAGACATGTATTCTGTTATGCTTACTAATGATAACACTTCAAGGTATTGGGAACCTGAGTTTTATGAGGCTATG
TACACACCGCATACAGTCTTACAGGCTGTTGGGGCTTGTGTTCTTTGCAATTCACAGACTTCATTAAGATGTGGTGCTTGCATA
CGTAGACCATTCTTATGTTGTAAATGCTGTTACGACCATGTCATATCAACATCACATAAATTAGTCTTGTCTGTTAATCCGTAT
GTTTGCAATGCTCCAGGTTGTGATGTCACAGATGTGACTCAACTTTACTTAGGAGGTATGAGCTATTATTGTAAATCACATAAA
CCACCCATTAGTTTTCCATTGTGTGCTAATGGACAAGTTTTTGGTTTATATAAAAATACATGTGTTGGTAGCGATAATGTTACT
GACTTTAATGCAATTGCAACATGTGACTGGACAAATGCTGGTGATTACATTTTAGCTAACACCTGTACTGAAAGACTCAAGCTT
TTTGCAGCAGAAACGCTCAAAGCTACTGAGGAGACATTTAAACTGTCTTATGGTATTGCTACTGTACGTGAAGTGCTGTCTGAC
AGAGAATTACATCTTTCATGGGAAGTTGGTAAACCTAGACCACCACTTAACCGAAATTATGTCTTTACTGGTTATCGTGTAACT
AAAAACAGTAAAGTACAAATAGGAGAGTACACCTTTGAAAAAGGTGACTATGGTGATGCTGTTGTTTACCGAGGTACAACAACT
TACAAATTAAATGTTGGTGATTATTTTGTGCTGACATCACATACAGTAATGCCATTAAGTGCACCTACACTAGTGCCACAAGAG
CACTATGTTAGAATTACTGGCTTATACCCAACACTCAATATCTCAGATGAGTTTTCTAGCAATGTTGCAAATTATCAAAAGGTT
GGTATGCAAAAGTATTCTACACTCCAGGGACCACCTGGTACTGGTAAGAGTCATTTTGCTATTGGCCTAGCTCTCTACTACCCT
TCTGCTCGCATAGTGTATACAGCTTGCTCTCATGCCGCTGTTGATGCACTATGTGAGAAGGCATTAAAATATTTGCCTATAGAT
AAATGTAGTAGAATTATACCTGCACGTGCTCGTGTAGAGTGTTTTGATAAATTCAAAGTGAATTCAACATTAGAACAGTATGTC
TTTTGTACTGTAAATGCATTGCCTGAGACGACAGCAGATATAGTTGTCTTTGATGAAATTTCAATGGCCACAAATTATGATTTG
AGTGTTGTCAATGCCAGATTACGTGCTAAGCACTATGTGTACATTGGCGACCCTGCTCAATTACCTGCACCACGCACATTGCTA
ACTAAGGGCACACTAGAACCAGAATATTTCAATTCAGTGTGTAGACTTATGAAAACTATAGGTCCAGACATGTTCCTCGGAACT
TGTCGGCGTTGTCCTGCTGAAATTGTTGACACTGTGAGTGCTTTGGTTTATGATAATAAGCTTAAAGCACATAAAGACAAATCA
GCTCAATGCTTTAAAATGTTTTATAAGGGTGTTATCACGCATGATGTTTCATCTGCAATTAACAGGCCACAAATAGGCGTGGTA
AGAGAATTCCTTACACGTAACCCTGCTTGGAGAAAAGCTGTCTTTATTTCACCTTATAATTCACAGAATGCTGTAGCCTCAAAG
ATTTTGGGACTACCAACTCAAACTGTTGATTCATCACAGGGCTCAGAATATGACTATGTCATATTCACTCAAACCACTGAAACA
GCTCACTCTTGTAATGTAAACAGATTTAATGTTGCTATTACCAGAGCAAAAGTAGGCATACTTTGCATAATGTCTGATAGAGAC
CTTTATGACAAGTTGCAATTTACAAGTCTTGAAATTCCACGTAGGAATGTGGCAACTTTACAAGCTGAAAATGTAACAGGACTC
TTTAAAGATTGTAGTAAGGTAATCACTGGGTTACATCCTACACAGGCACCTACACACCTCAGTGTTGACACTAAATTCAAAACT
GAAGGTTTATGTGTTGACATACCTGGCATACCTAAGGACATGACCTATAGAAGACTCATCTCTATGATGGGTTTTAAAATGAAT
TATCAAGTTAATGGTTACCCTAACATGTTTATCACCCGCGAAGAAGCTATAAGACATGTACGTGCATGGATTGGCTTCGATGTC
GAGGGGTGTCATGCTACTAGAGAAGCTGTTGGTACCAATTTACCTTTACAGCTAGGTTTTTCTACAGGTGTTAACCTAGTTGCT
GTACCTACAGGTTATGTTGATACACCTAATAATACAGATTTTTCCAGAGTTAGTGCTAAACCACCGCCTGGAGATCAATTTAAA
CACCTCATACCACTTATGTACAAAGGACTTCCTTGGAATGTAGTGCGTATAAAGATTGTACAAATGTTAAGTGACACACTTAAA
AATCTCTCTGACAGAGTCGTATTTGTCTTATGGGCACATGGCTTTGAGTTGACATCTATGAAGTATTTTGTGAAAATAGGACCT
GAGCGCACCTGTTGTCTATGTGATAGACGTGCCACATGCTTTTCCACTGCTTCAGACACTTATGCCTGTTGGCATCATTCTATT
GGATTTGATTACGTCTATAATCCGTTTATGATTGATGTTCAACAATGGGGTTTTACAGGTAACCTACAAAGCAACCATGATCTG
TATTGTCAAGTCCATGGTAATGCACATGTAGCTAGTTGTGATGCAATCATGACTAGGTGTCTAGCTGTCCACGAGTGCTTTGTT
AAGCGTGTTGACTGGACTATTGAATATCCTATAATTGGTGATGAACTGAAGATTAATGCGGCTTGTAGAAAGGTTCAACACATG
GTTGTTAAAGCTGCATTATTAGCAGACAAATTCCCAGTTCTTCACGACATTGGTAACCCTAAAGCTATTAAGTGTGTACCTCAA
GCTGATGTAGAATGGAAGTTCTATGATGCACAGCCTTGTAGTGACAAAGCTTATAAAATAGAAGAATTATTCTATTCTTATGCC
ACACATTCTGACAAATTCACAGATGGTGTATGCCTATTTTGGAATTGCAATGTCGATAGATATCCTGCTAATTCCATTGTTTGT
AGATTTGACACTAGAGTGCTATCTAACCTTAACTTGCCTGGTTGTGATGGTGGCAGTTTGTATGTAAATAAACATGCATTCCAC
ACACCAGCTTTTGATAAAAGTGCTTTTGTTAATTTAAAACAATTACCATTTTTCTATTACTCTGACAGTCCATGTGAGTCTCAT
GGAAAACAAGTAGTGTCAGATATAGATTATGTACCACTAAAGTCTGCTACGTGTATAACACGTTGCAATTTAGGTGGTGCTGTC
TGTAGACATCATGCTAATGAGTACAGATTGTATCTCGATGCTTATAACATGATGATCTCAGCTGGCTTTAGCTTGTGGGTTTAC
AAACAATTTGATACTTATAACCTCTGGAACACTTTTACAAGACTTCAGAGTTTAGAAAATGTGGCTTTTAATGTTGTAAATAAG
GGACACTTTGATGGACAACAGGGTGAAGTACCAGTTTCTATCATTAATAACACTGTTTACACAAAAGTTGATGGTGTTGATGTA
GAATTGTTTGAAAATAAAACAACATTACCTGTTAATGTAGCATTTGAGCTTTGGGCTAAGCGCAACATTAAACCAGTACCAGAG
GTGAAAATACTCAATAATTTGGGTGTGGACATTGCTGCTAATACTGTGATCTGGGACTACAAAAGAGATGCTCCAGCACATATA
TCTACTATTGGTGTTTGTTCTATGACTGACATAGCCAAGAAACCAACTGAAACGATTTGTGCACCACTCACTGTCTTTTTTGAT
GGTAGAGTTGATGGTCAAGTAGACTTATTTAGAAATGCCCGTAATGGTGTTCTTATTACAGAAGGTAGTGTTAAAGGTTTACAA
CCATCTGTAGGTCCCAAACAAGCTAGTCTTAATGGAGTCACATTAATTGGAGAAGCCGTAAAAACACAGTTCAATTATTATAAG
AAAGTTGATGGTGTTGTCCAACAATTACCTGAAACTTACTTTACTCAGAGTAGAAATTTACAAGAATTTAAACCCAGGAGTCAA
ATGGAAATTGATTTCTTAGAATTAGCTATGGATGAATTCATTGAACGGTATAAATTAGAAGGCTATGCCTTCGAACATATCGTT
TATGGAGATTTTAGTCATAGTCAGTTAGGTGGTTTACATCTACTGATTGGACTAGCTAAACGTTTTAAGGAATCACCTTTTGAA
TTAGAAGATTTTATTCCTATGGACAGTACAGTTAAAAACTATTTCATAACAGATGCGCAAACAGGTTCATCTAAGTGTGTGTGT
TCTGTTATTGATTTATTACTTGATGATTTTGTTGAAATAATAAAATCCCAAGATTTATCTGTAGTTTCTAAGGTTGTCAAAGTG
ACTATTGACTATACAGAAATTTCATTTATGCTTTGGTGTAAAGATGGCCATGTAGAAACATTTTACCCAAAATTACAATCTAGT
CAAGCGTGGCAACCGGGTGTTGCTATGCCTAATCTTTACAAAATGCAAAGAATGCTATTAGAAAAGTGTGACCTTCAAAATTAT
GGTGATAGTGCAACATTACCTAAAGGCATAATGATGAATGTCGCAAAATATACTCAACTGTGTCAATATTTAAACACATTAACA
TTAGCTGTACCCTATAATATGAGAGTTATACATTTTGGTGCTGGTTCTGATAAAGGAGTTGCACCAGGTACAGCTGTTTTAAGA
CAGTGGTTGCCTACGGGTACGCTGCTTGTCGATTCAGATCTTAATGACTTTGTCTCTGATGCAGATTCAACTTTGATTGGTGAT
TGTGCAACTGTACATACAGCTAATAAATGGGATCTCATTATTAGTGATATGTACGACCCTAAGACTAAAAATGTTACAAAAGAA
AATGACTCTAAAGAGGGTTTTTTCACTTACATTTGTGGGTTTATACAACAAAAGCTAGCTCTTGGAGGTTCCGTGGCTATAAAG
ATAACAGAACATTCTTGGAATGCTGATCTTTATAAGCTCATGGGACACTTCGCATGGTGGACAGCCTTTGTTACTAATGTGAAT
GCGTCATCATCTGAAGCATTTTTAATTGGATGTAATTATCTTGGCAAACCACGCGAACAAATAGATGGTTATGTCATGCATGCA
AATTACATATTTTGGAGGAATACAAATCCAATTCAGTTGTCTTCCTATTCTTTATTTGACATGAGTAAATTTCCCCTTAAATTA
AGGGGTACTGCTGTTATGTCTTTAAAAGAAGGTCAAATCAATGATATGATTTTATCTCTTCTTAGTAAAGGTAGACTTATAATT
AGAGAAAACAACAGAGTTGTTATTTCTAGTGATGTTCTTGTTAACAACTAAACGAACAATGTTTGTTTTTCTTGTTTTATTGCC
ACTAGTCTCTAGTCAGTGTGTTAATCTTACAACCAGAACTCAATTACCCCCTGCATACACTAATTCTTTCACACGTGGTGTTTA
TTACCCTGACAAAGTTTTCAGATCCTCAGTTTTACATTCAACTCAGGACTTGTTCTTACCTTTCTTTTCCAATGTTACTTGGTT
CCATGCTATACATGTCTCTGGGACCAATGGTACTAAGAGGTTTGATAACCCTGTCCTACCATTTAATGATGGTGTTTATTTTGC
TTCCACTGAGAAGTCTAACATAATAAGAGGCTGGATTTTTGGTACTACTTTAGATTCGAAGACCCAGTCCCTACTTATTGTTAA
TAACGCTACTAATGTTGTTATTAAAGTCTGTGAATTTCAATTTTGTAATGATCCATTTTTGGGTGTTTATTACCACAAAAACAA
CAAAAGTTGGATGGAAAGTGAGTTCAGAGTTTATTCTAGTGCGAATAATTGCACTTTTGAATATGTCTCTCAGCCTTTTCTTAT
GGACCTTGAAGGAAAACAGGGTAATTTCAAAAATCTTAGGGAATTTGTGTTTAAGAATATTGATGGTTATTTTAAAATATATTC
TAAGCACACGCCTATTAATTTAGTGCGTGATCTCCCTCAGGGTTTTTCGGCTTTAGAACCATTGGTAGATTTGCCAATAGGTAT
TAACATCACTAGGTTTCAAACTTTACTTGCTTTACATAGAAGTTATTTGACTCCTGGTGATTCTTCTTCAGGTTGGACAGCTGG
TGCTGCAGCTTATTATGTGGGTTATCTTCAACCTAGGACTTTTCTATTAAAATATAATGAAAATGGAACCATTACAGATGCTGT
AGACTGTGCACTTGACCCTCTCTCAGAAACAAAGTGTACGTTGAAATCCTTCACTGTAGAAAAAGGAATCTATCAAACTTCTAA
CTTTAGAGTCCAACCAACAGAATCTATTGTTAGATTTCCTAATATTACAAACTTGTGCCCTTTTGGTGAAGTTTTTAACGCCAC
CAGATTTGCATCTGTTTATGCTTGGAACAGGAAGAGAATCAGCAACTGTGTTGCTGATTATTCTGTCCTATATAATTCCGCATC
ATTTTCCACTTTTAAGTGTTATGGAGTGTCTCCTACTAAATTAAATGATCTCTGCTTTACTAATGTCTATGCAGATTCATTTGT
AATTAGAGGTGATGAAGTCAGACAAATCGCTCCAGGGCAAACTGGAAAGATTGCTGATTATAATTATAAATTACCAGATGATTT
TACAGGCTGCGTTATAGCTTGGAATTCTAACAATCTTGATTCTAAGGTTGGTGGTAATTATAATTACCTGTATAGATTGTTTAG
GAAGTCTAATCTCAAACCTTTTGAGAGAGATATTTCAACTGAAATCTATCAGGCCGGTAGCACACCTTGTAATGGTGTTGAAGG
TTTTAATTGTTACTTTCCTTTACAATCATATGGTTTCCAACCCACTAATGGTGTTGGTTACCAACCATACAGAGTAGTAGTACT
TTCTTTTGAACTTCTACATGCACCAGCAACTGTTTGTGGACCTAAAAAGTCTACTAATTTGGTTAAAAACAAATGTGTCAATTT
CAACTTCAATGGTTTAACAGGCACAGGTGTTCTTACTGAGTCTAACAAAAAGTTTCTGCCTTTCCAACAATTTGGCAGAGACAT
TGCTGACACTACTGATGCTGTCCGTGATCCACAGACACTTGAGATTCTTGACATTACACCATGTTCTTTTGGTGGTGTCAGTGT
TATAACACCAGGAACAAATACTTCTAACCAGGTTGCTGTTCTTTATCAGGATGTTAACTGCACAGAAGTCCCTGTTGCTATTCA
TGCAGATCAACTTACTCCTACTTGGCGTGTTTATTCTACAGGTTCTAATGTTTTTCAAACACGTGCAGGCTGTTTAATAGGGGC
TGAACATGTCAACAACTCATATGAGTGTGACATACCCATTGGTGCAGGTATATGCGCTAGTTATCAGACTCAGACTAATTCTCC
TCGGCGGGCACGTAGTGTAGCTAGTCAATCCATCATTGCCTACACTATGTCACTTGGTGCAGAAAATTCAGTTGCTTACTCTAA
TAACTCTATTGCCATACCCACAAATTTTACTATTAGTGTTACCACAGAAATTCTACCAGTGTCTATGACCAAGACATCAGTAGA
TTGTACAATGTACATTTGTGGTGATTCAACTGAATGCAGCAATCTTTTGTTGCAATATGGCAGTTTTTGTACACAATTAAACCG
TGCTTTAACTGGAATAGCTGTTGAACAAGACAAAAACACCCAAGAAGTTTTTGCACAAGTCAAACAAATTTACAAAACACCACC
AATTAAAGATTTTGGTGGTTTTAATTTTTCACAAATATTACCAGATCCATCAAAACCAAGCAAGAGGTCATTTATTGAAGATCT
ACTTTTCAACAAAGTGACACTTGCAGATGCTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATGAAATGATTGCTCAATACACTTCTGCACT
GTTAGCGGGTACAATCACTTCTGGTTGGACCTTTGGTGCAGGTGCTGCATTACAAATACCATTTGCTATGCAAATGGCTTATAG
GTTTAATGGTATTGGAGTTACACAGAATGTTCTCTATGAGAACCAAAAATTGATTGCCAACCAATTTAATAGTGCTATTGGCAA
AATTCAAGACTCACTTTCTTCCACAGCAAGTGCACTTGGAAAACTTCAAGATGTGGTCAACCAAAATGCACAAGCTTTAAACAC
GCTTGTTAAACAACTTAGCTCCAATTTTGGTGCAATTTCAAGTGTTTTAAATGATATCCTTTCACGTCTTGACAAAGTTGAGGC
TGAAGTGCAAATTGATAGGTTGATCACAGGCAGACTTCAAAGTTTGCAGACATATGTGACTCAACAATTAATTAGAGCTGCAGA
AATCAGAGCTTCTGCTAATCTTGCTGCTACTAAAATGTCAGAGTGTGTACTTGGACAATCAAAAAGAGTTGATTTTTGTGGAAA
GGGCTATCATCTTATGTCCTTCCCTCAGTCAGCACCTCATGGTGTAGTCTTCTTGCATGTGACTTATGTCCCTGCACAAGAAAA
GAACTTCACAACTGCTCCTGCCATTTGTCATGATGGAAAAGCACACTTTCCTCGTGAAGGTGTCTTTGTTTCAAATGGCACACA
CTGGTTTGTAACACAAAGGAATTTTTATGAACCACAAATCATTACTACAGACAACACATTTGTGTCTGGTAACTGTGATGTTGT
AATAGGAATTGTCAACAACACAGTTTATGATCCTTTGCAACCTGAATTAGACTCATTCAAGGAGGAGTTAGATAAATATTTTAA
GAATCATACATCACCAGATGTTGATTTAGGTGACATCTCTGGCATTAATGCTTCAGTTGTAAACATTCAAAAAGAAATTGACCG
CCTCAATGAGGTTGCCAAGAATTTAAATGAATCTCTCATCGATCTCCAAGAACTTGGAAAGTATGAGCAGTATATAAAATGGCC
ATGGTACATTTGGCTAGGTTTTATAGCTGGCTTGATTGCCATAGTAATGGTGACAATTATGCTTTGCTGTATGACCAGTTGCTG
TAGTTGTCTCAAGGGCTGTTGTTCTTGTGGATCCTGCTGCAAATTTGATGAAGACGACTCTGAGCCAGTGCTCAAAGGAGTCAA
ATTACATTACACATAAACGAACTTATGGATTTGTTTATGAGAATCTTCACAATTGGAACTGTAACTTTGAAGCAAGGTGAAATC
AAGGATGCTACTCCTTCAGATTTTGTTCGCGCTACTGCAACGATACCGATACAAGCCTCACTCCCTTTCGGATGGCTTATTGTT
GGCGTTGCACTTCTTGCTGTTTTTCAGAGCGCTTCCAAAATCATAACCCTCAAAAAGAGATGGCAACTAGCACTCTCCAAGGGT
GTTCACTTTGTTTGCAACTTGCTGTTGTTGTTTGTAACAGTTTACTCACACCTTTTGCTCGTTGCTGCTGGCCTTGAAGCCCCT
TTTCTCTATCTTTATGCTTTAGTCTACTTCTTGCAGAGTATAAACTTTGTAAGAATAATAATGAGGCTTTGGCTTTGCTGGAAA
TGCCGTTCCAAAAACCCATTACTTTATGATGCCAACTATTTTCTTTGCTGGCATACTAATTGTTACGACTATTGTATACCTTAC
AATAGTGTAACTTCTTCAATTGTCATTACTTCAGGTGATGGCACAACAAGTCCTATTTCTGAACATGACTACCAGATTGGTGGT
TATACTGAAAAATGGGAATCTGGAGTAAAAGACTGTGTTGTATTACACAGTTACTTCACTTCAGACTATTACCAGCTGTACTCA
ACTCAATTGAGTACAGACACTGGTGTTGAACATGTTACCTTCTTCATCTACAATAAAATTGTTGATGAGCCTGAAGAACATGTC
CAAATTCACACAATCGACGGTTCATCCGGAGTTGTTAATCCAGTAATGGAACCAATTTATGATGAACCGACGACGACTACTAGC
GTGCCTTTGTAAGCACAAGCTGATGAGTACGAACTTATGTACTCATTCGTTTCGGAAGAGACAGGTACGTTAATAGTTAATAGC
GTACTTCTTTTTCTTGCTTTCGTGGTATTCTTGCTAGTTACACTAGCCATCCTTACTGCGCTTCGATTGTGTGCGTACTGCTGC
AATATTGTTAACGTGAGTCTTGTAAAACCTTCTTTTTACGTTTACTCTCGTGTTAAAAATCTGAATTCTTCTAGAGTTCCTGAT
CTTCTGGTCTAAACGAACTAAATATTATATTAGTTTTTCTGTTTGGAACTTTAATTTTAGCCATGGCAGATTCCAACGGTACTA
TTACCGTTGAAGAGCTTAAAAAGCTCCTTGAACAATGGAACCTAGTAATAGGTTTCCTATTCCTTACATGGATTTGTCTTCTAC
AATTTGCCTATGCCAACAGGAATAGGTTTTTGTATATAATTAAGTTAATTTTCCTCTGGCTGTTATGGCCAGTAACTTTAGCTT
GTTTTGTGCTTGCTGCTGTTTACAGAATAAATTGGATCACCGGTGGAATTGCTATCGCAATGGCTTGTCTTGTAGGCTTGATGT
GGCTCAGCTACTTCATTGCTTCTTTCAGACTGTTTGCGCGTACGCGTTCCATGTGGTCATTCAATCCAGAAACTAACATTCTTC
TCAACGTGCCACTCCATGGCACTATTCTGACCAGACCGCTTCTAGAAAGTGAACTCGTAATCGGAGCTGTGATCCTTCGTGGAC
ATCTTCGTATTGCTGGACACCATCTAGGACGCTGTGACATCAAGGACCTGCCTAAAGAAATCACTGTTGCTACATCACGAACGC
TTTCTTATTACAAATTGGGAGCTTCGCAGCGTGTAGCAGGTGACTCAGGTTTTGCTGCATACAGTCGCTACAGGATTGGCAACT
ATAAATTAAACACAGACCATTCCAGTAGCAGTGACAATATTGCTTTGCTTGTACAGTAAGTGACAACAGATGTTTCATCTCGTT
GACTTTCAGGTTACTATAGCAGAGATATTACTAATTATTATGAGGACTTTTAAAGTTTCCATTTGGAATCTTGATTACATCATA
AACCTCATAATTAAAAATTTATCTAAGTCACTAACTGAGAATAAATATTCTCAATTAGATGAAGAGCAACCAATGGAGATTGAT
TAAACGAACATGAAAATTATTCTTTTCTTGGCACTGATAACACTCGCTACTTGTGAGCTTTATCACTACCAAGAGTGTGTTAGA
GGTACAACAGTACTTTTAAAAGAACCTTGCTCTTCTGGAACATACGAGGGCAATTCACCATTTCATCCTCTAGCTGATAACAAA
TTTGCACTGACTTGCTTTAGCACTCAATTTGCTTTTGCTTGTCCTGACGGCGTAAAACACGTCTATCAGTTACGTGCCAGATCA
GTTTCACCTAAACTGTTCATCAGACAAGAGGAAGTTCAAGAACTTTACTCTCCAATTTTTCTTATTGTTGCGGCAATAGTGTTT
ATAACACTTTGCTTCACACTCAAAAGAAAGACAGAATGATTGAACTTTCATTAATTGACTTCTATTTGTGCTTTTTAGCCTTTC
TGCTATTCCTTGTTTTAATTATGCTTATTATCTTTTGGTTCTCACTTGAACTGCAAGATCATAATGAAACTTGTCACGCCTAAA
CGAACATGAAATTTCTTGTTTTCTTAGGAATCATCACAACTGTAGCTGCATTTCACCAAGAATGTAGTTTACAGTCATGTACTC
AACATCAACCATATGTAGTTGATGACCCGTGTCCTATTCACTTCTATTCTAAATGGTATATTAGAGTAGGAGCTAGAAAATCAG
CACCTTTAATTGAATTGTGCGTGGATGAGGCTGGTTCTAAATCACCCATTCAGTACATCGATATCGGTAATTATACAGTTTCCT
GTTTACCTTTTACAATTAATTGCCAGGAACCTAAATTGGGTAGTCTTGTAGTGCGTTGTTCGTTCTATGAAGACTTTTTAGAGT
ATCATGACGTTCGTGTTGTTTTAGATTTCATCTAAACGAACAAACTAAAATGTCTGATAATGGACCCCAAAATCAGCGAAATGC
ACCCCGCATTACGTTTGGTGGACCCTCAGATTCAACTGGCAGTAACCAGAATGGAGAACGCAGTGGGGCGCGATCAAAACAACG
TCGGCCCCAAGGTTTACCCAATAATACTGCGTCTTGGTTCACCGCTCTCACTCAACATGGCAAGGAAGACCTTAAATTCCCTCG
AGGACAAGGCGTTCCAATTAACACCAATAGCAGTCCAGATGACCAAATTGGCTACTACCGAAGAGCTACCAGACGAATTCGTGG
TGGTGACGGTAAAATGAAAGATCTCAGTCCAAGATGGTATTTCTACTACCTAGGAACTGGGCCAGAAGCTGGACTTCCCTATGG
TGCTAACAAAGACGGCATCATATGGGTTGCAACTGAGGGAGCCTTGAATACACCAAAAGATCACATTGGCACCCGCAATCCTGC
TAACAATGCTGCAATCGTGCTACAACTTCCTCAAGGAACAACATTGCCAAAAGGCTTCTACGCAGAAGGGAGCAGAGGCGGCAG
TCAAGCCTCTTCTCGTTCCTCATCACGTAGTCGCAACAGTTCAAGAAATTCAACTCCAGGCAGCAGTAGGGGAACTTCTCCTGC
TAGAATGGCTGGCAATGGCGGTGATGCTGCTCTTGCTTTGCTGCTGCTTGACAGATTGAACCAGCTTGAGAGCAAAATGTCTGG
TAAAGGCCAACAACAACAAGGCCAAACTGTCACTAAGAAATCTGCTGCTGAGGCTTCTAAGAAGCCTCGGCAAAAACGTACTGC
CACTAAAGCATACAATGTAACACAAGCTTTCGGCAGACGTGGTCCAGAACAAACCCAAGGAAATTTTGGGGACCAGGAACTAAT
CAGACAAGGAACTGATTACAAACATTGGCCGCAAATTGCACAATTTGCCCCCAGCGCTTCAGCGTTCTTCGGAATGTCGCGCAT
TGGCATGGAAGTCACACCTTCGGGAACGTGGTTGACCTACACAGGTGCCATCAAATTGGATGACAAAGATCCAAATTTCAAAGA
TCAAGTCATTTTGCTGAATAAGCATATTGACGCATACAAAACATTCCCACCAACAGAGCCTAAAAAGGACAAAAAGAAGAAGGC
TGATGAAACTCAAGCCTTACCGCAGAGACAGAAGAAACAGCAAACTGTGACTCTTCTTCCTGCTGCAGATTTGGATGATTTCTC
CAAACAATTGCAACAATCCATGAGCAGTGCTGACTCAACTCAGGCCTAAACTCATGCAGACCACACAAGGCAGATGGGCTATAT
AAACGTTTTCGCTTTTCCGTTTACGATATATAGTCTACTCTTGTGCAGAATGAATTCTCGTAACTACATAGCACAAGTAGATGT
AGTTAACTTTAATCTCACATAGCAATCTTTAATCAGTGTGTAACATTAGGGAGGACTTGAAAGAGCCACCACATTTTCACCGAG
GCCACGCGGAGTACGATCGAGTGTACAGTGAACAATGCTAGGGAGAGCTGCCTATATGGAAGAGCCCTAATGTGTAAAATTAAT
TTTAGTAGTGCTATCCCCATGTGATTTTAATAGCTTCTTAGGAGAATGACAAAAAAAAAAAAAAAAAAAAAAAAAAAAAAAAA.

The viral genome of SARS-CoV-2 comprises multiple genes that can be targeted by the system and method as disclosed herein, such as the S gene, the N gene, or the E gene. In further embodiments, an open reading frame that encodes a peptide and is a fragment of the gene may be targeted by the system and method as disclosed herein.

In some embodiments, the SARS-CoV-2 gene comprises, or consists essentially of, or yet further consists of an ORF1ab gene. In further embodiments, the OR-Flab gene comprises, or consists essentially of, or yet further consists of nt 266 to nt 21555 of SEQ ID NO: 1. In yet further embodiments, the ORF1ab gene encodes: a leader protein (NCBI Reference Sequence: YP_009725297.1, which is also referred to as nsp1, encoded by nt 266 to nt 805 of SEQ ID NO: 1), nsp2 (NCBI Reference Sequence: YP_009725298.1, encoded by nt 806 to nt 2719 of SEQ ID NO: 1), nsp3 (NCBI Reference Sequence: YP_009725299.1, encoded by nt 2720 to nt 8554 of SEQ ID NO: 1), nsp4 (NCBI Reference Sequence: YP_009725300.1, encoded by nt 8555 to nt 10054 of SEQ ID NO: 1), 3C-like proteinase (NCBI Reference Sequence: YP_009725301.1, encoded by nt 10055 to nt 10972 of SEQ ID NO: 1), nsp6 (NCBI Reference Sequence: YP_009725302.1, encoded by nt 10973 to nt 11842 of SEQ ID NO: 1), nsp7 (NCBI Reference Sequence: YP_009725303.1, encoded by nt 11843 to nt 12091 of SEQ ID NO: 1), nsp8 (NCBI Reference Sequence: YP_009725304.1, encoded by nt 12092 to nt 12685 of SEQ ID NO: 1), nsp9 (NCBI Reference Sequence: YP_009725305.1, encoded by nt 12686 to nt 13024 of SEQ ID NO: 1), nsp10 (NCBI Reference Sequence: YP_009725306.1, encoded by nt 13025 to nt 13441 of SEQ ID NO: 1), nsp12 (NCBI Reference Sequence: YP_009725307.1, encoded by nt 13442 to nt 13468 and nt 13468 to nt 16236 of SEQ ID NO: 1), nsp13 (NCBI Reference Sequence: YP_009725308.1, encoded by nt 16237 to nt 18039 and nt 13468 to nt 16236 of SEQ ID NO: 1), 3′-to-5′ exonuclease (NCBI Reference Sequence: YP_009725309.1, encoded by nt 18040 to nt 19620 of SEQ ID NO: 1), endoRNAse (NCBI Reference Sequence: YP_009725310.1, encoded by nt 19621 to nt 20658 of SEQ ID NO: 1), or 2′-O-ribose methyltransferase (NCBI Reference Sequence: YP_009725311.1, encoded by nt 20659 to nt 21552 of SEQ ID NO: 1). In some embodiments, the ORF1ab gene comprises, or consists essentially of, or yet further consists of nt 266 to nt 13483 of SEQ ID NO: 1. In further embodiments, the ORF1ab gene encodes leader protein (NCBI Reference Sequence: YP_009742608.1, encoded by nt 266 to nt 805 of SEQ ID NO: 1), nsp2 (NCBI Reference Sequence: YP_009742609.1, encoded by nt 806 to nt 2719 of SEQ ID NO: 1), nsp3 (NCBI Reference Sequence: YP_009742610.1, encoded by nt 2720 to nt 8554 of SEQ ID NO: 1), nsp4 (NCBI Reference Sequence: YP_009742611.1, encoded by nt 8555 to nt 10054 of SEQ ID NO: 1), 3C-like proteinase (NCBI Reference Sequence: YP_009742612.1, encoded by nt 10055 to nt 10972 of SEQ ID NO: 1), nsp6 (NCBI Reference Sequence: YP_009742613.1, encoded by nt 10973 to nt 11842 of SEQ ID NO: 1), nsp7 (NCBI Reference Sequence: YP_009742614.1, encoded by nt 11843 to nt 12091 of SEQ ID NO: 1), nps8 (NCBI Reference Sequence: YP_009742615.1, encoded by nt 12092 to nt 12685 of SEQ ID NO: 1), nsp9 (NCBI Reference Sequence: YP_009742616.1, encoded by nt 12686 to nt 13024 of SEQ ID NO: 1), nsp10 (NCBI Reference Sequence: YP_009742617.1, encoded by nt 13025 to nt 13441 of SEQ ID NO: 1), or nsp11 (NCBI Reference Sequence: YP_009725312.1, encoded by nt 13442 to nt 13480 of SEQ ID NO: 1).

In some embodiments, the SARS-CoV-2 gene comprises, or consists essentially of, or yet further consists of an S gene. In further embodiments, the S gene comprises, or consists essentially of, or yet further consists of nt 21563 to nt 25384 of SEQ ID NO: 1. In yet further embodiments, the S gene encodes a spike (S) glycoprotein (NCBI Reference Sequence: YP_009724390.1).

In some embodiments, the SARS-CoV-2 gene comprises, or consists essentially of, or yet further consists of an ORF3a gene. In further embodiments, the ORF3a gene comprises, or consists essentially of, or yet further consists of nt 25393 to nt 26220 of SEQ ID NO: 1. In yet further embodiments, the ORF3a gene encodes an ORF3a protein (NCBI Reference Sequence: YP_009724391.1).

In some embodiments, the SARS-CoV-2 gene comprises, or consists essentially of, or yet further consists of an E gene. In further embodiments, the E gene comprises, or consists essentially of, or yet further consists of nt 26245 to nt 26472 of SEQ ID NO: 1. In yet further embodiments, the E gene encodes an envelope (E) protein (NCBI Reference Sequence: YP_009724392.1).

In some embodiments, the SARS-CoV-2 gene comprises, or consists essentially of, or yet further consists of an M gene. In further embodiments, the M gene comprises, or consists essentially of, or yet further consists of nt 26523 to nt 27191 of SEQ ID NO: 1. In yet further embodiments, the M gene encodes a membrane (M) glycoprotein (NCBI Reference Sequence: YP_009724393.1).

In some embodiments, the SARS-CoV-2 gene comprises, or consists essentially of, or yet further consists of an ORF6 gene. In further embodiments, the ORF6 gene comprises, or consists essentially of, or yet further consists of nt 27202 to nt 27387 of SEQ ID NO: 1. In yet further embodiments, the ORF6 gene encodes an ORF6 protein (NCBI Reference Sequence: YP_009724394.1).

In some embodiments, the SARS-CoV-2 gene comprises, or consists essentially of, or yet further consists of an ORF7a gene. In further embodiments, the ORF7a gene comprises, or consists essentially of, or yet further consists of nt 27394 to nt 27759 of SEQ ID NO: 1. In yet further embodiments, the ORF7a gene encodes an ORF7a protein (NCBI Reference Sequence: YP_009724395.1).

In some embodiments, the SARS-CoV-2 gene comprises, or consists essentially of, or yet further consists of an ORF7b gene. In further embodiments, the ORF7b gene comprises, or consists essentially of, or yet further consists of nt 27756 to nt 27887 of SEQ ID NO: 1. In yet further embodiments, the ORF7b gene encodes an ORF7b protein (NCBI Reference Sequence: YP_009725318.1).

In some embodiments, the SARS-CoV-2 gene comprises, or consists essentially of, or yet further consists of an ORF8 gene. In further embodiments, the ORF8 gene comprises, or consists essentially of, or yet further consists of nt 27894 to nt 28259 of SEQ ID NO: 1. In yet further embodiments, the ORF8 gene encodes an ORF8 protein (NCBI Reference Sequence: YP_009724396.1).

In some embodiments, the SARS-CoV-2 gene comprises, or consists essentially of, or yet further consists of an N gene. In further embodiments, the N gene comprises, or consists essentially of, or yet further consists of nt 28274 to nt 29533 of SEQ ID NO: 1. In yet further embodiments, the N gene encodes an N protein (NCBI Reference Sequence: YP_009724397.2).

In some embodiments, the SARS-CoV-2 gene comprises, or consists essentially of, or yet further consists of an ORF10 gene. In further embodiments, the ORF10 gene comprises, or consists essentially of, or yet further consists of nt 29558 to nt 29674 of SEQ ID NO: 1. In yet further embodiments, the ORF10 gene encodes an ORF10 protein (NCBI Reference Sequence: YP_009725255.1).

As used herein, vaccine refers to a substance, such as a peptide or a polynucleotide, used to stimulate an immune response, such as production of antibodies, and provide immunity against one or several diseases. Vaccination or a grammatical variation thereof refers to administration of a vaccine to a subject to help the immune system develop protection from a disease.

As used herein, the term “sample” and “biological sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen. In some embodiments, the sample is a swab sample, such as an anterior nasal swab sample, a pharyngeal swab sample, or an anal swab sample. In further embodiments, the sample is a buffer that immersed the swab. In some embodiments, the sample is a sputum sample. In some embodiments, the sample is a stool sample.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

As used herein, the term “library” when used in context of a nucleic acid refers to a collection of nucleic acids used for a specified use. Generally, the term “construct” and “vector” are used interchangeably herein to refer to a recombinant vector that retains the ability to infect and transduce non-dividing and/or slowly-dividing cells and, optionally, integrate into the target cell's genome. The vector may be derived from a virus, such as a lentivirus. Libraries generally consist of multiple vectors.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, (3-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, CASCADE BLUE™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “reporting reagent” refers to a reagent which is able to generate a detectable signal (such as fluorescence appearance/disappearance or color change) when a polynucleotide in the sample is cleaved by a CRISPR enzyme.

CRISPR enzyme in a complex with guide is activated upon binding to its target and subsequently cleaves any nearby ssRNA (i.e. “collateral” or “bystander” effects). It is shown here that a Cas13 enzyme as disclosed herein, once primed by its target, can cleave other (non-complementary) RNA molecules. Accordingly, the non-complementary RNA (referred to herein as a probe or a collateral cleavage probe) can be used as a reporting reagent. In some embodiments, the reporting reagent comprises, or consists essentially of, or yet further consists of a probe and a purification or detectable marker that generates a detectable signal once the probe is cleaved.

One non-limiting example of the reporting reagent is a probe conjugated to a fluorescence marker and a quencher (for example at the two opposite terminus of the probe). Prior to the cleavage of the probe, the quencher is close enough to absorb, decrease or abolish the fluorescent signal generated by the fluorescence marker (i.e., the quencher is in close proximity to the fluorescence marker). Furthermore, after the probe cleavage, the fluorescence marker and the quencher are with different cleaved products of the probe. Accordingly, when a target is present to activate the Cas13 enzyme, such enzyme cleaves the probe, releases the fluorescence marker from the close proximity of the quencher, and thus generates a detectable fluorescent signal. In some embodiments, the fluorescence marker is a fluorophore, such as 6-FAM (also referred to as 6-Carboxyfluorescein) or any one listed in www.thermofisher.com/us/en/home/life-science/cell-analysis/fluorophores.html accessible on May 3, 2021, www.abcam.com/ps/pdf/protocols/Fluorophore%20table.pdf accessible on May 3, 2021, or www.bio-rad.com/webroot/web/pdf/lsr/literature/Bulletin_2421.pdf on May 3, 2021. Additionally or alternatively, the quencher is an IOWA BLACK® quencher, such as IABkFQ (IOWA BLACK® quencher FQ), IOWA BLACK® quencher RQ, Dabsyl (dimethylaminoazobenzenesulfonic acid), Black Hole Quenchers, Qxl quenchers, or IRDye QC-1.

One non-limiting example of the reporting reagent is a probe conjugated to a detectable or purification marker and a binding moiety (for example at the two opposite terminus of the probe). After the probe cleavage, the detectable or purification marker and the binding moiety are with different cleaved products of the probe. Furthermore, the ligand of the binding moiety is used to catch the probe (if not cleaved) or the cleaved product comprising the binding moiety (if cleaved). Accordingly, when a target is present to activate the Cas13 enzyme, such enzyme cleaves the probe, and thus the ligand catches the cleaved product not comprising the detectable or purification marker, while the ligand catches the probe having the detectable or purification marker indicates there is no target. In some embodiments, the binding moiety is biotin. In further embodiments, the ligand is streptavidin. In yet further embodiments, the detectable or purification marker is a protein which can be recognized by an antibody conjugated to a colored particle (such as latex particle or gold nanoparticle).

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

A “composition” as used herein, refers to an active agent, such as a compound as disclosed herein and a carrier, inert or active. The carrier can be, without limitation, solid such as a bead or resin, or liquid, such as phosphate buffered saline

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

### Modes for Carrying Out the Disclosure

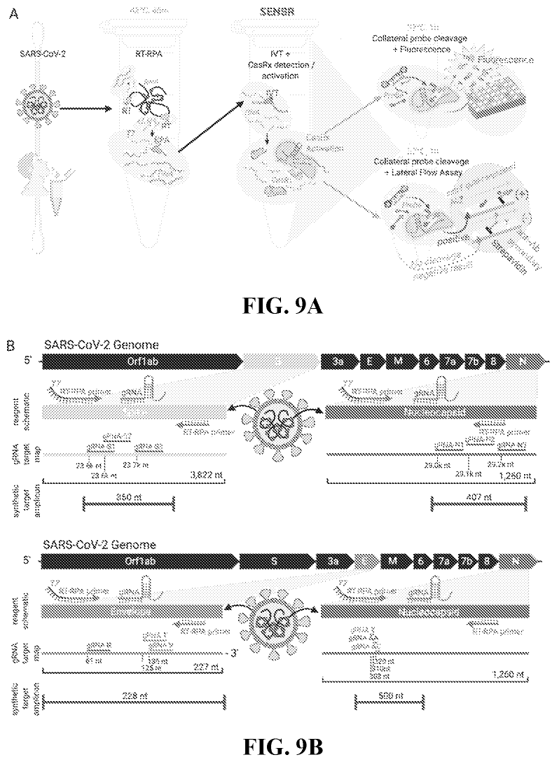

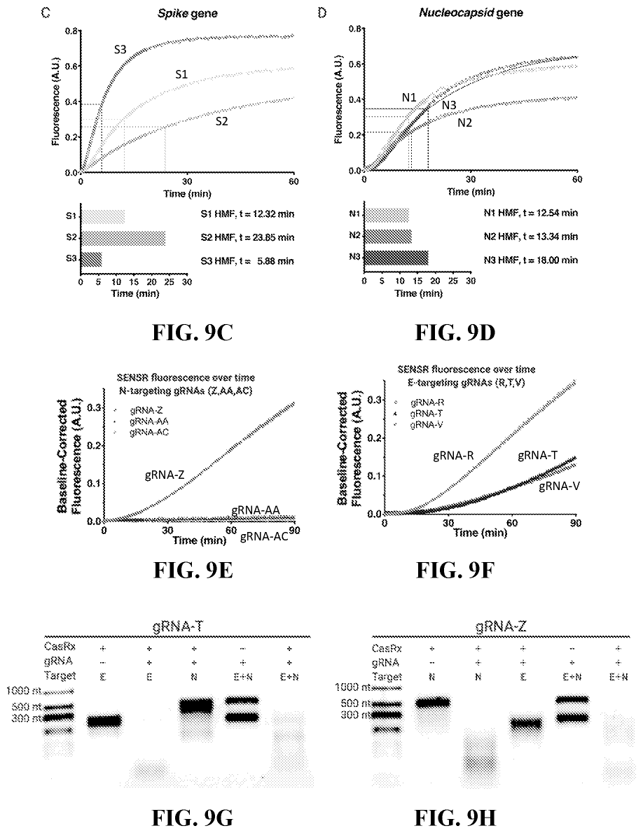

Applicant has reported the first use of CasRx (Konermann et al., Cell 173, 665-676.e14 (2018)) as a molecular diagnostic, developing a unique system referred to herein as SENSR (Sensitive Enzymatic Nucleic-acid Sequence Reporter) and demonstrated robust detection of SARS-CoV-2 viral sequences (FIG. 9A). Following on-target cleavage, CasRx exhibits collateral cleavage of off-target nucleic acids (Konermann et al., Cell 173, 665-676.e14 (2018); and Buchman et al., CRISPR J 3, 164-176 (2020)), a feature shared by Cas nucleases used in other CRISPRDx systems (Gootenberg et al., Science vol. 356 438-442 (2017); Gootenberg et al., Science 360, 439-444 (2018); Chen et al., Science 360, 436-439 (2018); and Li et al., Cell Discov 4, 20 (2018)). The collateral cleavage activity of CasRx was tested to detect SARS-CoV-2 in both synthetic templates as well as in patient-derived samples via fluorescence-based readout, and paper-based lateral flow assay. To maximize specificity, an extensive bioinformatic analysis was performed to identify novel conserved and specific viral targets to minimize false-negative and false-positive rates, respectively. It was demonstrated that SENSR facilitates attomolar sensitivity in just under two hours total reaction time. The detection limit of SENSR is comparable to, though slightly less than, previously established CRISPRDx systems (Patchsung et al., Nat Biomed Eng (2020) doi:10.1038/s41551-020-00603-x; Broughton et al., Nat. Biotechnol. (2020) doi:10.1038/s41587-020-0513-4; and Zhang et al., A protocol for detection of COVID-19 using CRISPR diagnostics. (2020)) and shows promise for improvement.

To establish a reliable method of viral detection in the absence of patient samples, Applicant designed two synthetic gene fragments containing segments of SARS-CoV-2 envelope (E) and nucleocapsid (N) genes consistent with RT-PCR identification established by the CDC and WHO (Corman et al. 2020, Euro Surveillance: Bulletin Europeen Sur Les Maladies Transmissibles=European Communicable Disease Bulletin 25 (3); Broughton et al. 2020, Nat. Biotechnol. (2020) doi:10.1038/s41587-020-0513-4), summarized herein. To mimic the RNA viral genome, Applicant included an upstream T7 promoter sequence permitting in vitro transcription (IVT) of the synthetic gene fragments. These results were further validated by Applicant's collaborator using their RT-PCR verified positive patient-derived nasal swab samples. RT-RPA amplification of viral template sequences along with the template is further discussed herein.

A CRISPR system is provided that comprises, or consists essentially of, or yet further consists of a SARS-CoV-2 gene guide RNA (also referred to herein as a guide or a gRNA), such as an envelope (E) gene gRNA, a nucleocapsid (N) gRNA, or a spike (S) gRNA; and CRISPR reagents necessary to detect the SARS-CoV-2 gene (such as E gene, N gene, S gene, or any combination thereof) in a sample. In one aspect, the system also comprises a promoter sequence permitting in vitro transcription of the SARS-CoV-2 gene (such as E, or S, or N gene, or any combination thereof), an example of which is a T7 promoter. In a further aspect, the CRISPR system comprises, or consists essentially of, or yet further consists of an E gene gRNA and an N gene gRNA. In yet a further aspect, the CRISPR system comprises, or consists essentially of, or yet further consists of an S gene gRNA and an N gene gRNA. In yet a further aspect, the CRISPR system comprises, or consists essentially of, or yet further consists of an S gene gRNA, an E gene gRNA, and an N gene gRNA. Non-limiting examples of such gRNAs are disclosed herein.

In one aspect, provided is a clustered regularly interspaced short palindromic repeats (CRISPR) system. In some embodiments, the system comprises, or consists essentially of, or yet further consists of: a gRNA targeting a target sequence and CRISPR reagents necessary to detect the SARS-CoV-2 sequence in a sample.

### Targets

In one embodiment, the target sequence is an RNA. In a further embodiment, the target sequence is a genomic RNA sequence (for example a gene). In some embodiments, the target sequence comprises, or consists essentially of, or yet further consists of a nucleotide isolated from a pathogen. In some embodiments, the target sequence comprises, or consists essentially of, or yet further consists of a nucleotide transcribed, or reverse-transcribed, or amplified from a pathogen nucleotide. In another embodiment, the target sequence is a DNA. In yet another embodiment, the target sequence is a hybrid of DNA and RNA. In some embodiments, the target sequence is a pathogen sequence (DNA or RNA or a hybrid thereof), for example, a sequence of bunyaviruses, zoonotic viruses such as Ebola, hanta, and Lassa, arboviruses such as dengue, chikungunya, and Zika; coronaviruses such as MERS, SARS-CoV-1, SARS-CoV-2; or other pathogen as disclosed herein. In some embodiments, the target sequence is a severe acute respiratory syndrome coronavirus 2 (SARS-CoV-2) sequence. In further embodiments, the target sequence is selected from one or more of an envelope (E) gene, a nucleocapsid (N) gene, an Orf1ab gene, a Spike (S) gene, an Orf3a gene, an M matrix protein gene, an Orf6 gene, an Orf7a gene, an Orf7b gene, an Orf8 gene, any ORF gene listed herein, such as in Table 2, or a fragment of each thereof. In some embodiments, the gene is in a RNA viral genome, thus is an RNA sequence. Exemplified target sequences are provided herein, see, e.g., Tables 3-5.

TABLE 2
Analysis identifying 30nt CasRx gRNA target sites conserved across, and
specific to, the SARS-CoV-2 genome.

| | | specific&conserved_ | |
|---|---|---|---|
| | | targets | |
| | | Total number of | |
| | | putative target | |
| | total_ | sequences conserved | |
| | targets | within 433 available | |
| | (30nt) | SARS-CoV-2 | |
| | Total | genomes, and | |
| | number | uniquely specific to | |
| | putative | SARS-CoV-2 when | |
| | target | compared to the | percent_ |
| | segments | 3164 publicly | specific&conserved = |
| | (30nt) | available | [specific&conserved_ |
| | identified | Coronavirus | targets]/ |
| ORF id | per gene. | genomes. | [total_targets] (%) |
| endoRNAse | 1009 | 483 | 47.869 |
| S | 3793 | 1568 | 41.339 |
| nsp7 | 220 | 90 | 40.909 |
| nsp4 | 1471 | 574 | 39.021 |
| 3C-like | 889 | 334 | 37.57 |
| proteinase | | | |
| nsp3 | 5806 | 2117 | 36.462 |
| M | 640 | 231 | 36.094 |
| nsp6 | 841 | 261 | 31.034 |
| 2′-O-ribose | 865 | 268 | 30.983 |
| methyltransferase | | | |
| nsp10 | 388 | 112 | 28.866 |
| 3′-to-5′ | 1552 | 432 | 27.835 |
| exonuclease | | | |
| nsp2 | 1885 | 495 | 26.26 |
| RNA-dependent | 2766 | 672 | 24.295 |
| RNA polymerase | | | |
| helicase | 1774 | 428 | 24.126 |
| ORF7a | 337 | 79 | 23.442 |
| ORF8 | 337 | 75 | 22.255 |
| nsp8 | 565 | 105 | 18.584 |
| ORF3a | 799 | 127 | 15.895 |
| leader protein | 511 | 64 | 12.524 |
| N | 1231 | 150 | 12.185 |
| no_gene | 1335 | 146 | 10.936 |
| nsp9 | 310 | 30 | 9.677 |
| ORF7b | 103 | 5 | 4.854 |
| E | 199 | 0 | 0 |
| ORF10 | 88 | 0 | 0 |
| ORF6 | 157 | 0 | 0 |
| Totals | 29714 | 8846 | NA |

In some embodiments, the target sequence is about 25 nt long to about 35 nt long. In some embodiments, the target sequence is about 25 nt long, about 26 nt long, about 27 nt long, about 28 nt long, about 29 nt long, about 30 nt long, about 31 nt long, about 32 nt long, about 33 nt long, about 34 nt long, or about 35 nt long. In one embodiment, the target sequence is about 30 nt long. In some embodiments, the target sequence is not adjacent to a PAM or PFS in the genome or the pathogen to be detected or a RNA (genomic or messenger RNA) of the pathogen.

In some embodiments, the target sequence comprises, or consists essentially of, or yet further consists of one or more of the ones disclosed herein, such as those listed in Tables 3 and 4 and the ones complementary to the gRNA disclosed herein, such as in Table 5.

In some embodiments, a target sequence is selected if having a high specificity to the pathogen to be detected, such as SARS-CoV-2. Additionally or alternatively, a target sequence is selected if conserved among the variants of the pathogen to be detected.

TABLE 3
Unique and conserved 30nt CasRx gRNA target sequences to SARS-
CoV-2. The target sequences are provided below via identifying their
starting nucleotide on SEQ ID NO: 1. In some embodiments, each
target sequence is 30 nt long, i.e., consists of the sequences from the
starting nucleotide at nt N of SEQ ID NO: 1 to nt (N + 29) of SEQ ID
NO 1.

| | Start on SEQ ID NO: 1 |
|---|---|
| Gene: ORF1ab; Peptide: leader | Any one of nt 412 to nt 435 |
| protein | Any one of nt 578 to nt 583 |
| | Any one of nt 742 to nt 775 |
| Gene: ORF1ab | Any one of nt 776 to nt 801 |
| Gene: ORF1ab; Peptide: nsp2 | nt 853 |
| | Any one of nt 932 to nt 942 |
| | Any one of nt 967 to nt 975 |
| | Any one of nt 1063 to nt 1071 |
| | Any one of nt 1102 to nt 1103 |
| | Any one of nt 1156 to nt 1160 |
| | Any one of nt 1258 to nt 1317 |
| | Any one of nt 1348 to nt 1354 |
| | Any one of nt 1401 to nt 1409 |
| | Any one of nt 1469 to nt 1498 |
| | Any one of nt 1548 to nt 1592 |
| | Any one of nt 1623 to nt 1626 |
| | Any one of nt 1691 to nt 1713 |
| | Any one of nt 1727 to nt 1754 |
| | Any one of nt 1815 to nt 1836 |
| | Any one of nt 1912 to nt 1947 |
| | Any one of nt 1963 to nt 1974 |
| | Any one of nt 2007 to nt 2010 |
| | Any one of nt 2110 to nt 2114 |
| | Any one of nt 2145 to nt 2169 |
| | Any one of nt 2197 to nt 2201 |
| | Any one of nt 2311 to nt 2340 |
| | Any one of nt 2368 to nt 2385 |
| | Any one of nt 2446 to nt 2484 |
| | Any one of nt 2563 to nt 2599 |
| | Any one of nt 2632 to nt 2643 |
| | Any one of nt 2680 to nt 2686 |
| Gene: ORF1ab | Any one of nt 2717 to nt 2718 |
| Gene: ORF1b; Peptide: nsp3 | Any one of nt 2719 to nt 2781 |
| | Any one of nt 2810 to nt 2839 |
| | Any one of nt 2891 to nt 2940 |
| | Any one of nt 2974 to nt 3006 |
| | Any one of nt 3037 to nt 3068 |
| | Any one of nt 3110 to nt 3146 |
| | Any one of nt 3177 to nt 3188 |
| | Any one of nt 3199 to nt 3228 |
| | Any one of nt 3259 to nt 3268 |
| | Any one of nt 3299 to nt 3338 |
| | Any one of nt 3411 to nt 3441 |
| | Any one of nt 3451 to nt 3487 |
| | Any one of nt 3518 to nt 3562 |
| | Any one of nt 3593 to nt 3645 |
| | Any one of nt 3661 to nt 3699 |
| | Any one of nt 3706 to nt 3707 |
| | Any one of nt 3738 to nt 3747 |
| | Any one of nt 3784 to nt 3787 |
| | Any one of nt 3808 to nt 3837 |
| | Any one of nt 3843 to nt 3924 |
| | Any one of nt 3992 to nt 4029 |
| | Any one of nt 4050 to nt 4079 |
| | Any one of nt 4087 to nt 4116 |
| | Any one of nt 4132 to nt 4161 |
| | Any one of nt 4174 to nt 4205 |
| | Any one of nt 4402 to nt 4482 |
| | Any one of nt 4495 to nt 4500 |
| | Any one of nt 4513 to nt 4593 |
| | Any one of nt 4603 to nt 4611 |
| | Any one of nt 4702 to nt 4731 |
| | Any one of nt 4762 to nt 4764 |
| | Any one of nt 4809 to nt 4821 |
| | Any one of nt 4840 to nt 4850 |
| | Any one of nt 4881 to nt 4896 |
| | Any one of nt 4908 to nt 4915 |
| | Any one of nt 4946 to nt 4959 |
| | Any one of nt 5000 to nt 5001 |
| | nt 5021 |
| | Any one of nt 5098 to nt 5139 |
| | Any one of nt 5218 to nt 5247 |
| | Any one of nt 5298 to nt 5308 |
| | Any one of nt 5318 to nt 5362 |
| | Any one of nt 5393 to nt 5415 |
| | Any one of nt 5419 to nt 5421 |
| | Any one of nt 5433 to nt 5449 |
| | Any one of nt 5498 to nt 5511 |
| | Any one of nt 5515 to nt 5541 |
| | Any one of nt 5572 to nt 5632 |
| | Any one of nt 5663 to nt 5685 |
| | Any one of nt 5806 to nt 5814 |
| | Any one of nt 5845 to nt 5906 |
| | Any one of nt 5914 to nt 5936 |
| | Any one of nt 6100 to nt 6110 |
| | Any one of nt 6217 to nt 6255 |
| | Any one of nt 6333 to nt 6361 |
| | Any one of nt 6424 to nt 6445 |
| | Any one of nt 6501 to nt 6525 |
| | Any one of nt 6584 to nt 6605 |
| | Any one of nt 6636 to nt 6645 |
| | nt 6649 |
| | Any one of nt 6723 to nt 6735 |
| | Any one of nt 7030 to nt 7051 |
| | Any one of nt 7082 to nt 7104 |
| | nt 7129 |
| | Any one of nt 7132 to nt 7161 |
| | Any one of nt 7237 to nt 7278 |
| | Any one of nt 7363 to nt 7419 |
| | Any one of nt 7441 to nt 7448 |
| | Any one of nt 7518 to nt 7527 |
| | Any one of nt 7582 to nt 7621 |
| | Any one of nt 7699 to nt 7734 |
| | Any one of nt 7915 to nt 7944 |
| | Any one of nt 8001 to nt 8016 |
| | nt 8047 |
| | Any one of nt 8128 to nt 8139 |
| | Any one of nt 8236 to nt 8265 |
| | Any one of nt 8312 to nt 8319 |
| | Any one of nt 8388 to nt 8406 |
| | nt 8425 |
| | Any one of nt 8434 to nt 8478 |
| | Any one of nt 8506 to nt 8524 |
| Gene: ORF1ab | Any one of nt 8525 to nt 8535 |
| Gene: ORF1ab; Peptide: nsp4 | Any one of nt 8569 to nt 8571 |
| | Any one of nt 8602 to nt 8622 |
| | Any one of nt 8681 to nt 8682 |
| | Any one of nt 8698 to nt 8700 |
| | Any one of nt 8788 to nt 8817 |
| | Any one of nt 8840 to nt 8877 |
| | Any one of nt 8945 to nt 8955 |
| | Any one of nt 8987 to nt 8994 |
| | Any one of nt 9043 to nt 9082 |
| | Any one of nt 9195 to nt 9225 |
| | Any one of nt 9274 to nt 9285 |
| | Any one of nt 9289 to nt 9318 |
| | Any one of nt 9328 to nt 9364 |
| | Any one of nt 9367 to nt 9443 |
| | Any one of nt 9601 to nt 9603 |
| | Any one of nt 9634 to nt 9642 |
| | Any one of nt 9663 to nt 9691 |
| | Any one of nt 9735 to nt 9741 |
| | Any one of nt 9773 to nt 9885 |
| | nt 9924 |
| | Any one of nt 9937 to nt 10005 |
| Gene: ORF1ab | Any one of nt 10036 to nt 10041 |
| Gene: ORF1ab; Peptide: 3C-like | Any one of nt 10083 to nt 10098 |
| proteinase | Any one of nt 10129 to nt 10161 |
| | Any one of nt 10177 to nt 10188 |
| | Any one of nt 10195 to nt 10201 |
| | Any one of nt 10232 to nt 10233 |
| | Any one of nt 10243 to nt 10288 |
| | Any one of nt 10323 to nt 10377 |
| | Any one of nt 10411 to nt 10454 |
| | Any one of nt 10572 to nt 10592 |
| | Any one of nt 10632 to nt 10659 |
| | nt 10716 |
| | Any one of nt 10748 to nt 10749 |
| | Any one of nt 10756 to nt 10785 |
| | Any one of nt 10818 to nt 10824 |
| | Any one of nt 10826 to nt 10855 |
| Gene: ORF1ab | Any one of nt 10951 to nt 10971 |
| Gene: ORF1ab; Peptide: nsp6 | Any one of nt 10972 to nt 11011 |
| | Any one of nt 11051 to nt 11052 |
| | Any one of nt 11083 to nt 11100 |
| | Any one of nt 11122 to nt 11130 |
| | Any one of nt 11161 to nt 11176 |
| | Any one of nt 11242 to nt 11250 |
| | Any one of nt 11266 to nt 11289 |
| | Any one of nt 11320 to nt 11328 |
| | Any one of nt 11356 to nt 11379 |
| | Any one of nt 11425 to nt 11439 |
| | Any one of nt 11602 to nt 11623 |
| | Any one of nt 11710 to nt 11719 |
| | Any one of nt 11750 to nt 11812 |
| Gene: ORF1ab | Any one of nt 11813 to nt 11823 |
| | Any one of nt 11839 to nt 11841 |
| Gene: ORF1ab; Peptide: nsp7 | Any one of nt 11842 to nt 11868 |
| | Any one of nt 11839 to nt 11868 |
| | Any one of nt 11872 to nt 11885 |
| | Any one of nt 11956 to nt 11958 |
| | Any one of nt 11986 to nt 12010 |
| | Any one of nt 12041 to nt 12061 |
| Gene: ORF1ab | Any one of nt 12062 to nt 12069 |
| Gene: ORF1ab; Peptide: nsp8 | Any one of nt 12122 to nt 12129 |
| | Any one of nt 12160 to nt 12168 |
| | Any one of nt 12232 to nt 12261 |
| | Any one of nt 12292 to nt 12303 |
| | Any one of nt 12310 to nt 12312 |
| | Any one of nt 12409 to nt 12433 |
| | Any one of nt 12534 to nt 12541 |
| | Any one of nt 12600 to nt 12609 |
| Gene: ORF1ab; Peptide: nsp9 | Any one of nt 12943 to nt 12972 |
| Gene: ORF1ab | Any one of nt 13006 to nt 13018 |
| Gene: ORF1ab; Peptide: nsp10 | Any one of nt 13034 to nt 13041 |
| | Any one of nt 13072 to nt 13110 |
| | Any one of nt 13171 to nt 13194 |
| | Any one of nt 13226 to nt 13254 |
| | Any one of nt 13297 to nt 13308 |
| Gene: ORF1ab; Peptide: RNA- | Any one of nt 13513 to nt 13532 |
| dependent RNA polymerase | Any one of nt 13555 to nt 13559 |
| | nt 13584 |
| | Any one of nt 13587 to nt 13622 |
| | Any one of nt 13713 to nt 13717 |
| | Any one of nt 13845 to nt 13850 |
| | Any one of nt 13872 to nt 13877 |
| | Any one of nt 13887 to nt 13919 |
| | Any one of nt 14002 to nt 14039 |
| | Any one of nt 14046 to nt 14054 |
| | Any one of nt 14073 to nt 14078 |
| | Any one of nt 14083 to nt 14135 |
| | Any one of nt 14148 to nt 14189 |
| | Any one of nt 14229 to nt 14231 |
| | Any one of nt 14235 to nt 14261 |
| | Any one of nt 14358 to nt 14377 |
| | Any one of nt 14408 to nt 14423 |
| | Any one of nt 14569 to nt 14573 |
| | Any one of nt 14604 to nt 14626 |
| | Any one of nt 14757 to nt 14774 |
| | Any one of nt 14836 to nt 14846 |
| | Any one of nt 15193 to nt 15278 |
| | Any one of nt 15285 to nt 15293 |
| | Any one of nt 15357 to nt 15374 |
| | Any one of nt 15489 to nt 15494 |
| | Any one of nt 15510 to nt 15539 |
| | Any one of nt 15684 to nt 15689 |
| | Any one of nt 15771 to nt 15779 |
| | Any one of nt 15810 to nt 15812 |
| | Any one of nt 15927 to nt 15959 |
| | Any one of nt 15978 to nt 16016 |
| | Any one of nt 16075 to nt 16124 |
| Gene: ORF1ab; Peptide: helicase | Any one of nt 16468 to nt 16481 |
| | Any one of nt 16488 to nt 16517 |
| | Any one of nt 16527 to nt 16574 |
| | Any one of nt 16605 to nt 16631 |
| | Any one of nt 16662 to nt 16685 |
| | Any one of nt 16716 to nt 16724 |
| | Any one of nt 16836 to nt 16846 |
| | Any one of nt 17055 to nt 17066 |
| | Any one of nt 17089 to nt 17118 |
| | Any one of nt 17124 to nt 17135 |
| | Any one of nt 17142 to nt 17153 |
| | Any one of nt 17280 to nt 17295 |
| | Any one of nt 17326 to nt 17336 |
| | Any one of nt 17377 to nt 17379 |
| | Any one of nt 17424 to nt 17438 |
| | Any one of nt 17490 to nt 17501 |
| | Any one of nt 17520 to nt 17549 |
| | Any one of nt 17589 to nt 17608 |
| | Any one of nt 17673 to nt 17678 |
| | Any one of nt 17703 to nt 17716 |
| | Any one of nt 17747 to nt 17762 |
| | Any one of nt 17825 to nt 17827 |
| | Any one of nt 17884 to nt 17888 |
| | Any one of nt 17904 to nt 17933 |
| | Any one of nt 17991 to nt 18008 |
| Gene: ORF1ab; Peptide: 3′-to-5′ | Any one of nt 18060 to nt 18098 |
| exonuclease | Any one of nt 18189 to nt 18194 |
| | Any one of nt 18201 to nt 18206 |
| | Any one of nt 18249 to nt 18305 |
| | Any one of nt 18310 to nt 18368 |
| | Any one of nt 18401 to nt 18402 |
| | Any one of nt 18433 to nt 18500 |
| | Any one of nt 18525 to nt 18572 |
| | Any one of nt 18603 to nt 18614 |
| | Any one of nt 18627 to nt 18642 |
| | Any one of nt 18736 to nt 18743 |
| | Any one of nt 18843 to nt 18846 |
| | Any one of nt 18898 to nt 18926 |
| | nt 18987 |
| | Any one of nt 19065 to nt 19094 |
| | Any one of nt 19401 to nt 19412 |
| | Any one of nt 19443 to nt 19457 |
| | Any one of nt 19560 to nt 19579 |
| Gene: ORF1ab; Peptide: endoRNAse | Any one of nt 19645 to nt 19653 |
| | Any one of nt 19684 to nt 19694 |
| | Any one of nt 19716 to nt 19745 |
| | Any one of nt 19761 to nt 19805 |
| | Any one of nt 19845 to nt 19874 |
| | Any one of nt 19931 to nt 19968 |
| | Any one of nt 19999 to nt 20000 |
| | Any one of nt 20061 to nt 20090 |
| | Any one of nt 20103 to nt 20184 |
| | Any one of nt 20190 to nt 20225 |
| | Any one of nt 20317 to nt 20348 |
| | Any one of nt 20363 to nt 20406 |
| | Any one of nt 20421 to nt 20450 |
| | Any one of nt 20514 to nt 20549 |
| | Any one of nt 20598 to nt 20603 |
| | Any one of nt 20607 to nt 20628 |
| Gene: ORF1ab | Any one of nt 20629 to nt 20633 |
| Gene: ORF1ab; Peptide: 2′-O-ribose | Any one of nt 20703 to nt 20721 |
| methyltransferase | Any one of nt 20823 to nt 20843 |
| | Any one of nt 20850 to nt 20855 |
| | Any one of nt 20868 to nt 20876 |
| | Any one of nt 20880 to nt 20897 |
| | Any one of nt 20936 to nt 20939 |
| | Any one of nt 21027 to nt 21056 |
| | Any one of nt 21060 to nt 21106 |
| | Any one of nt 21162 to nt 21164 |
| | Any one of nt 21168 to nt 21179 |
| | Any one of nt 21255 to nt 21263 |
| | Any one of nt 21276 to nt 21285 |
| | Any one of nt 21333 to nt 21355 |
| | Any one of nt 21386 to nt 21389 |
| | Any one of nt 21393 to nt 21421 |
| | Any one of nt 21486 to nt 21509 |
| | Any one of nt 21533 to nt 21544 |
| Gene: S; Peptide: S | Any one of nt 21575 to nt 21611 |
| | Any one of nt 21648 to nt 21660 |
| | Any one of nt 21784 to nt 21819 |
| | Any one of nt 21850 to nt 21862 |
| | Any one of nt 22165 to nt 22176 |
| | Any one of nt 22224 to nt 22246 |
| | Any one of nt 22348 to nt 22374 |
| | Any one of nt 22432 to nt 22573 |
| | Any one of nt 22606 to nt 22695 |
| | Any one of nt 22717 to nt 22754 |
| | Any one of nt 22785 to nt 22788 |
| | Any one of nt 22843 to nt 22853 |
| | Any one of nt 23144 to nt 23154 |
| | Any one of nt 23185 to nt 23199 |
| | Any one of nt 23271 to nt 23286 |
| | Any one of nt 23341 to nt 23372 |
| | Any one of nt 23404 to nt 23461 |
| | Any one of nt 23492 to nt 23637 |
| | Any one of nt 23653 to nt 23865 |
| | Any one of nt 23906 to nt 23921 |
| | Any one of nt 23953 to nt 23958 |
| | Any one of nt 24034 to nt 24048 |
| | Any one of nt 24076 to nt 24142 |
| | Any one of nt 24163 to nt 24243 |
| | Any one of nt 24256 to nt 24258 |
| | Any one of nt 24368 to nt 24381 |
| | Any one of nt 24460 to nt 24501 |
| | Any one of nt 24523 to nt 24570 |
| | Any one of nt 24598 to nt 24600 |
| | Any one of nt 24637 to nt 24663 |
| | Any one of nt 24694 to nt 24698 |
| | Any one of nt 24737 to nt 24759 |
| | Any one of nt 24802 to nt 24852 |
| | Any one of nt 24856 to nt 24897 |
| | Any one of nt 24928 to nt 24935 |
| | Any one of nt 24961 to nt 25033 |
| | Any one of nt 25064 to nt 25065 |
| | Any one of nt 25105 to nt 25125 |
| | Any one of nt 25156 to nt 25176 |
| | Any one of nt 25214 to nt 25221 |
| | Any one of nt 25252 to nt 25306 |
| | Any one of nt 25383 to nt 25385 |
| Gene: ORF3a; Peptide: ORF3a | Any one of nt 25452 to nt 25463 |
| | Any one of nt 25587 to nt 25613 |
| | Any one of nt 25615 to nt 25624 |
| | Any one of nt 25655 to nt 25657 |
| | Any one of nt 25704 to nt 25718 |
| | Any one of nt 25798 to nt 25819 |
| | Any one of nt 25850 to nt 25853 |
| | Any one of nt 25944 to nt 25948 |
| | Any one of nt 26037 to nt 26046 |
| | Any one of nt 26049 to nt 26051 |
| | Any one of nt 26152 to nt 26167 |
| Gene: M; Peptide: M | Any one of nt 26526 to nt 26532 |
| | Any one of nt 26541 to nt 26566 |
| | Any one of nt 26580 to nt 26605 |
| | Any one of nt 26661 to nt 26698 |
| | Any one of nt 26730 to nt 26761 |
| | Any one of nt 26807 to nt 26833 |
| | Any one of nt 26855 to nt 26887 |
| | Any one of nt 26936 to nt 26941 |
| | Any one of nt 26948 to nt 26950 |
| | Any one of nt 27101 to nt 27133 |
| Gene: ORF7a; Peptide: ORF7a | Any one of nt 27398 to nt 27428 |
| | Any one of nt 27525 to nt 27527 |
| | Any one of nt 27576 to nt 27604 |
| | Any one of nt 27684 to nt 27691 |
| | Any one of nt 27722 to nt 27729 |
| | Any one of nt 27730 to nt 27754 |
| Gene: ORF7b; Peptide: ORF7b | Any one of nt 27755 to nt 27759 |
| Gene: ORF8; Peptide: ORF8 | Any one of nt 27925 to nt 27932 |
| | Any one of nt 27964 to nt 27968 |
| | Any one of nt 28001 to nt 28026 |
| | Any one of nt 28099 to nt 28113 |
| | Any one of nt 28148 to nt 28168 |
| Gene: N; Peptide: N | nt 28378 |
| | Any one of nt 28462 to nt 28491 |
| | Any one of nt 28570 to nt 28602 |
| | Any one of nt 29005 to nt 29054 |
| | Any one of nt 29110 to nt 29113 |
| | Any one of nt 29188 to nt 29199 |
| | Any one of nt 29263 to nt 29270 |
| | Any one of nt 29311 to nt 29322 |

### Guides

In some embodiments, a gRNA comprises, or consists essentially of, or yet consists of a nucleotide sequence (such as a RNA) complementary to a target sequence as disclosed herein, an RNA equivalent (i.e., replacing each T with a U) thereof, or a complementary nucleotide sequence thereof. In some embodiments, the nucleotide sequence is complementary to the target sequence. In some embodiments, the nucleotide sequence is essentially complementary to the target sequence but with about 1, or about 2, or about 3, or about 4, or about 5 mismatches. In further embodiments, the gRNA further comprises a direct repeat as disclosed herein.

In some embodiments, the gRNA comprises, or consists essentially of, or yet further consists of a direct repeat (also referred to herein as a DR) and a polynucleotide (such as RNA, DNA or a hybrid thereof) sequence complimentary to the target sequence optionally having 0, 1, 2 or 3 mismatches. In some embodiments, the direct repeat is a 5′ direct repeat.

In some embodiments, the mismatch between the gRNA and the target sequence does not significantly reduce the specificity of detecting a pathogen, such as a SARS-CoV-2. In further embodiments, the mismatch permits successful detection of a pathogen variant. See, for example, Table 4.

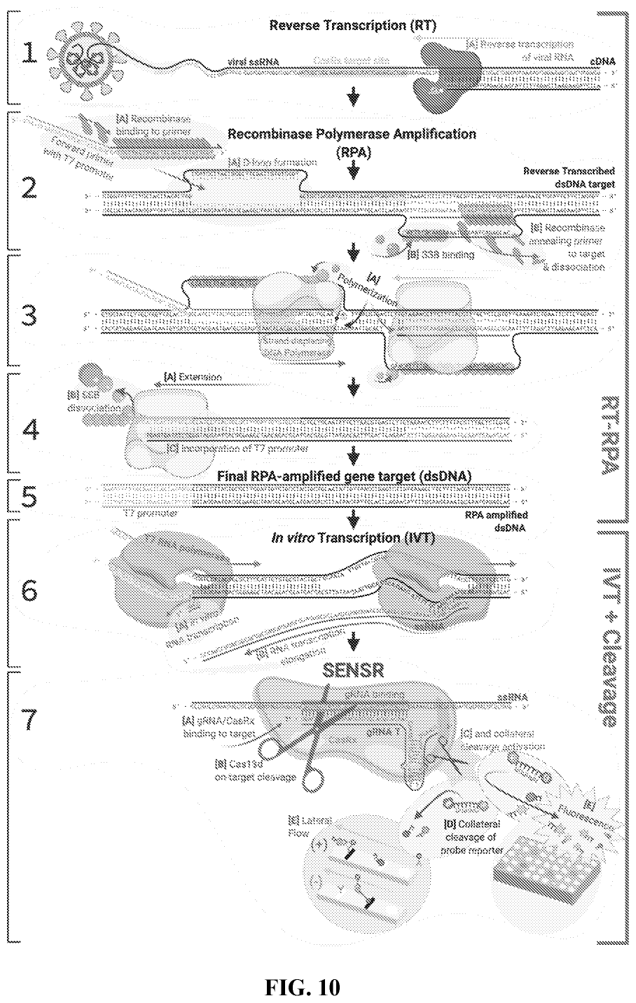

In a further embodiment, the direct repeat (DR) is as disclosed herein, such as in Table 5 or in FIG. 10. In yet a further embodiment, the direct repeat comprises, or consists essentially of, or yet further consists of gcaaguaaaccccuaccaacuggucgggguuugaaac (SEQ ID NO:) or Caaguaaaccccuaccaacuggucgggguuugaaac (SEQ ID NO:), or an equivalent thereof.

In some embodiments, the gRNA or the direct repeat are represented herein as a DNA sequence encoding the gRNA or the direct repeat. In another words, the gRNA or the direct repeat here also intend the polynucleotide (such as DNA, RNA or a hybrid thereof) encoded by a DNA sequence as provided herein.

As it would be understood by one of skill in the art, a gRNA as disclosed herein may be substituted by a polynucleotide encoding such gRNA, thereby the encoded gRNA can be used in a system or a method as disclosed herein. In one example, upon setting up a reaction where a sample or nucleotides isolated from the sample contact with the system as disclosed herein, a gRNA is added as a component of the system. In another example, upon setting up such the reaction, a polynucleotide encoding the gRNA is added along with other reagents necessary for transcribing the polynucleotide to the gRNA, such as RNA polymerase, ATP, GTP, UTP, CTP, a primer pair consisting a reverse primer and a forward primer, and a buffer suitable for the transcription, thus producing the gRNA. In further embodiments, such transcribing step is performed prior to the contacting reaction. In other embodiments, such transcribing step may be part of the contacting reaction. In some embodiments, a gRNA as disclosed herein may be substituted by a vector comprising, or consisting essentially of, or yet further consisting of the polynucleotide encoding such gRNA. In further embodiments, the vector is suitable for encoding the gRNA. In yet further embodiments, the vector further comprises a promoter or other elements suitable for use in encoding the gRNA. In some embodiments, the vector is a non-viral vector, such as a plasmid. In other embodiments, the vector is a viral vector, such as a retroviral vector, a lentiviral vector, an adenoviral vector, and an adeno-associated viral vector.

In some embodiments, gRNA-R targets CTTGCTTTCGTGGTATTCTTGCTAGTTACA, an RNA equivalent (i.e., replacing each T with a U) thereof, or a complementary nucleotide sequence thereof. In some embodiments, gRNA-T targets ACTGCTGCAATATTGTTAACGTGAGTCTTG, an RNA equivalent (i.e., replacing each T with a U) thereof, or a complementary nucleotide sequence thereof. In some embodiments, gRNA-V targets TATTGTTAACGTGAGTCTTGTAAAACCTTC, a RNA equivalent (i.e., replacing each T with a U) thereof, or a complementary nucleotide sequence thereof.

In some embodiments, gRNA-Z targets AAAGATCTCAGTCCAAGATGGTATTTCTAC, i.e., nt 28576 to nt 28605 of SEQ ID NO: 1, an RNA equivalent (i.e., replacing each T with a U) thereof, or a complementary nucleotide sequence thereof. In some embodiments, gRNA-AA targets CTCAGTCCAAGATGGTATTTCTACTACCTA, i.e., nt 28582 to nt 28611 of SEQ ID NO: 1, a RNA equivalent (i.e., replacing each T with a U) thereof, or a complementary nucleotide sequence thereof. In some embodiments, gRNA-AC targets GATGGTATTTCTACTACCTAGGAACTGGGC, i.e., nt 28592 to nt 28621 of SEQ ID NO: 1, a RNA equivalent (i.e., replacing each T with a U) thereof, or a complementary nucleotide sequence thereof.

In some embodiments, gRNA-S1 targets AAATTCAGTTGCTTACTCTAATAACTCTAT, an RNA equivalent (i.e., replacing each T with a U) thereof, or a complementary nucleotide sequence thereof. In some embodiments, gRNA-S2 targets ACTCTAATAACTCTATTGCCATACCCACAA, a RNA equivalent (i.e., replacing each T with a U) thereof, or a complementary nucleotide sequence thereof. In some embodiments, gRNA-S3 targets TTACTATTAGTGTTACCACAGAAATTCTAC, a RNA equivalent (i.e., replacing each T with a U) thereof, or a complementary nucleotide sequence thereof.

In some embodiments, gRNA-N1 targets CGGCAGACGTGGTCCAGAACAAACCCAAGG, an RNA equivalent (i.e., replacing each T with a U) thereof, or a complementary nucleotide sequence thereof. In some embodiments, gRNA-N2 targets GGGGACCAGGAACTAATCAGACAAGGAACT, a RNA equivalent (i.e., replacing each T with a U) thereof, or a complementary nucleotide sequence thereof. In some embodiments, gRNA-N3 targets GCCCCCAGCGCTTCAGCGTTCTTCGGAATG, a RNA equivalent (i.e., replacing each T with a U) thereof, or a complementary nucleotide sequence thereof.

In some embodiments, a gRNA is disclosed herein as a DNA coding the gRNA. See, for example Table 5. In some embodiments, a gRNA is disclosed herein as a DNA coding the gRNA. See, for example Table 5.

In some embodiments, a gRNA-R comprises, or consists essentially of, or yet further consists of CUUGCUUUCGUGGUAUUCUUGCUAGUUACAGUUUCAAACCCCGACCAGU (SEQ ID NO:) or ACUGGUCGGGGUUUGAAACUGUAACUAGCAAGAAUACCACGAAAGCAAG (SEQ ID NO:) or GCAAGUAAACCCCUACCAACUGGUCGGGGUUUGAAACUGUAACUAGCAAGAA UACCACGAAAGCAAG (SEQ ID NO:). In some embodiments, a gRNA-T comprises, or consists essentially of, or yet further consists of ACUGCUGCAAUAUUGUUAACGUGAGUCUUGGUUUCAAACCCCGACCAGU (SEQ ID NO:) or ACUGGUCGGGGUUUGAAACCAAGACUCACGUUAACAAUAUUGCAGCAGU (SEQ ID NO:) or GCAAGUAAACCCCUACCAACUGGUCGGGGUUUGAAACCAAGACUCACGUUAAC AAUAUUGCAGCAGU (SEQ ID NO:). In some embodiments, a gRNA-V comprises, or consists essentially of, or yet further consists of UAUUGUUAACGUGAGUCUUGUAAAACCUUCGUUUCAAACCCCGACCAGU (SEQ ID NO:) or ACUGGUCGGGGUUUGAAACGAAGGUUUUACAAGACUCACGUUAACAAUA (SEQ ID NO:), or GCAAGUAAACCCCUACCAACUGGUCGGGGUUUGAAACGAAGGUUUUACAAGA CUCACGUUAACAAUA (SEQ ID NO:).

In some embodiments, a gRNA-Z comprises, or consists essentially of, or yet further consists of AAAGAUCUCAGUCCAAGAUGGUAUUUCUACGUUUCAAACCCCGACCAGU (SEQ ID NO:) or ACUGGUCGGGGUUUGAAACGUAGAAAUACCAUCUUGGACUGAGAUCUUU (SEQ ID NO:) or CAAGUAAACCCCUACCAACUGGUCGGGGUUUGAAACGUAGAAAUACCAUCUUG GACUGAGAUCUUU (SEQ ID NO:). In some embodiments, a gRNA-AA comprises, or consists essentially of, or yet further consists of CUCAGUCCAAGAUGGUAUUUCUACUACCUAGUUUCAAACCCCGACCAGU (SEQ ID NO:) or ACUGGUCGGGGUUUGAAACUAGGUAGUAGAAAUACCAUCUUGGACUGAG (SEQ ID NO:) or CAAGUAAACCCCUACCAACUGGUCGGGGUUUGAAACUAGGUAGUAGAAAUAC CAUCUUGGACUGAG (SEQ ID NO:). In some embodiments, a gRNA-AC comprises, or consists essentially of, or yet further consists of GAUGGUAUUUCUACUACCUAGGAACUGGGCGUUUCAAACCCCGACCAGU (SEQ ID NO:) or ACUGGUCGGGGUUUGAAACGCCCAGUUCCUAGGUAGUAGAAAUACCAUC (SEQ ID NO:) or CAAGUAAACCCCUACCAACUGGUCGGGGUUUGAAACGCCCAGUUCCUAGGUAG UAGAAAUACCAUC (SEQ ID NO:).

In some embodiments, a gRNA-S1 comprises, or consists essentially of, or yet further consists of auagaguuauuagaguaagcaacugaauuu (SEQ ID NO:) or caaguaaaccccuaccaacuggucgggguuugaaacauagaguuauuagaguaagcaacugaauuu (SEQ ID NO:). In some embodiments, a gRNA-S2 comprises, or consists essentially of, or yet further consists of uuguggguauggcaauagaguuauuagagu (SEQ ID NO:) or caaguaaaccccuaccaacuggucgggguuugaaacuuguggguauggcaauagaguuauuagagu (SEQ ID NO:). In some embodiments, a gRNA-S3 comprises, or consists essentially of, or yet further consists of guagaauuucugugguaacacuaauaguaa (SEQ ID NO:) or caaguaaaccccuaccaacuggucgggguuugaaacguagaauuucugugguaacacuaauaguaa (SEQ ID NO:

In some embodiments, a gRNA-N1 comprises, or consists essentially of, or yet further consists of ccuuggguuuguucuggaccacgucugccg (SEQ ID NO:) or caaguaaaccccuaccaacuggucgggguuugaaacccuuggguuuguucuggaccacgucugccg (SEQ ID NO:). In some embodiments, a gRNA-N2 comprises, or consists essentially of, or yet further consists of aguuccuugucugauuaguuccuggucccc (SEQ ID NO:) or caaguaaaccccuaccaacuggucgggguuugaaacaguuccuugucugauuaguuccuggucccc (SEQ ID NO:). In some embodiments, a gRNA-N3 comprises, or consists essentially of, or yet further consists of cauuccgaagaacgcugaagcgcugggggc (SEQ ID NO:) or caaguaaaccccuaccaacuggucgggguuugaaaccauuccgaagaacgcugaagcgcugggggc (SEQ ID NO:).

Developing diagnostic tests which reduce the probability of false negatives is critical for successful widespread deployment. Because the SARS-Cov-2 genome is actively evolving, either by positive selection or by random synonymous mutagenesis, identifying and targeting genomic sites which remain highly conserved is crucial to develop a robust diagnostic. In line with this, both the primers used to amplify the genomic target sequences as well as the gRNAs used to recognize them need to target conserved yet specific sequences within the genome. Applicant first analyzed the reagents previously validated for their conservation and specificity using the most up to date genomic sequencing data available. To do this, Applicant compared the primers and gRNA target sites targeting the E and N genes to the first 433 available SARS-Cov-2 genomic sequences available on GenBank using. From this analysis, Applicant found the primers targeting the E gene were conserved across 430 or 433 of all available genomes, and the primers targeting the N gene were conserved across 426 or 433 of all total genomes. Each gRNA designed targeted sequences conserved across all 433 available genomes, suggesting these reagents will yield a robust test.

TABLE 4
Analysis of Inter-SARS-CoV-2 Conservation (433 genomes) and
Pan-coronavirus Specificity (3164 genomes) on the 6 gRNAs (R, S, T, U, V, W).

| | | | | (Pan-coronavirus- |
|---|---|---|---|---|
| | | | | specificity) Non-SARS- |
| | | | (Inter-SARS2-conservation) | CoV-2 coronaviruses with |
| | SARS- | | SARS-CoV-2 genome isolates | shared sequence homology, |
| | CoV2 | SARS- | lacking conservation to gRNA | potential off-targets |
| gRNA | Gene | CoV2-Target | target (out of 433 total genomes, | (out of 3164 total genomes, |
| name | target | sequence | GenBank, as of 4/7/2020) | GenBank, as of 4/7/2020) |
| | | | 3 missing genomes: | 10 matching genomes: |
| 1136R | E | 5′-CTTGCTTT | MT276328\|Severe acute | gb: DQ071615\|Organism: Bat |
| | | CGTGGTATTCT | respiratory syndrome coronavirus | SARS CoV Rp3/2004\|Strain |
| | | TGCTAGTtAC | 2 isolate SARS-CoV- | Name: Rp3\|Segment: null\| |
| | | A-3′ | 2/human/USA/OR_2656/2020\| | Host: Bat |
| | | (SEQ ID | complete genome | |
| | | NO: ) | MT159722\|Severe acute | gb: AY502923\|Organism: |
| | | | respiratory syndrome coronavirus | SARS coronavirus |
| | | | 2 isolate 2019-nCoV/USA- | TW10\|Strain |
| | | | CruiseA-6/2020\|complete genome | Name: TW10\|Segment: null\| |
| | | | | Host: Human |
| | | | MT159705\|Severe acute | gb: AY502924\|Organism: |
| | | | respiratory syndrome coronavirus | SARS coronavirus |
| | | | 2 isolate 2019-nCoV/USA- | TW11\|Strain |
| | | | CruiseA-7/2020\|complete genome | Name: TW11\|Segment: null\| |
| | | | | Host: Human |
| | | | | gb: AY502932\|Organism: |
| | | | | SARS coronavirus TW9\|Strain |
| | | | | Name: TW9\|Segment: null\| |
| | | | | Host: Human |
| | | | | gb: AP006558\|Organism: |
| | | | | SARS coronavirus TWJ\|Strain |
| | | | | Name: TWJ\|Segment: null\| |
| | | | | Host: Human |
| | | | | gb: AP006559\|Organism: |
| | | | | SARS coronavirus TWK\|Strain |
| | | | | Name: TWK\|Segment: null\| |
| | | | | Host: Human |
| | | | | gb: AP006560\|Organism: |
| | | | | SARS coronavirus TWS\|Strain |
| | | | | Name: TWS\|Segment: null\| |
| | | | | Host: Human |
| | | | | gb: AP006561\|Organism: |
| | | | | SARS coronavirus TWY\|Strain |
| | | | | Name: TWY\|Segment: null\| |
| | | | | Host: Human |
| | | | | gb: AY338175\|Organism: |
| | | | | SARS coronavirus Taiwan |
| | | | | TC2\|Strain |
| | | | | Name: TC2\|Segment: null\| |
| | | | | Host: Unknown |
| | | | | gb: AY348314\|Organism: |
| | | | | SARS coronavirus Taiwan |
| | | | | TC3\|Strain |
| | | | | Name: TC3\|Segment: null\| |
| | | | | Host: Unknown |
| 1136T | E | 5′ ACTGCTGC | 0 missing genomes: | 4 matching genomes: |
| | | AATATTGTTAA | | gb: MT084071\|Organism: |
| | | CGTGAGTcTtG | | Pangolin coronavirus\|Strain |
| | | 3′ | | Name: MP789\|Segment: null |
| | | (SEQ ID | | Host: Unknown |
| | | NO: ) | | gb: MN996532\|Organism: |
| | | | | Bat coronavirus RaTG13\|Strain |
| | | | | Name: RaTG13\|Segment: null\| |
| | | | | Host: Bat |
| | | | | gb: MG772933\|Organism: |
| | | | | Bat SARS-like |
| | | | | coronavirus\|Strain |
| | | | | Name: bat-SL- |
| | | | | CoVZC45\|Segment: null\| |
| | | | | Host: Bat |
| | | | | gb: MG772934\|Organism: |
| | | | | Bat SARS-like |
| | | | | coronavirus\|Strain |
| | | | | Name: bat-SL- |
| | | | | CoVZXC21\|Segment: null\| |
| | | | | Host: Bat |
| 1136V | E | 5′-TATTGTTA | 0 missing genomes: | 3 matching genomes: |
| | | ACGTGAGTcTt | | gb: MN996532\|Organism: |
| | | GTAAAACCtt | | Bat coronavirus RaTG13\|Strain |
| | | C-3′ | | Name: RaTG13\|Segment: null\| |
| | | (SEQ ID | | Host: Bat |
| | | NO: ) | | gb: MG772933\|Organism: |
| | | | | Bat SARS-like |
| | | | | coronavirus\|Strain |
| | | | | Name: bat-SL- |
| | | | | CoVZC45\|Segment: null\| |
| | | | | Host: Bat |
| | | | | gb: MG772934\|Organism: |
| | | | | Bat SARS-like |
| | | | | coronavirus\|Strain |
| | | | | Name: bat-SL- |
| | | | | CoVZXC21\|Segment: null\| |
| | | | | Host: Bat |
| 1136S | N | 5′-ACAAAGAC | 7 missing genomes: | 1 matching genome: |
| | | GGCATCATATG | MT293160\|Severe acute | gb: MN996532\|Organism: |
| | | GGTTGCAACTG | respiratory syndrome coronavirus | Bat coronavirus RaTG13\|Strain |
| | | 3′ | 2 isolate SARS-CoV- | Name: RaTG13\|Segment: null: |
| | | (SEQ ID | 2/human/USAWA-UW395/2020\| | Host: Bat |
| | | NO: ) | complete genome | |
| | | | MT292574\|Severe acute | |
| | | | respiratory syndrome coronavirus | |
| | | | 2 isolate SARS-CoV- | |
| | | | 2/human/ESP/Valencia15/2020\| | |
| | | | complete genome | |
| | | | MT292573\|Severe acute | |
| | | | respiratory syndrome coronavirus | |
| | | | 2 isolate SARS-CoV- | |
| | | | 2/human/ESP/Valencia14/2020\| | |
| | | | complete genome | |
| | | | MT292571\|Severe acute | |
| | | | respiratory syndrome coronavirus | |
| | | | 2 isolate SARS-CoV- | |
| | | | 2/human/ESP/Valencia12/2020\| | |
| | | | complete genome | |
| | | | MT233523\|Severe acute | |
| | | | respiratory syndrome coronavirus | |
| | | | 2 isolate SARS-CoV- | |
| | | | 2/human/ESP/Valencia8/2020\| | |
| | | | complete genome | |
| | | | MT233519\|Severe acute | |
| | | | respiratory syndrome coronavirus | |
| | | | 2 isolate SARS-CoV- | |
| | | | 2/human/ESP/Valencia5/2020\| | |
| | | | complete genome | |
| | | | MT198652\|Severe acute | |
| | | | respiratory syndrome coronavirus | |
| | | | 2 isolate SARS-CoV- | |
| | | | 2/human/ESP/Valencia003/2020\| | |
| | | | complete genome | |
| 1136U | N | 5′-CGCAATCC | 0 missing genomes: | 3 matching genomes: |
| | | TgcTAACAATG | | gb: MN996532\|Organism: |
| | | CTGCaAtCGTG | | Bat coronavirus RaTG13\|Strain |
| | | 3′ | | Name: RaTG13\|Segment: null\| |
| | | (SEQ ID | | Host: Bat |
| | | NO: ) | | gb: MG772933\|Organism: |
| | | | | Bat SARS-like |
| | | | | coronavirus Strain |
| | | | | Name: bat-SL- |
| | | | | CoVZC45\|Segment: null\| |
| | | | | Host:Bat |
| | | | | gb: MG772934\|Organism: |
| | | | | Bat SARS-like |
| | | | | coronavirus\|Strain |
| | | | | Name:bat-SL- |
| | | | | CoVZXC21\|Segment: null\| |
| | | | | Host: Bat |
| 1136W | N | 5′ TGCTGCaA | 0 missing genomes: | 10 matching genomes: |
| | | tCGTGCTACAA | | gb: MT084071\|Organism: |
| | | CTTCCTCCAAG | | Pangolin coronavirus\|Strain |
| | | G 3′ | | Name: MP789\|Segment: null\| |
| | | (SEQ ID | | Host: Unknown |
| | | NO: ) | | gb: MT040334\|Organism: |
| | | | | Pangolin coronavirus\|Strain |
| | | | | Name: PCoV_GX- |
| | | | | P1E\|Segment: null\| |
| | | | | HostPangolin |
| | | | | gb: MT072864\|Organism: |
| | | | | Pangolin coronavirus Strain |
| | | | | Name: PCoV_GX- |
| | | | | P2V\|Segment: null\|Host: |
| | | | | Anteater |
| | | | | gb: MT072865\|Organism: |
| | | | | Pangolin coronavirus\|Strain |
| | | | | Name:PCoV_GX- |
| | | | | P3B\|Segment: null\|Host: |
| | | | | Anteater |
| | | | | gb: MT040333\|Organism: |
| | | | | Pangolin coronavirus\|Strain |
| | | | | Name:PCoV_GX- |
| | | | | P4L\|Segment: null\| |
| | | | | HostPangolin |
| | | | | gb: MT040336\|Organism: |
| | | | | Pangolin coronavirus\|Strain |
| | | | | Name: PCoV_GX- |
| | | | | P5E\|Segment: null\| |
| | | | | HostPangolin |
| | | | | gb: MT040335\|Organism: |
| | | | | Pangolin coronavirus\|Strain |
| | | | | Name:PCoV_GX- |
| | | | | P5L\|Segment: null\| |
| | | | | HostPangolin |
| | | | | gb: MN996532\|Organism: |
| | | | | Bat coronavirus RaTG13\|Strain |
| | | | | Name: RaTG13\|Segment: null\| |
| | | | | Host: Bat |
| | | | | gb: MG772933\|Organism: |
| | | | | Bat SARS-like |
| | | | | coronavirus\|Strain |
| | | | | Name: bat-SL- |
| | | | | CoVZC45\|Segment: null\| |
| | | | | Host: Bat |
| | | | | gb: MG772934\|Organism: |
| | | | | Bat SARS-like |
| | | | | coronavirus\|Strain |
| | | | | Name:bat-SL- |
| | | | | CoVZXC21\|Segment: null\| |
| | | | | Host: Bat |

To determine if these sequences were specific to SARS-Cov-2 and not other coronaviruses, Applicant compared these sequences to the compendium of viral genomic sequencing data available on ViPR (Virus Pathogen Resource). Applicant found that overall these sequences were highly specific, with only a single probe targeting gene E which may have some crossreactivity with other human coronaviruses, and the remainder having minimal cross reactivity with other mammalian viruses.

TABLE 5
Listing of sequences and reagents, such as primers for cloning, gRNA prep ,and RT-RPA,
as well as gRNA sequences, viral gene templates, plasmid sequences and probes.

| | | |
|---|---|---|
| | Archival | |
| gRNA synthesis | Name | DNA sequence (5′-3′) |

Primers for gRNA synthesis

| The forward primer | 1136B1 | Gaaattaatacgactcactataggcaagtaaacccctaccaactggtcgggg | |
|---|---|---|---|
| that contains the | | tttgaaac (SEQ ID NO: ), wherein the bold letters (i.e, | |
| CasRx direct repeat | | Gaaattaatacgactcactatagg) indicate the T7 Promoter | |
| which was used for | | Sequence or a DNA sequence encoding thereof, and the | |
| synthesis of all | | underlined letters (i.e., | |
| gRNAs by | | caagtaaacccctaccaactggtcggggtttgaaac) indicate a CasRx | |
| templateless PCR | | gRNA DR Sequence or a DNA sequence encoding thereof. | |
| Reverse primer paired | 1136R1 | Cttgctttcgtggtattcttgctagttacagtttcaaaccccgaccagt (SEQ | |
| with 1136B1, used to | | ID NO: ), wherein the bold letters (i.e., | |
| generate gRNA R via | | Cttgctttcgtggtattcttgctagttaca) indicate a Covid-19 | |
| templateless PCR. | | Target Sequence or a DNA sequence encoding thereof | |
| gRNA R targets the E | | and the underlined letters (i.e., gtttcaaaccccgaccagt) | |
| gene. | | indicate a CasRx gRNA DR Sequence or a DNA | |
| | | sequence encoding thereof. | |
| Reverse primer paired | 1136S1 | Acaaagacggcatcatatgggttgcaactggtttcaaaccccgaccagt | |
| with 1136B1, used to | | (SEQ ID NO: ), wherein the bold letters (i.e., | |
| generate gRNA S via | | Acaaagacggcatcatatgggttgcaactg) indicate a Covid-19 | |
| templateless PCR. | | Target Sequence or a DNA sequence encoding thereof | |
| gRNA S targets the N | | and the underlined letters (i.e., gtttcaaaccccgaccagt) | |
| gene | | indicate a CasRx gRNA DR Sequence or a DNA | |
| | | sequence encoding thereof. | |
| Reverse primer paired | 1136T1 | Actgctgcaatattgttaacgtgagtcttggtttcaaaccccgaccagt | |
| with 1136B1, used to | | (SEQ ID NO: ), wherein the bold letters (i.e., | |
| generate gRNA T via | | Actgctgcaatattgttaacgtgagtcttg) indicate a Covid-19 | |
| templateless PCR. | | Target Sequence or a DNA sequence encoding thereof | |
| gRNA T targets the E | | and the underlined letters (i.e., gtttcaaaccccgaccagt) | |
| gene. | | indicate a CasRx gRNA DR Sequence or a DNA | |
| | | sequence encoding thereof. | |
| Reverse primer paired | 1136U1 | Cgcaatcctgctaacaatgctgcaatcgtggtttcaaaccccgaccagt | |
| with 1136B1, used to | | (SEQ ID NO: ), wherein the bold letters (i.e., | |
| generate gRNA U via | | Cgcaatcctgctaacaatgctgcaatcgtg) indicate a Covid-19 | |
| templateless PCR. | | Target Sequence or a DNA sequence encoding thereof | |
| gRNA U targets the N | | and the underlined letters (i.e., gtttcaaaccccgaccagt) | |
| gene. | | indicate a CasRx gRNA DR Sequence or a DNA | |
| | | sequence encoding thereof. | |
| Reverse primer paired | 1136V1 | Tattgttaacgtgagtcttgtaaaaccttcgtttcaaaccccgaccagt | |
| with 1136B1, used to | | (SEQ ID NO: ), wherein the bold letters (i.e., | |
| generate gRNA V via | | Tattgttaacgtgagtcttgtaaaaccttc) indicate a Covid-19 | |
| templateless PCR. | | Target Sequence or a DNA sequence encoding thereof | |
| gRNA V targets the E | | and the underlined letters (i.e., gtttcaaaccccgaccagt) | |
| gene | | indicate a CasRx gRNA DR Sequence or a DNA | |
| | | sequence encoding thereof. | |
| Reverse primer paired | 1136W1 | Tgctgcaatcgtgctacaacttcctcaagggtttcaaaccccgaccagt | |
| with 1136B1, used to | | (SEQ ID NO: ), wherein the bold letters (i.e., | |
| generate gRNA W via | | Tgctgcaatcgtgctacaacttcctcaagg) indicate a Covid-19 | |
| templateless PCR. | | Target Sequence or a DNA sequence encoding thereof | |
| gRNA W targets the N | | and the underlined letters (i.e., gtttcaaaccccgaccagt) | |
| gene. | | indicate a CasRx gRNA DR Sequence or a DNA | |
| | | sequence encoding thereof. | |
| Reverse primer paired | 1136Z1 | Aaagatctcagtccaagatggtatttctacgtttcaaaccccgaccagt | |
| with 1136B1, used to | | (SEQ ID NO: ), wherein the bold letters (i.e., | |
| generate gRNA Z via | | Aaagatctcagtccaagatggtatttctac) indicate a Covid-19 | |
| templateless PCR. | | Target Sequence or a DNA sequence encoding thereof | |
| gRNA Z targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AA1 | Ctcagtccaagatggtatttctactacctagtttcaaaccccgaccagt | |
| with 1136B1, used to | | (SEQ ID NO: ), wherein the bold letters (i.e., | |
| generate gRNA AA | | Ctcagtccaagatggtatttctactaccta) indicate a Covid-19 | |
| via templateless PCR. | | Target Sequence or a DNA sequence encoding thereof | |
| gRNA AA targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AB1 | Ttctactacctaggaactgggccagaagctgtttcaaaccccgaccagt | |
| with 1136B1, used to | | (SEQ ID NO: ), wherein the bold letters (i.e., | |
| generate gRNA AB | | Ttctactacctaggaactgggccagaagct) indicate a Covid-19 | |
| via templateless PCR. | | Target Sequence or a DNA sequence encoding thereof | |
| gRNA AB targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AC1 | Gatggtatttctactacctaggaactgggcgtttcaaaccccgaccagt | |
| with 1136B1, used to | | (SEQ ID NO: ), wherein the bold letters (i.e., | |
| generate gRNA AC | | Gatggtatttctactacctaggaactgggc) indicate a Covid-19 | |
| via templateless PCR. | | Target Sequence or a DNA sequence encoding thereof | |
| gRNA AC targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AQ1 | Aaattcagttgcttactctaataactctatgtttcaaaccccgaccagt (SEQ ID | |
| with 1136B1, used to | | NO: ), wherein the bold letters (i.e., | |
| generate gRNA-Sl via | | Aaattcagttgcttactctaataactctat) indicate a Covid-19 Target | |
| templateless PCR. | | Sequence or a DNA sequence encoding thereof and the | |
| gRNA-Sl targets the S | | underlined letters (i.e., gtttcaaaccccgaccagt) indicate a | |
| gene. | | CasRx gRNA DR Sequence or a DNA sequence encoding | |
| | | thereof. | |
| Reverse primer paired | 1136AP1 | Actctaataactctattgccatacccacaagtttcaaaccccgaccagt (SEQ ID | |
| with 1136B1, used to | | NO: ), wherein the bold letters (i.e., | |
| generate gRNA-S2 via | | Actctaataactctattgccatacccacaa) indicate a Covid-19 | |
| templateless PCR. | | Target Sequence or a DNA sequence encoding thereof | |
| gRNA-S2 targets the S | | and the underlined letters (i.e., gtttcaaaccccgaccagt) | |
| gene. | | indicate a CasRx gRNA DR Sequence or a DNA | |
| | | sequence encoding thereof. | |
| Reverse primer paired | 1136AR1 | Ttactattagtgttaccacagaaattctacgtttcaaaccccgaccagt (SEQ ID | |
| with 1136B1, used to | | NO: ), wherein the bold letters (i.e., | |
| generate gRNA-S3 via | | Ttactattagtgttaccacagaaattctac) indicate a Covid-19 Target | |
| templateless PCR. | | Sequence or a DNA sequence encoding thereof and the | |
| gRNA-S3 targets the S | | underlined letters (i.e., gtttcaaaccccgaccagt) indicate a | |
| gene | | CasRx gRNA DR Sequence or a DNA sequence encoding | |
| | | thereof. | |
| Reverse primer paired | 1136AS1 | Cggcagacgtggtccagaacaaacccaagggtttcaaaccccgaccagt (SEQ | |
| with 1136B1, used to | | ID NO: ), wherein the bold letters (i.e., | |
| generate gRNA-N l via | | Cggcagacgtggtccagaacaaacccaagg) indicate a Covid-19 | |
| templateless PCR. | | Target Sequence or a DNA sequence encoding thereof | |
| gRNA-N 1 targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AT1 | Actctaataactctattgccatacccacaagtttcaaaccccgaccagt (SEQ ID | |
| with 1136B1, used to | | NO: ), wherein the bold letters (i.e., | |
| generate gRNA-N2 | | Actctaataactctattgccatacccacaa) indicate a Covid-19 | |
| via templateless PCR. | | Target Sequence or a DNA sequence encoding thereof | |
| gRNA-N2 targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| Reverse primer paired | 1136AU1 | Gcccccagcgcttcagcgttcttcggaatggtttcaaaccccgaccagt (SEQ ID | |
| with 1136B1, used to | | NO: ), wherein the bold letters (i.e., | |
| generate gRNA-N3 via | | Gcccccagcgcttcagcgttcttcggaatg) indicate a Covid-19 | |
| templateless PCR. | | Target Sequence or a DNA sequence encoding thereof | |
| gRNA-N3 targets a | | and the underlined letters (i.e., gtttcaaaccccgaccagt) | |
| SARS2-specifc and | | indicate a CasRx gRNA DR Sequence or a DNA | |
| conserved sequence in | | sequence encoding thereof. | |
| the N gene. | | | |
| | | | RNA product (5′-3′) (Viral |
| | Archival | | gene mimetic or gRNA |
| RT-RPA reagents | Name | DNA sequence (5′-3′) | sequence) |

E gene target (ORF 4)

| Primers. PCR | 1136Q-F | gaaattaatacgactcactata | |
|---|---|---|---|
| Synthetic viral (E) | | gggatg | |
| gene target, specific | | | |
| to SARS-CoV-2) | | | |
| including addition of | | | |
| a T7 promoter to | | | |
| facilitate IVT | | | |
| production of the of | | | |
| synthetic viral RNA | | | |
| template | | | |
| corresponding to ORF | | | |
| (E) | | | |
| amplifies the (1136Q- | 1136Q-R | ttagaccagaagatcaggaactc | |
| (Synthetic viral (E) | 1136Q | gaaattaatacgactcactata | gauguacucauucguuucggaag |
| gene target, specific | | gggatgtactcattcgtttcggaa | agacagguacguuaauaguuaau |
| to SARS-CoV-2) | | gagacaggtacgttaatagttaat | agcguacuucuuuuucuugcuuu |
| Encompasses entire | | agcgtacttctttttcttgctttcgtg | cgugguauucuugcuaguuacac |
| coding sequence for | | gtattcttgctagttacactagcca | uagccauccuuacugcgcuucga |
| this gene/ORF | | tccttactgcgcttcgattgtgtgc | uugugugcguacugcugcaauau |
| (MN908947.3, 228bp) | | gtactgctgcaatattgttaacgtg | uguuaacgugagucuuguaaaac |
| | | agtcttgtaaaaccttctttttacgt | cuucuuuuuacguuuacucucgu |
| | | ttactctcgtgttaaaaatctgaatt | guuaaaaaucugaauucuucuag |
| | | cttctagagttcctgatcttctggt | aguuccugaucuucuggucuaa |
| | | ctaa | |
| (gRNA R) gRNA | 1136R | gaaattaatacgactcactata | gcaaguaaaccccuaccaacuggu |
| targeting E gene. | | ggcaagtaaacccctaccaactg | cgggguuugaaacuguaacuagc |
| Overlaps a known | | gtcggggtttgaaactgtaactag | aagaauaccacgaaagcaag |
| polymorphism in the | | caagaataccacgaaagcaag | |
| 4th pb of the target | | | |
| site. Pairs with RPA | | | |
| primers RPA-R-F, and | | | |
| RPA-R-R | | | |
| (RPA-R-F) primer. | 1136R-F | gaaattaatacgactcactata | |
| For RPA amplification | | ggggtacgttaatagttaatagcg | |
| of the E gene target | | tacttcttttt | |
| sequence | | | |
| encompassing the | | | |
| gRNA R target site. | | | |
| Adds a T7 for | | | |
| subsequent IVT | | | |
| (RPA-R-R) primer. | 1136R-R | acacaatcgaagcgcagtaagg | |
| For RPA amplification | | atggctag | |
| of the E gene target | | | |
| sequence | | | |
| encompassing the | | | |
| gRNA R target site. | | | |
| (gRNA T) gRNA | 1136T | gaaattaatacgactcactata | gcaaguaaaccccuaccaacuggu |
| targeting E gene. | | ggcaagtaaacccctaccaactg | cgggguuugaaaccaagacucac |
| Overlaps two known | | gtcggggtttgaaaccaagactc | guuaacaauauugcagcagu |
| polymorphisms in the | | acgttaacaatattgcagcagt | |
| 2nd and 4th base pairs. | | | |
| Pairs with RPA | | | |
| primers RPA-T-F and | | | |
| RPA-T-R | | | |
| (RPA-T-F) primer. | 1136T-F | gaaattaatacgactcactata | |
| For RPA amplification | | gggccatccttactgcgcttcgat | |
| of the E gene target | | tgtgtgcgt | |
| sequence | | | |
| encompassing the | | | |
| gRNA T target site. | | | |
| Adds a T7 for | | | |
| subsequent IVT | | | |
| (RPA-T-R) primer. | 1136T-R | cacgagagtaaacgtaaaagaa | |
| For RPA amplification | | ggtttta | |
| of the E gene target | | | |
| sequence | | | |
| encompassing the | | | |
| gRNA T target site. | | | |
| (gRNA V) gRNA | 1136V | gaaattaatacgactcactata | gcaaguaaaccccuaccaacuggu |
| targeting E gene. | | ggcaagtaaacccctaccaactg | cgggguuugaaacgaagguuuua |
| Targets a slightly | | gtcggggtttgaaacgaaggtttt | caagacucacguuaacaaua |
| overlapping sequence | | acaagactcacgttaacaata | |
| with gRNA T, | | | |
| encompassing some of | | | |
| the same | | | |
| polymorphisms. | | | |
| Overlaps four known | | | |
| polymorphisms in the | | | |
| 2nd, 3rd, 12th and | | | |
| 14th base pairs. Pairs | | | |
| with the RPA primers | | | |
| RPA-W-F and RPA- | | | |
| V-R | | | |
| (RPA-V-F) primer. | 1136V-F | gaaattaatacgactcactata | |
| For RPA amplification | | gggtgcgcttcgattgtgtgcgta | |
| of the E gene target | | ctgctgcaa | |
| sequence | | | |
| encompassing the | | | |
| gRNA V target site. | | | |
| Adds a T7 for | | | |
| subsequent IVT | | | |
| (RPA-V-R) primer. | 1136V-R | cacgagagtaaacgtaaaaaga | |
| For RPA amplification | | aggtttta | |
| of the E gene target | | | |
| sequence | | | |
| encompassing the | | | |
| gRNA V target site. | | | |

N gene target (ORF 9)

| | | | |
|---|---|---|---|
| Primers. PCR | 1136X-F | gaaattaatacgactcactata | |
| Synthetic viral (N) | | gggacaaggcgttccaattaaca | |
| gene target, specific | | | |
| to SARS-CoV-2) | | | |
| including addition of | | | |
| a T7 promoter to | | | |
| facilitate IVT | | | |
| production of the of | | | |
| synthetic viral RNA | | | |
| template | | | |
| corresponding to ORF | | | |
| (N) | | | |
| amplifies the (1136X- | 1136X-R | agacattttgctctcaagctg | |
| (Synthetic viral (N) | 1136X | gaaattaatacgactcactata | gacaaggcguuccaauuaacacca |
| gene target, specific | | gggacaaggcgttccaattaaca | auagcaguccagaugaccaaauu |
| to SARS-CoV-2) | | ccaatagcagtccagatgaccaa | ggcuacuaccgaagagcuaccaga |
| Encompasses entire | | attggctactaccgaagagctac | cgaauucgugguggugacgguaa |
| coding sequence for | | cagacgaattcgtggtggtgacg | aaugaaagaucucaguccaagau |
| this gene/ORF | | gtaaaatgaaagatctcagtcca | gguauuucuacuaccuaggaacu |
| (MN908947.3, | | agatggtatttctactacctagga | gggccagaagcuggacuucccua |
| 500bp). Was | | actgggccagaagctggacttcc | uggugcuaacaaagacggcauca |
| generated by PCR | | ctatggtgctaacaaagacggca | uauggguugcaacugagggagcc |
| amplification of (N) | | tcatatgggttgcaactgaggga | uugaauacaccaaaagaucacauu |
| gene fragment from | | gccttgaatacaccaaaagatca | ggcacccgcaauccugcuaacaau |
| IDT (10006625, | | cattggcacccgcaatcctgcta | gcugcaaucgugcuacaacuucc |
| 500bp) | | acaatgctgcaatcgtgctacaa | ucaaggaacaacauugccaaaagg |
| | | cttcctcaaggaacaacattgcc | cuucuacgcagaagggagcagag |
| | | aaaaggcttctacgcagaaggg | gcggcagucaagccucuucucgu |
| | | agcagaggcggcagtcaagcct | uccucaucacguagucgcaacag |
| | | cttctcgttcctcatcacgtagtcg | uucaagaaauucaacuccaggcag |
| | | caacagttcaagaaattcaactcc | caguaggggaacuucuccugcua |
| | | aggcagcagtaggggaacttct | gaauggcuggcaauggcggugau |
| | | cctgctagaatggctggcaatgg | gcugcucuugcuuugcugcugcu |
| | | cggtgatgctgctcttgctttgctg | ugacagauugaaccagcuugaga |
| | | ctgcttgacagattgaaccagctt | gcaaaaugucu |
| | | gagagcaaaatgtct | |
| (gRNA S) gRNA | 1136S | Gaaattaatacgactcactat | gcaaguaaaccccuaccaacuggu |
| targeting N gene. | | aggCaagtaaacccctaccaac | cgggguuugaaaccaguugcaac |
| Overlaps no known | | tggtcggggtttgaaaccagttg | ccauaugaugccgucuuugu |
| polymorphic sites. | | caacccatatgatgccgtctttgt | |
| Pairs with RNA | | | |
| primers-RPA-S-F, | | | |
| and RPA-S-R | | | |
| (RPA-S-F) primer. For | 1136S-F | Gaaattaatacgactcactat | |
| RPA amplification of | | aggggccagaagctggacttcc | |
| the N gene target | | ctatggtgcta | |
| sequence | | | |
| encompassing the | | | |
| gRNA S target site. | | | |
| Adds a T7 for | | | |
| subsequent IVT | | | |
| (RPA-S-R) primer. | 1136S-R | TGTGATCTTTTGGTG | |
| For RPA amplification | | TATTCAAGGCTCCC | |
| of the N gene target | | T | |
| sequence | | | |
| encompassing the | | | |
| gRNA S target site. | | | |
| (gRNA U) gRNA | 1136U | gaaattaatacgactcactata | gcaaguaaaccccuaccaacuggu |
| targeting the N gene. | | ggcaagtaaacccctaccaactg | cgggguuugaaaccacgauugca |
| Overlaps four known | | gtcggggtttgaaaccacgattg | gcauuguuagcaggauugcg |
| polymorphisms, in the | | cagcattgttagcaggattgcg | |
| 5th, 7th, 20th and 21st | | | |
| sites. Pairs with RPA | | | |
| primers RPA-U-F, and | | | |
| RPA-U-R. | | | |
| (RPA-U-F) primer. | 1136U-F | gaaattaatacgactcactata | |
| For RPA amplification | | gggttgaatacaccaaaagatca | |
| of the N gene target | | cattggcacc | |
| sequence | | | |
| encompassing the | | | |
| gRNA U target site. | | | |
| Adds a T7 for | | | |
| subsequent IVT | | | |
| (RPA-U-R) primer. | 1136U-R | tggcaatgttgttccttgaggaag | |
| For RPA amplification | | ttgtag | |
| of the N gene target | | | |
| sequence | | | |
| encompassing the | | | |
| gRNA U target site. | | | |
| (gRNA W) gRNA | 1136W | gaaattaatacgactcactata | gcaaguaaaccccuaccaacuggu |
| targeting the N gene. | | ggcaagtaaacccctaccaactg | cgggguuugaaacccuugaggaa |
| Targets a slightly | | gtcggggtttgaaacccttgagg | guuguagcacgauugcagca |
| overlapping sequence | | aagttgtagcacgattgcagca | |
| with gRNA U, | | | |
| encompassing some of | | | |
| the same | | | |
| polymorphisms. | | | |
| Overlaps two known | | | |
| polymorphisms, in the | | | |
| 22nd and 24th base | | | |
| pairs. Pairs with RPA | | | |
| primers RPA-W-F, | | | |
| and RPA-W-R. | | | |
| (RPA-W-F) primer. | 1136W-F | gaaattaatacgactcactata | |
| For RPA amplification | | gggtcacattggcacccgcaatc | |
| of the N gene target | | ctgctaacaa | |
| sequence | | | |
| encompassing the | | | |
| gRNA W target site. | | | |
| Adds a T7 for | | | |
| subsequent IVT | | | |
| (RPA-W-R) primer. | 1136W-R | tctgcgtagaagccttttggcaat | |
| For RPA amplification | | gttgtt | |
| of the N gene target | | | |
| sequence | | | |
| encompassing the | | | |
| gRNA W target site. | | | |
| (gRNA Z) gRNA | 1136Z | gaaattaatacgactcactata | caaguaaaccccuaccaacugguc |
| targeting the N gene. | | ggcaagtaaacccctaccaactg | gggguuugaaacguagaaauacc |
| Targets a slightly | | gtcggggtttgaaacgtagaaat | aucuuggacugagaucuuu |
| overlapping sequence | | accatcttggactgagatcttt | |
| with gRNAs AA, AB, | | | |
| and AC. Designed | | | |
| following | | | |
| conservation analysis | | | |
| to be specific to | | | |
| SARS-CoV-2 with no | | | |
| homology to other | | | |
| coronaviruses. | | | |
| (RPA-Z-F) primer. | 1136Z-F | gaaattaatacgactcactata | |
| For RPA amplification | | gggagacgaattcgtggtggtg | |
| of the N gene target | | acggtaaaatg | |
| sequence | | | |
| encompassing the | | | |
| gRNA Z target site. | | | |
| Adds a T7 for | | | |
| subsequent IVT | | | |
| (RPA-Z-R) primer. | 1136Z-R | aagtccagcttctggcccagttcc | |
| For RPA amplification | | taggta | |
| of the N gene target | | | |
| sequence | | | |
| encompassing the | | | |
| gRNA Z target site. | | | |
| (gRNA AA) gRNA | 1136AA | gaaattaatacgactcactata | caaguaaaccccuaccaacugguc |
| targeting the N gene. | | ggcaagtaaacccctaccaactg | gggguuugaaacuagguaguaga |
| Targets a slightly | | gtcggggtttgaaactaggtagt | aauaccaucuuggacugag |
| overlapping sequence | | agaaataccatcttggactgag | |
| with gRNAs Z, AB, | | | |
| and AC. Designed | | | |
| following | | | |
| conservation analysis | | | |
| to be specific to | | | |
| SARS-CoV-2 with no | | | |
| homology to other | | | |
| coronaviruses. | | | |
| (RPA-AA-F) primer. | 1136AA-F | gaaattaatacgactcactata | |
| For RPA amplification | | gggattcgtggtggtgacggtaa | |
| of the N gene target | | aatgaaagat | |
| sequence | | | |
| encompassing the | | | |
| gRNA AA target site. | | | |
| Adds a T7 for | | | |
| subsequent IVT | | | |
| (RPA-AA-R) primer. | 1136AA-R | atagggaagtccagcttctggcc | |
| For RPA amplification | | cagttcc | |
| of the N gene target | | | |
| sequence | | | |
| encompassing the | | | |
| gRNA AA target site. | | | |
| (gRNA AB) gRNA | 1136AB | gaaattaatacgactcactata | caaguaaaccccuaccaacugguc |
| targeting the N gene. | | ggcaagtaaacccctaccaactg | gggguuugaaacagcuucuggcc |
| Targets a slightly | | gtcggggtttgaaacagcttctg | caguuccuagguaguagaa |
| overlapping sequence | | gcccagttcctaggtagtagaa | |
| with gRNAs Z, AA, | | | |
| and AC. Designed | | | |
| following | | | |
| conservation analysis | | | |
| to be specific to | | | |
| SARS-CoV-2 with no | | | |
| homology to other | | | |
| coronaviruses. | | | |
| (RPA-AB-F) primer. | 1136AB-F | gaaattaatacgactcactata | |
| For RPA amplification | | gggaaaatgaaagatctcagtcc | |
| of the N gene target | | aagatggtat | |
| sequence | | | |
| encompassing the | | | |
| gRNA AB target site. | | | |
| Adds a T7 for | | | |
| subsequent IVT | | | |
| (RPA-AB-R) primer. | 1136AB-R | gccgtctttgttagcaccataggg | |
| For RPA amplification | | aagtcc | |
| of the N gene target | | | |
| sequence | | | |
| encompassing the | | | |
| gRNA AB target site. | | | |
| (gRNA AC) gRNA | 1136AC | gaaattaatacgactcactata | caaguaaaccccuaccaacugguc |
| targeting the N gene. | | ggcaagtaaacccctaccaactg | gggguuugaaacgcccaguuccu |
| Targets a slightly | | gtcggggtttgaaacgcccagtt | agguaguagaaauaccauc |
| overlapping sequence | | cctaggtagtagaaataccatc | |
| with gRNAs Z, AA, | | | |
| and AB. Designed | | | |
| following | | | |
| conservation analysis | | | |
| to be specific to | | | |
| SARS-CoV-2 with no | | | |
| homology to other | | | |
| coronaviruses. | | | |
| (RPA-AC-F) primer. | 1136AC-F | gaaattaatacgactcactata | |
| For RPA amplification | | ggggtgacggtaaaatgaaaga | |
| of the N gene target | | tctcagtccaa | |
| sequence | | | |
| encompassing the | | | |
| gRNA AC target site. | | | |
| Adds a T7 for | | | |
| subsequent IVT | | | |
| (RPA-AC-R) primer. | 1136AC-R | tgttagcaccatagggaagtcca | |
| For RPA amplification | | gcttctg | |
| of the N gene target | | | |
| sequence | | | |
| encompassing the | | | |
| gRNA AC target site. | | | |

S gene target (Spike)

| | | | |
|---|---|---|---|
| Primers. PCR | 1136AE-F | gaaattaatacgactcactata | |
| -Synthetic viral (S) | | gggaaac | |
| gene target, specific to | 1136AE-R | acaaaaactgccatattgcaaca | |
| SARS-CoV-2) | | | |
| including addition of a | | | |
| T7 promoter to | | | |
| facilitate IVT | | | |
| production of the of | | | |
| synthetic viral RNA | | | |
| template | | | |
| corresponding to ORF | | | |
| (S) | | | |
| amplifies the (1136AE | | | |
| (Synthetic viral (S) | 1136AE | gaaattaatacgactcactata | aaacacgugcaggcuguuuaaua |
| gene target, specific to | | gggaaacacgtgcaggctgttta | ggggcugaacaugucaacaacuc |
| SARS-CoV-2) | | ataggggctgaacatgtcaacaa | auaugagugugacauacccauug |
| Encompasses entire | | ctcatatgagtgtgacatacccat | gugcagguauaugcgcuaguuau |
| coding sequence for | | tggtgcaggtatatgcgctagtta | cagacucagacuaauucuccucg |
| this gene/ORF | | tcagactcagactaattctcctcg | gcgggcacguaguguagcuaguc |
| (MN908947.3, | | gcgggcacgtagtgtagctagtc | aauccaucauugccuacacuaug |
| 3822nt) | | aatecatcattgcctacactatgtc | ucacuuggugcagaaaauucagu |
| | | acttggtgcagaaaattcagttgc | ugcuuacucuaauaacucuauug |
| | | ttactctaataactctattgccatac | ccauacccacaaauuuuacuauua |
| | | ccacaaattttactattagtgttac | guguuaccacagaaauucuacca |
| | | cacagaaattctaccagtgtctat | gugucuaugaccaagacaucagu |
| | | gaccaagacatcagtagattgta | agauuguacaauguacauuugug |
| | | caatgtacatttgtggtgattcaac | gugauucaacugaaugcagcaau |
| | | tgaatgcagcaatcttttgttgcaa | cuuuuguugcaauauggcaguuu |
| | | tatggcagtttttgt | uugu |
| (gRNA-S1) gRNA | 1136AQ | caagtaaacccctaccaactggt | caaguaaaccccuaccaacugguc |
| targeting S gene. Pairs | | cggggtttgaaacatagagttatt | gggguuugaaacauagaguuauu |
| with RPA primers | | agagtaagcaactgaattt | agaguaagcaacugaauuu |
| RPA-S1-F, and RPA- | | | |
| S1-R | | | |
| (RPA-S1-F) primer. | 1136AQ-F | gaaattaatacgactcactata | |
| For RPA amplification | | gggcattgcctacactatgtcact | |
| of the S gene target | | tggtgcaga | |
| sequence | | | |
| encompassing the | | | |
| gRNA-Sl target site. | | | |
| Adds a T7 for | | | |
| subsequent IVT | | | |
| (RPA-SI-R) primer. | 1136AQ-R | acactaatagtaaaatttgtgggt | |
| For RPA amplification | | atggca | |
| of the S gene target | | | |
| sequence | | | |
| encompassing the | | | |
| gRNA-S1 target site. | | | |
| (gRNA-S2) gRNA | 1136AP | caagtaaacccctaccaactggt | caaguaaaccccuaccaacugguc |
| targeting S gene. Pairs | | cggggtttgaaacttgtgggtatg | gggguuugaaacuuguggguau |
| with RPA primers | | gcaatagagttattagagt | ggcaauagaguuauuagagu |
| RPA-S2-F and RPA- | | | |
| S2-R | | | |
| (RPA-S2-F) primer. | 1136AP-F | gaaattaatacgactcactata | |
| For RPA amplification | | gggtgtcacttggtgcagaaaatt | |
| of the S gene target | | cagttgctt | |
| sequence | | | |
| encompassing the | | | |
| gRNA-S2 target site. | | | |
| Adds a T7 for | | | |
| subsequent IVT | | | |
| (RPA-S2-R) primer. | 1136AP-R | gaatttctgtggtaacactaatagt | |
| For RPA amplification | | aaaat | |
| of the S gene target | | | |
| sequence | | | |
| encompassing the | | | |
| gRNA-S2 target site. | | | |
| (gRNA-S3) gRNA | 1136AR | caagtaaacccctaccaactggt | caaguaaaccccuaccaacugguc |
| targeting S gene. Pairs | | cggggtttgaaacgtagaatttct | gggguuugaaacguagaauuucu |
| with the RPA primers | | gtggtaacactaatagtaa | gugguaacacuaauaguaa |
| RPA-S3-F and RPA- | | | |
| S3-R | | | |
| (RPA-S3-F) primer. | 1136AR-F | gaaattaatacgactcactata | |
| For RPA amplification | | gggctaataactctattgccatac | |
| of the S gene target | | ccacaaatt | |
| sequence | | | |
| encompassing the | | | |
| gRNA-S3 target site. | | | |
| Adds a T7 for | | | |
| subsequent IVT | | | |
| (RPA-S3-R) primer. | 1136AR-R | aatctactgatgtcttggtcataga | |
| For RPA amplification | | cactg | |
| of the S gene target | | | |
| sequence | | | |
| encompassing the | | | |
| gRNA-S3 target site. | | | |

N gene target (Nucleocapsid)

| | | | |
|---|---|---|---|
| Primers. PCR | 1136X2-F | gaaattaatacgactcactata | |
| -Synthetic viral (N) | | gggtctggtaaaggccaacaac | |
| gene target, specific to | 1136X2-R | ttttaggctctgttggtggg | |
| SARS-CoV-2) | | | |
| including addition of a | | | |
| T7 promoter to | | | |
| facilitate IVT | | | |
| production of the of | | | |
| synthetic viral RNA | | | |
| template | | | |
| corresponding to ORF | | | |
| (N) | | | |
| amplifies the (1136X2 | | | |
| (Synthetic viral (N) | 1136X2 | gaaattaatacgactcactata | gucugguaaaggccaacaacaaca |
| gene target, specific | | gggtctggtaaaggccaacaac | aggccaaacugucacuaagaaauc |
| to SARS-CoV-2) | | aacaaggccaaactgtcactaag | ugcugcugaggcuucuaagaagc |
| Encompasses coding | | aaatctgctgctgaggcttctaag | cucggcaaaaacguacugccacua |
| sequence for N | | aagcctcggcaaaaacgtactg | aagcauacaauguaacacaagcuu |
| gene/ORF | | ccactaaagcatacaatgtaaca | ucggcagacgugguccagaacaa |
| (MN908947.3, 407nt). | | caagctttcggcagacgtggtcc | acccaaggaaauuuuggggacca |
| Was generated by | | agaacaaacccaaggaaattttg | ggaacuaaucagacaaggaacuga |
| PCR amplification of | | gggaccaggaactaatcagaca | uuacaaacauuggccgcaaauug |
| (N) gene fragment | | aggaactgattacaaacattggc | cacaauuugcccccagcgcuucag |
| from IDT (10006625, | | cgcaaattgcacaatttgccccc | cguucuucggaaugucgcgcauu |
| 407bp) | | agcgcttcagcgttcttcggaatg | ggcauggaagucacaccuucggg |
| | | tcgcgcattggcatggaagtcac | aacgugguugaccuacacaggug |
| | | accttcgggaacgtggttgacct | ccaucaaauuggaugacaaagauc |
| | | acacaggtgccatcaaattggat | caaauuucaaagaucaagucauu |
| | | gacaaagatccaaatttcaaaga | uugcugaauaagcauauugacgc |
| | | tcaagtcattttgctgaataagcat | auacaaaacauucccaccaacaga |
| | | attgacgcatacaaaacattccca | gccuaaaa |
| | | ccaacagagcctaaaa | |
| (gRNA-N1) gRNA | 1136AS | caagtaaacccctaccaactggt | caaguaaaccccuaccaacugguc |
| targeting the N gene. | | cggggtttgaaacccttgggtttg | gggguuugaaacccuuggguuug |
| Pairs with RPA | | ttctggaccacgtctgccg | uucuggaccacgucugccg |
| primers RPA-N1-F, | | | |
| and RPA-N1-R | | | |
| (RPA-N1-F) primer. | 1136AS-F | gaaattaatacgactcactata | |
| For RPA amplification | | gggcactaaagcatacaatgtaa | |
| of the N gene target | | cacaagcttt | |
| sequence | | | |
| encompassing the | | | |
| gRNA-N1 target site. | | | |
| Adds a T7 for | | | |
| subsequent IVT | | | |
| (RPA-N1-R) primer. | 1136AS-R | tgtctgattagttcctggtccccaa | |
| For RPA amplification | | aattt | |
| of the N gene target | | | |
| sequence | | | |
| encompassing the | | | |
| gRNA-N1 target site. | | | |
| (gRNA-N2) gRNA | 1136AT | caagtaaacccctaccaactggt | caaguaaaccccuaccaacugguc |
| targeting the N gene. | | cggggtttgaaacagttccttgtc | gggguuugaaacaguuccuuguc |
| Pairs with RPA | | tgattagttcctggtcccc | ugauuaguuccuggucccc |
| primers RPA-N2-F, | | | |
| and RPA-N2-R | | | |
| (RPA-N2-F) primer. | 1136AT-F | gaaattaatacgactcactata | |
| For RPA amplification | | gggcgtggtccagaacaaaccc | |
| of the N gene target | | aaggaaatttt | |
| sequence | | | |
| encompassing the | | | |
| gRNA-N2 target site. | | | |
| Adds a T7 for | | | |
| subsequent IVT | | | |
| (RPA-N2-R) primer. | 1136AT-R | ttgtgcaatttgcggccaatgtttg | |
| For RPA amplification | | taatc | |
| of the N gene target | | | |
| sequence | | | |
| encompassing the | | | |
| gRNA-N2 target site. | | | |
| (gRNA-N3) gRNA | 1136AU | caagtaaacccctaccaactggt | caaguaaaccccuaccaacugguc |
| targeting the N gene. | | cggggtttgaaaccattccgaag | gggguuugaaaccauuccgaaga |
| Pairs with RPA | | aacgctgaagcgctgggggc | acgcugaagcgcugggggc |
| primers RPA-N3-F, | | | |
| and RPA-N3-R | | | |
| (RPA-N3-F) primer. | 1136AU | gaaattaatacgactcactata | |
| For RPA amplification | -F | gggtacaaacattggccgcaaat | |
| of the N gene target | | tgcacaattt | |
| sequence | | | |
| encompassing the | | | |
| gRNA-N3 target site. | | | |
| Adds a T7 for | | | |
| subsequent IVT | | | |
| RPA-N3-R primer. | 1136AU-R | cgaaggtgtgacttecatgccaa | |
| For RPA amplification | | tgcgcga | |
| of the N gene target | | | |
| sequence | | | |
| encompassing the | | | |
| gRNA-N3 target site. | | | |

KEY:
T7 Promoter Sequence (bold);
Covid-19 Target Sequence (underlined);
gRNA scaffold (DR)
CasRx expression plasmid and cloning

| Primers. PCR | 1136I.C1 | cgaggaaaacctgtacttccaatc |
|---|---|---|
| cloning into protein | | caatatcgaaaaaaaaaagtcc |
| expression backbone, | | |
| pET-His6-MBP-tev- | | |
| yORF, generating the | | |
| final pET-6xHis- | | |
| MBP-TEV-CasRx | | |
| amplifies CasRx for | 1136I.C2 | gctcgagtgcggccgcaagcttgt |
| | | cgacttaggaattgccggacac |
| | | ct |

KEY:
CasRx CDS Gibson cloning Homology (bold);
Gibson Cloning homology to pET-His6-MBP-tev-yORF (underlined)

| CasRx protein | | |
|---|---|---|
| sequence. Derived | | ARLEKIVEGDSIRSVNEGEAFSAEMADKNAGYKI |
| from a Cas protein in | | GNAKFSHPKGYAVVANNPLYTGPVQQDMLGLK |
| Ruminococcus | | ETLEKRYFGESADGNDNICIQVIHNILDIEKILAEY |
| flavefaciens, codon | | ITNAAYAVNNISGLDKDIIGFGKFSTVYTYDEFKD |
| optimized for | | PEHHRAAFNNNDKLINAIKAQYDEFDNFLDNPRL |
| expression in human | | GYFGQAFFSKEGRNYIINYGNECYDILALLSGLR |
| cells. | | HWVVHNNEEESRISRTWLYNLDKNLDNEYISTL |
| | | NYLYDRITNELTNSFSKNSAANVNYIAETLGINPA |
| | | EFAEQYFRFSIMKEQKNLGFNITKLREVMLDRKD |
| | | MSEIRKNHKVFDSIRTKVYTMMDFVIYRYYIEED |
| | | AKVAAANKSLPDNEKSLSEKDIFVINLRGSFNDD |
| | | QKDALYYDEANRIWRKLENIMHNIKEFRGNKTR |
| | | EYKKKDAPRLPRILPAGRDVSAFSKLMYALTMFL |
| | | DGKEINDLLTTLINKFDNIQSFLKVMPLIGVNAKF |
| | | VEEYAFFKDSAKIADELRLIKSFARMGEPIADARR |
| | | AMYIDAIRILGTNLSYDELKALADTFSLDENGNK |
| | | LKKGKHGMRNFIINNVISNKRFHYLIRYGDPAHL |
| | | HEIAKNEAVVKFVLGRIADIQKKQGQNGKNQIDR |
| | | YYETCIGKDKGKSVSEKVDALTKIITGMNYDQFD |
| | | KKRSVIEDTGRENAEREKFKKIISLYLTVIYHILKN |
| | | IVNINARYVIGFHCVERDAQLYKEKGYDINLKKL |
| | | EEKGFSSVTKLCAGIDETAPDKRKDVEKEMAER |
| | | AKESIDSLESANPKLYANYIKYSDEKKAEEFTRQI |
| | | NREKAKTALNAYLRNTKWNVIIREDLLRIDNKTC |
| | | TLFRNKAVHLEVARYVHAYINDIAEVNSYFQLY |
| | | HYIMQRIIMNERYEKSSGKVSEYFDAVNDEKKY |
| | | NDRLLKLLCVPFGYCIPRFKNLSIEALFDRNEAAK |
| | | FDKEKKKVSGNS |
| KEY: | (bold & underlined) | |
| CasRx HEPN domain | | |
| pET-His-MBP-TEV- | Addgene | caaggagatggcgcccaacagtcccccggccacggggcctgccaccatac |
| CasRx | plasmid | ccacgccgaaacaagcgctcatgagcccgaagtggcgagcccgatcttccc |
| | # | catcggtgatgtcggcgatataggcgccagcaaccgcacctgtggcgccgg |
| | 153023 | tgatgccggccacgatgcgtccggcgtagaggatcgagatctcgatcccgc |
| | | gaaattaatacgactcactataggggaattgtgagcggataacaattcccctct |
| | | agaaataattttgtttaactttaagaaggagatataccATGggttcttctcacc |
| | | atcaccatcaccatggttcttctatgaaaatcgaagaaggtaaactggtaa |
| | | tctggattaacggcgataaaggctataacggtctcgctgaagtcggtaa |
| | | gaaattcgagaaagataccggaattaaagtcaccgttgagcatccggat |
| | | aaactggaagagaaattcccacaggttgcggcaactggcgatggccct |
| | | gacattatcttctgggcacacgaccgctttggtggctacgctcaatctggc |
| | | ctgttggctgaaatcaccccggacaaagcgttccaggacaagctgtatc |
| | | cgtttacctgggatgccgtacgttacaacggcaagctgattgcttacccg |
| | | atcgctgttgaagcgttatcgctgatttataacaaagatctgctgccgaac |
| | | ccgccaaaaacctgggaagagatcccggcgctggataaagaactgaa |
| | | agcgaaaggtaagagcgcgctgatgttcaacctgcaagaaccgtacttc |
| | | acctggccgctgattgctgctgacgggggttatgcgttcaagtatgaaaa |
| | | cggcaagtacgacattaaagacgtgggcgtggataacgctggcgcgaa |
| | | agcgggtctgaccttcctggttgacctgattaaaaacaaacacatgaatg |
| | | agcgatgaccatcaacggcccgtgggcatggtccaacatcgacaccag |
| | | caaagtgaattatggtgtaacggtactgccgaccttcaagggtcaaccat |
| | | ccaaaccgttcgttggcgtgctgagcgcaggtattaacgccgccagtccg |
| | | aacaaagagctggcaaaagagttcctcgaaaactatctgctgactgatg |
| | | aaggtctggaagcggttaataaagacaaaccgctgggtgccgtagcgct |
| | | gaagtcttacgaggaagagttggcgaaagatccacgtattgccgccact |
| | | atggaaaacgcccagaaaggtgaaatcatgccgaacatcccgcagatg |
| | | tccgctttctggtatgccgtgcgtactgcggtgatcaacgccgccagcggt |
| | | cgtcagactgtcgatgaagccctgaaagacgcgcagactaatgggatcg |
| | | aggaaaacctgtacttccaatccaatATCGAAAAAAAAAAGT |
| | | CCTTCGCCAAGGGCATGGGCGTGAAGTCCA |
| | | CACTCGTGTCCGGCTCCAAAGTGTACATGAC |
| | | AACCTTCGCCGAAGGCAGCGACGCCAGGCT |
| | | GGAAAAGATCGTGGAGGGCGACAGCATCAG |
| | | GAGCGTGAATGAGGGCGAGGCCTTCAGCGC |
| | | TGAAATGGCCGATAAAAACGCCGGCTATAA |
| | | GATCGGCAACGCCAAATTCAGCCATCCTAA |
| | | GGGCTACGCCGTGGTGGCTAACAACCCTCT |
| | | GTATACAGGACCCGTCCAGCAGGATATGCT |
| | | CGGCCTGAAGGAAACTCTGGAAAAGAGGTA |
| | | CTTCGGCGAGAGCGCTGATGGCAATGACAA |
| | | TATTTGTATCCAGGTGATCCATAACATCCTG |
| | | GACATTGAAAAAATCCTCGCCGAATACATTA |
| | | CCAACGCCGCCTACGCCGTCAACAATATCTC |
| | | CGGCCTGGATAAGGACATTATTGGATTCGG |
| | | CAAGTTCTCCACAGTGTATACCTACGACGAA |
| | | TTCAAAGACCCCGAGCACCATAGGGCCGCT |
| | | TTCAACAATAACGATAAGCTCATCAACGCCA |
| | | TCAAGGCCCAGTATGACGAGTTCGACAACTT |
| | | CCTCGATAACCCCAGACTCGGCTATTTCGGC |
| | | CAGGCCTTTTTCAGCAAGGAGGGCAGAAAT |
| | | TACATCATCAATTACGGCAACGAATGCTATG |
| | | ACATTCTGGCCCTCCTGAGCGGACTGAGGC |
| | | ACTGGGTGGTCCATAACAACGAAGAAGAGT |
| | | CCAGGATCTCCAGGACCTGGCTCTACAACCT |
| | | CGATAAGAACCTCGACAACGAATACATCTCC |
| | | ACCCTCAACTACCTCTACGACAGGATCACCA |
| | | ATGAGCTGACCAACTCCTTCTCCAAGAACTC |
| | | CGCCGCCAACGTGAACTATATTGCCGAAACT |
| | | CTGGGAATCAACCCTGCCGAATTCGCCGAA |
| | | CAATATTTCAGATTCAGCATTATGAAAGAGC |
| | | AGAAAAACCTCGGATTCAATATCACCAAGCT |
| | | CAGGGAAGTGATGCTGGACAGGAAGGATAT |
| | | GTCCGAGATCAGGAAAAATCATAAGGTGTT |
| | | CGACTCCATCAGGACCAAGGTCTACACCAT |
| | | GATGGACTTTGTGATTTATAGGTATTACATC |
| | | GAAGAGGATGCCAAGGTGGCTGCCGCCAAT |
| | | AAGTCCCTCCCCGATAATGAGAAGTCCCTGA |
| | | GCGAGAAGGATATCTTTGTGATTAACCTGAG |
| | | GGGCTCCTTCAACGACGACCAGAAGGATGC |
| | | CCTCTACTACGATGAAGCTAATAGAATTTGG |
| | | AGAAAGCTCGAAAATATCATGCACAACATCA |
| | | AGGAATTTAGGGGAAACAAGACAAGAGAGT |
| | | ATAAGAAGAAGGACGCCCCTAGACTGCCCA |
| | | GAATCCTGCCCGCTGGCCGTGATGTTTCCG |
| | | CCTTCAGCAAACTCATGTATGCCCTGACCAT |
| | | GTTCCTGGATGGCAAGGAGATCAACGACCT |
| | | CCTGACCACCCTGATTAATAAATTCGATAAC |
| | | ATCCAGAGCTTCCTGAAGGTGATGCCTCTCA |
| | | TCGGAGTCAACGCTAAGTTCGTGGAGGAAT |
| | | ACGCCTTTTTCAAAGACTCCGCCAAGATCGC |
| | | CGATGAGCTGAGGCTGATCAAGTCCTTCGC |
| | | TAGAATGGGAGAACCTATTGCCGATGCCAG |
| | | GAGGGCCATGTATATCGACGCCATCCGTATT |
| | | TTAGGAACCAACCTGTCCTATGATGAGCTCA |
| | | AGGCCCTCGCCGACACCTTTTCCCTGGACG |
| | | AGAACGGAAACAAGCTCAAGAAAGGCAAGC |
| | | ACGGCATGAGAAATTTCATTATTAATAACGT |
| | | GATCAGCAATAAAAGGTTCCACTACCTGATC |
| | | AGATACGGTGATCCTGCCCACCTCCATGAG |
| | | ATCGCCAAAAACGAGGCCGTGGTGAAGTTC |
| | | GTGCTCGGCAGGATCGCTGACATCCAGAAA |
| | | AAACAGGGCCAGAACGGCAAGAACCAGATC |
| | | GACAGGTACTACGAAACTTGTATCGGAAAG |
| | | GATAAGGGCAAGAGCGTGAGCGAAAAGGTG |
| | | GACGCTCTCACAAAGATCATCACCGGAATG |
| | | AACTACGACCAATTCGACAAGAAAAGGAGC |
| | | GTCATTGAGGACACCGGCAGGGAAAACGCC |
| | | GAGAGGGAGAAGTTTAAAAAGATCATCAGC |
| | | CTGTACCTCACCGTGATCTACCACATCCTCA |
| | | AGAATATTGTCAATATCAACGCCAGGTACGT |
| | | CATCGGATTCCATTGCGTCGAGCGTGATGCT |
| | | CAACTGTACAAGGAGAAAGGCTACGACATC |
| | | AATCTCAAGAAACTGGAAGAGAAGGGATTC |
| | | AGCTCCGTCACCAAGCTCTGCGCTGGCATT |
| | | GATGAAACTGCCCCCGATAAGAGAAAGGAC |
| | | GTGGAAAAGGAGATGGCTGAAAGAGCCAAG |
| | | GAGAGCATTGACAGCCTCGAGAGCGCCAAC |
| | | CCCAAGCTGTATGCCAATTACATCAAATACA |
| | | GCGACGAGAAGAAAGCCGAGGAGTTCACCA |
| | | GGCAGATTAACAGGGAGAAGGCCAAAACCG |
| | | CCCTGAACGCCTACCTGAGGAACACCAAGT |
| | | GGAATGTGATCATCAGGGAGGACCTCCTGA |
| | | GAATTGACAACAAGACATGTACCCTGTTCAG |
| | | AAACAAGGCCGTCCACCTGGAAGTGGCCAG |
| | | GTATGTCCACGCCTATATCAACGACATTGCC |
| | | GAGGTCAATTCCTACTTCCAACTGTACCATT |
| | | ACATCATGCAGAGAATTATCATGAATGAGAG |
| | | GTACGAGAAAAGCAGCGGAAAGGTGTCCGA |
| | | GTACTTCGACGCTGTGAATGACGAGAAGAA |
| | | GTACAACGATAGGCTCCTGAAACTGCTGTGT |
| | | GTGCCTTTCGGCTACTGTATCCCCAGGTTTA |
| | | AGAACCTGAGCATCGAGGCCCTGTTCGATA |
| | | GGAACGAGGCCGCCAAGTTCGACAAGGAGA |
| | | AAAAGAAGGTGTCCGGCAATTCCtaagtcgacaagc |
| | | ttgcggccgcactcgagcaccaccaccaccaccactgagatccggctgcta |
| | | acaaagcccgaaaggaagctgagttggctgctgccaccgctgagcaataac |
| | | tagcataaccccttggggcctctaaacgggtcttgaggggttttttgctgaaag |
| | | gaggaactatatccggattggcgaatgggacgcgccctgtagcggcgcatta |
| | | agcgcggcgggtgtggtggttacgcgcagcgtgaccgctacacttgccagc |
| | | gccctagcgcccgctcctttcgctttcttcccttcctttctcgccacgttcgccgg |
| | | ctttccccgtcaagctctaaatcgggggctccctttagggttccgatttagtgctt |
| | | tacggcacctcgaccccaaaaaacttgattagggtgatggttcacgtagtggg |
| | | ccatcgccctgatagacggtttttcgccctttgacgttggagtccacgttctttaa |
| | | tagtggactcttgttccaaactggaacaacactcaaccctatctcggtctattctt |
| | | ttgatttataagggattttgccgatttcggcctattggttaaaaaatgagctgattt |
| | | aacaaaaatttaacgcgaattttaacaaactagtaacgtttacaatttcaggtgg |
| | | cacttttcggggaaatgtgcgcggaacccctatttgtttatttttctaaatacattc |
| | | aaatatgtatccgctcatgaattaattcttagaaaaactcatcgagcatcaaatg |
| | | aaactgcaatttattcatatcaggattatcaataccatatttttgaaaaagccgttt |
| | | ctgtaatgaaggagaaaactcaccgaggcagttccataggatggcaagatcc |
| | | tggtatcggtctgcgattccgactcgtccaacatcaatacaacctattaatttcc |
| | | cctcgtcaaaaataaggttatcaagtgagaaatcaccatgagtgacgactgaa |
| | | tccggtgagaatggcaaaagtttatgcatttctttccagacttgttcaacaggcc |
| | | agccattacgctcgtcatcaaaatcactcgcatcaaccaaaccgttattcattcg |
| | | tgattgcgcctgagcgagacgaaatacgcgatcgctgttaaaaggacaatta |
| | | caaacaggaatcgaatgcaaccggcgcaggaacactgccagcgcatcaac |
| | | aatgttttcacctgaatcaggatattcttctaatacctggaatgctgttttcccggg |
| | | gatcgcagtggtgagtaaccatgcatcatcaggagtacggataaaatgcttga |
| | | tggtcggaagaggcataaattccgtcagccagtttagtctgaccatctcatctg |
| | | taacatcattggcaacgctacctttgccatgtttcagaaacaactctggcgcatc |
| | | gggcttcccatacaatcgatagattgtcgcacctgattgcccgacattatcgcg |
| | | agcccatttatacccatataaatcagcatccatgttggaatttaatcgcggccta |
| | | gagcaagacgtttcccgttgaatatggctcataacaccccttgtattactgtttat |
| | | gtaagcagacagttttattgttcatgaccaaaatcccttaacgtgagttttcgttc |
| | | cactgagcgtcagaccccgtagaaaagatcaaaggatcttcttgagatcctttt |
| | | tttctgcgcgtaatctgctgcttgcaaacaaaaaaaccaccgctaccagcggt |
| | | ggtttgtttgccggatcaagagctaccaactctttttccgaaggtaactggcttc |
| | | agcagagcgcagataccaaatactgtccttctagtgtagccgtagttaggcca |
| | | ccacttcaagaactctgtagcaccgcctacatacctcgctctgctaatcctgtta |
| | | ccagtggctgctgccagtggcgataagtcgtgtcttaccgggttggactcaag |
| | | acgatagttaccggataaggcgcagcggtcgggctgaacggggggttcgtg |
| | | cacacagcccagcttggagcgaacgacctacaccgaactgagatacctaca |
| | | gcgtgagctatgagaaagcgccacgcttcccgaagggagaaaggcggaca |
| | | ggtatccggtaagcggcagggtcggaacaggagagcgcacgagggagctt |
| | | ccagggggaaacgcctggtatctttatagtcctgtcgggtttcgccacctctga |
| | | cttgagcgtcgatttttgtgatgctcgtcaggggggcggagcctatggaaaaa |
| | | cgccagcaacgcggcctttttacggttcctggccttttgctggccttttgctcac |
| | | atgttctttcctgcgttatcccctgattctgtggataaccgtattaccgcctttgag |
| | | tgagctgataccgctcgccgcagccgaacgaccgagcgcagcgagtcagt |
| | | gagcgaggaagcggaagagcgcctgatgcggtattttctccttacgcatctgt |
| | | gcggtatttcacaccgcaatggtgcactctcagtacaatctgctctgatgccgc |
| | | atagttaagccagtatacactccgctatcgctacgtgactgggtcatggctgcg |
| | | ccccgacacccgccaacacccgctgacgcgccctgacgggcttgtctgctc |
| | | ccggcatccgcttacagacaagctgtgaccgtctccgggagctgcatgtgtc |
| | | agaggttttcaccgtcatcaccgaaacgcgcgaggcagctgcggtaaagctc |
| | | atcagcgtggtcgtgaagcgattcacagatgtctgcctgttcatccgcgtcca |
| | | gctcgttgagtttctccagaagcgttaatgtctggcttctgataaagcgggccat |
| | | gttaagggcggttttttcctgtttggtcactgatgcctccgtgtaagggggatttc |
| | | tgttcatgggggtaatgataccgatgaaacgagagaggatgctcacgatacg |
| | | ggttactgatgatgaacatgcccggttactggaacgttgtgagggtaaacaac |
| | | tggcggtatggatgcggcgggaccagagaaaaatcactcagggtcaatgcc |
| | | agcgcttcgttaatacagatgtaggtgttccacagggtagccagcagcatcct |
| | | gcgatgcagatccggaacataatggtgcagggcgctgacttccgcgtttcca |
| | | gactttacgaaacacggaaaccgaagaccattcatgttgttgctcaggtcgca |
| | | gacgttttgcagcagcagtcgcttcacgttcgctcgcgtatcggtgattcattct |
| | | gctaaccagtaaggcaaccccgccagcctagccgggtcctcaacgacagg |
| | | agcacgatcatgcgcacccgtggggccgccatgccggcgataatggcctgc |
| | | ttctcgccgaaacgtttggtggcgggaccagtgacgaaggcttgagcgagg |
| | | gcgtgcaagattccgaataccgcaagcgacaggccgatcatcgtcgcgctc |
| | | cagcgaaagcggtcctcgccgaaaatgacccagagcgctgccggcacctg |
| | | tcctacgagttgcatgataaagaagacagtcataagtgcggcgacgatagtc |
| | | atgccccgcgcccaccggaaggagctgactgggttgaaggctctcaaggg |
| | | catcggtcgagatcccggtgcctaatgagtgagctaacttacattaattgcgtt |
| | | gcgctcactgcccgctttccagtcgggaaacctgtcgtgccagctgcattaat |
| | | gaatcggccaacgcgcggggagaggcggtttgcgtattgggcgccagggt |
| | | ggtttttcttttcaccagtgagacgggcaacagctgattgcccttcaccgcctg |
| | | gccctgagagagttgcagcaagcggtccacgctggtttgccccagcaggcg |
| | | aaaatcctgtttgatggtggttaacggcgggatataacatgagctgtcttcggt |
| | | atcgtcgtatcccactaccgagatatccgcaccaacgcgcagcccggactcg |
| | | gtaatggcgcgcattgcgcccagcgccatctgatcgttggcaaccagcatcg |
| | | cagtgggaacgataccctcattcagcatttgcatggtttgttgaaaaccggaca |
| | | tggcactccagtcgccttcccgttccgctatcggctgaatttgattgcgagtga |
| | | gatatttatgccagccagccagacgcagacgcgccgagacagaacttaatg |
| | | ggcccgctaacagcgcgatttgctggtgacccaatgcgaccagatgctccac |
| | | gcccagtcgcgtaccgtcttcatgggagaaaataatactgttgatgggtgtctg |
| | | gtcagagacatcaagaaataacgccggaacattagtgcaggcagcttccaca |
| | | gcaatggcatcctggtcatccagcggatagttaatgatcagcccactgacgc |
| | | gttgcgcgagaagattgtgcaccgccgctttacaggcttcgacgccgcttcgt |
| | | tctaccatcgacaccaccacgctggcacccagttgatcggcgcgagatttaat |
| | | cgccgcgacaatttgcgacggcgcgtgcagggccagactggaggtggcaa |
| | | cgccaatcagcaacgactgtttgcccgccagttgttgtgccacgcggttggga |
| | | atgtaattcagctccgccatcgccgcttccactttttcccgcgttttcgcagaaa |
| | | cgtggctggcctggttcaccacgcgggaaacggtctgataagagacaccgg |
| | | catactctgcgacatcgtataacgttactggtttcacattcaccaccctgaattg |
| | | actctcttccgggcgctatcatgccataccgcgaaaggttttgcgccattcgat |
| | | ggtgtccgggatctcgacgctctcccttatgcgactcctgcattaggaagcag |
| | | cccagtagtaggttgaggccgttgagcaccgccgccgcaaggaatggtgca |
| | | tg |

KEY:
START CODON (CAPS, bold);
His Tag * (bold);
MBP protein (bold & underlined)
TEV (underlined)
CASRX CDS (CAPS, bold & underlined)
stop codon (taa following CasRx CDS)
plasmid backbone (others)
Probes (Detection Probe for Collateral Cleavage)

| FQ-Fluorescence | FQ | /56- |
|---|---|---|
| probe. Poly-U probe | | FAM/rUrUrUrUrUrU/3IABkFQ/ |
| conjugated to | | |
| fluorescine and a | | |
| quencher for use in | | |
| Fluorescence Assay- | | |
| Ordered from IDT | | |
| FRU-Fluorescence | FRU | /56- |
| Reporter-Uracil; | | FAM/rUrUrUrUrUrU/3IABkFQ/ |
| Poly-U probe | | |
| modified with 5′ 6- | | |
| Carboxyfluoroscein | | |
| and a fluorescence | | |
| quencher for use in | | |
| fluorescence detection | | |
| -Custom ordered from | | |
| IDT | | |
| FRA-Fluorescence | FRA | /56- |
| Reporter-Adenosine; | | FAM/rArArArArArA/3IABkFQ/ |
| Poly-A probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRG-Fluorescence | FRG | /56- |
| Reporter-Uracil; GG | | FAM/TArGrGAT/3IABkFQ/ |
| probe modified with 5′ | | |
| 6-Carboxyfluoroscein | | |
| and a fluorescence | | |
| quencher for use in | | |
| fluorescence detection | | |
| -Custom ordered from | | |
| IDT | | |
| FRC-Fluorescence | FRC | /56- |
| Reporter-Adenosine; | | FAM/rCrCrCrCrCrC/3IABkFQ/ |
| Poly-C probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRAU-Fluorescence | FRAU | /56- |
| Reporter-Adenosine; | | FAM/rArUrArUrArU/3IABkFQ/ |
| AU/UA probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRAG-Fluorescence | FRAG | /56- |
| Reporter-Adenosine; | | FAM/rArGrArGrArG/3IABkFQ/ |
| AG/GA probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRAC-Fluorescence | FRAC | /56- |
| Reporter-Adenosine; | | FAM/rArCrArCrArC/3IABkFQ/ |
| AC/CA probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRGU-Fluorescence | FRGU | /56- |
| Reporter-Adenosine; | | FAM/rGrUrGrUrGrU/3IABkFQ/ |
| GU/UG probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRCU-Fluorescence | FRCU | /56- |
| Reporter-Adenosine; | | FAM/rCrUrCrUrCrU/3IABkFQ/ |
| CU/UC probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FRGC-Fluorescence | FRGC | /56- |
| Reporter-Adenosine; | | FAM/rGrCrGrCrGrC/3IABkFQ/ |
| GC/CG probe | | |
| modified with 5′ 6- | | |
| Carboxyfluoroscein | | |
| and a 3′ fluorescence | | |
| quencher for use in | | |
| fluorescence detection | | |
| -Custom ordered from | | |
| IDT | | |
| FB-Lateral flow | FB | /56- |
| probe.; Poly-U probe | | FAM/rUrUrUrUrUrU/3Bio/ |
| conjugated to | | |
| fluorescine and biotin | | |
| for use in the Lateral | | |
| Flow Assay-Ordered | | |
| from IDT | | |
| LFRU-Lateral Flow | LFRU | /56- |
| Reporter-Uracil; | | FAM/rUrUrUrUrUrU/3Bio/ |
| Poly-U probe | | |
| modified with 5′ 6- | | |
| Carboxyfluorescein | | |
| and 3′ biotin for use in | | |
| the lateral flow | | |
| detection-Custom | | |
| ordered from IDT | | |

In some embodiments, the system further comprises a reagent for reverse transpiration (RT) of the RNA target sequence(s) in the sample. In further embodiments, the RT reagent is selected from one or both of a reverse transcriptase and a buffer suitable for the reverse transpiration.

In some embodiments, the system further comprises reagents for amplifying the target sequences from the sample. In a further embodiment, the target sequences is amplified to double-stranded DNA (dsDNA) amplicons. Additionally or alternatively, the amplification is selected from reverse transcriptase recombinase polymerase amplification (RT-RPA) or reverse transcriptase isothermal amplification, such as Reverse transcription loop-mediated isothermal amplification, RT-LAMP. In some embodiments, the RT-RPA reagent(s) is or are selected from one or more of: RT-PRA primers amplifying a sequence comprising the target sequences and/or gRNA spacer regions, a Reverse Transcriptase, a recombinase, a single strand binding protein, and a buffer suitable for the application. In some embodiments, the RT-PRA primer comprises or consists essentially of, or yet further consists of a promoter sequence and a primer. In a further embodiment, the promoter sequence is a T7 promoter, such as the one disclosed herein. In yet a further embodiment, the primer is capable of annealing to the target sequence or a contiguous sequence in the gene.

In some embodiments, the method further comprises in vitro transcription (IVT) reagents. In further embodiments, the IVT reagents are selected from one or more of: RNA polymerase, ATP, GTP, UTP, CTP, and a buffer suitable for the IVT. In some embodiments, the buffer is also suitable for the CRISPR reagents. In further embodiments, the IVT step may be performed with the CRISPR step at the same time and in the same reaction.

In some embodiments, the system comprises one or more of: an E gene gRNA (such as a gRNA-T as disclosed herein), an N gene gRNA (such as a gRNA-Z as disclosed herein), or an S gene gRNA. In some embodiments, the gRNA is as disclosed herein. In further embodiments, the gRNA is disclosed herein as its corresponding target sequence. For example, a target sequence is disclosed herein, and the corresponding gRNA comprises or consists essentially of, or yet further consists of a sequence complementary to the target sequence or a fragment thereof and optionally having 0, or 1, or 2, or 3 mismatch(es). Another example is that a target sequence is disclosed herein, and the corresponding gRNA comprises, or consists essentially of, or yet further consists of a sequence of the target sequence or a fragment thereof if the target sequence is an RNA and optionally having 0, or 1, or 2, or 3 mismatch(es). Yet another example is that a target sequence is disclosed herein, and the corresponding gRNA comprises, or consists essentially of, or yet further consists of a sequence of the target sequence or a fragment thereof having the T residue(s) replaced with U residue(s) if the target sequence is not an RNA, such as a DNA or a hybrid of DNA and RNA and optionally having 0, or 1, or 2, or 3 mismatch(es). In a further embodiment, the corresponding gRNA further comprises a direct repeat, optionally a 5′ direct repeat. In yet a further embodiment, the direct repeat is as disclosed herein. Additionally or alternatively, the direct repeat is about 10 to about 50, including any integer therebetween, nt long. In some embodiments, the target sequence or a fragment thereof is about 10 to about 50, including any integer therebetween, such as about 25 nt long to about 35 nt long, or about 30 nt long.

In some embodiments, the system and/or the CRISPR reagents comprise or consist essentially of, or yet further consist of a Cas13 enzyme. In further embodiments, the Cas13 enzyme is a Cas13d enzyme. In some embodiment, the Cas13d is Ruminococcus flavefaciens Cas13d (CasRx). In some embodiments, the system and/or the CRISPR reagents comprise, or consist essentially of, or yet further consist of a fusion protein comprising, or alternatively consisting essentially of, or yet further consisting of the Cas13d enzyme, an optional protein cleavage site (such as a TEV protease cleavage sequence), a purification marker or tag (such as a 6×His tag), and an optional Maltose-binding protein or a fragment thereof. In yet further embodiment, the system and/or the CRISPR reagents further comprise an accessory protein comprising, or alternatively consisting essentially of, or yet further consisting of a WYL1-domain.

In some embodiments, the method further comprises a reporting reagent. In some embodiments, the reporting reagent is a probe. In further embodiments, the reporting reagent is a probe conjugated with one or more purification or detectable markers (such as radioisotopes, fluorochromes, chemiluminescent compounds, dyes, and proteins, including enzymes). In some embodiments, the reporting reagent comprises, or consists essentially of, or yet further consists of a fluorophore and a quencher. In further embodiments, the fluorophore can be placed in close proximity to the quencher. In yet further embodiments, the system permits release of the fluorophore from the close proximity to the quencher upon detection of the target sequence. In some embodiments, the probe is a collateral cleavage probe, for example, the probe can be cleaved due to the collateral cleavage activity of the Cas13 enzyme as disclosed herein. In some embodiment, such cleavage allowing releasing of the purification or detectable markers. In further embodiments, the probe comprises, or consists essentially of, or yet further consists of a poly U sequence, such as having about 4 to about 20 U residues. In one embodiment, the probe comprises or consists essentially of, or yet further consists of a 6-nt poly-U. In some embodiments, the reporting reagent comprises, or consists essentially of, or yet further consists of a probe (optionally a poly U as disclosed herein) conjugated to a fluorescence maker (such as a 5′ fluorescent marker and/or a 6-FAM) and a quencher (such as a 3′ quencher and/or optionally an IABlkFQ). In some embodiments, the reporting reagent comprises, or consists essentially of, or yet further consists of a probe (optionally a poly U as disclosed herein) conjugated to a biotin and/or a fluorescent marker). In some embodiments, the CasRx or Cas13d facilitates fluorescence-based readouts of RNase activity. In some embodiments, the system further comprises a means for visual indication of activity, such as to be read out visually under UV, or quantitatively by a fluorometer. In some embodiments, the CasRx enzyme is modified to detect SARS-Cov-2 genetic material by lateral flow assay. Further non-limiting examples of reporting reagents are provided in Table 5.

In another aspect, the system further comprises CasRx or Cas13. In a yet further aspect, the CasRx or Cas13 facilitates fluorescence-based readouts of RNase activity. In another aspect, the CasRx enzyme is modified to detect SARS-Cov-2 genetic material by lateral flow assay.

One of skill in the art may understand that CasRx or Cas13 as disclosed herein may be substituted with a cell producing CasRx or Cas13, or a vector (plasmid or viral) encoding CasRx or Cas13 for expression in a cell. Such cells and vectors can be used to produce the CasRx or Cas13, which in turn function in a system or a method as disclosed herein.

In another embodiment, the system further comprises a fluorophore and a quencher, wherein optionally the fluorophore can be placed in close proximity to the quencher.

In another aspect, the system further comprises a means for visual indication of activity, optionally to be read out visually under UV, or quantitatively by a fluorometer.

The system is useful in a method to detect SARS-CoV-2 in a sample, by contacting the sample with the system as described herein. Non-limiting examples are disclosed herein and include samples isolated from one or more of the lungs, oral cavity or nasal cavity of a subject. In one embodiment, the subject is a mammal that is susceptible to infection by SARS-CoV-2, e.g., a bat, a simian, a human, a feline, or a canine. The method also comprises detecting the presence of SARS-CoV-2, in the sample by detecting the presence of the pathogen (such as SARS-CoV-2) gene, such as the E gene, the S gene, and/or the N gene or alternatively the presence of the E gene and the N gene.

In some embodiments, the system is provided as a fluorescence assay system. For example, the fluorescence assay system may comprise, or consist essentially of, or yet further consist of a gRNA targeting a target sequence, a Cas enzyme, and a reporting agent comprising, or consisting essentially of, or yet further consisting of a probe conjugated to a fluorescence marker and a quencher. Other suitable buffers may be further included.

In some embodiments, the system is provided as a lateral flow assay (LFA) system. For example, the fluorescence assay system may comprise, or consist essentially of, or yet further consist of a gRNA targeting a target sequence, a Cas enzyme, a reporting agent comprising, or consisting essentially of, or yet further consisting of a probe conjugated to a detectable or purification marker and a binding moiety, and an immobilized ligand of the binding moiety. Other suitable buffers may be further included. In some embodiments, the lateral flow assay system comprises, or consists essentially of, or yet further consists of a carrier that allows a lateral flow to occur wherein either the sample or the detection reagent is displaced from one location on the carrier to another, and wherein the latter location of the carrier immobilized with the ligand. There are many formats of lateral flow assays suitable for use, and the skilled person will readily know how to select and optimize a particular format. An example of a lateral flow test strip comprises, or consists essentially of, or yet further consists of, for example, the following components: a sample pad—an absorbent pad onto which the test sample is applied; conjugate or reagent pad—this contains the reporting reagent, the gRNA and the Cas enzyme; reaction membrane—typically a hydrophobic nitrocellulose or cellulose acetate membrane onto which ligands are immobilized in a line across the membrane as a capture zone or test line (a control zone may also be present, containing antibodies specific for the conjugate antibodies); and wick or waste reservoir—a further absorbent pad designed to draw the sample across the reaction membrane by capillary action and collect it.

### Methods

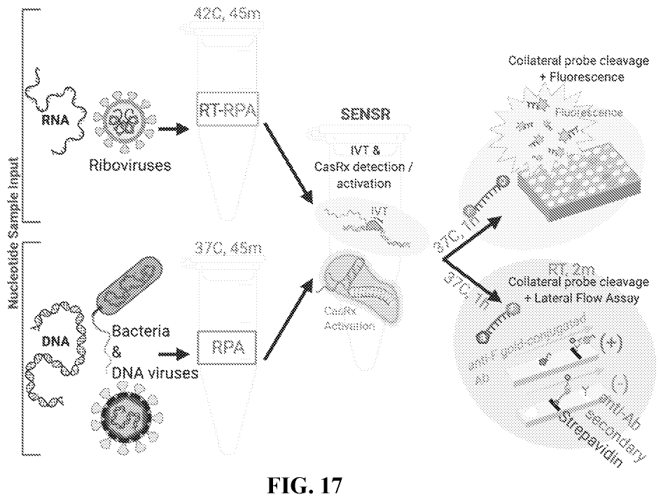

CasRx-based diagnostic systems may present a worthy advancement for CRISPRDx due to the fundamental characteristics of the Cas13d family. Like LwaCas13a, Cas13d is more flexible than most other Cas enzymes because it lacks a protospacer flanking sequence (PFS) requirement (Freije et al., 2019; Konermann et al., 2018; and Yan et al., 2018), permitting targeting of any sequence without constraint. In addition, some native Cas13d systems include a WYL1-domain-containing accessory protein, which has been demonstrated to increase the on-target and collateral cleavage efficiency of the Cas13d effectors (Yan et al., 2018; and Zhang et al., Nucleic Acids Res. 47, 5420-5428 (2019)), suggesting potential for future implementation. Furthermore, because they target RNA, next-generation Cas13-based systems may be capable of direct recognition of RNA, possibly at the single molecule level, without need for a prior reverse transcription (RT) and/or amplification step. This property could enable direct detection of many emerging viral threats including, but not limited to; bunyaviruses (Noronha et al., 2017), zoonotic viruses such as Ebola, hanta, and Lassa (Wang et al., 2014); arboviruses such as dengue, chikungunya, and Zika (Gootenberg et al., 2018; Gould et al., 2017; and Charrel et al. Emerg. Infect. Dis. 11, 1657-1663 (2005)), and other coronaviruses such as MERS, SARS-CoV-1, as well as those yet undiscovered (Li et al., 2005; and Guarner et al., 2020). CasRx-based diagnostics systems could detect endemic pathogens capable of zoonotic transmission through livestock and wild animals such as influenza or other coronaviruses (Li et al., 2005; Torremorell et al., Transbound. Emerg. Dis. 59 Suppl 1, 68-84 (2012); and Shi et al., Cell Res. 27, 1409-1421 (2017)) which may have been able to prevent past pandemics (Mena et al. Elife 5, (2016)), and avert mass herd culling resulting in billions of dollars of losses (MacKenzie, New Scientist vol. 244 6 (2019); and Parry. Bull. World Health Organ. 85, 3-4 (2007)). Beyond detection in patients and livestock, SENSR could be adapted to detect pathogens in insect disease vectors as well as infected individuals (Lee et al. Proceedings of the National Academy of Sciences 202010196 (2020) doi:10.1073/pnas.2010196117), facilitating rapid one-pot field detection of mosquito-borne pathogens in areas lacking laboratory infrastructure (Choumet et al., Rev. Sci. Tech. 34, 473-8, 467-72 (2015)). However, SENSR is not limited to detection of RNA species, and could also be used to detect pathogen DNA (FIG. 17). By including an RNA polymerization step, this same technology could be harnessed to track evidence of insecticide resistance alleles (Faucon et al. Genome Res. 25, 1347-1359 (2015)), released transgenic cargoes (Esvelt et al., 2014; and Li et al., 2020), or the presence of Lyme (Borrelia burgdorferi) in a tick plucked from a hiker's leg.

Pushing the boundaries of viral sequence recognition with CRISPR-Cas nucleases is not only of interest for genetic engineering and diagnostics, but also for therapeutics as well. The adaptability of CasRx RNA-targeting has recently been demonstrated to be a potentially powerful anti-COVID therapeutic (Abbott et al., 2020) as well as for other viruses (Blanchard et al. 2020, bioRxiv doi:10.1101/2020.04.24.060418). Together with acute diagnostics, these technologies could promise a new mode of response to future viral outbreaks via a ‘plug-n-chug’ model, in which complementary diagnostics and therapeutics could be systematically rolled out almost immediately after completion of a viral genome sequence. Similar to LwaCas13a, CasRx could also be adapted to massively multiplexed arrays to facilitate identification of viral pathogens on a large scale (Ackerman et al. 2020). Establishing these tools and frameworks now, could expedite response times and help prevent future outbreaks, avoiding the economic and health consequences which have resulted from poor preparedness to the current pandemic.

In one aspect, provided is a method to detect SARS-CoV-2 in a sample. In some embodiments, the method comprises, or consists essentially of, or yet further consists of contacting the sample with the system as disclosed herein. In some embodiments, the sample is isolated from one or more of the lungs, oral cavity or nasal cavity of a subject. In some embodiments, the subject is a mammal that is susceptible to infection by SARS-CoV-2. In some embodiments, the mammal is a bat, a simian, a human, a feline, or a canine, a murine, a rat, a rabbit, a bovine, an ovine, a porcine, an equine, and a primate. In some embodiments, the method further comprises detecting the presence of the pathogen, such as SARS-CoV-2, in the sample by detecting the presence of the target sequence, such as the S gene, the E gene and/or the N gene. In some embodiments, the method further comprises detecting the presence of SARS-CoV-2, in the sample by detecting the presence of the E gene and the N gene. In some embodiments, the limit of detection (LOD) of the method about 10 to about 1000 copies (optionally 100 copies) per RT-RPA reaction or per microliter, for example of the reaction system. In some embodiments, the specificity and/or the concordance of the method is at least about 50%, or at least about 60%, or at least about 70%, or at least about 80%, or at least about 90%, or at least about 91%, or at least about 92%, or at least about 93%, or at least about 94%, or at least about 95%, or at least about 96%, or at least about 97%, or at least about 98%, or at least about 99%, or about 100%.

In some embodiments, a method as disclosed herein comprises, or consists essentially of, or yet further consists of one or more of the following steps: isolating nucleotides from a sample; reverse transcribing the nucleotides if such nucleotides are RNA; amplifying DNA comprising, or consisting essentially of, or yet further consisting of a target sequence or a complementary sequence thereof, for example by recombinase polymerase amplification; transcribing the amplified DNA to RNA; incubating the RNA with a system as disclosed herein, such as those comprising, or consisting essentially of, or yet further consisting of a gRNA as disclosed herein, a CRISPR enzyme, such as CasRx or Cas13, and a reporting reagent.

In some embodiments, a method as disclosed herein further comprises treating the subject detected with SARS-CoV-2 with an anti-SARS-CoV-2 therapeutic composition. In further embodiments, such therapeutic composition may comprise, or consist essentially of, or yet further consist of bamlanivimab, etesevimab, casirivimab, imdevimab, remdesivir, dexamethasone, tocilizumab, anti-inflammatory agent, or any combination thereof. Other therapeutic composition is available at www.covid19treatmentguidelines.nih.gov/therapeutic-management/ and www.drugs.com/condition/covid-19.html.

In some embodiments, a method as disclosed herein further comprises treating the subject not detected with SARS-CoV-2 with an anti-SARS-CoV-2 vaccine, see, for example, those listed on www.cdc.gov/coronavirus/2019-ncov/vaccines/different-vaccines.html.

In some embodiments, SARS-CoV-2 as disclosed herein can be substituted with another pathogen and the gRNA(s) as disclosed in the systems and methods may be updated based on the genome of the pathogen. In further embodiments, other components of the system as disclosed herein remain the same. As used herein, a pathogen is a microorganism that can cause a disease, including a RNA virus (i.e., a virus that has RNA as its genetic material), a DNA virus (i.e., a virus that has DNA as its genetic material, such as herpes), a bacterium, or a fungi. In some embodiments, the pathogen is a riboviruse. In further embodiments, the riboviruse comprises, or consists essentially of, or yet further consists of coronavirus (such as MERS, SARS-CoV-1, or SARS-CoV-2), ebola virus, HIV, influenza virus (such as H1N1), hantavirus, lassa virus, bunyavirale, zika virus, Dengue virus, Toscana phlebovirus (TOSV), Chikungunya virus (CHIKV), Nairovirus or rabie virus. Additionally or alternatively, the pathogen may be an arbovirus, such as Dengue virus, Japanese encephalitis virus, Rift Valley fever virus, Tick-borne encephalitis virus, West Nile virus, or Yellow fever virus.

In some embodiments, the sample has been purified to comprise, or consist essentially of, or yet further consist of nucleotides of a pathogen if any. In further embodiments, the nucleotides of the pathogen have been isolated. In further embodiments, the nucleotides of the pathogen have been enriched. In yet further embodiments, the nucleotides of the pathogen have been amplified. In some embodiments, DNA-based sample (such as those for detecting a DNA virus, a bacterium, or a fungi) can be input directly into the RPA amplification reaction, negating the need for a simultaneous reverse transcription (RT) reaction as is required for RNA-based samples.

In some embodiments, following extraction of viral RNA, the method comprises, or consists essentially of, or yet further consists of any one, or any two, or all three of the following steps/reactions. In some embodiments, the last step differs based on desired output detection method. In the first reaction, specific target sequences within the viral RNA are reverse transcribed (RT) into cDNA and amplified, for example, by RPA at 42° C. for 45 min, while also adding T7 promoter sequences to the 5′ terminus (T7). In the next reaction, in vitro transcription occurs simultaneously with CasRx collateral cleavage activation by recognition and cleavage of the target RNA sequence through the sequence-specific targeting activity of the gRNA. In this third reaction, addition of a probe conjugated to fluorescein and a quencher can facilitate readout by fluorescence following probe cleavage. Alternatively, addition of a probe conjugated to fluorescein and biotin facilitates readout by lateral flow assay (bottom right).

Another example is illustrated in FIG. 10. In step 1, viral single strand RNA (ssRNA) is extracted and reverse transcribed into cDNA. Reverse transcriptase actively reverse transcribes the viral template into cDNA. In step 2, in the simultaneous RPA reaction, recombinase binds to the primer with D-loops formed at binding sites. Recombinase helps anneal the primer to the target site, and single strand binding protein (SSB) begins annealing to single strand DNA (ssDNA) to stabilize the strand and reaction. In step 3, strand-displacing DNA polymerase amplifies the target DNA, with continued binding of SSB to ssDNA for stabilization. In step 4, DNA amplicon extension is completed, while polymerase simultaneously dislodges SSB, and the T7 promoter region is added to the amplicon via primer extension. In step 5, the final product of the RT-RPA reaction is a small amplified fragment of target DNA encompassing the CasRx target site, with a T7 promoter added for subsequent IVT. In step 6, T7 RNA polymerase-based in vitro transcription occurs, initiated from the T7 promoter in order to generate ssRNA required as the activation substrate of CasRx. Elongation of the ssRNA template takes place. In step 7, CasRx detection of on-target sequence, activation of collateral cleavage activity, and detection. The CasRx/gRNA complex recognize, bind to, and cleave the ssRNA viral target template. This action activates the collateral cleavage property of CasRx. Following activation, collateral cleavage of an included small ssRNA probe can be analyzed via fluorescence or lateral flow assay.

To Identify Target Sites with Even Fluorescence Based Detection of Cleavage

CasRx has been shown to confer collateral cleavage of off-target RNA molecules activated specifically following on-target cleavage (Konermann et al. 2018; Buchman et al. 2020), a feature shared by other Cas13 ribonucleases (Abudayyeh et al. 201; Gootenberg et al. 2017; East-Seletsky et al. 2016, Nature 538 (7624): 270-73; Yan et al. 2018, Mol. Cell 70, 327-339.e5 (2018); Smargon et al. 2017, Molecular Cell. doi.org/10.1016/j.molcel.2016.12.023; Meeske, et al. 2019, Nature 570 (7760): 241-45). Applicant therefore harnessed this tandem RNase activity to act as a reporter indicating the presence of a sequence in a sample corresponding to the SARS-CoV-2 genome (see, for example, SEQ ID NO: 1). The RNASEALERT LAB TEST KIT™ (Thermo Fisher Scientific) uses a modified RNA molecule containing a fluorophore in close proximity to a quencher, whose cleavage thus facilitates fluorescence-based readouts of RNase activity. In the presence of RNase activity, cleavage accumulates and fluorescence compounds, providing a visual indication of activity which can be read out visually under UV, or quantitatively by a fluorometer (Kellner et al. 2019, Nature Protocols 14 (10): 2986-3012).

To determine if, and the sensitivity by which, CasRx could detect the presence of viral genomic sequences by fluorescence, Applicant combined CasRx, (E)- or (N)-gene targeting gRNAs, and viral-genome mimic RNA at varying concentrations into a modified RNASEALERT reaction. Applicant demonstrated that robust detection can be achieved by recognition with both gRNAs, in samples with as low as minimal copies per L after minimal incubation time, and in as little as minutes when provided multitudes of viral genomic equivalents per μL. Because CasRx maintains a sequence preference for collateral cleavage of poly-X transcripts, Applicant designed two probes each composed of 6 bp of Adenine or uracil, each conjugated on the end with a moiety.

### Lateral Flow

The collateral cleavage properties of the CasRx enzyme can also be modified to detect SARS-Cov-2 genetic material by lateral flow assay, facilitating detection via test strip and negating the need for more complex laboratory equipment. Applicant developed a lateral flow assay which can detect CasRx cleavage of a target, much like the lateral flow assay developed for DNA/RNA detection via SHERLOCK (Kellner et al. 2019, cited above). This assay detects the presence of viral RNA through the CasRx collateral RNAse activity activated following recognition of the viral genomic sequence. To do this, Applicant modified a HybriDetect lateral flow strip to detect evidence of CasRx-based collateral cleavage of a secondary RNA reporter following activation by recognition of a viral genomic sequence. This reporter is conjugated on opposite ends with biotin or an oligo-based gold-bound probe, such that cleavage separates these factors permitting separate binding of these moieties to different epitopes embedded on the flow strip. Following incubation of CasRx, gRNAs, and the probe in vitro, the reaction was run on a lateral-flow dipstick, whose capillary action carries the cleaved or un-cleaved RNA reporter up the membrane. As expected, absence of reporter cleavage resulted in binding of probe to the lower band through biotin conjugation, and cleavage resulted in separation and therefore probe binding to the upper band.

With an ever increasingly interconnected world and expanding global population, future pandemics originating from zoonotic crossover of viruses into human populations is inevitable. The current pandemic of Covid-19, caused by the SARS-Cov2 virus, is well underway and could have been better controlled in many areas of the world if diagnostic tests had been developed, expedited, and deployed widely during early stages of transmission. CRISPR-based diagnostic tests are flexible, easy to optimize, and quick to develop and manufacture, making them ideal test candidates towards these ends. While CRISPR-based tests such as SHERLOCK and DETECTR have been developed since the onset of the current outbreak, earlier development and wide-spread manufacture and implementation of these technologies may have been able to help contain disease spread. Although these tests have yet to be widely implemented, they are promising candidates for future use as widespread implementation of efficient diagnostic tests would help greatly in better understanding disease spread. Therefore, now is an important time for the scientific community to use this impetus to develop an expansive toolkit which can be modified and co-opted efficiently and quickly, to be able to be deployed for diagnosis and treatment of future pandemics which emerge at exponential timescales. Therefore here Applicant outlined the development of an alternative RNA-targeting CRISPR enzyme, CasRx, to facilitate detection of SARS-Cov2 viral RNA sequences by both fluorescence as well as lateral-flow assay. Applicant demonstrated CasRx can detect evidence of SARS-Cov-2 genetic material in in-vitro synthesized as well as patient-derived samples down to the molecular level of detection, making this test sufficiently sensitive to detect as few as about 10 to about 1000 (such as 100) copies of the viral genome per μl of sample. This system can be adapted to recognize a wide range of riboviruses including, but not limited to; those of zoonotic origin such as nipah, ebola, hanta, and lassa fever (Wang and Crameri 2014, Rev. Sci. Tech. 33, 569-581 (2014)); vector-borne arboviruses such as chikungunya, Zika, Toscana, Crimean-Congo hemorrhagic fever (Gould et al. 2017, One Health 4, 1-13 (2017); Charrel et al. Emerg. Infect. Dis. 11, 1657-1663 (2005)), bunyavirales such as Rift Valley and Cache Valley fever (Noronha and Wilson 2017, Curr. Opin. Virol. 27, 36-41); in addition to other coronaviruses such as MERS, and SARS-Cov-1 and many more (Li et al. 2005, Science 310, 676-679; and Guarner 2020, Am. J. Clin. Pathol. 153, 420-421).

Pushing the boundaries of CRISPR proteins' abilities to recognize viral sequences is not only of interest for genetic engineering and diagnostics, but also for therapeutics as well. The adaptability of CasRx's RNA-targeting capabilities has also been recently demonstrated to be a potentially powerful anti-Covid therapeutic (Abbott et al. 2020, bioRxiv. doi.org/10.1101/2020.03.13.991307). Together with diagnostics, these technologies could promise a new mode of response to future riboviral outbreaks via a ‘plug-n-chug’ model, in which complementary diagnostics and therapeutics could be formulaically rolled out almost immediately after completion of the viral genome sequence. Establishing these tools and frameworks now, could expedite response times for future outbreaks, avoiding the disastrous economic and fatal consequences which have resulted from poor preparedness to the current pandemic. Developing, manufacturing, and distributing a wide range of diagnostics capable of detecting cases of Covid-19 promises to be one of the most effective methods to return society to a more economically normal state, and may help avoid this outcome in future outbreaks.

With an increasingly interconnected world and expanding global population, future pandemics are inevitable. The COVID-19 pandemic spread prolifically in the early months of 2020, with containment elusive in part due to the scarcity of point-of-care diagnostics. The seemingly infinite adaptability of CRISPR has, or promises to, accelerate the development of everything from life-saving gene therapies (Xu et al. Blood 133, 2255-2262 (2019); Maeder et al. Nat. Med. 25, 229-233 (2019); and Inc., K. N. & Kernel Networks Inc. Single Ascending Dose Study in Participants With LCA10. Case Medical Research (2019) doi:10.31525/ct1-nct03872479) and pig-to-human organ donations (Niu et al. Science vol. 357 1303-1307 (2017)); to disease-eradicating gene drives (Esvelt et al. Elife 3, e03401 (2014); Li et al. eLife vol. 9 (2020); and Champer et al. Nat. Rev. Genet. 17, 146-159 (2016)) and possibly the re-animation of the Woolly Mammoth (Church, G. Sci. Am. 309, 12 (2013); and the Woolly Mammoth Revival. Assessable at reviverestore.org/projects/woolly-mammoth/)—with CRISPR-based diagnostics (CRISPRDx) being no exception. Though still nascent, CRISPRDx, like other CRISPR technologies, has proven fast to develop, highly flexible, capable of multiplexing, making it the ideal toolkit from which to develop expeditious future point-of-care diagnostics. The CRISPRDx technologies developed prior to the COVID-19 pandemic, such as SHERLOCK and DETECTR, may have helped halt disease transmission had they been deployed earlier and implemented more widely. Therefore, it is important to prepare now, well in advance of the next pandemic, by perfecting and expanding the CRISPRDx toolkit to the bounds of its capabilities.

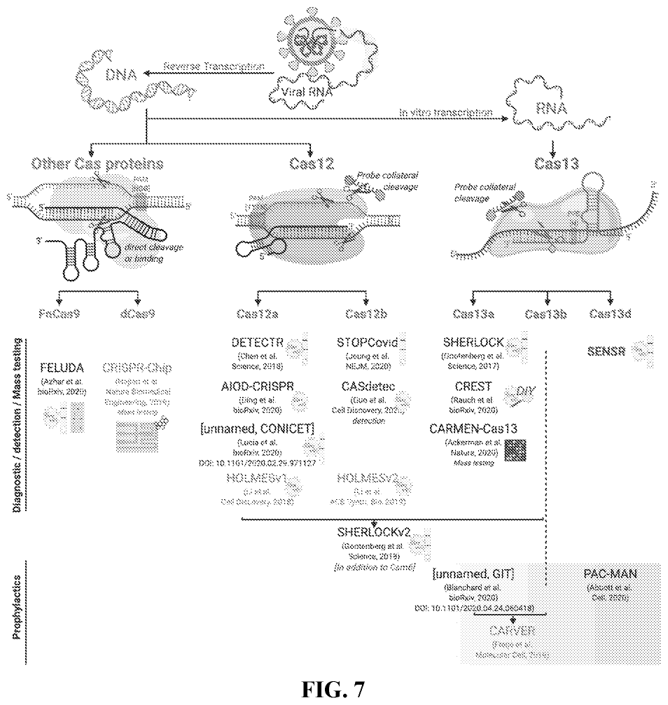

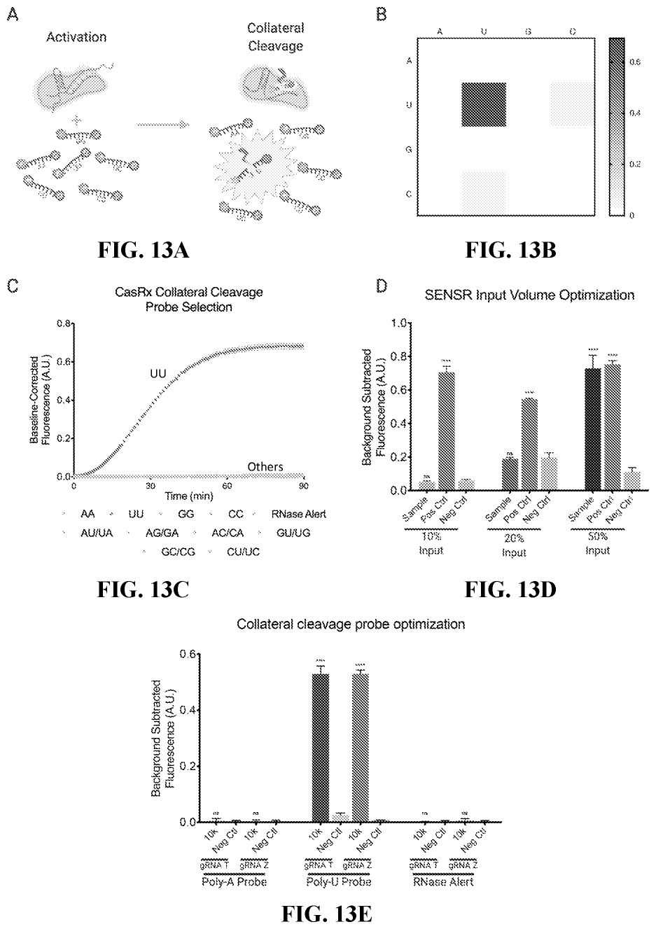

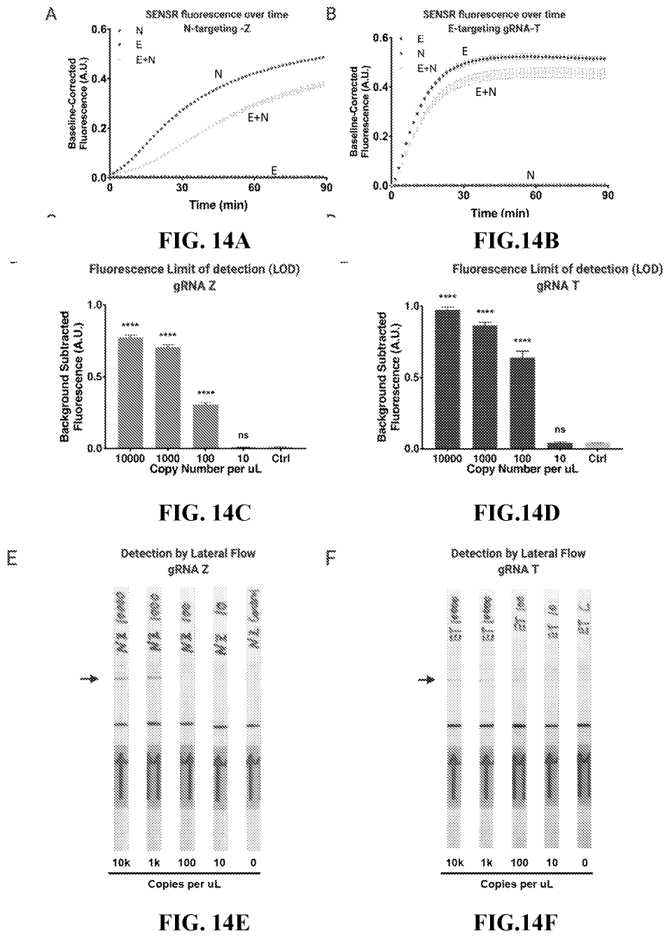

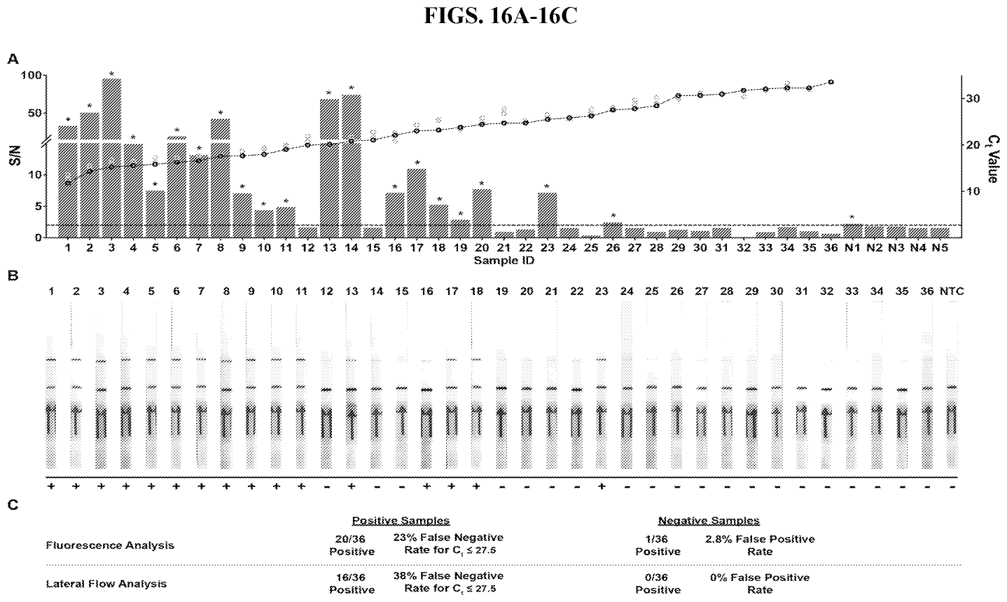

Complementing the rapidly expanding CRISPRDx toolkit (FIG. 7, Table 1), the use of RfxCas13d (CasRx) was outlined to detect SARS-CoV-2 using both synthetic targets and patient derived samples. SENSR amplifies nucleic acid sequences through an isothermal alternative to PCR then detects the target sequences by exploiting the native collateral cleavage activity of CasRx (FIG. 9A), providing proof-of-principle that Cas13d can be adapted as a point-of-care diagnostic. Further evidence shows that CasRx cleavage results in activation of an off-target collateral cleavage property (FIGS. 9E-9F) (Konermann, 2018; and Buchman, A. et al. Programmable RNA Targeting using CasRx in Flies. bioRxiv 2020.04.03.023606 (2020) doi:10.1101/2020.04.03.023606), with a preference for poly-U over poly-A stretches (FIG. 13A). This feature can be harnessed to detect viral sequences in a single reaction or possibly even in a multiplexed reaction combined with other Cas enzymes lacking a poly-U preference (Gootenberg et al. 2018; Kellner et al, 2019). Highly conserved and specific targets to SARS-CoV-2 were identified to prevent false-negatives (FIG. 11, Tables 2-3), and detection with attomolar sensitivity was demonstrated by both fluorescence and lateral flow readouts comparable to other previously developed systems (FIGS. 14C-14F) (Patchsung et al, 2020; Zhang et al, 2020; Metsky et al. 2020; and Broughton et al. 2020). It was further demonstrated that SENSR has the potential to be so specific to SARS-CoV-2, and analytical evidence was provided showing this system can be adapted to detect viral RNA (vRNA) in infected patient isolates (FIGS. 16A-16C).

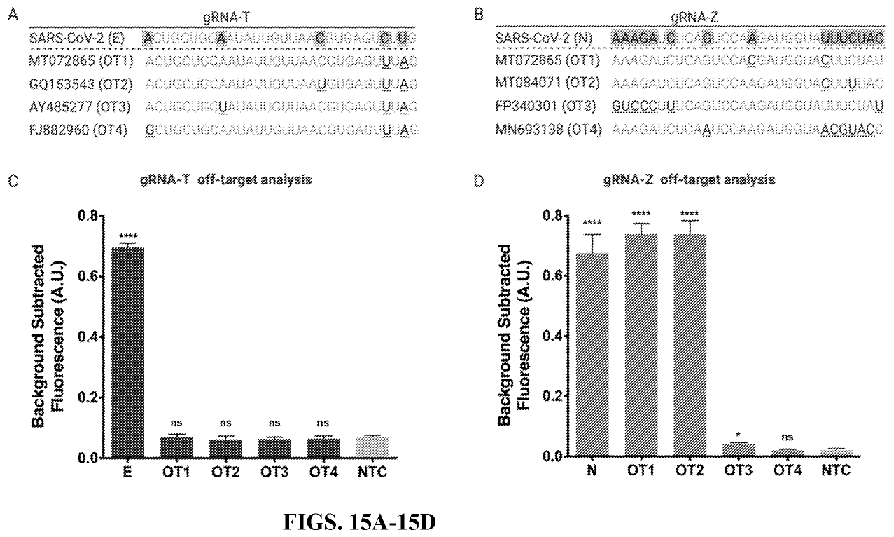

SENSR provides a robust proof-of-principle of viral detection by CasRx (such as Cas13d), however, it requires optimization in advance of deployment. Optimizing SENSR diagnostics can be pursued through a number of avenues. While some groups have improved specificity by selectively generating synthetic mismatches in guide sequences (Gootenberg et al. 2017), the gRNAs tested herein have moderate analytical specificity (FIG. 15). RT-RPA also exhibits nonspecific amplification (Daher et al. Mol. Cell. Probes 29, 116-121 (2015); Daher et al. Clin. Chem. 62, 947-958 (2016); and Luo et al. Anal. Biochem. 575, 36-39 (2019)), and therefore, alternative isothermal amplification technologies, such as RT-LAMP (Howson et al. J. Virol. Methods 249, 102-110 (2017); and Notomi et al. Nucleic Acids Res. 28, E63 (2000)), could be implemented to improve specificity. Although RT-LAMP is likely to improve specificity as well as sensitivity, the requirement of two separate reactions remains problematic, increasing the likelihood of contamination due to sample transfers (Sefers et al. Sefers, S. & Schmitz, J. E. Molecular Contamination and Amplification Product Inactivation. in Advanced Techniques in Diagnostic Microbiology: Volume 1: Techniques (eds. Tang, Y.-W. & Stratton, C. W.) 505-526 (Springer International Publishing, 2018). RT-LAMP and CasRx operate optimally at divergent temperatures (60-65° C. and 37° C., respectively), which may be incompatible within a one-step molecular diagnostic, though advances have been made in this sphere (Joung et al. Infectious Diseases (except HIV/AIDS) (2020) doi:10.1101/2020.05.04.20091231; Cai et al. Anal. Chem. 90, 8290-8294 (2018); and Joung et al. N. Engl. J. Med. (2020) doi:10.1056/NEJMc2026172). Thus alterations to existing isothermal amplification technologies or discovery of novel thermostable Cas nucleases with collateral cleavage activity could facilitate a more highly-sensitive Cas-based molecular diagnostic that operates in one reaction at a single temperature.

Beyond amplification, improvement to gRNA design criteria could drastically improve gRNA selection for detection and consequently the response time to future disease outbreaks. Currently, there remains no robust study attempting to characterize the in vitro collateral cleavage activity for varying Cas13 gRNA sequences, thus limiting efficient gRNA design and target selection for Cas13-based diagnostics. In this disclosure, it was observed significant variation in gRNA collateral cleavage activity, including two gRNAs (gRNA-AA and gRNA-AC) incapable of producing fluorescence signal (FIG. 14A) and kinetic variation exemplified by temporal differences in fluorescence signal accumulation for gRNA-T and gRNA-Z (FIGS. 9E-9F). It was also observed mild off-target activity for gRNA-Z (FIG. 15D) and found variation in reporter saturation for lateral flow between gRNAs (FIGS. 14E-14F), which has been found in other systems (Patchsung et al. 2020; and Zhang et al. 2020). Understanding gRNA-specific differences of Cas13 collateral cleavage allows the development of functional gRNA target sequence libraries for use in future pandemics. Furthermore, robust exploration for CasRx gRNA truncations and permutations should be undertaken to generate gRNAs intolerant of target site polymorphisms, or to even distinguish between strains (Gootenberg et al. 2017), thus improving the analytical specificity of SENSR. Optimizing workflow, deployment, and distribution, while taking steps to reduce the risk of contamination, is imperative to develop CasRx-based diagnostics to their full potential. It is demonstrated that detection with CasRx is a promising advancement for detecting viral infections, and could be improved to become a powerful molecular diagnostic with numerous applications.

### Kits

Further provided herein is a kit comprising, or consisting essentially of, or yet further consisting of the system as disclosed herein and instructions for use. In one aspect, the instructions are to perform the methods as disclosed herein. In a further aspect, the kit further comprising an anti-SARS-CoV-2 therapeutic (remdesivir (Gilead Sciences, Inc.)) or vaccine composition or therapeutic to treat symptoms of CoV-2 infection (e.g., an anti-inflammatory). In some embodiments, the kit further comprises one or more of: a negative control, a positive control (such as the synthetic viral (E) gene fragments as disclosed herein, e.g., Table 5), an off-target gRNA (such as those disclosed in Table 6) and an anti-SARS-CoV-2 therapeutic or vaccine composition.

The following examples are intended to illustrate, and not limit the embodiments disclosed herein.

### Experiment 1—Experimental Methods

CasRx Protein Expression and Purification Cloning

In this study, Applicant assembled the construct OA-1136J for CasRx protein expression, using the Gibson enzymatic assembly method (Nat Methods. 2009 May; 6(5):343-5). An empty vector containing His6-MBP-TEV fragment (obtained from Scott Gradia at UC Berkeley directly, unpublished. Also available on Addgene #29656) was used as backbone plasmids to clone in CasRx fragment. The restriction enzyme EcoRI was used to linearize the plasmid. The CasRx coding sequence as an insert fragment was amplified with primers 11361.C1 and 11361.C2 from plasmid OA-1050E (Addgene plasmid #132416).

To produce an expression plasmid for CasRx protein production Applicant cloned the CasRx coding sequence into the culture expression vector, pET-His6-MBP-tev-yORF (Series 1-M)(obtained from Scott Gradia at UC Berkeley directly, unpublished. Also available on Addgene #29656) using the Gibson assembly method (Gibson et al., 2009). In brief, the CasRx coding sequence was PCR amplified from plasmid OA-1050E (Addgene plasmid #132416) using primers 11361.C1 and 11361.C2. The fragment was purified and subcloned into the EcoRI site downstream of the His-MBP recombinant protein in pXR0021, generating the final pET-6×His-MBP-TEV-CasRx (1136J) plasmid.

Protein expression, culture, cell lysis, affinity and further downstream protein purification were performed as previously described in (Konermann et al. 2018). In brief, to facilitate protein expression in liquid culture, pET-His6-MBP-TEV-CasRx was transformed into Rosetta2(DE3) pLysS cells (Novagen, 71403). Starter cultures in LB were supplemented with kanamycin and chloramphenicol and incubated at 37° C. overnight. Secondary cultures were inoculated with 20 mL into 1 L of TB media supplemented with the same antibiotics. Cultures were allowed to grow until OD₆₀₀˜0.5, cooled on ice, induced with 200 mM IPTG, and then cultured for 20 hours at 18° C. Cells were then pelleted, freeze-thawed, lysed, and sonicated and clarified by centrifugation, followed by filtration with a 0.45 μM PVDF filter. Protein purification was performed by cation exchange chromatography through His-MBP, followed by gel filtration and fractionation, and separation of CasRX by TEV cleavage before final purification. A detailed step-by-step protocol for protein production and purification can be found in the Examples provided herein.

Production of Target SARS-Cov2 RNA and gRNAs

To detect viral genomic sequences, Applicant designed two synthetic dsDNA gene fragments containing a T7 promoter sequence upstream of gene segments corresponding to the SARS-CoV-2 envelope (E) and nucleocapsid (N) protein coding regions (MN908947.3). The E gene segment was ordered and synthesized as a custom GBLOCK® from Integrated DNA Technologies (IDT) and the N gene segment was amplified from a plasmid containing the entire N gene sequence ordered from IDT (1 h0006625) essentially as described in (Broughton et al. 2020), and outlined in the Table provided herein. The dsDNA gene fragments were amplified by PCR and purified using the MinElute PCR Purification Kit (QIAGEN #28004). Applicant also generated gRNAs targeting the synthetic viral RNA gene segments following a previously described templateless PCR protocol (M. Li, Akbari, and White 2018). Applicant then synthesized the synthetic viral RNA and gRNAs through in vitro transcription (IVT) using MEGASCRIP™ T7 Transcription Kit (INVITROGEN™ #AM1334), followed by DNaseI digestion and purification using the MEGACLEAR™ Transcription Clean-Up Kit (INVITROGEN™ #AM1908). Lastly, purified RNA was precipitated through standard NaAc and EtOH precipitation protocols. CasRx gRNAs were designed using the same criteria as outlined in (Buchman et al. 2020).

RT-RPA Amplification of Viral Genomic Sequences

Due to the unavailability of native viral genomic sequences from patient isolates, these protocols were initially developed and optimized on mock viral genome fragments. However this protocol was designed with the consideration of amplifying patient-derived viral genomic samples. To amplify the gRNA target sequences from the synthetic viral RNA, Applicant performed reverse transcriptase recombinase polymerase amplification (RT-RPA) as described in (Zhang, Abudayyeh, and Jonathan 2020). In short, RT primers were designed to amplify gRNA spacer regions from the synthetic viral RNA template and incorporate a T7 promoter sequence into the dsDNA gene fragments representative of the SARS-CoV-2 E and N genes. RT-RPA was performed at 42° C. by combining RevertAid Reverse Transcriptase (THERMO SCIENTIFIC™ #K1691) with TWISTAMP® Basic (TwistDx #TABAS03KIT). All RT-RPA primers sequences can be found in the Table provided herein.

Fluorescence-Based Detection of RNAse Activity by RNAaseALERT

To determine if the collateral RNAse activity of CasRx can be used to detect small quantities of viral genomic material, Applicant performed a modified RNAaseALERT V2 assay effectively as was done in (Kellner et al. 2019,). For these reactions, the CasRx, gRNAs prepared previously, in addition to the RNAaseALERT were thawed on ice under darkness. In short, the protocol was executed as follows: Pre-heat a heat block to 37° C., then prepare the reaction as follows: To 11.27 μL UltraPure water, add 0.4 μL of HEPES (pH 6.8, 1M), 0.18 μL MgCl₂ (1M), 0.8 μL of rNTP solution mix (25 mM each rNTP), 2 μL CasRx protein (60 ng/μL), 1 μL Murine RNase inhibitor (40 U/μL), 0.5 μL T7 RNA polymerase (5 U/μL), 1 μL gRNA (10 ng/μL), 1.25 μL RNaseALERT v2 (2μM), 1 μL of target DNA with T7 promoter. The reaction was incubated and the presence of fluorescence was read out by UV.

Lateral Flow-Based Detection of RNAse Activity

To determine if CasRx could be used to develop a point-of-care diagnostic, Applicant modified the HYBRIDETECT® system to detect evidence of SARS-CoV-2 viral-RNA induced CasRx collateral cleavage, essentially as was done in (Zhang et al. 2020) and outlined in detail in herein. In brief, Applicant designed a probe to have these properties. Following incubation of CasRx, gRNAs, T7 polymerase, rNTPs, and buffer components at 37° C. for 30 min, 80 μL of HybriDetect Assay buffer was added and each reaction mixed thoroughly. The completed reaction was placed at RT, and the lateral flow dipstick was inserted into the reaction, until the capillary actions to carry the solution up the filter membrane. The results were read out as two bands present representing positive and a single lower band a negative.

| | Volume per | Volume for four |
|---|---|---|
| Component | reaction (μL) | replicates (μL) |
| UltraPure water | 12.32 | 61.6 |
| HEPES, pH 6.8, 1M | 0.4 | 2 |
| MgCl₂, 1M | 0.18 | 0.9 |
| rNTP solution mix, 25 mM each | 0.8 | 4 |
| LwaCas13a in SB (63.3 μg/mL) | 2 | 10 |
| Murine RNase inhibitor, 40 U/μL | 1 | 5 |
| T7 RNA polymerase, 5 U/μL | 0.5 | 25 |
| crRNA (10 ng/μL) | 1 | 5 |
| LF-RNA reporter 1 (100 μM) | 0.2 | 1 |
| Total | 19 | 95 |

Recombinant Cas13d proteins were PCR amplified from genomic DNA extractions of cultured isolates or metagenomic samples and cloned into a pET-based vector with an N-terminal His-MBP fusion and TEV protease cleavage site. The resulting plasmids were transformed into Rosetta2(DE3) cells (Novagen), induced with 200 mM IPTG at OD₆₀₀ 0.5, and grown for 20 hours at 18° C. Cells were then pelleted, freeze-thawed, and resuspended in Lysis Buffer (50 mM HEPES, 500 mM NaCl, 2 mM MgCl₂, 20 mM Imidazole, 1% v/v Triton X-100, 1 mM DTT) supplemented with 1× protease inhibitor tablets, 1 mg/mL lysozyme, 2.5 U/mL Turbo DNase (Life Technologies), and 2.5 U/mL salt active nuclease (Sigma Aldrich). Lysed samples were then sonicated and clarified via centrifugation (18,000×g for 1 hour at 4° C.), filtered with 0.45 μM PVDF filter and incubated with 50 mL of Ni-NTA Superflow resin (QIAGEN) per 10 L of original bacterial culture for 1 hour. The bead-lysate mixture was applied to a chromatography column, washed with 5 column volumes of Lysis Buffer, and 3 column volumes of Elution Buffer (50 mM HEPES, 500 mM NaCl, 300 mM Imidazole, 0.01% v/v Triton X-100, 10% glycerol, 1 mM DT T). The samples were then dialyzed overnight into TEV Cleavage Buffer (50 mM Tris-HCl, 250 mM KCl, 7.5% v/v glycerol, 0.2 mM TCEP, 0.8 mM DTT, TEV protease) before cation exchange (HiTrap SP, GE Life Sciences) and gel filtration (Superdex 200 16/600, GE Life Sciences). Purified, eluted protein fractions were pooled and frozen at 4 mg/mL in Protein Storage Buffer (50 mM Tris-HCl, 1M NaCl, 10% glycerol, 2 mM DTT).

Materials and Equipment are listed below
Day 1. Bacteria Transformation: Micropipette and pipette tips (10, 200 and 500), Water bath or heat plate at 42° C., Incubator at 37° C.;
Day 2. Induction of protein overexpression: Volumetric pipettes and pipette aspirator, Shake flasks (500 ml, 1 L, 2 L), Refrigerated incubator Shaker;
Day 3. Cell lysis, his-affinity chromatography and TEV cleavage: Sonicator, Ultra Centrifuge max speed 20K×g, Gravity-flow chromatography columns, Dialysis cassettes/bags, 2 L beaker, Stir plate, Magnetic stir bar;
Day 4. Cation exchange and size exclusion chromatography: Akta, HiTrap SP, GE Life Sciences, Superdex 200 16/600, GE Life Sciences, Pre-cast Tris-Glycine polyacrylamide gels 10%
Reagents: LB and TB agar, LB and TB Broth, Kanamycin (100 μg/μl), Chloramphenicol (34 μg/μl), 1M Imidazole pH 8.0 (filtered), 1M Tris-HCl pH 7.4 (filtered), 5M NaCl (filtered), 1M HEPES pH 7.4 (filtered), 1M MgCl₂ (filtered), Triton X-100, 1M DTT (filtered), 1M IPTG (filtered), Glycerol, KCl (solid), TCEP (solid), 10 mg/mL lysozyme in 10 mM Tris-HCl pH 8.0 (filtered), TEV protease, Salt Active Nuclease, NiNTa superflow resin, Comassie blue solution, Destaining solution, Loading buffer, Page ruler molecular marker;
Buffers (Add reducing agents (DTT and TCEP) immediately prior to buffer usage):

Lysis Buffer—50 mM HEPES, 500 mM NaCl, 2 mM MgCl2,
20 mM Imidazole, 1% v/v Triton X-100, 1 mM DTT

| | Final | | | | | |
|---|---|---|---|---|---|---|
| Lysis Buffer | concentration | | 600 mL | | 1000 mL | |
| 1M HEPES pH 7.4 | 50 | mM | 30 | mL | 50 | mL |
| 5M NaCl | 500 | mM | 60 | mL | 100 | mL |
| 1M MgCl₂ | 2 | mM | 1.2 | mL | 2 | mL |
| 1M Imidazole | 20 | mM | 12 | mL | 20 | mL |
| Triton X-100 | 1% | v/v | 6 | mL | 10 | mL |
| DTT (solid) | 1 | mM | 92.55 | mg | 154.25 | mg |

Elution Buffer—50 mM HEPES, 500 mM NaCl, 300 mM imidazole,
0.01% v/v Triton X-100, 10% glycerol, 1 mM DTT

| | Final | | | | | |
|---|---|---|---|---|---|---|
| Elution Buffer | concentration | | 250 mL | | 1000 mL | |
| 1M HEPES pH 7.4 | 50 | mM | 12.5 | mL | 50 | mL |
| 5M NaCl | 500 | mM | 25 | mL | 100 | mL |
| 1M Imidazole | 300 | mM | 75 | mL | 300 | mL |
| Triton X-100 | 0.01% | v/v | 25 | uL | 100 | uL |
| Glycerol | 10% | v/v | 25 | mL | 100 | mL |
| 1M DTT | 1 | mM | 38.56 | mg | 154.25 | mg |

TEV Cleavage Buffer—50 mM Tris-HCl, 250 mM KCl,
7.5% v/v glycerol, 0.2 mM TCEP, 0.8 mM DTT

| | Final | | | |
|---|---|---|---|---|
| | concentration | | Volume | |
| TEV Cleavage Buffer | in 1 L buffer | | 1000 ml | |
| 1M Tris-HCl pH 7.4 | 50 | mM | 50 | mL |
| KCl (solid) | 250 | mM | 18.64 | g |
| glycerol | 7.5% | v/v | 75 | mL |
| TCEP (solid) | 0.2 | mM | 57.33 | mg |
| DTT (solid) | 0.8 | mM | 123.4 | mg |

Cation Exchange Buffer (CEB) A—50 mM Tris-HCl, 250 mM KCl,
7.5% v/v glycerol, 0.2 mM TCEP, 0.8 mM DTT

| Cation Exchange Buffer | Final | | | | | |
|---|---|---|---|---|---|---|
| (CEB) A | concentration | | 500 mL | | 1000 mL | |
| 1M Tris-HCl pH 7.4 | 50 | mM | 25 | mL | 50 | mL |
| KCl (solid) | 250 | mM | 9.32 | g | 18.64 | g |
| glycerol | 7.5% | v/v | 37.5 | mL | 75 | mL |
| DTT (solid) | 1 | mM | 77.13 | mg | 154.25 | mg |

Cation Exchange Buffer (CEB) B—50 mM Tris-HCl, 600 mM
KCl, 7.5% v/v glycerol, 0.2 mM TCEP, 0.8 mM DTT

| Cation Exchange Buffer | Final | | | | | |
|---|---|---|---|---|---|---|
| (CEB) B | concentration | | 500 mL | | 1000 mL | |
| 1M Tris-HCl pH 7.4 | 50 | mM | 25 | mL | 50 | mL |
| KCl (solid) | 800 | mM | 29.83 | g | 59.65 | g |
| glycerol | 7.5% | v/v | 37.5 | mL | 75 | mL |
| DTT (solid) | 1 | mM | 77.13 | mg | 154.25 | mg |

SEC Buffer/Storage Buffer—50 mM Tris-HCl, 1M NaCl, 10%
glycerol, 2 mM DTT

| SEC Buffer/Storage | | | | |
|---|---|---|---|---|
| Buffer | Final concentration | | 1000 mL | |
| 1M Tris-HCl pH 7.4 | 50 | mM | 50 | mL |
| 5M NaCl | 1M | | 58.44 | g |
| glycerol | 10% | v/v | 10 | mL |
| DTT (solid) | 2 | mM | 308.5 | mg |

Purification was performed at 4° C.

The following experimental step was performed.

On Day 1, plasmids was transformed into Rosetta2(DE3) cells. 2×10 mL starter cultures/1 L flask was prepared and grown overnight.

On Day 2, 1 mL overnight culture was added to each of 2×10 mL media and grown for 2 hrs. 2×10 mL was added per 1 L culture and allowed growing until OD600˜0.5 at 37° C. at 180 rpm. Cultures were taken off the shaker and placed on ice for ˜20 minutes. SDS Sample was collected. Cultures were induced with 0.2 mM IPTG and allowed growing overnight at 18° C. for 20 h.

On Day 3, SDS Sample was collected. Cells were spun down at 5k rpm for 15 min. SDS sample of supernatant was collected. Supernatant was discarded and pellets were stored at −80° C. After pellets had been frozen, they can be lysed and purified immediately. Cold lysis buffer was prepared by adding 1× protease inhibitor, 1 mM DTT, 1 mg/mL lysozyme, 2.5 U/mL Turbo DNase (Life Technologies), and 2.5 U/mL salt active nuclease (Sigma Aldrich). Pellet was resuspended in prepped cold lysis buffer until no clumps were visible. The sample was stirred on ice for 30 minutes. Solution became less viscous over time. SDS sample was collected. Resuspended cell pellets were sonicated for 6-10 minutes at 60 W. Cells were spun down at 18k×g for 1 h at 4° C. to clarify. SDS sample of supernatant was collected. Pellet was resuspended in equivalent volume and SDS sample was collected. The sample was filtered through a 0.45 m PVDF membrane. SDS sample was collected. In 50 mL falcon tubes, supernatant was incubated with Ni-NTA resin on rocker for 60-90 min at 4° C. For 1 L of growth, 5 mL of Ni-NTA resin was used in 50 mL falcon tube. Resin/lysis-supernatant mixture was applied onto a gravity column and FT was collected. SDS sample was collected. The column was washed with 5CV of Lysis buffer in 2 fractions. SDS samples were collected for each. The column was eluted with 3CV of elution buffer. SDS sample was collected. The sample was dialyzed overnight (O/N) into TEV Cleavage buffer (at least 100× volume).

On Day 4, the dialyzed sample was flown over Ni-NTA resin column and flow through (FT) was collected. SDS sample was collected. Cation exchange was performed using SP sepharose column: SP sepharose column was attached and washed with a few CV's MQ water if column was stored in 20% EtOH; SP Sepharose column was equilibrated on Akta Prime with 10 mL (1 CV) of buffer B at 3 mL/min; column was equilibrated with 40 mL (4 CV) of buffer A at 3 mL/min; once column was equilibrated, inject valve was set to load, and 5 mL of dissolved sample was loaded onto 5 mL loop, and the run was started with the following settings: 1 mL/min, % B=0, inject valve=inject, after 7 mL; inject valve was set to load; the rest of the sample was loaded onto 5 mL loop; inject valve was then set to inject; run was continued till UV detector stabilizes; at this step protein and DNA were bound to the SP Sepharose column; column was washed at 2 mL/min flow rate for the following volumes and % B setting was adjusted at the according volume: a. 20 mL at 0% B, b. 10 mL at 10% B, c. 10 mL at 20% B, d. 10 mL at 30% B (or until baseline is reached. Protein is likely not being eluted off at this time); gradient was set from 30% to 100% for 50 mL; fractions was collected in 2 mL; gel was run on fractions (7.50%); pure fractions were concentrated to at least 6 mL; and aggregate began to settle on top of column over multiple uses; after use, flow was reversed and injection loop and column was washed with 6M Guanidine buffer; the column was washed with 2-3 CVs of MQ water; and column was washed and stored in 20% EtOH. Gel filtration was performed via Superdex 200 16/600 (max—2 mL load) using SEC/Storage Buffer and repeated if needed. Gel was run on each fraction for analysis (7.50% gel). Pure samples were pooled. Concentration was obtained from nanodrop. The sample was then concentrated or diluted to 2 mg/mL, and flash frozen.

The following experimental step was performed.

On Day 1, plasmids were transformed into Rosetta2(DE3) cells. 20 mL LB was prepared with antibiotic (AB)—kanamycin and chloramphenicol per 1 L of growth. Media was inoculated with colony from transformed plate.

On Day 2, in the morning, 20 mL starter cultures were added to 1 L TB supplemented with AB and grown until OD600˜0.5 at 37° C. at 180 rpm. Cultures were taken off and placed on ice for ˜20 minutes. SDS Sample was collected. Cultures were induced w/0.2 mM IPTG and grown overnight (O/N) at 18° C. for 20 h.

On Day 3, cells were spun down at 5k rpm for 15 min. SDS sample of supernatant was collected. Supernatant was discarded and pellets were stored at −80° C. After pellets had been frozen (takes ˜10 minutes), they can be lysed and purified immediately. Cold lysis buffer was prepared by adding 1 mM PMSF (PMSF precipitated upon addition and dissolved entirely before adding other components), 1× protease inhibitor, 1 mM DTT, 1 mg/mL lysozyme, 2.5 U/mL Turbo DNase (Life Technologies), and 2.5 U/mL salt active nuclease (Sigma Aldrich). Components were dissolved entirely before next step. Pellet was resuspended in prepped cold lysis buffer until no clumps were visible, and was stirred on ice for 30 minutes. Solution became less viscous over time. SDS sample was collected. Resuspended cell pellets were sonicated for 6-10 minutes at 60 W. Cells were spun down at 18k×g for 1 h at 4° C. to clarify. SDS sample of supernatant was collected. Pellet was resuspended in equivalent volume and SDS sample was collected. Ni-NTA resin was equilibrated in lysis buffer during this step. In 50 mL falcon tubes, supernatant was incubated with equilibrated Ni-NTA resin on rocker for 60 min at 4° C. For 1 L of growth, 5 mL of Ni-NTA resin was used in 50 mL falcon tube. Resin/lysis-supernatant mixture was applied onto a gravity column and FT was collected. SDS sample was collected. The column was washed with 5CV of Lysis buffer in 2 fractions. SDS samples were collected for each. The column was eluted with 3CV of elution buffer. SDS sample was collected. Concentration was obtained from nanodrop. The column was stored at 4° C. in 20% ethanol for usage the following day. The sample was dialyzed O/N into TEV Cleavage buffer (at least 100× volume of elution). TEV was added to eluted sample at a 1:20 TEV:protein molar ratio. Concentration was difficult to assess due to nucleotide contamination. BCA Assay was optionally performed instead of nanodrop. ˜0.5 mg TEV was added with >90% cleavage).

On Day 4, dialyzed sample was flown over Ni-NTA resin column (same column used from Day 3) equilibrated with 3 CV of TEV Cleavage Buffer. Flow through (FT) was collected. The column was washed with 1CV TEV cleavage buffer and flow through was collected to ensure collection of all TEV-cleaved protein. SDS sample was collected. Flow through was diluted to 125 mM NaCl prior to cation exchange, if needed. Cation Exchange Buffer A was simultaneously added and stirred into sample immediately before cation exchange. Column had been prepared and equilibrated (see below step). Cation exchange was then performed via HiTrap SP HP: sample was loaded 5 mL at a time; superloop was used if available; HiTrap SP HP was attached and washed with a few CV's MQ water if column was stored in 20% EtOH; the column is equilibrate with 10 mL (1 CV) of buffer B at 1 mL/min; 8 mL buffer B was injected into loop to clean while injection mode=load; column was equilibrated with 40 mL (4 CV) of buffer A at 1 mL/min; 8 mL buffer A was injected into loop to equilibrate while injection valve=load; once column is equilibrated, inject valve was set to load; 5 mL of sample was loaded onto 5 mL loop; run was started with the following settings: 1 mL/min, % B=6.25%, inject valve=inject; after 7 mL, inject valve was set to load; the rest of the sample was loaded onto 5 mL loop; inject valve was then set to inject; the steps were repeated until all sample had been loaded. Run was continued till UV detector stabilized. At this step protein and DNA are bound to the column. Protein was eluted via gradient: a. Gradient—6.25% to 45% B in 15 mL (all samples and SDS sample were collected); b. Gradient—45% to 100% B in 15 mL (all samples in three fractions were collected as well as SDS sample for each); c. elution was continued until UV baseline had been reached. Aggregate began to settle on top of column over multiple uses. After use, flow was reversed and injection loop and column were washed with 6M Guanidine buffer. The column was washed with 2-3 CVs of MQ water. The column was washed and stored in 20% EtOH. Gel filtration was performed via Superdex 200 16/600 (max—2 mL load). Column was equilibrated in SEC/Storage solution in 2 mL. Gel was ran on each fraction. Samples were pooled. Concentration was obtained from nanodrop. And the samples were then concentrated or diluted to 2 mg/mL, and flash frozen. 1 L TB yielded ˜2 mg of ˜99% pure CasRx or ˜18000 fluorescence reactions. (Each fluorescent reaction needed 0.1108 ug).

In Vitro Activity of Purified CasRx

CasRx expressed from different plasmids was purified and tested for activity. Reactions were prepared as follows. CasRx protein was diluted at 55 ng/μl (0.5 μM) in storage buffer (Tris-HCl 7.5 mM, NaCl 100 mM, 10% glycerol, 2 mM DTT). 2 μl of the solution were mixed with 1 μl of gRNA at 32.64 ng/μl (1 μM). The mix was incubated at 37° C. for 10-15 min to favor the formation of ribonucleoprotein (RNP). After that 9 μl of a RNA template master mix (75 ng of 1136A template and 1×NEB Buffer 2.1.) were added to the RNP to be incubated at 37° C. for 1 hour. 1% agarose gel containing ethidium bromide was used to run the reactions (120V-18 min).

The reaction was set up as follows:

Make RNP

| | |
|---|---|
| CasRx (55 ng/μl) | 2 μl |
| gRNA (32.64 ng/μl) | 1 μl |
| Incubate 37° C. 10-15 minutes | |

Cleavage Assay

| RNP | 1 to 3 | μl |
|---|---|---|
| NEB Buffer 2.1 10× | 1 | μl |
| 1136A RNA template (75 ng/μl) | 1 | μl |
| Water | 7 | μl |
| Total | 10-12 | μl* |

Fluorescence Detection of Collateral Cleavage

Standard* reactions (CasRx:gRNA molar ratio 1:0.3) were prepared as follows: *In addition, non-standard protein: gRNA ratio of 1:2 was tested

| | |
|---|---|
| Water | 12.2 μl |
| HEPES pH 6.8 (1M) | 0.4 μl |
| MgCl₂ (1M) | 0.18 μl |
| CasRx (55 ng/μl) | 2 μl |
| RNAse inhibitor (40 U/μl) | 1 μl |
| gRNA-P (10 ng/μl) | 1 μl |
| RNaseAlert (2 μM) | 1.25 μl |
| Non target-Template (150 ng) | 1 μl |
| Template (1-1000 ng) | 1 μl |

Optimization was performed for nucleic acids detection using CasRx.

PCRs for gRNA was set up as follows

| | | |
|---|---|---|
| ×4 | T-° C. | Time |

KOD polymerase

| | | | | | | |
|---|---|---|---|---|---|---|
| Mastermix 2× | 25 | 100 | 95 | 2 | min | |
| Primer 1 | 1.5 | 6 | 95 | 20 | sec | ×25 |
| Primer 2 | 1.5 | 6 | 58 | 10 | sec | |
| Water | 22 | 88 | 70 | 5 | sec | |
| | 50 | 200 | 70 | 2 | min | |

| | |
|---|---|
| 12 | for ever |

Phusion polymerase

| 5× Buffer | 10 | 40 | 98 | 30 | sec | |
|---|---|---|---|---|---|---|
| dNTP | 1 | 4 | 98 | 5 | sec | ×25 |
| Primer 1 | 2.5 | 10 | 60 | 10 | sec | |
| Primer 2 | 2.5 | 10 | 72 | 15 | sec | |
| Phusion pol | 0.2 | 0.8 | 72 | 5 | min | |
| Water | 33.8 | 135.2 | 12 | for ever | | |
| | 50 | 200 | | | | |

Q5 polymerase

| 5× Buffer | 10 | 40 | 98 | 30 | sec | |
|---|---|---|---|---|---|---|
| dNTP | 1 | 4 | 98 | 5 | sec | ×25 |
| Primer 1 | 2.5 | 10 | 60 | 10 | sec | |
| Primer 2 | 2.5 | 10 | 72 | 15 | sec | |
| Q5 pol | 0.5 | 2 | 72 | 5 | min | |
| Water | 33.5 | 134 | 12 | for ever | | |
| | 50 | 200 | | | | |

PCR purification was performed as follows: The protocol on Qiagen PCR cleaning Minelute kit was followed. Except that eluting using 15 μl of water, the water was pipetted directly at the center of the column. The column was incubated at 42° C.-60° C. for 5 min.

Yield was >300 ng/μl (usually 400-500 ng/μl).

In vitro transcription with T7 Megascript was performed as follows: the purified DNA was used as template for the reaction. A mastermix was prepared as follows. The incubation was at 37° C. for 4-6 hours.

| | 4× |
|---|---|
| Component | Volume (μl) |
| Water | 28 |
| Buffer 10× | 8 |
| ATP | 8 |
| CTP | 8 |
| GTP | 8 |
| UTP | 8 |
| Template | 4 μl (1-1.2 μg) |
| Enzyme | 8 |
| Total | 80 |

RNA purification with Megaclear was performed as follows: the protocol that comes with the kit was followed. The elution was in water heated at 95° C. and the column was incubated for 1 minute at room temperature. Yield was typically >2000 ng/μl in 50 μl for the first elution. The second elution was collected in a different tube and used for several tests.

2% TBE Agarose gel for RNA electrophoresis was prepared as follows: TBE 10× was prepared using 54 g TRIS base, 27.5 g Boric acid, and 20 ml EDTA 0.5 M pH 8.0. 200 ml of TBE 1× was prepared and 4 g of agarose was added. The mixture was heated in the microwave for 2 minutes and 30 seconds. Once it was clear, the mixture was cooled down and 7 drops of ethidium bromide (0.625 μg/ml) was added. The mixture was poured using combs with wide wells.

10% Polyacrylamide TBE-Urea was made by mixing the following: Acrylamide 30%, 2.5 ml; Urea, 7.2 g; 10×TBE, 1.5 ml, and Water, 6 ml. 90 μl of TEMED and 75 μl of APS 10% were added.

Stop solution for in vitro cleavage reactions was prepared as follows: the stop solution was used for samples that were going to be loaded in polyacrylamide gels. Such solution is also used before loading to agarose gels, improving the quality of the results. A 2× Stop solution was made by mixing the following:

| Compound | Amount | | 2× [Final] |
|---|---|---|---|
| Urea | 4.804 | g | 8M |
| 0.5M EDTA pH 8.0 | 3.2 | ml | 160 mM |
| TrisHCl-pH 8.0 | 0.4 | ml | 40 mM |
| Water | Up to 10 | ml | — |

The 2× solution was diluted with equal volumes of Proteinase K stock (20 mg/ml) to prepare a 1×-Stop solution (4M Urea, 80 mM EDTA and 20 mM Tris-HCl with proteinase K at 10 mg/ml). 1 μl of this solution was added to each cleavage reaction and incubated at 37° C. for 15 min before proceeding to preparing the sample with denaturing loading dye.

The RNA sample was prepared using denaturing loading dye as follows: Gel Loading Buffer II (Denaturing PAGE) (95% Formamide, 18 mM EDTA, and 0.025% SDS, Xylene Cyanol, and Bromophenol Blue) was used. This solution was 2× and used as that to get best results. However less were also used, for example, as 4× to quick diagnostic of cleavage or comparisons between samples that were not critical. For Agarose gels: 1 μl of ethidium bromide (0.625 μg/ml) was added per 1000 μl of dye. 10 μl of the sample was mixed with 5-10 μg of loading dye. For polyacrylamide: 2-5 μl of the sample was mixed with 2-5 μl of loading dye. The samples were denatured at 85° C. during 5 minutes. 2-5 μl of sample was loaded.

RNA ladder was prepared as follows. 70 μl of water and 100 μl of Gel loading Buffer II were added to get a final volume of 200 μl.

| RNA ladder | [stock] ng/μl | Volume (μl) | [Final] ng/μl |
|---|---|---|---|
| 1500 nt | 3666 | 4 | 73.32 |
| 700 nt | 557 | 15 | 41.775 |
| 269 nt | 2741 | 3 | 41.115 |
| 96 nt | 1000 | 8 | 40 |
| | | 30 | |

The samples were ran in the gel as follows.

TBE-Agarose: the gel was ran at 120V for 15 min (a picture was taken) and ran for 20 min more. Extra time was applied if needed.

Polyacrylamide: the gels were prepared in the tank with 1×TBE buffer. The combs were removed and the gen was washed with 1×TBE to remove any particles present in the wells. Pre-run of the empty gel was performed at 150V for 10 min. 2-5 μl of denatured sample was loaded and ran at 150 V for 1 hour while keeping it cool at 4° C. The gel was stained for 15-30 min in a solution of ethidium bromide (0.5 μg/ml) and visualized under UV.

In vitro cleavage assay was performed to test CasRx protein and gRNAs using NEB buffer. CasRx protein was diluted at 55 ngR (0.5 μM) in storage buffer (Tris-HC 37.5 mM, NaCl 100 mM, 10% glycerol, 2 mM DTT). 2 μl of the solution were mixed with 1 μl of gRNA at 32.64 ng/(1 μM). The mix was incubated at 37° C. for 10-15 min to favor the formation of ribonucleoprotein (RNP). After that, 9 μl of RNA template master mix (75 ng of 1136A template and 1×NEB Buffer 2.1.) were added to the RNP to be incubated at 37° C. for 1 hour. 1% agarose gel containing ethidium bromide was used to run the reactions (120V-18 min).

| | Vol (μL) |
|---|---|
| RNP reaction | |
| CasRx (55 ng/μl) | 2 μl |
| gRNA (32.64 ng/μl) | 1 μl |
| Incubate 37° C. for | |
| 10-15 minutes | |
| Cleavage reaction | |
| RNP | 3 |
| NEB Buffer 2.1 10× | 1 |
| 1136A RNA template (75 ng/μl) | 1 |
| Water | 7 |
| Incubate 37° C. for 1 hour | |

Fluorescence detection of collateral cleavage was performed as follows: for detection using fluorescence the SHERLOCK standard conditions were used for reactions. A mastermix was prepared using the following:

Standard* reactions (CasRx:gRNA molar ratio 1:0.3) were prepared as follows: *Fluorescence detection of collateral cleavage using non-standard conditions

| | |
|---|---|
| Water | 12.2 μl |
| HEPES pH 6.8 (1M) | 0.4 μl |
| MgCl₂ (1M) | 0.18 μl |
| CasRx (55 ng/μl) | 2 μl |
| RNAse inhibitor (40 U/μl) | 1 μl |
| gRNA-P (10 ng/μl) | 1 μl |
| RNaseAlert (2 μM) | 1.25 μl |
| Non target-Template (150 ng) | 1 μl |
| Template (1-1000 ng) | 1 μl |

The protocol below was followed.

The amount of reactions were calculated and stocks of protein, gRNA, template and off-target template were prepared at the following concentrations:

For test 1: Standard 1:0.3 ratio

| Conditions tested | [CasRx stock] ng/μl | [gRNA stock] ng/μl |
|---|---|---|
| Standard | 56 | 10 |
| 5× | 280 | 50 |
| 10× | 560 | 100 |
| 20× | 1120 | 200 |

For Test 2: non-standard 1:3 ratio

| Conditions tested | [CasRx stock] ng/μl | [gRNA stock] ng/μl |
|---|---|---|
| Standard | 56 | 100 |
| 5× | 280 | 500 |
| 10× | 560 | 1000 |
| 20× | 1120 | 2000 |

For test 3: multiplexed gRNAs

| | | [gRNA mixture L, O, |
|---|---|---|
| Conditions tested | [CasRx stock] ng/μl | P stock] ng/μl |
| Standard | 56 | 10 |
| 5× | 280 | 50 |
| 10× | 560 | 150 |
| 20× | 1120 | 300 |

Template RNA was 1000 ng/μl and off-target template was 550 ng/μl.

A master mix was prepared on ice without the gRNA, CasRx and template as follows:
For 10×

| | | |
|---|---|---|
| Water | 12.42 μl | 124.2 |
| HEPES pH 6.8 (1M) | 0.4 μl | 4 |
| MgCl₂ (1M) | 0.18 μl | 18 |
| RNAse inhibitor (40 U/μl) | 1 μl | 10 |
| RNaseAlert (2 μM) | 1 μl | 10 |
| Template (1000 ng/μl) | 1 μl | 10μ |

The master mix was kept on ice and covered from light during the whole process.

16 μl of the mastermix was added to all the wells in the plate for later use. The wells were kept covered from light and on ice. Addition of the components were immediately started for each test, starting with the stocks of CasRx (2 μl), gRNA (1 μl), Non-target template (1 μl, switch this for 1 μl of water or 1 μl of probe). Alternatively, tests were ran using the template as the final component on individual reactions. In this case, 15 of master mix was pipetted and everything else was added to individual reactions.

Test 1 used standard molar ration 1:0.3 CasRx:gRNA with increased protein/gRNA amount and probe amount. Reactions (CasRx:gRNA molar ratio 1:0.3) were prepared as follows:

| Water | | | 12.42 μl | | |
|---|---|---|---|---|---|
| HEPES pH 6.8 (1M) | | | 0.4 μl | | |
| MgCl₂ (1M) | | | 0.18 μl | | |
| CasRx (see table below) | | | 2 μl | | |
| RNAse inhibitor (40 U/μl) | | | 1 μl | | |
| gRNA-P (See table below) | | | 1 μl | | |
| RNaseAlert (2 μM) see table below | | | 1 μl | | |
| Non target-Template (550 ng)* | | | 1 μl | | |
| see table | | | | | |
| Template (1000 ng) | | | 1 μl | | |
| | | | | | RNase alert |
| Conditions | [CasRx | | [gRNA | | Probe |
| tested | stock] ng/μl | Volume | stock] ng/μl | Volume | volume |
| Standard | 56 | 2 μl | 10 | 1 μl | 1 μl |
| 5× | 280 | 2 μl | 50 | 1 μl | 1 μl |
| 10× | 560 | 2 μl | 100 | 1 μl | 1 μl |
| 20× | 1120 | 2 μl | 200 | 1 μl | 1 μl |
| 5×* | 280 | 2 μl | 50 | 1 μl | 2 μl* |
| 20×* | 1120 | 2 μl | 200 | 1 μl | 2 μl* |

*For this conditions 1 additional μl of probe was used instead of Non-target template

Test 2 used non-standard molar ratio 1:3 CasRx:gRNA with increased protein/gRNA amount and probe amount. Reactions were prepared as follows:

| Water | | | 12.42 μl | | |
|---|---|---|---|---|---|
| HEPES pH 6.8 (1M) | | | 0.4 μl | | |
| MgCl₂ (1M) | | | 0.18 μl | | |
| CasRx (see table below) | | | 2 μl | | |
| RNAse inhibitor (40 U/μl) | | | 1 μl | | |
| gRNA-P (See table below) | | | 1 μl | | |
| RNaseAlert (2 μM) see table below | | | 1 μl | | |
| Non target-Template (550 ng)* | | | 1 μl | | |
| see table | | | | | |
| Template (1000 ng) | | | 1 μl | | |
| | | | | | RNase alert |
| Conditions | [CasRx | | [gRNA | | Probe |
| tested | stock] ng/μl | Volume | stock] ng/μl | Volume | volume |
| Standard | 56 | 2 μl | 100 | 1 μl | 1 μl |
| 5× | 280 | 2 μl | 500 | 1 μl | 1 μl |
| 10× | 560 | 2 μl | 1000 | 1 μl | 1 μl |
| 20× | 1120 | 2 μl | 2000 | 1 μl | 1 μl |
| 5×* | 280 | 2 μl | 500 | 1 μl | 2 μl* |
| 20×* | 1120 | 2 μl | 2000 | 1 μl | 2 μl* |

*For this conditions 1 additional μl of probe was used instead of Non-target template.

Test 3 used standard molar ratio 1:0.3 CasRx:gRNA multiplexed (L, O, P) with increased protein/gRNA amount and probe amount. Reactions were prepared as follows:

| Water | | | 12.42 μl | | |
|---|---|---|---|---|---|
| HEPES pH 6.8 (1M) | | | 0.4 μl | | |
| MgCl₂ (1M) | | | 0.18 μl | | |
| CasRx (see table below) | | | 2 μl | | |
| RNAse inhibitor (40 U/μl) | | | 1 μl | | |
| gRNA-P (See table below) | | | 1 μl | | |
| RNaseAlert (2 μM) see | | | 1 μl | | |
| table below | | | | | |
| Non target-Template | | | 1 μl | | |
| (550 ng)* see table | | | | | |
| Template (1000 ng) | | | 1 μl | | |
| | | | [gRNA | | |
| | | | mixture | | RNase |
| | [CasRx | | L, O, P | | alert |
| Conditions | stock] | | stock] | | Probe |
| tested | ng/μl | Volume | ng/μl | Volume | volume |
| Standard | 56 | 2 μl | 10 | 1 μl | 1 μl |
| 5× | 280 | 2 μl | 50 | 1 μl | 1 μl |
| 10× | 560 | 2 μl | 150 | 1 μl | 1 μl |
| 20× | 1120 | 2 μl | 300 | 1 μl | 1 μl |
| 5×* | 280 | 2 μl | 50 | 1 μl | 2 μl* |
| 20×* | 1120 | 2 μl | 300 | 1 μl | 2 μl* |

These ratios are (1.4:1) protein to gRNA.
*For this conditions 1 additional μl of probe was used instead of Non-target template.

CasRx-DCR-4 program was ran.

| Name | Reaction | Sequence (5′-3′) |
|---|---|---|
| 1136A-F | PCR | Gaaattaatacgactcactataggacaggtac |
| 1136A-R | PCR | Aaaaaagaggagcgagaagagg |
| 1136B1 | PCR | Gaaattaatacgactcactataggcaagtaaacccctaccaactggtcgg |
| | | ggtttgaaac |
| 1136B2 | PCR | Aaaaaaacactagccatccttactgcgcttcgattggtttcaaaccccga |
| | | ccagt |
| 1136B | IVT | gaaattaatacgactcactataggcaagtaaacccctaccaactggtcgg |
| | | ggtttgaaaccaatcgaagcgcagtaaggatggctagtgttttttt |
| 1136C1 | PCR | Aaaaaactacaacttcctcaaggaacaacattgccagtttcaaaccccga |
| | | ccagt |
| 1136C | IVT | gaaattaatacgactcactataggcaagtaaacccctaccaactggtcgg |
| | | ggtttgaaactggcaatgttgttccttgaggaagttgtagtttttt |
| 1136F.C1 | Cloning | Cgcggatccgaattcgagctccgtcgacaagcttgcggccgcatcgaaaa |
| | | aaaaaagtcc |
| 1136F.C2 | Cloning | Tctcagtggtggtggtggtggtgctcgagtgcggccgcttaggaattgcc |
| | | ggacacct |
| 11361.C1 | Cloning | Cgaggaaaacctgtacttccaatccaatatcgaaaaaaaaaagtcc |
| 11361.C2 | Cloning | Gctcgagtgcggccgcaagcttgtcgacttaggaattgccggacacct |
| 1136K1 | PCR | Acactagccatccttactgcgcttcgattggtttcaaaccccgaccagt |
| 1136K | IVT | gaaattaatacgactcactataggcaagtaaacccctaccaactggtcgg |
| | | ggtttgaaaccaatcgaagcgcagtaaggatggctagtgt |
| 1136L1 | PCR | Ctacaacttcctcaaggaacaacattgccagtttcaaaccccgaccagt |
| 1136L | IVT | gaaattaatacgactcactataggcaagtaaacccctaccaactggtcgg |
| | | ggtttgaaactggcaatgttgttccttgaggaagttgtag |
| 1136M1 | PCR | cttgctttcgtggtattcttgctagtcacagtttcaaaccccgaccagt |
| 1136M | IVT | gaaattaatacgactcactataggcaagtaaacccctaccaactggtcgg |
| | | ggtttgaaactgtgactagcaagaataccacgaaagcaag |
| 1136N1 | PCR | tgctgccaccgtgctacaacttcctcaagggtttcaaaccccgaccagt |
| 1136N | IVT | gaaattaatacgactcactataggcaagtaaacccctaccaactggtcgg |
| | | ggtttgaaacccttgaggaagttgtagcacggtggcagca |
| 1136O1 | PCR | gccatccttactgcgcttcgattgtgtgcggtttcaaaccccgaccagt |
| 1136O | IVT | gaaattaatacgactcactataggcaagtaaacccctaccaactggtcgg |
| | | ggtttgaaaccgcacacaatcgaagcgcagtaaggatggc |
| 1136P1 | PCR | cgcaatcctaataacaatgctgccaccgtggtttcaaaccccgaccagt |
| 1136P | IVT | gaaattaatacgactcactataggcaagtaaacccctaccaactggtcgg |
| | | ggtttgaaaccacggtggcagcattgttattaggattgcg |

### Experiment 2—a Sensitive, Rapid, and Portable CasRx-Based Diagnostic Assay for SARS

Applicant outlined the development of a CRISPR-based nucleic acid molecular diagnostic utilizing a Cas13d ribonuclease derived from Ruminococcus flavefaciens (CasRx) to detect SARS-CoV-2, an approach also referred to herein as SENSR (Sensitive Enzymatic Nucleic-acid Sequence Reporter). It was demonstrated that SENSR robustly detects SARS-CoV-2 sequences in both synthetic and patient-derived samples by lateral flow and fluorescence, thus expanding the available point-of-care diagnostics to combat current and future pandemics.

Development of the SENSR System and SENSR Workflow

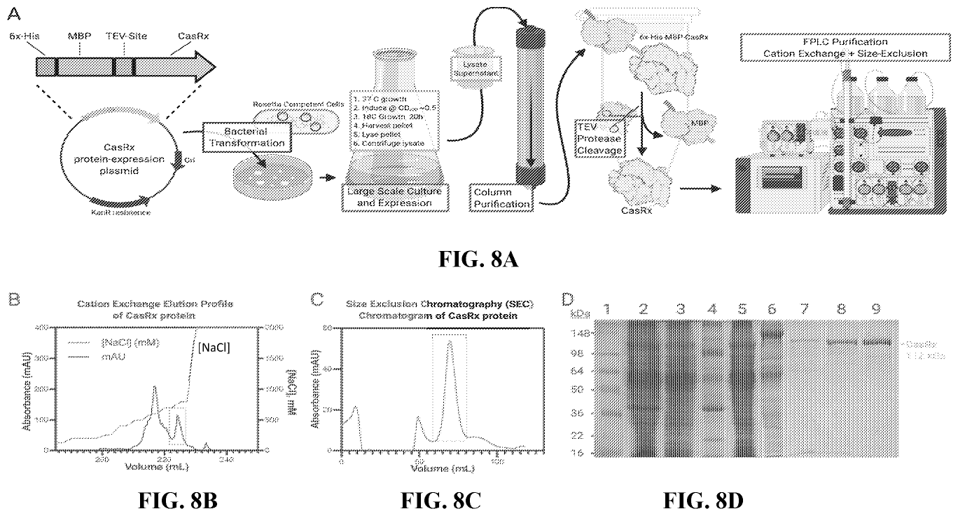

Derived from protocols originally developed for CRiISPRDx using Cas13a and Cas13b (FIG. 7, Table 1), SHERLOCK (Gootenberg et al., 2017; Gootenberg et al, 2018, Patchsung et al, 2020; and Kellner et al, 2019), designed and established herein is a two-step protocol for the detection of nucleic acids using a recombinant CasRx (e.g., RfxCas13d) (FIG. 8). Target sequences are first amplified in a 45-minute isothermal preamplification reaction by combining reverse transcription with recombinase polymerase amplification (RT-RPA) (Piepenburg et al. PLoS Biol. 4, e204 (2006)). The RT-RPA reaction produces a short dsDNA amplicon containing the CasRx target site and an upstream T7 promoter sequence. Next, the RT-RPA reaction is transferred into a second reaction containing a T7 polymerase and the CasRx ribonucleoprotein. The dsDNA amplicon is then transcribed into ssRNA transcripts which are then cleaved by CasRx. This initial cleavage event initiates the collateral cleavage activity of CasRx resulting in cleavage of a bystander ssRNA probe. Collateral cleavage of the probe is analyzed by either fluorescence or lateral flow readouts, thus indicating the initial presence or absence of SARS-CoV-2 genomic sequences (FIG. 9A, FIG. 10).

Target Selection and Reagent Validation

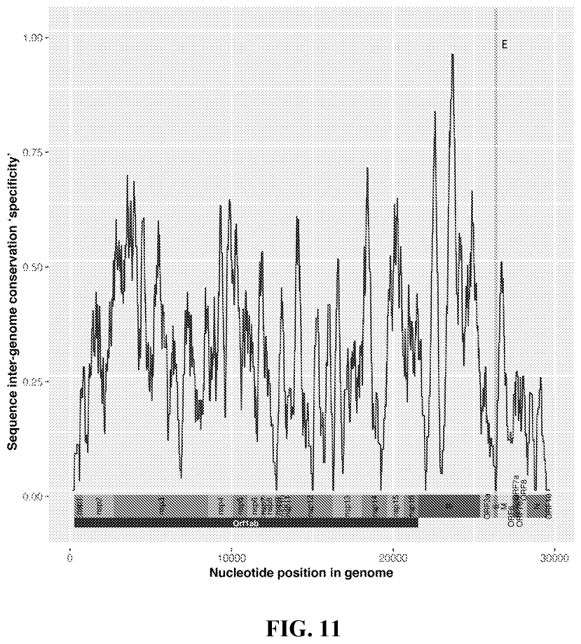

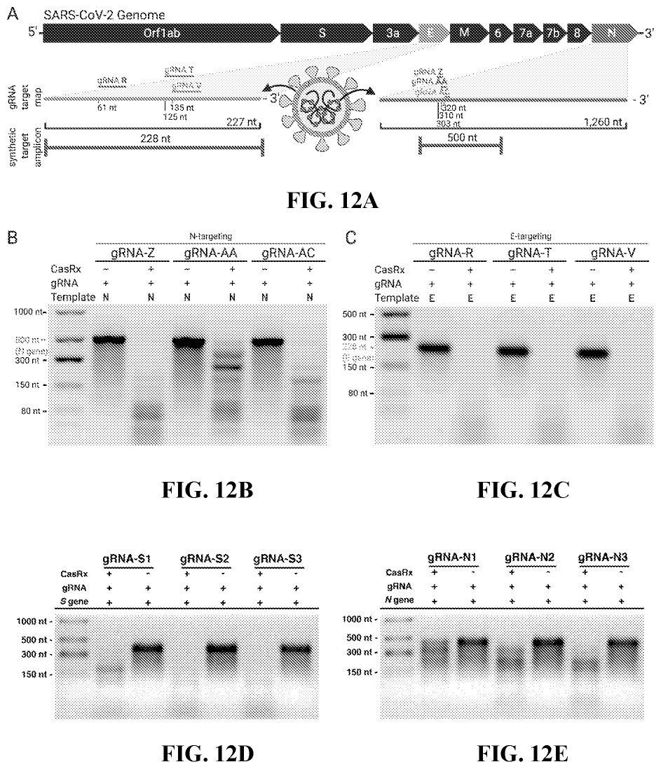

Diagnostics require high specificity to limit the probability of false positives from detection of random nucleic acids. To ensure high analytical specificity of the target sites, a bioinformatic pipeline was established and searched for 30 nt long sequences conserved across the first 433 published SARS-CoV-2 genomes (available at GenBank on Apr. 7, 2020), and without homology to other coronaviruses (ViPR, Virus Pathogen Resource, n=3,164). This search yielded a panel of gRNA target sites (n=8846) less likely to result in false positives or negatives due to sequence constraints (FIG. 11, Tables 2, and 3). Aligning with previously established diagnostics (e.g., RT-qPCR assays recommended by the WHO and CDC targeting the envelope (E) and nucleocapsid (N) genes within the SARS-CoV-2 genome), the Nucleocapsid (N), envelope (E) and Spike (S) genes were selected as the targets of SENSR for system validation (FIG. 9B) (Corman, et al., 2020; Lu, X. et al. Emerg. Infect. Dis. 26, (2020); Patchsung et al. 2020; and Broughton et al. 2020). The bioinformatic analysis revealed multiple specific sequences within the N-gene (n=150) and S-gene (n=1568), to which Applicant designed three guides for each gene (N: gRNA-N1, -N2, -N3, -Z, -AA, -AC; S: gRNA-S1, -S2, -S3) (Table 2, FIG. 9B, FIG. 12). However, the stringent bioinformatic search criteria did not identify targets specific to SARS-CoV-2 within the E-gene due to high sequence homology with the SARS-CoV-1 E-gene. Applicant therefore relaxed the criteria to include target sites sharing homology with SARS-CoV-1. From this, Applicant selected three E-targeting guides (gRNA-R, -T, -V), with two displaying complete (gRNA-T (433/433), gRNA-V (433/433)) and one with nearly complete (gRNA-R (430/433)) conservation among the available SARS-CoV-2 genomes (Table 4, FIG. 12). To validate these gRNAs against SARS-CoV-2, synthetic SARS-CoV-2 targets encompassing the E-, N- and S-genes (i.e., ssRNA gene fragments mimicking portions of the Envelope, Nucleocapsid and Spike genomic sequences) were generated (FIG. 9B, Table 5), and tested for in vitro cleavage activity with purified CasRx (FIG. 8). Initial characterization of on-target cleavage revealed significant degradation of target transcripts for all guides tested (FIGS. 12B-12E) motivating further assessment of all candidate guides.

To minimize overall time to detection, each gRNA was tested in a standard SENSR fluorescence reaction (FIG. 10) against a high-copy number of synthetic templates (10,000 copies/μL). Fluorescence data was then analyzed to determine the time each gRNA reached its half-maximum fluorescence (HMF) (FIGS. 9C-9F). HMF was calculated by fitting a non-linear regression to the fluorescence data acquired over 1 hour. The equation for the nonlinear regression was then used to solve for the time HMF occurred (see Experiment 3). From the analysis, gRNA-S3 (t=5.88 min) of the S-targeting group and gRNA-N1 (t=12.54 min) of the N-targeting group resulted in the fastest HMF times and were selected for downstream analysis (FIGS. 9C-9F).

To determine the most effective gRNAs for use in SENSR, fluorescence accumulation over time was monitored in an IVT-coupled cleavage reaction for each gRNA. All gRNAs induced robust fluorescence within minutes, with the exception of gRNA-AA and -AC which produced no signal (FIGS. 9C-9F). gRNA-T (E-targeting) and gRNA-Z (N-targeting) were selected for downstream analysis due to their robust cleavage activity as well as sequence conservation among all publicly available SARS-CoV-2 patient sample isolates (FIGS. 9B-9F, Tables 2-3).

Fluorescence-Based Detection of SARS-CoV-2 and Optimization of SENSR

It was demonstrated that on-target cleavage activates a secondary collateral cleavage property of CasRx (Konermann et al., 2018; and Buchman et al., 2020). The in vitro collateral cleavage activity of CasRx was initially evaluated with gRNA-T and gRNA-Z through gel electrophoresis. By incubating CasRx, gRNA-T or gRNA-Z, and varying the addition of synthetic templates, it was found that CasRx collateral cleavage was only activated when the synthetic template added complemented the gRNA target sequence (FIGS. 9G-9H). To exploit this tandem, collateral cleavage activity to cleave a bystander fluorescent probe in trans, facilitates detection of SARS-CoV-2 by fluorimetry (FIGS. 9A, 10, and 13A). A previous study demonstrated that each Cas13 enzyme exhibits select di-nucleotide sequence preferences for collateral cleavage (Gootenberg et al. 2018), however, no such analysis has been performed on CasRx. The quenched fluorescent reporter within RNaseAlert v2 was used to report collateral cleavage of Leptotrichia wadeii (LwaCas13a), Capnocytophaga canimorsus Cc5 (CcaCas13b), and PsmCas13b (Prevotella sp. P5-125) in the SHERLOCK systems (Gootenberg et al., 2017; Gootenberg et al., 2018; and Kellner et al., 2019), however the preliminary analysis of RNaseAlert v2 failed to yield fluorescence with CasRx in the presence of a synthetic target (Student's t-test gRNA-T: p=0.4294, gRNA-Z: p=0.1510) (FIG. 13E). Prior work suggested CasRx preferentially cleaves targets containing poly-U stretches (Konermann et al. 2018), with complementary work demonstrating collateral cleavage of targets rich in both poly-A and poly-U stretches (Yan et al. 2018).

To develop a probe cleavable by CasRx, ten custom 6 nucleotide ssRNA probes were generated, with variable di-nucleotide sequences, each conjugated to a 5′ fluorescent molecule (6-FAM) and a 3′ fluorescence quencher (FQ), whereupon separation following cleavage results in detectable fluorescence signal (FIG. 13, Table 5). Following incubation of CasRx in the presence or absence of the synthetic targets (E- or N-gene, the poly-U probe yielded significant and detectable fluorescence (e.g., for 10000 copies/μl) compared to the no-template control (NTC) (Student's t-test poly-U: p<0.0001), while the poly-A probe produced no detectable fluorescence signal (Student's t-test gRNA-T: p=0.5953, gRNA-Z: p=0.7935). All other di-nucleotide or commercial probes tested (ANOVA, Dunnett's test poly-U: p<0.0001). The results suggest a preference for poly-U stretches by CasRx, and motivating the use of the poly-U probe in the remainder of experiments (FIG. 13, Table 5).

Following probe selection, the reaction conditions were optimized for the amplification and cleavage reactions. It was first evaluated how varying the volume of sample input into the RT-RPA reaction impacted the detection of a target sequence. To do so, diluted synthetic ssRNA templates down to 1,000 copies/μL were added and the templates were added between 10%-52% RT-RPA reaction volume. Using HMF analysis it was found the 28.5% volume input group resulted in the fastest detection time compared to all other groups.

Accordingly, the collateral cleavage activity was evaluated in the context of fluorescence. It was determined if the gRNA incubated with the respective target sequence dictates the increase in fluorescence signal over time. To do so, CasRx, gRNA-T or gRNA-Z, the modified poly-U probe, and varied the addition of synthetic templates were incubated, while fluorescence data were acquired on a plate reader. It was observed that fluorescence signal only accumulated, and thus collateral cleavage activated, when the synthetic template added to the reaction complemented the gRNA target sequence (FIGS. 14A-14B FIG. 2A, B).

After optimizing preamplification reaction input volume using 100 copies/μL of synthetic RNA, and determining 50% preamplification reaction volume input to be optimal (FIG. 13), the limit of detection (LOD) was determined by fluorescence for gRNA-T and gRNA-Z titrated at log scale (via serial dilution of synthetic RNA input) from 10,000 to 0 copies/μL. After optimizing reaction parameters and for both gRNA-T and gRNA-Z, signal above noise was observed for 10,000, 1,000 and 100 copies compared to the NTC and the LOD was determined to be 100 copies/μL (FIGS. 14C-14D), indicating SENSR exhibits attomolar sensitivity comparable to other CRISPRDx systems (Gootenberg et al, 2017; and Chen et al., 2018). These results demonstrate that CasRx can robustly detect and report the presence of synthetic SARS-CoV-2 RNA via fluorescence readout.

Lateral Flow Assay Development

Collateral cleavage by CasRx can additionally be exploited to detect synthetic SARS-CoV-2 RNA by lateral flow assay which facilitates detection by simple paper test strip and eliminates the need for complicated and expensive laboratory equipment (FIGS. 9A and 10). Similar to assays developed by others (Gootenberg et al., 2018; and Kellner et al., 2019), a 6 nucleotide ssRNA probe was developed conjugated with 5′ 6-FAM and 3′ biotin (Bio) compatible with Millenia HybriDetect lateral flow strips (Table 5). In brief, collateral cleavage results in separation of 6-FAM from biotin, detectable following capillary action down a paper dipstick imprinted with streptavidin or anti-FAM secondary antibodies at distant ends (FIG. 9A). The absence or presence of the upper band therefore indicates a negative or positive result, respectively (Summarized in FIGS. 9A and 10). Using this protocol, it was demonstrated that SENSR can be adaptable to readout by lateral flow, and determined the LOD of synthetic SARS-CoV-2 RNA to be as low as ˜100 copies/μL, however with variability between guides (FIGS. 14E-14F). These results confirm that SENSR, like other CRISPRDx systems, can be adapted for read-out by lateral flow, indicating the potential for future application within point-of-care rapid diagnostic tests.

Specificity of SENSR Against Known Possible Off-Targets

Diagnostic assays require stringent specificity parameters to limit false-negatives/positives. Because many Cas effectors tolerate some degree of mismatch (Tambe et al., Cell Rep. 24, 1025-1036 (2018); Zheng et al., Sci. Rep. 7, 40638 (2017); and Teng et al., Genome Biol. 20, 132 (2019)), unintended false-positives can occur as a result of cleaving closely related off-target sequences. In a health-care setting, SENSR is unlikely to be exposed to randomly generated high-homology or high-identity sequences, and will more likely encounter closely related natural homologs. Therefore, the four highest-identity natural homologous sequences were identified to the gRNA-T and gRNA-Z target sites via BLAST. In each case, SARS-CoV-1 variants, Bat coronaviruses, and Pangolin coronaviruses were identified as the most closely related potential off-targets (OT), containing 2 or 3 mismatches, with gRNA-Z also targeting an additional unknown marine virus and a porcine genome sequence with 7 mismatches (FIGS. 15A-15B, Table 6). These sequences were synthesized as DNA templates containing a T7 promoter and assayed for off-target cleavage tolerance of gRNA-T and gRNA-Z in an IVT-coupled cleavage reaction analyzed via fluorescence detection. It was found that gRNA-T was sufficiently specific to exclusively recognize the synthetic E-gene fragment (ANOVA, p<0.001), precluding recognition of any off-targets, which showed no significant differences in fluorescent signal with the NTC (ANOVA, Dunnett's test OT1: p=0.9998, OT2: p=0.5242, OT3: p=0.6633, OT4: p=0.8475) (FIG. 1C). gRNA-Z demonstrated detection of the N-gene synthetic template and the most similar pangolin coronavirus off-target templates compared to the NTC (N, OT1, OT2: p<0.0001), thus presenting some degree of mismatch tolerance for gRNA-Z. Fluorescence signal for OT3 and OT4 did not differ from the NTC (ANOVA, Dunnett's test OT3: p=0.8877, OT4: p>0.9999) (FIG. 15D).

TABLE 6
Top four naturally-occurring off-target sequences for gRNA T and gRNA Z.

| | | | | | Full length | | Off-target synthetic | template |
|---|---|---|---|---|---|---|---|---|
| | | | | | 30 nt | | templates (gblocks) | to in |
| | | | | | homologous | | containing T7 (lower case) | vitro |
| | gRNA | | | Blast- | sequence | | followed by the off-target | transcribe |
| | sequence | | | returned | (lower case | # of | (Bold, lower case show mismatches) | RNA |
| | (Blast | Sequence | Off-target | off-target | shows | mis- | sequence (upper case) and its | synthetic |
| Name | input) | ID | Organism | sequences | mismatches) | matches | 40 flanking nucleotides. | template |
| gRNA | CAAGACTC | MT072865.1 | Pangolin | ACTCACGT | CtAaACTCA | 2 | gaaattaatacgactcactataggGTCACACTAGCCATCC | 1136- |
| T- | ACGTTAAC | | coronavirus | TAACAATA | CGTTAACA | | TTACTGCGCTTCGATTGTGTGCGTACTGCT | OFF-F |
| Off- | AATATTGC | | isolate | TTGCAGCA | ATATTGCA | | GCAATATTGTTAACGTGAGTtTaGTTAAAC | gaaattaat |
| target | AGCAGT | | PCoVGX- | GT | GCAGT | | CTTCTTTTTACGTCTACTCACGTGTTAAAAA | acgactcac |
| 1 | | | P3B genomic | | | | TCT | 1136- |
| | | | sequence | | | | | OFF-R1 |
| | | | (shared by | | | | | AGATT |
| | | | numerous | | | | | TTTAA |
| | | | other bat | | | | | CACGT |
| | | | coronaviruses, | | | | | GAG |
| | | | poangolin, and | | | | | |
| | | | many SARS | | | | | |
| | | | strains | | | | | |
| gRNA | CAAGACTC | GQ153543.1 | Bat SARS | ACTCACAT | CtAaACTCA | 3 | gaaattaatacgactcactataggGTCACAATAGCCATCC | 1136- |
| T- | ACGTTAAC | | coronavirus | TAACAATA | CaTTAACA | | TTACTGCGCTTCGATTGTGTGCGTACTGCT | OFF-F |
| Off- | AATATTGC | | HKU3-8, | TTGCAGCA | ATATTGCA | | GCAATATTGTTAAtGTGAGTtTaGTAAAAC | gaaattaat |
| target | AGCAGT | | complete | GT | GCAGT | | CAACAGTTTACGTTTACTCACGTGTTAAAAA | acgactcac |
| 2 | | | genome | | | | TCT | 1136- |
| | | | | | | | | OFF-R1 |
| | | | | | | | | AGATT |
| | | | | | | | | TTTAA |
| | | | | | | | | CACGT |
| | | | | | | | | GAG |
| gRNA | CAAGACTC | AY485277.1 | SARS | ACTCACGT | CtAaACTCA | 3 | gaaattaatacgactcactataggGTCACACTAGCCATCC | 1136- |
| T- | ACGTTAAC | | coronavirus | TAACAATA | CGTTAACA | | TTACTGCGCTTCGATTGTGTGCGTACTGCT | OFF-F |
| Off- | AATATTGC | | Sinol-11, | TTGCAGCA | ATATaGCA | | GCtATATTGTTAACGTGAGTtTaGTAAAAC | gaaattaat |
| target | AGCAGT | | complete | G | GCAGT | | CAACGGTTTACGTCTACTCGCGTGTTAAAA | acgactcac |
| 3 | | | genome | | | | ATCT | 1136- |
| | | | | | | | | OFF-R5 |
| | | | | | | | | AGATT |
| | | | | | | | | TTTAA |
| | | | | | | | | CACGC |
| | | | | | | | | GAG |
| gRNA | CAAGACTC | FJ882960.1 | SARS | ACTCACGT | CtAaACTCA | 3 | gaaattaatacgactcactataggTCACACTAGCCATCCT | 1136- |
| T- | ACGTTAAC | | coronavirus | TAACAATA | CGTTAACA | | TACTGCGCTTCGATTGTGTGCGTgCTGCTGC | OFF-F |
| Off- | AATATTGC | | ExoNI isolate | TAGCAGCA | ATATTGCA | | AATATTGTTAACGTGAGTtTaGTAAAACCA | gaaattaat |
| target | AGCAGT | | P3pp34, | GT | GCAGc | | ACGGTTTACGTCTACTCGCGTGTTAAAAATC | acgactcac |
| 4 | | | complete | | | | T | 1136- |
| | | | genome | | | | | OFF-R5 |
| | | | | | | | | AGATT |
| | | | | | | | | TTTAA |
| | | | | | | | | CACGC |
| | | | | | | | | GAG |
| gRNA | GTAGAAAT | MT072865.1 | Pangolin | TAGAAGTA | aTAGAAgT | 3 | gaaattaatacgactcactataggAAGAGCTACCAGACG | 1136- |
| ZOff | ACCATCTTG | | coronavirus | CCATCGTG | ACCATCgT | | AGTTCGTGGTGGTGACGGTAAAATGAAAGA | OFF-F |
| Target | GACTGAGA | | isolate | GACTGAG | GGACTGA | | TCTCAGTCCAcGATGGTAcTTCTAtTACCTT | gaaattaat |
| 1 | TCTTT | | PCoVGX- | ATCTTT | GATCTTT | | GGAACTGGGCCAGAAGCTGGACTTCCCTAT | acgactcac |
| | | | P3B genomic | | | | GGTG | 1136- |
| | | | sequence | | | | | OFF-R2 |
| | | | | | | | | CACCA |
| | | | | | | | | TAGGG |
| | | | | | | | | AAGTC |
| | | | | | | | | CA |
| gRNA | GTAGAAAT | MT084071.1 | Pangolin | TACCATCT | GTAaAAgT | 2 | gaaattaatacgactcactataggAAGAGCTACCAGACG | 1136- |
| Z Off | ACCATCTTG | | coronavirus | TGGACTGA | ACCATCTT | | AATTCGTGGTGGTGACGGTAAAATGAAAGA | OFF-F |
| Target | GACTGAGA | | isolate MP789 | GATCTTF | GGACTGA | | TCTCAGTCCAAGATGGTAcTTtTACTACCT | gaaattaat |
| 2 | TCTTT | | genomic | | GATCTTF | | AGGAACTGGGCCAGAAGCTGGACTTCCCTA | acgactcac |
| | | | sequence | | | | TGGTG | 1136- |
| | | | | | | | | OFF-R2 |
| | | | | | | | | CACCA |
| | | | | | | | | TAGGG |
| | | | | | | | | AAGTC |
| | | | | | | | | CA |
| gRNA | GTAGAAAT | FP340301.5 | Pig DNA | TAGAAATA | aTAGAAAT | 7 | gaaattaatacgactcactataggTTGGAAGCCTTATAAA | 1136- |
| Z Off | ACCATCTTG | | sequence from | CCATCTTG | ACCATCTT | | ACTCCTTTAATTCATCTTCTCTCTgtcccTtTCA | OFF-F |
| Target | GACTGAGA | | clone CH242- | GACTGA | GGACTGAa | | GTCCAAGATGGTATTTCTAtTGGTATAAGA | gaaattaat |
| 3 | TCTTT | | 201L14on | | Agggac | | TTCTAAAAATATTGTGGTGCACCCGCATGT | acgactcac |
| | | | chromosome | | | | | 1136- |
| | | | X, complete | | | | | OFF-R3 |
| | | | sequence | | | | | ACATG |
| | | | | | | | | CGGGT |
| | | | | | | | | GCACC |
| | | | | | | | | ACA |
| gRNA | GTAGAAAT | MN693138.1 | Marine virus | TACCATCT | GgtacgtTAC | 7 | gaaattaatacgactcactataggGTCTACCAAGCAGATA | 1136- |
| Z Off | ACCATCTTG | | AFVG25M14 | TGGATTGA | CATCTTGG | | CTTGTTAACGATATTCGTATTAGCAAAGAT | OFF-F |
| Target | GACTGAGA | | 5, complete | GATCTTF | AaTGAGAT | | CTCAaTCCAAGATGGTAacgtacCATAACAA | gaaattaat |
| 4 | TCTTT | | genome | | CTTT | | AAGACTAGGCAGAGAAATCTGCCAACCTTT | acgactcac |
| | | | | | | | TGT | 1136- |
| | | | | | | | | OFF-R4 |
| | | | | | | | | ACAAA |
| | | | | | | | | AGGTT |
| | | | | | | | | GGCAG |
| | | | | | | | | AT |

CasRx-Based Detection of SARS-CoV-2 from Patient Isolates

The capability of SENSR to detect SARS-CoV-2 from infected patient samples was determined and these results were directly compared to RT-qPCR-validated diagnostics. RT-qPCR analysis of patient samples was performed by targeting the N-, S-, and Orf1ab-genes (Table 7), and accordingly, gRNA-Z was selected to directly compare SENSR fluorescence detection to N-gene RT-qPCR Cₜ values. Fluorescence detection analysis was performed on 72 RT-qPCR validated positive (n=36) and negative (n=36) patient samples. By fluorescence, SENSR yielded one false-positive among negative patient samples demonstrating 98% analytical specificity (1/36), and obtained a conservative 56% concordance with confirmed positive samples (20/36) when the threshold for detection is set at S/N>2 (FIG. 16A-C, Table 7, see Experiment 3). For RT-qPCR analysis of viral infections, a lower Cₜ value indicates a higher viral load within an isolated sample. SENSR demonstrated robust detection of infected patient samples with Cₜ values ≤20, detecting SARS-CoV-2 vRNA in (11/12) such samples (Sample ID: 1-12) (FIG. 16A, Table 7). SENSR can detect SARS-CoV-2 vRNA up to a maximum Cₜ value ≤27.5 with a moderate 23% (6/26) false-negative rate (FIG. 16A-C, Table 7). Next, it was confirmed detection of SARS-CoV-2 can be achieved by lateral flow analysis. Lateral flow analysis was performed on the 36 positive and 36 negative samples to determine if SENSR can potentially operate as a point-of-care diagnostic. Using lateral flow, detection of 15/36 positive samples was observed with the Cₜ value maximum of 25 (FIG. 16B). These findings confirm SENSR can be successfully adapted for the rapid detection of SARS-CoV-2 from patient samples, however, further optimization is under investigation to improve sensitivity and consistency of results.

TABLE 7
Data from RT-qPCR and SENSR fluorescence analysis of patient samples for detection of SARS-CoV-2.

| | | RT-qPCR | | | | Background | | | gRNA-Z | | |
|---|---|---|---|---|---|---|---|---|---|---|---|
| Sample | | Ct Values | | | | Subtracted | | | S/N | | |
| ID | MS2 | N | S | Orf1a1b | Rep1 | Rep2 | Rep3 | Rep1 | Rep2 | Rep3 | Pos/Neg |
| P1 | −1 | 11.786 | 13.667 | 12.675 | 0.606005 | 0.5776573 | 0.5767323 | 34.00790518 | 32.4170835 | 32.36517417 | Pos |
| P2 | −1 | 14.328 | 15.524 | 14.746 | 0.686162 | 0.687889 | 0.69811 | 50.14838762 | 50.27460601 | 51.02161134 | Pos |
| P3 | −1 | 15.267 | 16.808 | 15.933 | 0.57610512 | 0.5699407 | 0.6132402 | 93.5524269 | 92.5513876 | 99.582696 | Pos |
| P4 | −1 | 15.524 | 16.565 | 15.334 | 0.0901051 | 0.0857951 | 0.0994038 | 14.6319644 | 13.9320732 | 16.1419594 | Pos |
| P5 | −1 | 15.861 | 17.206 | 16.475 | 0.1314311 | 0.1123513 | 0.1549719 | 7.375675757 | 6.304951869 | 8.6967429 | Pos |
| P6 | −1 | 16.246 | 17.202 | 16.15 | 0.2971524 | 0.2916083 | 0.2893692 | 19.35161278 | 18.99056143 | 18.84474334 | Pos |
| P7 | −1 | 16.602 | 17.704 | 16.752 | 0.1962849 | 0.1969599 | 0.2124139 | 12.78276528 | 12.82672366 | 13.83314267 | Pos |
| P8 | −1 | 17.563 | 17.792 | 17.333 | 0.250986 | 0.2595524 | 0.2717122 | 40.7570517 | 42.1481301 | 44.1227327 | Pos |
| P9 | −1 | 17.656 | 18.643 | 17.71 | 0.1195735 | 0.1182829 | 0.1394585 | 6.710248678 | 6.637822539 | 7.826158934 | Pos |
| P10 | −1 | 17.982 | 19.067 | 19.466 | 0.066082 | 0.0683389 | 0.0653296 | 4.303493009 | 4.4504703 | 4.254494066 | Pos |
| P11 | −1 | 19.042 | 19.757 | 20.022 | 0.084763 | 0.0921497 | 0.0823981 | 4.756746342 | 5.171274594 | 4.624032429 | Pos |
| P12 | −1 | 19.947 | 21.81 | 20.755 | 0.0211419 | 0.0267883 | 0.0268967 | 1.376835127 | 1.744548618 | 1.751608008 | Neg |
| P13 | −1 | 20.112 | 20.371 | 19.711 | 0.4459823 | 0.3796561 | 0.4404821 | 72.422062 | 61.6514997 | 71.5288969 | Pos |
| P14 | −1 | 20.804 | 21.769 | 21.046 | 0.4173842 | 0.5041225 | 0.4539074 | 67.7780809 | 81.8633182 | 73.7090012 | Pos |
| P15 | −1 | 21.088 | 22.811 | 21.72 | 0.0246669 | 0.0231586 | 0.0260512 | 1.606395565 | 1.508169747 | 1.696546065 | Neg |
| P16 | −1 | 22.087 | 22.597 | 20.88 | 0.0492173 | 0.0383944 | 0.0448272 | 7.99228658 | 6.23478021 | 7.27938812 | Pos |
| P17 | −1 | 22.996 | 24.28 | 23.01 | 0.1556888 | 0.1411352 | 0.1530135 | 11.37856991 | 10.31491501 | 11.18304469 | Pos |
| P18 | −1 | 23.198 | 25.289 | 25.425 | 0.0710378 | 0.0720095 | 0.0701659 | 5.191822237 | 5.262839268 | 5.128099123 | Pos |
| P19 | 38.228 | 23.768 | 23.793 | 23.258 | 0.0183832 | 0.0172001 | 0.0175235 | 2.98520648 | 2.79308553 | 2.84560173 | Pos |
| P20 | −1 | 24.405 | 25.839 | 24.854 | 0.0487277 | 0.0456584 | 0.0481863 | 7.91278154 | 7.41436482 | 7.82486481 | Pos |
| P21 | −1 | 24.698 | 27.748 | 26.825 | 0.0137071 | 0.0151346 | 0.0141273 | 0.892654717 | 0.985618554 | 0.920019624 | Neg |
| P22 | −1 | 24.741 | 25.294 | 24.727 | 0.0119783 | 0.0037724 | 0.0092635 | 1.94512918 | 0.61259155 | 1.50427892 | Neg |
| P23 | −1 | 25.526 | 26.533 | 25.658 | 0.1034063 | 0.0948163 | 0.0960323 | 7.557485279 | 6.929682152 | 7.018553933 | Pos |
| P24 | −1 | 25.831 | 25.838 | 25.449 | 0.0197675 | 0.0345274 | 0.0290896 | 1.109316368 | 1.937615276 | 1.63245577 | Neg |
| P25 | −1 | 26.244 | 27.71 | 26.793 | −0.0033385 | 0.0015358 | 0.008256 | −0.5421315 | 0.24939511 | 1.34067326 | Neg |
| P26 | −1 | 27.541 | 28.12 | 27.808 | 0.0288696 | 0.0369931 | 0.0357163 | 2.109944723 | 2.703653536 | 2.610338166 | Pos |
| P27 | 31.067 | 27.818 | 29.718 | 28.745 | 0.0301025 | 0.017808 | 0.035101 | 1.689297887 | 0.99935277 | 1.969804671 | Neg |
| P28 | 31.205 | 28.464 | 30.247 | 28.98 | 0.0160267 | 0.0179037 | 0.0097162 | 1.043715254 | 1.165952117 | 0.632753227 | Neg |
| P29 | 31.577 | 30.635 | 30.276 | 29.83 | 0.0128741 | 0.0062507 | 0.0040223 | 2.09059613 | 1.01503711 | 0.65317224 | Neg |
| P30 | 31.592 | 30.682 | 31.228 | 31.163 | 0.0317991 | 0.0159132 | 0.0088679 | 1.784508012 | 0.893020019 | 0.497650518 | Neg |
| P31 | 31.711 | 30.948 | 30.797 | 31.008 | 0.0229911 | 0.0193373 | 0.0222961 | 1.680312513 | 1.413273273 | 1.629518197 | Neg |
| P32 | 31.274 | 31.737 | 30.389 | 30.448 | 0.0033487 | 0.0005946 | −0.0033162 | 0.54378786 | 0.09655576 | −0.5385103 | Neg |
| P33 | 31.866 | 32.083 | 31.724 | 31.93 | 0.006035 | 0.0045976 | 0.0061752 | 0.98001007 | 0.74659392 | 1.00277683 | Neg |
| P34 | 32.013 | 32.285 | 31.988 | 33.321 | 0.0198926 | 0.0246496 | 0.0227939 | 1.453857566 | 1.80152456 | 1.665900083 | Neg |
| P35 | 31.937 | 32.29 | 32.097 | 31.839 | 0.0132493 | 0.0048572 | 0.0008508 | 2.15152401 | 0.78874978 | 0.1381595 | Neg |
| P36 | 31.81 | 33.585 | −1 | −1 | 0.0146407 | 0.0003782 | −0.0028297 | 2.37747032 | 0.06141505 | −0.4595086 | Neg |
| N1 | 31.249 | −1 | −1 | −1 | 0.0251667 | 0.0123719 | 0.0151005 | 4.08676377 | 2.009045 | 2.45213621 | Pos |
| N2 | 30.833 | −1 | −1 | −1 | 0.019825 | 0.0190483 | 0.0252977 | 2.049035395 | 1.72227081 | 1.719062364 | Neg |
| N3 | 30.787 | −1 | −1 | −1 | 0.0202161 | 0.0232261 | 0.0231817 | 1.535428122 | 1.606803271 | 2.276031173 | Neg |
| N4 | 30.862 | −1 | −1 | −1 | 0.0189817 | 0.0129044 | 0.0174106 | 1.448916997 | 1.392151608 | 1.848891173 | Neg |
| N5 | 31.583 | −1 | −1 | −1 | 0.0106594 | 0.0135349 | 0.0050256 | 1.73095598 | 2.19790195 | 0.81609587 | Neg |
| N6 | 30.918 | −1 | −1 | −1 | 0.0208999 | 0.0152205 | 0.0069848 | 1.527476436 | 1.112395518 | 0.510486529 | Neg |
| N7 | 30.504 | −1 | −1 | −1 | 0.0280362 | 0.0235652 | 0.0235213 | 1.387284124 | 0.943122547 | 1.272459736 | Neg |
| N8 | 31.137 | −1 | −1 | −1 | 0.0210087 | 0.0219853 | 0.0311421 | 0.915006614 | 1.292514355 | 1.442010432 | Neg |
| N9 | 30.793 | −1 | −1 | −1 | 0.0216917 | 0.0202449 | 0.028793 | 1.178917801 | 0.964616948 | 1.126559459 | Neg |
| N10 | 30.919 | −1 | −1 | −1 | 0.0125197 | 0.017685 | 0.0197305 | 1.271312754 | 1.209756709 | 0.973184857 | Neg |
| N11 | 30.965 | −1 | −1 | −1 | 0.0161307 | 0.0131985 | 0.0154143 | 1.163560213 | 1.286992177 | 1.400075947 | Neg |
| N12 | 30.748 | −1 | −1 | −1 | 0.0226542 | 0.0215573 | 0.0173417 | 1.232658543 | 1.291941802 | 1.179368708 | Neg |
| N13 | 30.991 | −1 | −1 | −1 | 0.0207341 | 0.0229336 | 0.0249487 | 0.863030457 | 0.973403718 | 1.363402708 | Neg |
| N14 | 31.048 | −1 | −1 | −1 | 0.0219654 | 0.0230218 | 0.0210158 | 1.306459578 | 0.935142334 | 1.372028074 | Neg |
| N15 | 30.83 | −1 | −1 | −1 | 0.0153788 | 0.0173456 | 0.0242952 | 0.917953332 | 0.748616687 | 1.155333286 | Neg |
| N16 | 31.286 | −1 | −1 | −1 | 0.0232805 | 0.0166638 | 0.0244489 | 1.030212164 | 1.479662767 | 1.091773821 | Neg |
| N17 | 31.325 | −1 | −1 | −1 | 0.0163575 | 0.01334 | 0.0205875 | 1.412640043 | 1.318419322 | 1.875101756 | Neg |
| N18 | 31.151 | −1 | −1 | −1 | 0.0183579 | 0.0263669 | 0.0194549 | 1.274252545 | 1.282334375 | 0.987389914 | Neg |
| N19 | 30.765 | −1 | −1 | −1 | 0.0195667 | 0.0196908 | 0.0151618 | 1.072480316 | 0.97785583 | 0.088541949 | Neg |
| N20 | 30.647 | −1 | −1 | −1 | 0.0164684 | 0.0150154 | 0.0013596 | 1.316543764 | 1.512565585 | 1.5096741 | Neg |
| N21 | 30.826 | −1 | −1 | −1 | 0.0197998 | 0.0211461 | 0.0175819 | 1.289432839 | 1.377108646 | 1.144995365 | Neg |
| N22 | 31.43 | −1 | −1 | −1 | 0.025215 | 0.02522 | 0.0139752 | 1.642089771 | 1.642415388 | 0.910114335 | Neg |
| N23 | 31.346 | −1 | −1 | −1 | 0.0209355 | 0.0163148 | 0.0176154 | 1.36339363 | 1.062477342 | 1.147177004 | Neg |
| N24 | 30.693 | −1 | −1 | −1 | 0.0088297 | 0.004199 | 0.0053273 | 1.43383511 | 0.68186616 | 0.86508826 | Neg |
| N25 | 30.966 | −1 | −1 | −1 | 0.0065916 | 0.0040638 | 0.0171718 | 1.07039509 | 0.65991134 | 2.78848996 | Neg |
| N26 | 31.055 | −1 | −1 | −1 | 0.0030496 | 0.00164 | 0.0089785 | 0.49521768 | 0.26631591 | 1.45799841 | Neg |
| N27 | 30.759 | −1 | −1 | −1 | 0.0078569 | −0.0044158 | 0.0062999 | 1.27586431 | −0.7170718 | 1.02302658 | Neg |
| N28 | 31.365 | −1 | −1 | −1 | 0.0032921 | 0.0098173 | 0.0015665 | 0.53459671 | 1.59420925 | 0.25438041 | Neg |
| N29 | 31.462 | −1 | −1 | −1 | −0.0031694 | 0.0047259 | 0.006721 | −0.5146717 | 0.76742827 | 1.09140806 | Neg |
| N30 | 30.744 | −1 | −1 | −1 | 0.0071775 | 0.0015977 | 0.0060283 | 1.16553807 | 0.25944691 | 0.97892207 | Neg |
| N31 | 31.173 | −1 | −1 | −1 | 0.0042923 | 0.0055564 | 0.0065477 | 0.69701694 | 0.90229129 | 1.06326627 | Neg |
| N32 | 30.667 | −1 | −1 | −1 | 0.0031968 | 0.0069867 | 0.0081678 | 0.51912116 | 1.13455449 | 1.32635066 | Neg |
| N33 | 30.594 | −1 | −1 | −1 | 0.002398 | 0.0015748 | 0.0018156 | 0.38940582 | 0.25572823 | 0.2948312 | Neg |
| N34 | 31.057 | −1 | −1 | −1 | 0.0040382 | 0.0150647 | 0.0002933 | 0.65575421 | 2.44632273 | 0.04762833 | Neg |
| N35 | 30.761 | −1 | −1 | −1 | 0.0024757 | 0.0010414 | −0.0014863 | 0.40202335 | 0.1691106 | −0.2413569 | Neg |
| N36 | 30.917 | −1 | −1 | −1 | 0.0019224 | −0.002116 | 0.0006321 | 0.31217421 | −0.3436125 | 0.1026453 | Neg |
| NTC | N/A | N/A | N/A | N/A | 0.0162639 | 0.0130615 | 0.0117225 | N/A | N/A | N/A | N/A |
| NTC | N/A | N/A | N/A | N/A | 0.0133696 | 0.0206042 | 0.0194848 | N/A | N/A | N/A | N/A |
| NTC | N/A | N/A | N/A | N/A | 0.0158801 | 0.0169301 | 0.0132561 | N/A | N/A | N/A | N/A |
| NTC | N/A | N/A | N/A | N/A | 0.0018932 | 0.0071546 | 0.0094265 | N/A | N/A | N/A | N/A |

### Experiment 3—Materials and Methods

CasRx Subcloning, Protein Expression and Purification

To produce an expression plasmid for CasRx protein production, the human codon optimized CasRx coding sequence was cloned into the expression vector, pET-His6-MBP-TEV-yORF (Series 1-M) (purchased from QB3 MacroLab, Berkeley) using the Gibson assembly method (Gibson et al., 2009). In brief, the CasRx coding sequence was PCR amplified from plasmid OA-1050E (Addgene plasmid #132416, Buchman et al., 2020) using primers 11361.C1 and 11361.C2 (Table 5). The fragment was purified and subcloned into the restriction enzyme cutting site EcoRI, downstream of the His-MBP recombinant protein in pET-His6-MBP-TEV-yORF, generating the final pET-6×His-MBP-TEV-CasRx (OA-1136J; Addgene plasmid #153023) plasmid.

Protein expression, culture, cell lysis, affinity and further downstream protein purification were performed as previously described (FIG. 8) (Konermann et al., 2018). In brief, to facilitate protein expression in liquid culture, pET-His6-MBP-TEV-CasRx was transformed into Rosetta2 (DE3) pLysS cells (Novagen, 71403). Starter cultures in LB were supplemented with kanamycin and chloramphenicol and incubated at 37° C. overnight. 20 mL of starter culture were used to inoculate 1 L of TB media supplemented with the same antibiotics. Cultures were allowed to grow until OD600˜0.5, cooled on ice, induced with 0.2 mM IPTG, and then grown for 20 hours at 18° C. Cells were then pelleted, freeze-thawed, resuspended, lysed via sonication and clarified by centrifugation. Protein purification was performed by gravity Ni-NTA affinity chromatography (THERMO SCIENTIFIC™ HISPUR™ Ni-NTA Resin) followed by removal of the 6×His-MBP tag by TEV protease concurrently with overnight dialysis. Further purification was achieved by cation exchange using a 5-mL HiTrap SP HP using a gradient of 125 mM to 2M NaCl in 50 mM Tris-HCl, 7.5% v/v glycerol, 1 mM DTT. The protein was finally purified by gel filtration chromatography using a HILOAD® 16/600 or in 50 mM Tris-HCl, 600 mM NaCl, 10% glycerol, 2 mM dithiothreitol on a SUPERDEX® 200 16/600 column. Fractions were pooled, concentrated to ˜2 mg/mL and stored at −80° C. in the same buffer.

Production of Target SARS-CoV-2 RNA and gRNAs

To detect viral genomic sequences, two synthetic dsDNA gene fragments were designed containing a T7 promoter sequence upstream of gene segments corresponding to the SARS-CoV-2 envelope (E) and nucleocapsid (N) protein coding regions (GenBank Accession #MN908947). The 253 bp SARS-CoV2 E-gene segment was ordered and synthesized as a custom GBLOCK® from Integrated DNA Technologies (IDT) and amplified using primers 1136Q-F and 1136Q-R (Table 5). A 500 bp SARS-CoV2 N-gene segment was amplified from a plasmid 1136Y (Catalog #10006625) (Broughton et al., 2020) using primers 1136X-F and 1136X-R (Table 5). These two SARS-CoV-2 gene targets were amplified using PCR and then purified using the MinElute PCR Purification Kit (QIAGEN #28004). Also designed were eight synthetic dsDNA templates containing nucleotide variations from the native SARS-CoV-2 E- and N-gene (4 synthetic targets each gene) that were used for gRNA off-target analysis and ordered as a GBLOCK® from IDT. Primers 1136-OFF-F and 1136-OFF-R1˜1136-OFF-R5 were used to amplify these sequences (Table 5). The synthetic targets were chosen based on sequence homology identified using NCBI BLAST searches against gRNA-T and gRNA-Z. 40 nt regions flanking the mismatch target sequences were included in the 5′ and 3′ ends of the 30 nt stretch in order to allow amplification analysis via RT-RPA.

gRNAs targeting the synthetic vRNA gene segments were designed using criteria previously outlined (FIG. 12) (Buchman et al., 2020) and generated following a previously described templateless PCR protocol (Li et al. 2018, Highly Efficient Site-Specific Mutagenesis in Malaria Mosquitoes Using CRISPR.” G3 8 (2): 653-58). The primers used to amplify these gRNAs, as well as their final sequence are outlined in Table 5. Both the synthetic vRNA and gRNAs were synthesized through in vitro transcription (IVT) using MEGASCRIPT™ T7 Transcription Kit (INVITROGEN™ #AM1334), followed by DNaseI digestion and purification using the MEGACLEAR™ Transcription Clean-Up Kit (INVITROGEN™ #AM1908).

In Vitro gRNA Cleavage Assays

To test the in vitro cleavage efficiency of gRNAs, preliminary in vitro cleavage assays were performed to test on-target cleavage, off-target cleavage, and collateral-cleavage properties. On-target cleavage assays were prepared with RNA templates for E-gene (1000 ng) or N-gene (1500 ng), followed by addition of CasRx (112 ng) and 10 ng of each gRNA in a 2:1 molar ratio. Reactions were prepared in 20 mM HEPES pH 7.2 and 9 mM MgCl₂, incubated at 37° C. for one hour, denatured at 85° C. for 10 min in 2×RNA loading dye (New England Biolabs, #B0363) and loaded on 2% 1×TBE agarose gel stained with SYBR™ gold nucleic acid staining (INVITROGEN™ #S11494). Off-target cleavage assays were assembled similarly with the non-targeting synthetic vRNA template. Collateral-cleavage assays were prepared with both synthetic vRNA templates simultaneously and same quantities of gRNA and CasRx described above.

Bioinformatics of SARS-CoV-2 SENSR Target Sites

433 SARS-CoV-2 genomes were downloaded from NCBI Virus (www.ncbi.nlm.nih.gov/labs/virus/vssi/#/virus?SeqType_s=Nucleotide&VirusLineage_ss=S ARS-CoV-2,%20taxid:2697049) and 3,164 non-SARS-CoV-2 Coronavirinae genomes were downloaded from Virus Pathogen Resource (www.viprbrc.org/brc/home.spg?decorator=corona_ncov) on Apr. 7, 2020. To assess the specificity of the probes (or guides), all possible 30 nt sequences were extracted from the two genome sets using a Perl script (data not provided) generating 52,712 and 8,338,305 unique fragments from SARS-CoV-2 and non-SARS-CoV-2 genomes, respectively. The probes (or guides) designed to target E and N genes based on Corman et al. 2020 (www.eurosurveillance.org/content/10.2807/1560-7917.ES.2020.25.3.2000045) were cross-referenced against the extracted sequences to identify numbers of targeted genomes in each set. Four of six probes perfectly matched sequences in all 433 SARS-CoV-2 genomes. Two others, 1136R-E-Protein-gRNA1 and 1136S-N-Protein-gRNA1, matched 430 and 426 SARS-CoV-2 genomes, respectively. Of 3,164 non-SARS-CoV-2 viruses, the probes (or guides) matched between 1 and 10 genomes, mostly from bat hosts (Summarized in Table 2). To identify a comprehensive set of possible targets that are specific to SARS-CoV-2 genomes, 16,645 30 nt sequences that perfectly matched all 433 SARS-CoV-2 genomes were filtered to remove the ones that were also found in any of the 3,164 non-SARS-CoV-2 genomes to produce a set of 8,846 SARS-CoV-2-specific sequences (Table 4). To check for possible cross-reactivity with human transcripts, the probes (or guides) were mapped to the human transcriptome (GRCh38, ENSEMBL release 99, ftp.ensembl.org/pub/release-99/fasta/homo_sapiens/) comprising both coding and non-coding RNAs using bowtie 1.2.3 allowing up to two mismatches (−v 2). None of the 8,846 sequences mapped to the human transcriptome to confirm their specificity to SARS-CoV-2. To visualize the distribution of the specific targets along the SARS-CoV-2 genome, probe density was calculated using a sliding window of 301 nt for each position of the reference SARS-CoV-2 genome NC_045512 (www.ncbi.nlm.nih.gov/nuccore/NC_045512) and plotted in R (FIG. 10). Viral genes that are affected by each probe were identified using the intersect function of bedtools (Table 3).

RT-RPA Amplification of Viral Genomic Sequences

Prior to all detection assays a pre-amplification step using RT-RPA was performed in order to amplify the SARS-CoV-2 target sequence. These protocols were initially developed and optimized on mock viral genome fragments and later validated against patient samples. To amplify the target sequences from the synthetic vRNA, RT-RPA was performed (Zhang et al., 2020) (protocol summarized in FIG. 10). In short, RT-RPA primers were designed to amplify 30 nt gRNA spacer regions flanked by 30 nt priming regions from the synthetic vRNA template while also incorporating a T7 promoter sequence into the 5′ end of the dsDNA gene fragment with +2 G's thus increasing transcription efficiency (FIG. 9B) (Brieba et al., Biochemistry 41, 5144-5149 (2002)). RT-RPA was performed at 42° C. for 45 min by combining M-MuLV-RT (NEB #M0253L) with TWISTAMP® Basic (TwistDx #TABAS03KIT). All RT-RPA primer sequences can be found in Table 1.

Fluorescence-Based Detection of SARS-CoV-2

For fluorescence-based detection, a simple in vitro transcription-coupled cleavage assay was developed with a fluorescence readout using 6-Carboxyfluorescein (6-FAM) as the fluorescent marker. To facilitate fluorescence detection, a 6 nt poly-U probe conjugated to a 5′-6-FAM and a 3′-IABlkFQ (FRU, Table 5) was developed and custom ordered from IDT. In total volumes of 15 μL, the following reaction mix was prepared; 5.62 μL water, 0.4 μL HEPES, pH 7.2 (1M), 0.18 μL MgCl₂ (1M), 3.2 μL rNTPs (25 mM each), 2 μL CasRx (55.4 ng/μL), 1 μL RNase inhibitor (40 U/μL), 0.6 μL T7 Polymerase (50 U/μL), 1 μL gRNA (10 ng/μL), and 1 μL FRU probe (2 μM). Alternatively, in total volumes of 15μL, the following reaction mix was prepared: 7.82 μL water, 0.4 μL HEPES, pH 7.2 (1M), 0.18μL MgCl₂ (1M), 1 μL rNTPs (25 mM each), 2 μL CasRx (55.4 ng/μL), 1 μL RNase inhibitor (40 U/μL), 0.6μL T7 Polymerase (50 U/μL), 1 μL gRNA (10 ng/μL), and 1 μL FRU probe (2 μM). This was followed by the addition of 5 μL (50% amplification vol) of the amplified target RNA from the RT-RPA pre-amplification mix (described above) or no-template control, which initiates the reaction following incubation at 37° C. for 90 min. Experiments were immediately run on a LIGHTCYCLER® 96 (Roche #05815916001) at 37° C. under 5 sec acquisition followed by 5 sec incubation for the first 15 min, followed by 5 sec acquisition and 55 sec incubation for up to 75 min. Fluorescence readouts were analyzed over-time by normalization to templateless controls at each respective time point or through background subtracted fluorescence by subtracting the initial fluorescence value from the final value.

Half-Maximum Fluorescence Analysis

Half-maximum fluorescence analysis was used to determine which gRNA cleaved the modified ssRNA probe fastest. The half-maximum fluorescence time-point was calculated by fitting a non-linear regression (y=Y_{M}−(Y_{M}−Y₀)^{(-k*x)}) to the averaged and normalized fluorescence over time data for each gRNA. The equation for the non-linear regression was then used to solve for x, or time (minutes) (x=((ln(Y_{M}−y)−ln(Y_{M}−Y₀))/−k), by entering in half of the maximum fluorescence value recorded for y.

Lateral Flow-Based Detection of SARS-CoV-2

For lateral flow-based detection, the HYBRIDETECT® system was modified to detect the presence of SARS-CoV-2 sequences using SENSR (Zhang et al., 2020). In brief, an ssRNA probe was designed composed of a 6 nt poly-U probe conjugated on opposite ends with a 5′-6-FAM and a 3′-biotin which was custom ordered from IDT (LFRU, Table 5). Following incubation of 5.22 μL water, 0.4 μL HEPES, pH 7.2 (1M), 0.18 μL MgCl₂ (1M), 2 μL CasRx (55.4 ng/μL), 1 μL gRNA (10 ng/μL), 5 μL RT-RPA reaction mix, 1 μL T7 polymerase (50 U/mL), 3.2 μL rNTPs (25 mM each), 1 μL LFRU probe (20 uM), at 37° C. for 60 min. 80 μL of HybriDetect Assay buffer was added to each reaction and mixed thoroughly. Next, the lateral flow dipstick was placed into the reaction and allowed to flow upwards by capillary action for a maximum of 2 min. The presence or absence of upper or lower bands was analyzed to detect evidence of SARS-CoV-2 by collateral cleavage. The presence of a solitary upper band or both an upper and lower band indicates a positive result, a solitary lower band with a faint upper band was interpreted as a negative result.

Limit of Detection Analysis

To determine the LOD of SENSR using both fluorescence and lateral flow analysis, serial dilutions of synthetic RNA templates on a logarithmic scale were performed. Fresh template stock concentrations were analyzed via nanodrop prior to dilutions to accurately achieve expected copies per L. Dilution scales were calculated using NEBioCalculator for each respective template. For fluorescence analysis, the LOD was determined by statistical significance of the lowest copy number experimental group compared to the NTC group. For lateral flow analysis, the LOD was determined by a noticeable saturation of the upper test band compared to the NTC.

Patient Samples Ethics Statement

Human samples from patients were collected under University of California San Diego's Human Research Protection Program protocol number 200470 for negatives, and under a waiver of consent from clinical samples from San Diego County for positives, as part of the SEARCH Alliance activities. Samples were de-identified as required by these protocols prior to testing and analysis under University of California San Diego Biological Use Authorization protocols R1806 and 2401.

RNA Extraction and Processing of Patient Samples

Patient nasopharyngeal samples were collected and RNA was extracted using Omega Bio-Tek Mag-Bind Viral DNA/RNA 96 Kit (Omega Cat. No. M6246-03), following the manufacturer's protocol for KingFisher Flex platform.

RT-qPCR Validation of SARS-CoV-2 Infection in Patient Samples

Patient samples were determined to be SARS-CoV-2 positive or negative TAQPATH™ COVID-19 Combo Kit RT-qPCR assay as described in (www.fda.gov/media/136112/download), and reducing the RT-qPCR reaction volumes to 3 μl and diluting the MS2 phage control to improve the limit of detection of the assay. The presence of SARS-CoV-2 viral RNA was analyzed using primers targeting the N, S, and Orf1ab genes with an MS2 control. All RT-qPCR assays were run using TAQPATH™ 1-Step RT-qPCR Master Mix (ThermoFisher #A15299) and thermocycling conditions were run following the CDC recommended protocol (www.fda.gov/media/136112/download). Fluorescence data were acquired on a QuantStudio 5 qPCR machine (Applied Biosystems).

SENSR Detection of Patient Samples

To detect the presence of SARS-CoV-2 in patient samples using SENSR, this system was tested against RT-qPCR validated samples. SARS-CoV-2 positive (N=36) and negative (N=36) samples were obtained and fluorescence analysis of these samples was run in triplicate. Samples were subject to pre-amplification using RT-RPA and incubated in an IVT-coupled cleavage reaction, as previously described. Data for analysis were acquired on LIGHTCYCLER® 96 (Roche #05815916001) following the protocol previously described. The data was processed by generating background subtracted fluorescence data for each replicate by subtracting the final (90 min) fluorescence value from the initial (0 min) fluorescence value. Noise was set as the average of the three no-template control (NTC) background subtracted values. S/N was then calculated by dividing the background subtracted value for each replicate by the noise. The S/N for each sample was then determined by taking the average of the three independent S/N ratios in the triplicates. An S/N=2 was determined to be the threshold by calculating 36 deviation from the mean for the negative samples (μ=1.12, 3σ=1.99). Samples were determined to be positive if S/N>2 and negative if S/N<2. Lateral flow analysis was run on samples that were determined as positives from the SENSR fluorescence analysis. The samples were assayed and analyzed following the previously described lateral flow methods and images were taken using a smartphone. Positives and negatives were determined in comparison to the NTC samples and using a positive control (synthetic template) as a standard.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein or attached hereto are expressly incorporated by reference in their entirety, to the same extent as if each were incorporated by reference individually. In case of conflict, the present specification, including definitions, will control.

Other embodiments are set forth within the following claims.

1. A clustered regularly interspaced short palindromic repeats (CRISPR) system, comprising: a gRNA targeting a severe acute respiratory syndrome coronavirus 2 (SARS-CoV-2) sequence and CRISPR reagents necessary to detect the SARS-CoV-2 sequence in a sample, optionally wherein the target sequence is selected from one or more of an envelope (E) gene, a nucleocapsid (N) gene, an Orf1ab gene, a Spike (S) gene, an Orf3a gene, an M matrix protein gene, an Orf6 gene, an Orf7a gene, an Orf7b gene, an Orf8 gene, an open reading frame (ORF) of endoRNAse, an ORF of nsp7, an ORF of nsp4, an ORF of 3C-like proteinase, an ORF of nsp3, an ORF of nsp6, an ORF of 2′-O-ribose methyltransferase, an ORF of nsp10, an ORF of 3′-to-5′ exonuclease, an ORF of nsp2, an ORF of RNA-dependent RNA polymerase, an ORF of helicase, an ORF of nsp8, an ORF of leader protein, an ORF of no-gen, an ORF of nsp9, an Orf10 gene, an Orf6 gene, or a fragment of each thereof, and optionally wherein the gene is an RNA sequence.

2. The system of claim 1, wherein the CRISPR system comprises a Cas13d enzyme and optionally an accessory protein comprising a WYL1-domain, optionally wherein the Cas13d is Ruminococcus flavefaciens Cas13d (CasRx), and optionally wherein the system comprises a fusion protein comprising the Cas13d enzyme, an optional protein cleavage site (optionally a TEV protease cleavage sequence), a purification tag (optionally a 6×His tag), and an optional Maltose-binding protein or a fragment thereof.

3. The system of claim 1, further comprising a reporting reagent, optionally selected from a probe conjugated with one or more purification or detectable markers (optionally radioisotopes, fluorochromes, chemiluminescent compounds, dyes, and proteins, including enzymes), optionally wherein the reporting reagent comprises a fluorophore and a quencher, wherein optionally the fluorophore can be placed in close proximity to the quencher, optionally wherein the probe is a collateral cleavage probe, optionally wherein the probe comprises a poly U sequence optionally a 6-nt poly-U, further optionally the reporting reagent comprises a probe (optionally a poly U) conjugated to a fluorescence maker (optionally a 5′ fluorescent marker, and optionally a 6-FAM) and a quencher (optionally a 3′ quencher, and optionally an IABlkFQ), and further optionally the reporting reagent comprises a probe (optionally a poly U) conjugated to a biotin and/or a fluorescent marker).

4. The system of claim 1, wherein the CRISPR system comprises a Cas13d enzyme and a reporting reagent, and wherein the reporting reagent comprises a poly U sequence conjugated with one or more purification or detectable markers.

5. The system of claim 1, wherein the target sequence is about 25 nt long to about 35 nt long, optionally about 30 nt long, optionally wherein the target sequence is not adjacent to a protospacer adjacent motif (PAM) or a protospacer flanking sequence (PFS) and optionally wherein the gRNA comprises a direct repeat (optionally a 5′ direct repeat and further optionally as disclosed herein such as in Table 5 or in FIG. 10) and a polynucleotide (such as RNA) sequence complimentary to the target sequence having 0, 1, 2 or 3 mismatches.

6. The system of claim 1, wherein the target sequence is selected from one or more of the ones disclosed herein, such as those listed in Tables 3 and 4 and the ones complementary to the gRNA disclosed herein (optionally in Table 5).

7. The system claim 1, further comprising a reagent for reverse transpiration of the RNA target sequence(s) in the sample, optionally a reverse transcriptase and a buffer suitable for the reverse transpiration.

8. The system of claim 1, further comprising reagents for amplifying the target sequences from the sample optionally to double-stranded DNA (dsDNA) amplicons, optionally wherein the amplification is selected from reverse transcriptase recombinase polymerase amplification (RT-RPA) or reverse transcriptase isothermal amplification (optionally Reverse transcription loop-mediated isothermal amplification, RT-LAMP), optionally wherein the RT-RPA reagent(s) is one or more of: RT-PRA primers amplifying a sequence comprising the target sequences and/or gRNA spacer regions, a Reverse Transcriptase, a recombinase, a single strand binding protein, and a buffer suitable for the application, optionally wherein the RT-PRA primer comprises a promoter sequence optionally a T7 promoter and a primer which is capable of annealing to the target sequence or a contiguous sequence in the gene.

9. The system claim 1, further comprising in vitro transcription (IVT) reagents, optionally selected from one or more of: RNA polymerase, ATP, GTP, UTP, CTP, and a buffer suitable for the IVT, optionally wherein the buffer is also suitable for the CRISPR reagents.

10. The system claim 1, comprising an E gene gRNA optionally a gRNA-T and an N gene gRNA optionally a gRNA-Z.

11. The system of claim 10, wherein the gRNA comprise one or more of
ACUGGUCGGGGUUUGAAACUGUAACUAGCAAGAAUACCACGAAAG
CAAG,
GCAAGUAAACCCCUACCAACUGGUCGGGGUUUGAAACUGUAACUA
GCAAGAAUACCACGAAAGCAAG,
ACUGGUCGGGGUUUGAAACCAAGACUCACGUUAACAAUAUUGCAG
CAGU,
GCAAGUAAACCCCUACCAACUGGUCGGGGUUUGAAACCAAGACUC
ACGUUAACAAUAUUGCAGCAGU,
ACUGGUCGGGGUUUGAAACGAAGGUUUUACAAGACUCACGUUAAC
AAUA,
GCAAGUAAACCCCUACCAACUGGUCGGGGUUUGAAACGAAGGUUU
UACAAGACUCACGUUAACAAUA,
ACUGGUCGGGGUUUGAAACGUAGAAAUACCAUCUUGGACUGAGAU
CUUU,
CAAGUAAACCCCUACCAACUGGUCGGGGUUUGAAACGUAGAAAUA
CCAUCUUGGACUGAGAUCUUU,
ACUGGUCGGGGUUUGAAACUAGGUAGUAGAAAUACCAUCUUGGAC
UGAG,
CAAGUAAACCCCUACCAACUGGUCGGGGUUUGAAACUAGGUAGUA
GAAAUACCAUCUUGGACUGAG,
ACUGGUCGGGGUUUGAAACGCCCAGUUCCUAGGUAGUAGAAAUAC
CAUC,
CAAGUAAACCCCUACCAACUGGUCGGGGUUUGAAACGCCCAGUUC
CUAGGUAGUAGAAAUACCAUC,
CAAGUAAACCCCUACCAACUGGUCGGGGUUUGAAACAUAGAGUUA
UUAGAGUAAGCAACUGAAUUU,
CAAGUAAACCCCUACCAACUGGUCGGGGUUUGAAACUUGUGGGUA
UGGCAAUAGAGUUAUUAGAGU,
CAAGUAAACCCCUACCAACUGGUCGGGGUUUGAAACGUAGAAUUU
CUGUGGUAACACUAAUAGUAA,
CAAGUAAACCCCUACCAACUGGUCGGGGUUUGAAACCCUUGGGUU
UGUUCUGGACCACGUCUGCCG,
CAAGUAAACCCCUACCAACUGGUCGGGGUUUGAAACAGUUCCUUG
UCUGAUUAGUUCCUGGUCCCC,
or
CAAGUAAACCCCUACCAACUGGUCGGGGUUUGAAACCAUUCCGAA
GAACGCUGAAGCGCUGGGGGC.

12. The system of claim 2, wherein the CasRx or Cas13d facilitates fluorescence-based readouts of RNase activity.

13. The system of claim 1, further comprising a means for visual indication of activity, optionally to be read out visually under UV, or quantitatively by a fluorometer.

14. The system of claim 2, wherein the CasRx enzyme is modified to detect SARS-Cov-2 genetic material by lateral flow assay.

15. A method to detect SARS-CoV-2 in a sample, comprising contacting the sample with the system of claim 1, optionally wherein the sample is isolated from one or more of the lungs, oral cavity, or nasal cavity of a subject.

16. (canceled)

17. The method of claim 15, wherein the subject is a mammal that is susceptible to infection by SARS-CoV-2, optionally wherein the mammal is a bat, a simian, a human, a feline, a canine, a murine, a rat, a rabbit, a bovine, an ovine, a porcine, an equine, or a primate.

18. (canceled)

19. The method of claim 15, further comprising detecting the presence of SARS-CoV-2, in the sample by detecting the presence of any one of more of the E gene, the S gene, the N gene.

20. (canceled)

21. The method of claim 15, wherein the limit of detection (LOD) about 10 to about 1000 copies (optionally 100 copies) per RT-RPA reaction or per microliter.

22. The method of claim 15, wherein the specificity and/or the concordance of the method is at least about 50%, or at least about 60%, or at least about 70%, or at least about 80%, or at least about 90%, or at least about 91%, or at least about 92%, or at least about 93%, or at least about 94%, or at least about 95%, or at least about 96%, or at least about 97%, or at least about 98%, or at least about 99%, or about 100%.

23. A kit comprising the system of claim 1, and instructions for use.

24. (canceled)

25. (canceled)
